(19) United States
(12) Patent Application Publication
(10) Pub. No.: US 2007/0225209 A1
(43) Pub. Date: Sep. 27, 2007

(54) COMPOSITIONS AND METHODS FOR TREATING NEUROLOGICAL DISORDERS AND DISEASES

(75) Inventors: Jean-Marc Roch, Salt Lake City, UT (US); Paul Bartel, Salt Lake City, UT (US); Karen Heichman, Salt Lake City, UT (US)

Correspondence Address: MYRIAD GENETICS INC.;INTELLECUTAL PROPERTY DEPARTMENT, 320 WAKARA WAY, SALT LAKE CITY, UT, 84108, US

(73) Assignee: Myriad Genetics, Incorporated, Salt Lake City, UT (US)

(21) Appl. No.: 11/669,939

(22) Filed: Jan. 31, 2007

Related U.S. Application Data
(63) Continuation-in-part of application No. 10/776,013, filed on Feb. 9, 2004, now abandoned.
Application No. 10/776,013 is a continuation-in-part of application No. 09/948,904, filed on Sep. 10, 2001, now abandoned.
Application No. 09/948,904 is a division of application No. 09/466,139, filed on Dec. 21, 1999, now abandoned.
Application No. 10/776,013 is a continuation-in-part of application No. 09/975,072, filed on Oct. 12, 2001, now abandoned.
Application No. 10/776,013 is a continuation-in-part of application No. 10/194,967, filed on Jul. 15, 2002, now abandoned.
(60) Provisional application No. 60/113,534, filed on Dec. 22, 1998.
(60) Provisional application No. 60/124,120, filed on Mar. 12, 1999.
(60) Provisional application No. 60/141,243, filed on Jun. 30, 1999.
(60) Provisional application No. 60/240,790, filed on Oct. 17, 2000.
(60) Provisional application No. 60/304,775, filed on Jul. 13, 2001.

(51) Int. Cl. A61K 38/04; C07K 4/12; G01N 33/53

(52) U.S. Cl. 514/2; 435/7.21; 530/328

(57) ABSTRACT

The present invention generally relates to methods and compositions for treating neurological disorders and diseases. In addition, methods for selecting therapeutic agents useful for treating neurological disorders and diseases are provided.

### CROSS-REFERENCE TO RELATED APPLICATIONS

This application is a continuation-in-part of U.S. patent application Ser. No. 10/776,013 filed Feb. 9, 2004, which is a continuation-in-part of U.S. patent application Ser. No. 09/948,904 filed Sep. 10, 2001, U.S. patent application Ser. No. 09/975,072 filed Oct. 12, 2001, and U.S. patent application Ser. No. 10/194,967 filed Jul. 15, 2002, each of which is hereby incorporated by reference in its entirety.

U.S. patent application Ser. No. 09/948,904 filed Sep. 10, 2001 is a divisional of U.S. patent application Ser. No. 09/466,139 filed Dec. 21, 1999, which claims the benefit of U.S. provisional patent application Ser. No. 60/113,534 filed Dec. 22, 1998, U.S. provisional patent application Ser. No. 60/124,120 filed Mar. 12, 1999, and U.S. provisional patent application Ser. No. 60/141,243 filed Jun. 30, 1999 each of which is hereby incorporated by reference in its entirety.

U.S. patent application Ser. No. 09/948,904 filed Sep. 10, 2001 is related to U.S. patent application Ser. No. 09/949,143 filed Sep. 10, 2001, Ser. No. 09/971,677 filed Oct. 9, 2001, Ser. No. 09/970,666 filed Oct. 5, 2001, Ser. No. 09/970,898 filed Oct. 5, 2001, Ser. No. 09/970,814 filed Oct. 5, 2001, Ser. No. 09/971,675 filed Oct. 9, 2001, and Ser. No. 09/949,084 filed Sep. 10, 2001 each of which is hereby incorporated by reference in its entirety.

U.S. patent application Ser. No. 09/975,072 filed Oct. 12, 2001 claims the benefit of U.S. provisional patent application Ser. No. 60/240,790 filed Oct. 17, 2000, which is hereby incorporated by reference in its entirety. U.S. provisional patent application Ser. No. 60/240,790 is related to U.S. patent application Ser. No. 09/972,038 filed Oct. 9, 2001, Ser. No. 09/971,782 filed Oct. 9, 2001, Ser. No. 09/972,757 filed Oct. 9, 2001, Ser. No. 09/973,064 filed Oct. 10, 2001, Ser. No. 09/973,063 filed Oct. 10, 2001, and Ser. No. 09/973,077 filed Oct. 10, 2001, each of which is hereby incorporated by reference in its entirety.

U.S. patent application Ser. No. 10/194,967 filed Jul. 15, 2002 claims the benefit of U.S. provisional patent application Ser. No. 60/304,775 filed Jul. 13, 2001, which is hereby incorporated by reference in its entirety. U.S. provisional patent application Ser. No. 60/304,775 is related to U.S. patent application Ser. No. 09/973,963 filed Oct. 11, 2001 (now U.S. Pat. No. 6,653,102, issued Nov. 25, 2003), Ser. No. 09/973,941 filed Oct. 11, 2001, Ser. No. 09/973,965 filed Oct. 11, 2001, and Ser. No. 09/973,964 filed Oct. 11, 2001, each of which is hereby incorporated by reference in its entirety.

### SEQUENCE LISTING

The instant application was filed with a formal Sequence Listing submitted electronically as a text file. This text file, which was named “1600-25-2006-11-30-SEQ-LIST-ST25.txt”, was created on Nov. 30, 2006, and is 191,122 bytes in size. Its contents are incorporated by reference herein in their entirety.

### FIELD OF THE INVENTION

The present invention generally relates to methods and compositions for treating diseases, particularly to methods of using and modulating specific proteins and protein-protein interactions for purposes of drug screening and treatment of diseases.

### BACKGROUND OF THE INVENTION

Most drug discovery efforts today employ approaches that empirically identify small molecules that bind particular biological targets in vitro. These approaches generally involve “primary” high throughput screens designed to search vast combinatorial libraries of small molecules for “lead compounds” that often show a relatively weak affinity for the chosen target. However, once such lead compounds are identified in a “primary” high throughput screen, they can be subjected to iterative rounds of chemical modification and further testing by the process known to medicinal chemists as Structure Activity Relationship, or SAR. Generally, after several rounds of SAR-guided modification and in vitro screening, a set of optimized and related drug candidate compounds are subjected to the next phase of testing. This next phase generally involves the in vivo screening of the drug candidates in cell-based assays specifically designed to test the efficacy, toxicity and bioavailablity of the candidates. If desired effects are obtained with reasonable dosages in these cell-based assays, animal studies are then initiated to determine whether the drug candidates have the desired activity in vivo. Only after careful study in well-defined animal models will a drug candidate be administered to humans in carefully regulated clinical trials.

The success or failure of a drug discovery program is heavily dependent on the identification and selection of druggable targets. In addition, once an appropriate drug target has been identified, an efficient—preferably high throughput—screening assay needs to be established to screen against that particular drug target. The development of such screens is often problematic. The present invention provides novel drug targets for neurological disorders, ailments and diseases, including mild cognitive impairment, depression, schizophrenia, obsessive-compulsive disorder, bipolar disorder, and neurodegenerative diseases and disorders and motor neuron diseases and disorders such as Alzheimer's disease, Parkinson's disease, dementia with Lewy bodies, amyotrophic lateral sclerosis or Lou Gehrig's disease, Alpers' disease, Leigh's disease, Pelizaeus-Merzbacher disease, Olivopontocerebellar atrophy, Friedreich's ataxia, leukodystrophies, Rett syndrome, Ramsay Hunt syndrome type II, and Down's syndrome, and discloses screening assays for identifying potential drugs that may be effective for treating the symptoms of these diseases through the modulation of the drug targets identified.

### BRIEF SUMMARY OF THE INVENTION

The present invention is based on the discovery of novel interactions between the pairs of proteins described in the tables below. The specific interactions lead to the identification of desirable novel drug targets. Specifically, the interactions implicate several newly discovered interacting proteins (interactors) in neurodegenerative disorders and neurodegenerative disease pathways, and suggest that modulation of such interactors may lead to alleviation of symptoms, delay of onset of symptoms, or treatment of the diseases or symptoms of the diseases. In addition, the protein-protein interactions described can facilitate the formation of protein complexes both in vitro and in vivo. This enables novel approaches for drug screening to select not only drug candidates that modulate the well-known drug targets employed in the interaction discovery process, but also drug candidates that modulate either the newly discovered interactor proteins or the protein-protein interactions themselves. For example, screening assays can be established based on the interaction between a protein known to be involved in a disease pathway and one of its newly discovered protein interactors. Compounds that modulate or interact with the known target protein can be selected based on their ability to either compete with a newly discovered interactor for interaction with the target protein, or promote the interaction between the target protein and the interactor.

Thus, in accordance with a first aspect of the present invention, isolated protein complexes are provided that are formed by the protein-protein interactions disclosed in the tables. In addition, homologues, derivatives, and fragments of the interacting proteins may also be used in forming protein complexes. In a specific embodiment, fragments of an interacting pair of proteins described in the tables, and containing regions responsible for the protein-protein interaction, are used in forming a protein complex of the present invention. In another embodiment, at least one interacting protein member in a protein complex of the present invention is a fusion protein containing a protein in the tables, or a homologue, derivative, or fragment thereof. In yet another embodiment, a protein complex is provided containing a hybrid protein, which comprises, covalently linked together, either directly, or through a linker, a pair of interacting proteins described in the tables, or homologues, derivatives, or fragments thereof. In addition, nucleic acids encoding the hybrid protein are also provided.

In yet another aspect, the present invention also provides a method for making the protein complexes disclosed herein. The method includes the steps of providing the first protein and the second protein in the protein complexes of the present invention, and contacting said first protein with said second protein. In addition, the protein complexes can be prepared by isolation or purification from tissues and cells, or produced by recombinant expression of their individual protein members. The protein complexes can be incorporated into a protein microchip or microarray, which are useful in large-scale high throughput screening assays involving the protein complexes.

In accordance with a second aspect of the invention, antibodies are provided that are immunoreactive with a protein complex of the present invention. In one embodiment, an antibody is selectively immunoreactive with a protein complex of the present invention. In another embodiment, a bifunctional antibody is provided that has two different antigen binding sites, each being specific to a different interacting protein member in a protein complex of the present invention. The antibodies of the present invention can take various forms including polyclonal antibodies, monoclonal antibodies, chimeric antibodies, antibody fragments such as Fv fragments, single-chain Fv fragments (scFv), Fab′ fragments, and F(ab′)₂ fragments. Preferably, the antibodies are partially or fully humanized antibodies. The antibodies of the present invention can be readily prepared using procedures generally known in the art. For example, recombinant libraries such as phage display libraries and ribosome display libraries may be used to screen for antibodies with desirable specificities. In addition, various mutagenesis techniques, such as site-directed mutagenesis and PCR diversification, may be used in combination with the screening assays to create antibodies with new or improved immunoreactivity.

The present invention also provides detection methods for determining whether there is any aberration in a patient with respect to a protein complex formed by one or more interactions provided in accordance with this invention. In one embodiment, the method comprises detecting an aberrant concentration of the protein complexes of the present invention. Alternatively, the concentrations of one or more interacting protein members (at the protein or cDNA/mRNA level) of a protein complex of the present invention are measured. In addition, the cellular localization, or tissue or organ distribution of a protein complex of the present invention is determined to detect any aberrant localization or distribution of the protein complex. In another embodiment, mutations in one or more interacting protein members of a protein complex of the present invention can be detected. In particular, it is desirable to determine whether the interacting protein members have any mutations that will lead to, or are associated with, changes in the functional activity of the proteins or changes in their binding affinity for other interacting protein members in forming a protein complex of the present invention. In yet another embodiment, the binding constant of the interacting protein members of one or more protein complexes is determined. A kit may be used for conducting the detection methods of the present invention. Typically, the kit contains reagents useful in any of the above-described embodiments of the detection methods, including, e.g., antibodies specific to a protein complex of the present invention or interacting members thereof, and oligonucleotides selectively hybridizable to the cDNAs or mRNAs encoding one or more interacting protein members of a protein complex. The detection methods can be useful in diagnosing a disease or disorder such as neurological disorders, ailments and diseases, including mild cognitive impairment, depression, schizophrenia, obsessive-compulsive disorder, bipolar disorder, and neurodegenerative diseases and disorders and motor neuron diseases and disorders such as Alzheimer's disease, Parkinson's disease, dementia with Lewy bodies, amyotrophic lateral sclerosis or Lou Gehrig's disease, Alpers' disease, Leigh's disease, Pelizaeus-Merzbacher disease, Olivopontocerebellar atrophy, Friedreich's ataxia, leukodystrophies, Rett syndrome, Ramsay Hunt syndrome type II, and Down's syndrome, staging the disease or disorder, or identifying a predisposition to the disease or disorder.

The present invention also provides screening methods for selecting modulators of a protein complex provided according to the present invention. Screening methods are also provided for selecting modulators of the individual interacting proteins. The compounds identified in the screening methods of the present invention can be useful in modulating the functions or activities of the individual interacting proteins, or the protein complexes of the present invention. They may also be effective in modulating the cellular processes involving the proteins and protein complexes, and in preventing or ameliorating diseases or disorders such as neurological disorders, ailments and diseases, including mild cognitive impairment, depression, schizophrenia, obsessive-compulsive disorder, bipolar disorder, and neurodegenerative diseases and disorders and motor neuron diseases and disorders such as Alzheimer's disease, Parkinson's disease, dementia with Lewy bodies, amyotrophic lateral sclerosis or Lou Gehrig's disease, Alpers' disease, Leigh's disease, Pelizaeus-Merzbacher disease, Olivopontocerebellar atrophy, Friedreich's ataxia, leukodystrophies, Rett syndrome, Ramsay Hunt syndrome type II, and Down's syndrome.

Thus, test compounds may be screened in in vitro binding assays to identify compounds capable of binding a protein complex of the present invention, or its individual interacting protein members. The assays may include the steps of contacting the protein complex with a test compound and detecting the interaction between the interacting partners. In addition, in vitro dissociation assays may also be employed to select compounds capable of dissociating or destabilizing the protein complexes identified in accordance with the present invention. For example, the assays may entail (1) contacting the interacting members of a protein complex with each other in the presence of a test compound; and (2) detecting the interaction between the interacting members. Additionally, an in vitro screening assay may also be used to identify compounds that trigger, initiate the formation of, or stabilize, a protein complex of the present invention.

In preferred embodiments, the present invention provides screening methods for selecting modulators of a protein complex provided according to the present invention that directly or indirectly reduce Aβ production within cells, Aβ secretion by cells, or, more generally, Aβ concentrations within the human body, and particularly within the plasma and/or brain. Compounds revealed by such screening methods can be further tested for their therapeutic efficacy in (1) lowering Aβ secretion in cell-based assays, (2) lowering Aβ concentrations in the brain or plasma, of animals, and during pre-clinical trails using animal models for specific neurodegenerative diseases, (3) lowering Aβ concentrations in the or plasma of humans during clinical trails.

In highly preferred embodiments, the present invention provides screening methods for selecting modulators of a protein complex provided according to the present invention that directly or indirectly reduce Aβ42 production within cells, Aβ42 secretion by cells, or, more generally, Aβ42 concentrations within the human body, and particularly within the human plasma and/or brain. Compounds revealed by such screening methods can be further tested for their therapeutic efficacy in (1) lowering Aβ42 secretion in cell-based assays, (2) lowering Aβ42 concentrations in the brain, or plasma, of animals, and during pre-clinical trails using animal models for specific neurodegenerative diseases, (3) lowering Aβ42 concentrations in the plasma of humans during clinical trails.

Compounds revealed by such screening methods can be further tested for their therapeutic efficacy in delaying the onset of symptoms of neurodegenerative diseases and disorders, treating the symptoms of neurodegenerative diseases and disorders, or treating the neurodegenerative diseases and disorders themselves, in human patients in need of such treatments. Compounds revealed by such screening methods can also be subjected to iterative rounds of “structure-activity relationship” (SAR) analysis, wherein specific features of the original, “lead,” compound(s) are slightly altered or modified, and the resulting set of modified compounds can be further tested using the same methods that were used to identify the lead compound. Promising modified compounds showing desired results in such screens can also be further tested for their therapeutic efficacy in delaying the onset of symptoms of neurodegenerative diseases and disorders, treating the symptoms of neurodegenerative diseases and disorders, or treating or slowing the progression of the neurodegenerative diseases and disorders themselves, in human patients in need of such treatments.

In preferred embodiments, in vivo assays such as two-hybrid assays and various derivatives thereof, preferably reverse two-hybrid assays, are utilized in identifying compounds that interfere with or disrupt the protein-protein interactions discovered according to the present invention. In addition, systems such as two-hybrid assays are also useful in selecting compounds capable of triggering or initiating, enhancing or stabilizing the protein-protein interactions provided in the tables. In a specific embodiment, the screening method includes: (a) providing in a host cell a first fusion protein having a first protein of an interacting protein pair, or a homologue, derivative or fragment thereof, and a second fusion protein having the second protein of the pair, or a homologue, derivative or fragment thereof, wherein a DNA binding domain is fused to one of the first and second proteins while a transcription-activating domain is fused to the other of said first and second proteins; (b) providing in the host cell a reporter gene, wherein the transcription of the reporter gene is determined by the interaction between the first protein and the second protein; (c) allowing the first and second fusion proteins to interact with each other within the host cell in the presence of a test compound; and (d) determining the presence or absence of expression of the reporter gene.

In addition, the present invention also provides a method for selecting a compound capable of modulating a protein-protein interaction in accordance with the present invention, comprising the steps of (1) contacting a test compound with an interacting protein disclosed in the tables, or a homologue, derivative or fragment thereof; and (2) determining whether said test compound is capable of binding said protein. In a preferred embodiment, the method further includes testing a selected test compound capable of binding said interacting protein for its ability to interfere with a protein-protein interaction according to the present invention involving said interacting protein, and optionally further testing the selected test compound for its ability to modulate cellular activities associated with said interacting protein and/or said protein-protein interaction.

The present invention also relates to virtual screening methods for providing compounds capable of modulating the interaction between the interacting members in a protein complex of the present invention. In one embodiment, the method comprises the steps of providing atomic coordinates defining a three-dimensional structure of a protein complex of the present invention, and designing or selecting compounds, based on said atomic coordinates, capable of interfering with the interaction between the interacting protein members of the protein complex. In another embodiment, the method comprises the steps of providing atomic coordinates defining a three-dimensional structure of an interacting protein described in the tables, and designing or selecting compounds capable of binding the interacting protein based on said atomic coordinates. In preferred embodiments, the method further includes testing a selected test compound for its ability to interfere with a protein-protein interaction provided in accordance with the present invention involving said interacting protein, and optionally further testing the selected test compound for its ability to modulate cellular activities associated with the interacting protein.

The present invention further provides a composition having two expression vectors. One vector contains a nucleic acid encoding a protein of an interacting protein pair according to the present invention, or a homologue, derivative or fragment thereof. Another vector contains the other protein of the interacting pair, or a homologue, derivative or fragment thereof. In addition, an expression vector is also provided containing (1) a first nucleic acid encoding one protein of an interacting protein pair of the present invention, or a homologue, derivative or fragment thereof, and (2) a second nucleic acid encoding the other protein of the interacting pair, or a homologue, derivative or fragment thereof.

Host cells are also provided containing the first and second nucleic acids, or comprising the expression vector(s). In addition, the present invention also provides a host cell having two expression cassettes. One expression cassette includes a promoter operably linked to a nucleic acid encoding one protein of an interacting pair of the present invention, or a homologue, derivative or fragment thereof. Another expression cassette includes a promoter operably linked to a nucleic acid encoding the other protein of the interacting pair, or a homologue, derivative or fragment thereof. Preferably, the expression cassettes are chimeric expression cassettes with heterologous promoters included.

In specific embodiments of the host cells or expression vectors, one of the two nucleic acids is linked to a nucleic acid encoding a DNA binding domain, and the other is linked to a nucleic acid encoding a transcription-activation domain, whereby two fusion proteins can be encoded.

In accordance with yet another aspect of the present invention, methods are provided for modulating the functions and activities of a protein complex of the present invention, or interacting protein members thereof. The methods may be used in treating or preventing neurological disorders, ailments and diseases, including mild cognitive impairment, depression, schizophrenia, obsessive-compulsive disorder, bipolar disorder, and neurodegenerative diseases and disorders and motor neuron diseases and disorders such as Alzheimer's disease, Parkinson's disease, dementia with Lewy bodies, amyotrophic lateral sclerosis or Lou Gehrig's disease, Alpers' disease, Leigh's disease, Pelizaeus-Merzbacher disease, Olivopontocerebellar atrophy, Friedreich's ataxia, leukodystrophies, Rett syndrome, Ramsay Hunt syndrome type II, and Down's syndrome, or in treating the symptoms, or delaying the onset of the symptoms of these diseases and disorders. In one embodiment, the method comprises reducing a protein complex concentration and/or inhibiting the functional activities of the protein complex. Alternatively, the concentration and/or activity of one or more interacting members of a protein complex may be reduced or inhibited. Thus, the methods may include administering to a patient in need of such treatment an antibody specific to a protein complex or an interacting protein member thereof, or an siNA or antisense oligo or ribozyme selectively hybridizable to a gene or mRNA encoding an interacting member of the protein complex. Also useful is a compound identified in a screening assay of the present invention capable of disrupting the interaction between two interacting members of a protein complex, or inhibiting the activities of an interacting member of the protein complex. Consequently, the methods may include administering to a patient in need of such treatment a compound identified in a screening assay of the present invention, which is capable of disrupting or stabilizing the interaction between two interacting members of a protein complex, or inhibiting or promoting the activities of an individual interacting member of the protein complex. In addition, gene therapy methods may also be used in reducing the expression of the gene(s) encoding one or more interacting protein members of a protein complex.

In another embodiment, the methods for modulating the functions and activities of a protein complex of the present invention or interacting protein members thereof comprise increasing the protein complex concentration and/or activating the functional activities of the protein complex. Alternatively, the concentration and/or activity of one or more interacting members of a protein complex of the present invention may be increased. Thus, one or more interacting protein members of a protein complex of the present invention may be administered directly to a patient. Or, exogenous genes or transgenes encoding one or more protein members of a protein complex of the present invention may be introduced into a patient by gene therapy techniques. In addition, compounds identified in a screening assay of the present invention that are capable of triggering or initiating, enhancing or stabilizing a protein-protein interaction of the present invention may be administered to a patient needing such treatment or preventive or prophylactic measures.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

FIGS. 1-59 depict the structures of siRNA compounds designed to reduce the expression of specific proteins that comprise the protein complexes of the present invention.

### DETAILED DESCRIPTION OF THE INVENTION

### 1. Definitions

The terms “polypeptide,” “protein,” and “peptide” are used herein interchangeably to refer to amino acid chains in which the amino acid residues are linked by peptide bonds or modified peptide bonds. The amino acid chains can be of any length of greater than two amino acids. Unless otherwise specified, the terms “polypeptide,” “protein,” and “peptide” also encompass various modified forms thereof. Such modified forms may be naturally occurring modified forms or chemically modified forms. Examples of modified forms include, but are not limited to, glycosylated forms, phosphorylated forms, myristoylated forms, palmitoylated forms, ribosylated forms, acetylated forms, ubiquitinated forms, etc. Modifications also include intra-molecular crosslinking and covalent attachment to various moieties such as lipids, flavin, biotin, polyethylene glycol or derivatives thereof, etc. In addition, modifications may also include cyclization, branching and cross-linking. Further, amino acids other than the conventional twenty amino acids encoded by genes may also be included in a polypeptide.

The term “isolated polypeptide” as used herein is defined as a polypeptide molecule that is present in a form other than that found in nature. Thus, an isolated polypeptide can be a non-naturally occurring polypeptide. For example, an “isolated polypeptide” can be a “hybrid polypeptide.” An “isolated polypeptide” can also be a polypeptide derived from a naturally occurring polypeptide by additions or deletions or substitutions of amino acids. An isolated polypeptide can also be a “purified polypeptide” which is used herein to mean a specified polypeptide in a substantially homogeneous preparation substantially free of other cellular components, other polypeptides, viral materials, or culture medium, or when the polypeptide is chemically synthesized, chemical precursors or by-products associated with the chemical synthesis. A “purified polypeptide” can be obtained from natural or recombinant host cells by standard purification techniques, or by chemically synthesis, as will be apparent to skilled artisans.

The terms “hybrid protein,” “hybrid polypeptide,” “hybrid peptide,” “fusion protein,” “fusion polypeptide,” and “fusion peptide” are used herein interchangeably to mean a non-naturally occurring polypeptide or isolated polypeptide having a specified polypeptide molecule covalently linked to one or more other polypeptide molecules that do not link to the specified polypeptide in nature. Thus, a “hybrid protein” may be two naturally occurring proteins or fragments thereof linked together by a covalent linkage. A “hybrid protein” may also be a protein formed by covalently linking two artificial polypeptides together. Typically, but not necessarily, the two or more polypeptide molecules are linked or “fused” together by a peptide bond forming a single non-branched polypeptide chain.

As used herein, the term “interacting” or “interaction” means that two protein domains, fragments or complete proteins exhibit sufficient physical affinity to each other so as to bring the two “interacting” protein domains, fragments or proteins physically close to each other. An extreme case of interaction is the formation of a chemical bond that results in continual and stable proximity of the two entities. Interactions that are based solely on physical affinities, although usually more dynamic than chemically bonded interactions, can be equally effective in co-localizing two proteins. Examples of physical affinities and chemical bonds include but are not limited to, forces caused by electrical charge differences, hydrophobicity, hydrogen bonds, van der Waals force, ionic force, covalent linkages, and combinations thereof. The state of proximity between the interaction domains, fragments, proteins or entities may be transient or permanent, reversible or irreversible. In any event, it is in contrast to and distinguishable from contact caused by natural random movement of two entities. Typically, although not necessarily, an “interaction” is exhibited by the binding between the interaction domains, fragments, proteins, or entities. Examples of interactions include specific interactions between antigen and antibody, ligand and receptor, enzyme and substrate, and the like.

An “interaction” between two protein domains, fragments or complete proteins can be determined by a number of methods. For example, an interaction is detectable by any commonly accepted approaches, including functional assays such as the two-hybrid systems. Protein-protein interactions can also be determined by various biophysical and biochemical approaches based on the affinity binding between the two interacting partners. Such biochemical methods generally known in the art include, but are not limited to, protein affinity chromatography, affinity blotting, immuno-precipitation, and the like. The binding constant for two interacting proteins, which reflects the strength or quality of the interaction, can also be determined using methods known in the art. See Phizicky and Fields, Microbiol Rev, 59:94-123 (1995).

As used herein, the term “protein complex” means a composite unit that is a combination of two or more proteins formed by interaction between the proteins. Typically but not necessarily, a “protein complex” is formed by the binding of two or more proteins together through specific non-covalent binding affinities. However, covalent bonds may also be present between the interacting partners. For instance, the two interacting partners can be covalently crosslinked so that the protein complex becomes more stable.

The term “isolated protein complex” means a naturally occurring protein complex present in a composition or environment that is different from that found in its native or original cellular or biological environment in nature. An “isolated protein complex” may also be a protein complex that is not found in nature.

The term “protein fragment” as used herein means a polypeptide that represents a portion of a protein. When a protein fragment exhibits interactions with another protein or protein fragment, the two entities are said to interact through interaction domains that are contained within the entities.

As used herein, the term “domain” means a functional portion, segment or region of a protein, or polypeptide. “Interaction domain” refers specifically to a portion, segment or region of a protein, polypeptide or protein fragment that is responsible for the physical affinity of that protein, protein fragment or isolated domain for another protein, protein fragment or isolated domain.

The term “isolated” when used in reference to nucleic acids (which include gene sequences) of this invention is intended to mean that a nucleic acid molecule is present in a form other than that found in nature.

Thus, an isolated nucleic acid can be a non-naturally occurring nucleic acid. For example, the term “isolated nucleic acid” encompasses “recombinant nucleic acid” which is used herein to mean a hybrid nucleic acid produced by recombinant DNA technology having the specified nucleic acid molecule covalently linked to one or more nucleic acid molecules that are not the nucleic acids naturally flanking the specified nucleic acid in the naturally existing chromosome. One example of recombinant nucleic acid is a hybrid nucleic acid encoding a fusion protein. Another example is an expression vector having the specified nucleic acid inserted in a vector.

The term “isolated nucleic acid” also encompasses nucleic acid molecules that are present in a form other than that found in its original environment in nature with respect to its association with other molecules. In this respect, an “isolated nucleic acid” as used herein means a nucleic acid molecule having only a portion of the nucleic acid sequence in the chromosome but not one or more other portions present on the same chromosome. Thus, an isolated nucleic acid present in a form other than that found in its original environment in nature with respect to its association with other molecules typically includes no more than 10 kb of the naturally occurring nucleic acid sequences that immediately flank the gene in the naturally existing chromosome or genomic DNA. Thus, the term “isolated nucleic acid” encompasses the term “purified nucleic acid,” which means an isolated nucleic acid in a substantially homogeneous preparation substantially free of other cellular components, other nucleic acids, viral materials, or culture medium, or chemical precursors or by-products associated with chemical reactions for chemical synthesis of nucleic acids. Typically, a “purified nucleic acid” can be obtained by standard nucleic acid purification methods, as will be apparent to skilled artisans.

An isolated nucleic acid can be in a vector. However, it is noted that an “isolated nucleic acid” as used herein is distinct from a clone in a conventional library such as a genomic DNA library or a cDNA library in that the clones in a library are still in admixture with almost all the other nucleic acids from a chromosome or a cell.

The term “high stringency hybridization conditions,” when used in connection with nucleic acid hybridization, means hybridization conducted overnight at 42° C. in a solution containing 50% formamide, 5×SSC (750 mM NaCl, 75 mM sodium citrate), 50 mM sodium phosphate, pH 7.6, 5× Denhardt's solution, 10% dextran sulfate, and 20 microgram/ml denatured and sheared salmon sperm DNA, with hybridization filters washed in 0.1×SSC at about 65° C. The term “moderate stringent hybridization conditions,” when used in connection with nucleic acid hybridization, means hybridization conducted overnight at 37° C. in a solution containing 50% formamide, 5×SSC (750 mM NaCl, 75 mM sodium citrate), 50 mM sodium phosphate, pH 7.6, 5× Denhardt's solution, 10% dextran sulfate, and 20 microgram/ml denatured and sheared salmon sperm DNA, with hybridization filters washed in 1×SSC at about 50° C. It is noted that many other hybridization methods, solutions and temperatures can be used to achieve comparable stringent hybridization conditions as will be apparent to skilled artisans.

As used herein, the term “homologue,” when used in connection with a first native protein or fragment thereof that is discovered, according to the present invention, to interact with a second native protein or fragment thereof, means a polypeptide that exhibits a sufficient amino acid sequence homology (greater than 20%) and structural resemblance to the first native interacting protein, or to one of the interacting domains of the first native protein, such that it is capable of interacting with the second native protein. Typically, a protein homologue of a native protein may have an amino acid sequence that is at least about 50%, 55%, 60%, 65% or 70%, preferably at least about 75%, more preferably at least about 80%, 85%, 86%, 87%, 88% or 89%, even more preferably at least 90%, 91%, 92%, 93% or 94%, and most preferably about 95%, 96%, 97%, 98% or 99% identical to the native protein. Examples of homologues may be the orthologous proteins of other species including animals, plants, yeast, bacteria, and the like. Homologues may also be obtained by, e.g., mutagenizing a native protein. For example, homologues may be identified by site-specific mutagenesis in combination with assays for detecting protein-protein interactions, e.g., the yeast two-hybrid system described below, as will be apparent to skilled artisans apprised of the present invention. Other techniques for detecting protein-protein interactions include, e.g., protein affinity chromatography, affinity blotting, in vitro binding assays, and the like.

For the purpose of comparing two different nucleic acid or polypeptide sequences, one sequence (test sequence) may be described to be a specific “percent identical to” another sequence (reference sequence) in the present disclosure. In this respect, the percentage identity is determined by the algorithm of Karlin and Altschul, Proc Natl Acad Sci USA, 90:5873-5877 (1993), which is incorporated into various BLAST programs. Specifically, the percentage identity is determined by the “BLAST 2 Sequences” tool, which is available at the National Library of Medicine's National Center for Biotechnological Information's website. See Tatusova and Madden, FEMS Microbiol. Lett., 174(2):247-250 (1999). For pairwise DNA-DNA comparison, the BLASTN 2.1.2 program is used with default parameters (Match: 1; Mismatch: −2; Open gap: 5 penalties; extension gap: 2 penalties; gap x_dropoff: 50; expect: 10; and word size: 11, with filter). For pairwise protein-protein sequence comparison, the BLASTP 2.1.2 program is employed using default parameters (Matrix: BLOSUM62; gap open: 11; gap extension: 1; x_dropoff: 15; expect: 10.0; and wordsize: 3, with filter). Percent identity of two sequences is calculated by aligning a test sequence with a reference sequence using BLAST 2.1.2., determining the number of amino acids or nucleotides in the aligned test sequence that are identical to amino acids or nucleotides in the same position of the reference sequence, and dividing the number of identical amino acids or nucleotides by the number of amino acids or nucleotides in the reference sequence. When BLAST 2.1.2 is used to compare two sequences, it aligns the sequences and yields the percent identity over defined, aligned regions. If the two sequences are aligned across their entire length, the percent identity yielded by the BLAST 2.1.1 is the percent identity of the two sequences. If BLAST 2.1.2 does not align the two sequences over their entire length, then the number of identical amino acids or nucleotides in the unaligned regions of the test sequence and reference sequence is considered to be zero and the percent identity is calculated by adding the number of identical amino acids or nucleotides in the aligned regions and dividing that number by the length of the reference sequence.

The term “derivative,” when used in connection with a first native protein (or fragment thereof) that is discovered, according to the present invention, to interact with a second native protein (or fragment thereof), means a modified form of the first native protein prepared by modifying the side chain groups of the first native protein without changing the amino acid sequence of the first native protein. The modified form, i.e., the derivative, should be capable of interacting with the second native protein. Examples of modified forms include glycosylated forms, phosphorylated forms, myristylated forms, ribosylated forms, ubiquitinated forms, and the like. Derivatives also include hybrid or fusion proteins containing a native protein or a fragment thereof. Methods for preparing such derivative forms should be apparent to skilled artisans. The prepared derivatives can be easily tested for their ability to interact with the native interacting partner using techniques known in the art, e.g., protein affinity chromatography, affinity blotting, in vitro binding assays, yeast two-hybrid assays, and the like.

The term “antibody” as used herein encompasses both monoclonal and polyclonal antibodies that fall within any antibody classes, e.g., IgG, IgM, IgA, IgE, or derivatives thereof. The term “antibody” also includes antibody fragments including, but not limited to, Fab, F(ab′)₂, and conjugates of such fragments, and single-chain antibodies comprising an antigen recognition epitope. In addition, the term “antibody” also means humanized antibodies, including partially or fully humanized antibodies. An antibody may be obtained from an animal, or from a hybridoma cell line producing a monoclonal antibody, or obtained from cells or libraries recombinantly expressing a gene encoding a particular antibody.

The term “selectively immunoreactive” as used herein means that an antibody is reactive thus binds to a specific protein or protein complex, but not other similar proteins or fragments or components thereof.

The term “activity” when used in connection with proteins or protein complexes means any physiological or biochemical activities displayed by or associated with a particular protein or protein complex including but not limited to activities exhibited in biological processes and cellular functions, ability to interact with or bind another molecule or a moiety thereof, binding affinity or specificity to certain molecules, in vitro or in vivo stability (e.g., protein degradation rate, or in the case of protein complexes, the ability to maintain the form of a protein complex), antigenicity and immunogenicity, enzymatic activities, etc. Such activities may be detected or assayed by any of a variety of suitable methods as will be apparent to skilled artisans.

The term “compound” as used herein encompasses all types of organic or inorganic molecules, including but not limited proteins, peptides, polysaccharides, lipids, nucleic acids, small organic molecules, inorganic compounds, and derivatives thereof.

As used herein, the term “interaction antagonist” means a compound that interferes with, blocks, disrupts or destabilizes a protein-protein interaction; blocks or interferes with the formation of a protein complex; or destabilizes, disrupts or dissociates an existing protein complex.

The term “interaction agonist” as used herein means a compound that triggers, initiates, propagates, nucleates, or otherwise enhances the formation of a protein-protein interaction; triggers, initiates, propagates, nucleates, or otherwise enhances the formation of a protein complex; or stabilizes an existing protein complex.

Unless otherwise specified, the term “PS1(467)” as used herein means the human PS1 protein, and, in particular, the splice form that comprises 467 amino acid residues. The usage for naming other proteins will be similar unless otherwise specified in the present disclosure.

Generally the protein complexes of the present invention are said to comprise a “first protein” interacting with a “second protein,” wherein the first and second proteins are respectively identified as the “bait protein” and “prey protein” in one of the tables below. Unless indicated otherwise, the interacting first and second proteins of the present invention comprise not only the full length human proteins identified in the tables, but they also comprise those fragments of the identified proteins that retain the ability to interact. Additionally, the interacting first and second proteins of the present invention further comprise proteins that (a) are homologous to the identified human first and second proteins, (b) retain the ability to interact, and (c) possess 75%, 80%, 85%, 90%, 95%, 96%, 97%, 98%, 99% or greater percent identity to the full-length identified human first and second proteins. Finally, the interacting first and second proteins of the present invention also comprise fragments of the homologous proteins described above, so long as the fragments of the homologous proteins retain the ability to interact.

### 2. Protein Complexes

Novel protein-protein interactions have been discovered. The protein-protein interactions are provided in the tables below. Specific fragments capable of conferring interacting properties on the interacting proteins have been identified. The GenBank reference numbers for the cDNA sequences encoding the interacting proteins are also noted in the tables.

### TABLES

TABLE 1
Binding Regions of HLA-B-associated transcript
3 (BAT3) and glypican 1 (GLYP)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| BAT3 | 271 | 480 | GLYP | 400 | 483 |
| (GenBank | | | (GenBank | | |
| Accession No. | | | Accession No. | | |
| M33519) | | | X54232) | | |

TABLE 2
Binding Regions of HLA-B-associated transcript 3 (BAT3) and
low-density lipoprotein receptor-related protein 2 (LRP2)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| BAT3 | 740 | 1040 | LRP2 | 1 | 304 |
| (GenBank | | | (GenBank | 1 | 217 |
| Accession No. | | | Accession No. | | |
| M33519) | | | U33837) | | |

TABLE 3
Binding Regions of HLA-B-associated transcript 3 (BAT3) and
low-density lipoprotein receptor-related protein 2 (LRPAP1)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| BAT3 | 740 | 1040 | LRPAP1 | 11 | 361 |
| (GenBank | | | (GenBank | | |
| Accession No. | | | Accession No. | | |
| M33519) | | | M63959) | | |

TABLE 4
Binding Regions of HLA-B-associated transcript 3 (BAT3)
and transthyretin (prealbumin, amyloidosis type I) (TTR)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| BAT3 | 740 | 1040 | TTR | 6 | 147 |
| (GenBank | | | (GenBank | 6 | 148 |
| Accession No. | | | Accession No. | | |
| M33519) | | | NM 000371) | | |

TABLE 5
Binding Regions of amyloid beta (A4) precursor protein-binding,
family B, member 1, isoform E9 (710) (Fe65 or APBB1(710))
and novel protein PN7740

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| APBB1(710) | 360 | 552 | PN7740 | 27 | 321 |
| (GenBank | | | (GenBank | | |
| Accession No. | | | Accession No.) | | |
| L77864) | | | | | |

TABLE 6
Binding Regions of amyloid beta (A4) precursor protein-binding,
family A, member 1 (X11) (APBA1 or Mint 1) and KIAA0427

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| APBA1 | 447 | 758 | KIAA0427 | 364 | 589 |
| (GenBank | | | (GenBank | | |
| Accession No. | | | Accession No. | | |
| AF047347) | | | AB007887) | | |

TABLE 7
Binding Regions of presenilin 1, alt. transcript (467)
(PS1(467)) and amyloid beta (A4) precursor protein-binding,
family A, member 1 (X11) (APBA1 or Mint 1)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| PS1(467) | 1 | 91 | APBA1 | 471 | 822 |
| (GenBank | | | (GenBank | | |
| Accession No. | | | Accession No. | | |
| L42110) | | | AF047347) | | |

TABLE 8
Binding Regions of amyloid beta (A4) precursor protein-binding,
family A, member 1 (X11) (APBA1 or Mint 1) and glutamate
ammonia ligase (GS or GLUL)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| APBA1 | 739 | 837 | GLUL | 49 | 212 |
| (GenBank | | | (GenBank | | |
| Accession No. | | | Accession No. | | |
| AF047347) | | | X59834) | | |

TABLE 9
Binding Regions of calcium/calmodulin-dependent
serine protein kinase (CASK) and dystrophin,
Dp427m isoform (3685) (DMD(3685))

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| CASK | 306 | 574 | DMD(3685) | 909 | 1280 |
| (GenBank | | | (GenBank | | |
| Accession No. | | | Accession No. | | |
| AF032119) | | | M18533) | | |

TABLE 10
Binding Regions for calcium and integrin binding
protein (CIB) and site-1 protease (S1P)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| CIB | 1 | 191 | S1P | 442 | 619 |
| (GenBank | | | (GenBank | | |
| Accession No. | | | Accession No. | | |
| U82226) | | | D42053) | | |

TABLE 11
Binding Regions of amyloid beta (A4) precursor
protein-binding, family A, member 2 (X11-like)
(Mint 2 or APBA2) and site-1 protease (S1P)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| APBA2 | 1 | 210 | S1P | 765 | 959 |
| (GenBank | | | (GenBank | | |
| Accession No. | | | Accession No. | | |
| AF047348) | | | D42053) | | |

TABLE 12
Binding Regions of presenilin 1, alt. transcript (467)
(PS1(467)) and 3-phosphoglycerate dehydrogenase (PGAD)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| PS1(467) | 1 | 91 | PGAD | 1 | 266 |
| (GenBank | | | (GenBank | | |
| Accession No. | | | Accession No. | | |
| L42110) | | | AF006043) | | |

TABLE 13
Binding Regions of presenilin 1, alt. transcript (467) (PS1(467))
and electron transfer flavoprotein, beta (ETFB or beta-ETF)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| PS1(467) | 1 | 91 | ETFB | 31 | 242 |
| (GenBank | | | (GenBank | | |
| Accession No. | | | Accession No. | | |
| L42110) | | | X71129) | | |

TABLE 14
Binding Regions of presenilin 1, alt. transcript
(467) (PS1(467)) and glyceraldehyde-3-phosphate
dehydrogenase (GAPD or GAPDH)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| PS1(467) | 1 | 91 | GAPD | 2 | 190 |
| (GenBank | | | (GenBank | | |
| Accession No. | | | Accession No. | | |
| L42110) | | | M17851) | | |

TABLE 15
Binding Regions of presenilin 2, isoform 1 (448) (PS2(448))
and glyceraldehyde-3-phosphate dehydrogenase (GAPD or GAPDH)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| PS2(448) | 1 | 97 | GAPD | 2 | 190 |
| (GenBank | | | (GenBank | | |
| Accession No. | | | Accession No. | | |
| L44577) | | | M17851) | | |

TABLE 16
Binding Regions of calcium and integrin binding
protein (CIB) and ATP synthase, beta chain, mitochondrial,
F1 complex, alt. transcript 1 (ATPMB)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| CIB | 1 | 137 | ATPMB | 229 | 459 |
| (GenBank | 1 | 191 | (GenBank | 334 | 539 |
| Accession No. | | | Accession No. | 378 | 539 |
| U82226) | | | X03559) | 111 | 460 |
| | | | | 271 | 434 |
| | | | | 374 | 499 |

TABLE 17
Binding Regions of G protein-coupled receptor-associated
sorting protein (GASP or KIAA0443) and phosphatidylinositol
4-kinase (PI4K)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| GASP | 901 | 1200 | PI4K | 567 | 854 |
| (GenBank | | | (GenBank | | |
| Accession No. | | | Accession No. | | |
| AB007903) | | | L36151) | | |

TABLE 18
Binding Regions of G protein-coupled receptor-associated
sorting protein (GASP or KIAA0443) and 5-hydroxytryptamine
(serotonin) receptor 2A (5HT-2A)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| GASP | 901 | 1200 | 5HT-2A | 27 | 132 |
| (GenBank | | | (GenBank | | |
| Accession No. | | | Accession No. | | |
| AB007903) | | | X57830) | | |

TABLE 19
Binding Regions of hypothetical protein KIAA0351 and triple
function protein, alt. transcript (2861) (TRIO(2861))

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| KIAA0351 | 301 | 557 | TRIO(2861) | 475 | 733 |
| (GenBank | | | (GenBank | | |
| Accession No. | | | Accession No. | | |
| AB002349) | | | U42390) | | |

TABLE 20
Binding Regions of calcium and integrin binding protein (CIB)
and mitogen-activated protein kinase kinase kinase 10, alt. transcript
(954) (MAP3K10; or mixed lineage kinase 2, MLK2)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| CIB | 1 | 191 | MAP3K10 | 305 | 549 |
| (GenBank | | | (GenBank | | |
| Accession No. | | | Accession No. | | |
| U82226) | | | X90846) | | |

TABLE 21
Binding Regions of BCL2-associated X protein, isoform
beta (218) (BAX-beta) and potassium large conductance
calcium-activated channel, subfamily M, alpha member
1(KCNMA1 or slo K⁺ channel)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| BAX-beta | 50 | 107 | KCNMA1 | 643 | 993 |
| (GenBank | | | (GenBank | | |
| Accession No. | | | Accession No. | | |
| L22474) | | | U13913) | | |

TABLE 22
Binding Regions of focal adhesion kinase 2 (FAK2)
and ATP-binding cassette transporter, sub-family
C (CFTR/MRP), member 8 (ABCC8 or SUR1)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| FAK2 | 673 | 866 | ABCC8 | 121 | 270 |
| (GenBank | | | (GenBank | | |
| Accession No. | | | Accession No. | | |
| L49207) | | | AF087138) | | |

TABLE 23
Binding Regions of amyloid beta (A4) precursor protein-binding,
family A, member 2 (X11-like) (Mint 2 or APBA2)
and phosphodiesterase 9A (PDE9A)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| APBA2 | 1 | 210 | PDE9A | 269 | 593 |
| (GenBank | | | (GenBank | | |
| Accession No. | | | Accession No. | | |
| AF047348) | | | AF048837) | | |

TABLE 24
Binding Regions of calcium and integrin binding protein
(CIB) and stearoyl-CoenzymeA desaturase 1 (SCD1)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| CIB | 1 | 191 | SCD1 | 320 | 359 |
| (GenBank | | | (GenBank | | |
| Accession No. | | | Accession No. | | |
| U82226) | | | Y13647.1) | | |

TABLE 25
Binding Regions of guanine nucleotide-binding protein
rab11a (RAB11A) and focal adhesion kinase (FAK)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| RAB11A | 1 | 137 | FAK | 726 | 1003 |
| (GenBank | | | (GenBank | 726 | 1014 |
| Accession No. | | | Accession No. | 818 | 1014 |
| X56740) | | | L13616) | | |

TABLE 26
Binding Regions of focal adhesion kinase (FAK)
and casein kinase II, alpha 2 (CSNK2A2)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| FAK | 724 | 1052 | CSNK2A2 | 264 | 351 |
| (GenBank | | | (GenBank | | |
| Accession No. | | | Accession No. | | |
| L13616) | | | M55268) | | |

TABLE 27
Binding Regions of focal adhesion kinase (FAK) and
glutathione S-transferase M3 (GTM3 or GST trans M3)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| FAK | 724 | 1052 | GTM3 | 15 | 226 |
| (GenBank | | | (GenBank | | |
| Accession No. | | | Accession No. | | |
| L13616) | | | J05459) | | |

TABLE 28
Binding Regions of breakpoint cluster region, alt. transcript
1 (BCR) and postsynaptic density protein 95 (PSD95)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| BCR | 1206 | 1271 | PSD95 | 110 | 266 |
| (GenBank | | | (GenBank | 56 | 331 |
| Accession No. | | | Accession No. | | |
| M24603) | | | NM 001365) | | |

TABLE 29
Binding Regions of breakpoint cluster region,
alt. transcript 1 (BCR) and discs, large (Drosophila)
homolog 3 (neuroendocrine-dlg) (DLG3)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| BCR | 1206 | 1271 | DLG3 | 94 | 506 |
| (GenBank | | | (GenBank | | |
| Accession No. | | | Accession No. | | |
| M24603) | | | U49089) | | |

TABLE 30
Binding Regions of breakpoint cluster region, alt.
transcript 1 (BCR) and semaphorin 4C, alt. transcript
(821) (SEMA4C(821) or Semaphorin F)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| BCR | 856 | 1226 | SEMA4C(821) | 671 | 821 |
| (GenBank | | | (GenBank | | |
| Accession No. | | | Accession No. | | |
| M24603) | | | NM 017789) | | |

TABLE 31
Binding Regions of breakpoint cluster region, alt. transcript
1 (BCR) and transcription factor HTF4A (HTF4)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| BCR | 1206 | 1271 | HTF4 | 296 | 494 |
| (GenBank | 1134 | 1271 | (GenBank | | |
| Accession No. | 1057 | 1226 | Accession No. | | |
| M24603) | 856 | 1226 | M83233) | | |

TABLE 32
Binding Regions of breakpoint cluster region, alt. transcript
1 (BCR) and SNF2-related CBP activator protein (SRCAP)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| BCR | 1134 | 1271 | SRCAP | 1916 | 2088 |
| (GenBank | | | (GenBank | | |
| Accession No. | | | Accession No. | | |
| M24603) | | | AF143946) | | |

TABLE 33
Binding Regions of postsynaptic density protein
95 (PSD95) and novel protein PN7740 (PN7740)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| PSD95 | 149 | 255 | PN7740 | 27 | 321 |
| (GenBank | | | | | |
| Accession No. | | | | | |
| NM 001365) | | | | | |

TABLE 34
Binding Regions of presenilin 1, alt. transcript (467)
(PS1(467)) and FK506-binding protein 25 (FKBP25)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| PS1(467) | 1 | 91 | FKBP25 | 166 | 224 |
| (GenBank | | | (GenBank | 150 | 224 |
| Accession No. | | | Accession No. | | |
| L42110) | | | M90309) | | |

TABLE 35
Binding Regions of FK506-binding protein 25 (FKBP25)
and calcium and integrin binding protein (CIB)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| FKBP25 | 1 | 224 | CIB | 1 | 191 |
| (GenBank | | | (GenBank | | |
| Accession No. | | | Accession No. | | |
| M90309) | | | U82226) | | |

TABLE 36
Binding Regions of presenilin 1, alt. transcript (467) (PS1(467))
and guanine nucleotide-binding protein rab11a (RAB11A)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| PS1(467) | 1 | 91 | RAB11A | 106 | 216 |
| (GenBank | | | (GenBank | | |
| Accession No. | | | Accession No. | | |
| L42110) | | | X56740) | | |

TABLE 37
Binding Regions of amyloid A-beta protein precursor, alt. transcript
1 (695) (APP(695)) and HLA-B-associated transcript 3 (BAT3)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| APP(695) | 639 | 696 | BAT3 | 603 | 1132 |
| (GenBank | | | (GenBank | | |
| Accession No. | | | Accession No. | | |
| NM 000484) | | | M33519) | | |

TABLE 38
Binding Regions of HLA-B-associated transcript
3 (BAT3) and adaptor-related protein complex
3, delta 1 (AP3D1 or δ-adaptin)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| BAT3 | 1 | 241 | AP3D1 | 1062 | 1153 |
| (GenBank | | | (GenBank | | |
| Accession No. | | | Accession No. | | |
| M33519) | | | AF002163) | | |

TABLE 39
Binding Regions of amyloid A-beta protein precursor,
alt. transcript 1 (695) (APP(695)) and tyrosine
phosphatase, zeta polypeptide (PTPZ)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| APP(695) | 306 | 500 | PTPZ | 1052 | 1128 |
| (GenBank | | | (GenBank | | |
| Accession No. | | | Accession No. | | |
| NM 000484) | | | M93426) | | |

TABLE 40
Binding Regions of amyloid A-beta protein precursor,
alt. transcript 1 (695) (APP(695)) and hypothetical
protein KIAA0351 (KIAA0351)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| APP(695) | 306 | 500 | KIAA0351 | 213 | 557 |
| (GenBank | | | (GenBank | 213 | 312 |
| Accession No. | | | Accession No. | | |
| NM 000484) | | | AB002349) | | |

TABLE 41
Binding Regions of amyloid A-beta protein precursor,
alt. transcript 1 (695) (APP(695)) and prostaglandin
D synthase (PGD-synt or PGDS)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| APP(695) | 306 | 500 | PGD-synt | 1 | 190 |
| (GenBank | | | (GenBank | | |
| Accession No. | | | Accession No. | | |
| NM 000484) | | | M98539) | | |

TABLE 42
Binding Regions of acetylcholinesterase (YT
blood group), hydrophilic form (614) (ACHE(614))
and calpain 4, small subunit (CAPN4)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| ACHE(614) | 31 | 137 | CAPN4 | 1 | 268 |
| (GenBank | | | (GenBank | | |
| Accession No. | | | Accession No. | | |
| M55040) | | | X04106) | | |

TABLE 43
Binding Regions of acetylcholinesterase (YT blood
group), hydrophilic form (614) (ACHE(614)) and
hypothetical protein KIAA0436 (KIAA0436)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| ACHE(614) | 31 | 136 | KIAA0436 | 200 | 638 |
| (GenBank | 266 | 354 | (GenBank | 246 | 638 |
| Accession No. | | | Accession No. | 206 | 638 |
| M55040) | | | AB007896) | 246 | 638 |

TABLE 44
Binding Regions of acetylcholinesterase (YT blood group), hydrophilic
form (614) (ACHE(614)) and endosulfine, alpha (ALPEND)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| ACHE(614) | 31 | 136 | ALPEND | 24 | 121 |
| (GenBank | | | (GenBank | | |
| Accession No. | | | Accession No. | | |
| M55040) | | | X99906) | | |

TABLE 45
Binding Regions of acetylcholinesterase (YT blood
group), hydrophilic form (614) (ACHE(614)) and
RGS-GAIP interacting protein GIPC (GIPC)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| ACHE(614) | 31 | 136 | GIPC | 67 | 332 |
| (GenBank | | | (GenBank | | |
| Accession No. | | | Accession No. | | |
| M55040) | | | AF089816) | | |

TABLE 46
Binding Regions of acetylcholinesterase (YT blood group),
hydrophilic form (614) (ACHE(614)) and catenin (cadherin-associated
protein), delta 2 (neural plakophilin-related arm-repeat
protein) (CTNND2 or δ-catenin)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| ACHE(614) | 63 | 534 | CTNND2 | 265 | 792 |
| (GenBank | 355 | 517 | (GenBank | | |
| Accession No. | 355 | 614 | Accession No. | | |
| M55040) | | | U96136) | | |

TABLE 47
Binding Regions of catenin (cadherin-associated protein), delta
2 (neural plakophilin-related arm-repeat protein) (CTNND2 or
δ-catenin) and RGS-GAIP interacting protein GIPC (GIPC)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| CTNND2 | 1006 | 1225 | GIPC | 67 | 332 |
| (GenBank | | | (GenBank | | |
| Accession No. | | | Accession No. | | |
| U96136) | | | AF089816) | | |

TABLE 48
Binding Regions of catenin (cadherin-associated protein),
delta 2 (neural plakophilin-related arm-repeat protein) (CTNND2
or δ-catenin) and clathrin, heavy chain 1 (CLTC)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| CTNND2 | 516 | 833 | CLTC | 1311 | 1676 |
| (GenBank | | | (GenBank | | |
| Accession No. | | | Accession No. | | |
| U96136) | | | D21260) | | |

TABLE 49
Binding Regions of catenin (cadherin-associated protein), delta 2
(neural plakophilin-related arm-repeat protein) (CTNND2 or δ-catenin)
and plakophilin 2, alt. transcript b (881) (PKP2(881))

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| CTNND2 | 516 | 833 | PKP2(881) | 649 | 817 |
| (GenBank | | | (GenBank | | |
| Accession No. | | | Accession No. | | |
| U96136) | | | X97675) | | |

TABLE 50
Binding Regions of catenin (cadherin-associated protein), delta 2
(neural plakophilin-related arm-repeat protein) (CTNND2 or δ-catenin)
and breakpoint cluster region, alt. transcript 1 (BCR)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| CTNND2 | 516 | 833 | BCR | 1100 | 1227 |
| (GenBank | | | (GenBank | | |
| Accession No. | | | Accession No. | | |
| U96136) | | | M24603) | | |

TABLE 51
Binding Regions of catenin (cadherin-associated protein),
delta 2 (neural plakophilin-related arm-repeat protein)
(CTNND2 or δ-catenin) and 14-3-3 protein, beta,
transcript variant 1 (246) 14-3-3b(246)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| CTNND2 | 1006 | 1158 | 14-3-3b(246) | 1 | 245 |
| (GenBank | | | (GenBank | | |
| Accession No. | | | Accession No. | | |
| U96136) | | | NM 003404) | | |

TABLE 52
Binding Regions of catenin (cadherin-associated protein),
delta 2 (neural plakophilin-related arm-repeat protein) (CTNND2
or δ-catenin) and 14-3-3 protein, zeta (14-3-3z)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| CTNND2 | 1006 | 1158 | 14-3-3z | 1 | 245 |
| (GenBank | | | (GenBank | | |
| Accession No. | | | Accession No. | | |
| U96136) | | | M86400) | | |

TABLE 53
Binding Regions of catenin (cadherin-associated protein),
delta 2 (neural plakophilin-related arm-repeat protein) (CTNND2
or δ-catenin) and focal adhesion kinase 2 (FAK2)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| CTNND2 | 1006 | 1158 | FAK2 | 625 | 1009 |
| (GenBank | | | (GenBank | | |
| Accession No. | | | Accession No. | | |
| U96136) | | | L49207) | | |

TABLE 54
Binding Regions of focal adhesion kinase 2 (FAK2) and catenin
(cadherin-associated protein), delta 2 (neural plakophilin-related
arm-repeat protein) (CTNND2 or δ-catenin)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| FAK2 | 673 | 866 | CTNND2 | 325 | 638 |
| (GenBank | | | (GenBank | | |
| Accession No. | | | Accession No. | | |
| L49207) | | | U96136) | | |

TABLE 55
Binding Regions of catenin (cadherin-associated protein),
delta 2 (neural plakophilin-related arm-repeat protein)
(CTNND2 or δ-catenin) and epidermal growth factor
receptor kinase substrate 8 (EPS8)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| CTNND2 | 516 | 833 | EPS8 | 343 | 822 |
| (GenBank | | | (GenBank | | |
| Accession No. | | | Accession No. | | |
| U96136) | | | U12535) | | |

TABLE 56
Binding Regions of catenin (cadherin-associated protein),
delta 2 (neural plakophilin-related arm-repeat protein)
(CTNND2 or δ-catenin) and G protein-coupled receptor-associated
sorting protein (GASP or KIAA0443)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| CTNND2 | 1006 | 1158 | GASP | 1161 | 1245 |
| (GenBank | 1006 | 1225 | (GenBank | 1161 | 1395 |
| Accession No. | | | Accession No. | | |
| U96136) | | | AB007903) | | |

TABLE 57
Binding Regions of synuclein, alpha, isoform NACP140
(140) (SNCA(140)) and catenin (cadherin-associated
protein), delta 2 (neural plakophilin-related arm-repeat
protein) (CTNND2 or δ-catenin)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| SNCA(140) | 1 | 140 | CTNND2 | 256 | 792 |
| (GenBank | | | (GenBank | | |
| Accession No. | | | Accession No. | | |
| L08850) | | | U96136) | | |

TABLE 58
Binding Regions of amyloid beta-peptide binding protein (ERAB
or hydroxyacyl-Coenzyme A dehydrogenase, type II) and catenin
(cadherin-associated protein), delta 2 (neural plakophilin-related
arm-repeat protein) (CTNND2 or δ-catenin)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| ERAB | 1 | 261 | CTNND2 | 257 | 792 |
| (GenBank | | | (GenBank | | |
| Accession No. | | | Accession No. | | |
| AF069134) | | | U96136) | | |

TABLE 59
Binding Regions of Bcl2-alpha and catenin (cadherin-associated
protein), delta 2 (neural plakophilin-related arm-repeat
protein) (CTNND2 or δ-catenin)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| Bcl2-alpha | 1 | 75 | CTNND2 | 690 | 1225 |
| (GenBank | 1 | 100 | (GenBank | 257 | 792 |
| Accession No. | 1 | 74 | Accession No. | 257 | 792 |
| M13994) | | | U96136) | | |

TABLE 60
Binding Regions of presenilin 1, alt. transcript
(467) (PS1(467)) and enolase, alpha (ENOA)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| PS1(467) | 1 | 91 | ENOA | 135 | 433 |
| (GenBank | | | (GenBank | | |
| Accession No. | | | Accession No. | | |
| L42110) | | | M14328) | | |

TABLE 61
Binding Regions of axin (AXIN) and citrate synthase (cit-synt)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| AXIN | 301 | 600 | cit-synt | 1 | 123 |
| (GenBank | | | (GenBank | | |
| Accession No. | | | Accession No. | | |
| AF009674) | | | AF047042) | | |

TABLE 62
Binding Regions of axin (AXIN) and aldolase
C, fructose-bisphosphate (ALDOC)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| AXIN | 301 | 600 | ALDOC | 210 | 364 |
| (GenBank | | | (GenBank | | |
| Accession No. | | | Accession No. | | |
| AF009674) | | | NM 005165) | | |

TABLE 63
Binding Regions of axin (AXIN) and creatine kinase, brain (CKB)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| AXIN | 1 | 300 | CKB | 13 | 303 |
| (GenBank | | | (GenBank | 219 | 381 |
| Accession No. | | | Accession No. | 252 | 381 |
| AF009674) | | | X15334) | | |

TABLE 64
Binding Regions of axin (AXIN) and neurogranin (NRGN)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| AXIN | 301 | 600 | NRGN | 1 | 78 |
| (GenBank | | | (GenBank | | |
| Accession No. | | | Accession No. | | |
| AF009674) | | | U89165) | | |

TABLE 65
Binding Regions of axin (AXIN) and guanine
nucleotide-binding protein rab3a (RAB3A)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| AXIN | 301 | 600 | RAB3A | −44 | 110 |
| (GenBank | | | (GenBank | | |
| Accession No. | | | Accession No. | | |
| AF009674) | | | NM 002866) | | |

TABLE 66
Binding Regions of axin (AXIN) and peroxiredoxin 3 (PRDX3)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| AXIN | 451 | 740 | PRDX3 | 1 | 256 |
| (GenBank | 451 | 750 | (GenBank | 1 | 258 |
| Accession No. | 301 | 600 | Accession No. | 1 | 256 |
| AF009674) | | | D49396) | | |

TABLE 67
Binding Regions of axin (AXIN) and survival
of motor neuron 1, telomeric (SMN1)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| AXIN | 301 | 600 | SMN1 | 5 | 294 |
| (GenBank | | | (GenBank | | |
| Accession No. | | | Accession No. | | |
| AF009674) | | | U18423) | | |

TABLE 68
Binding Regions of axin (AXIN) and splicing factor,
arginine/serine-rich 9 (SFRS9 or SRp30c)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| AXIN | 301 | 600 | SFRS9 | 175 | 221 |
| (GenBank | | | (GenBank | | |
| Accession No. | | | Accession No. | | |
| AF009674) | | | U30825) | | |

TABLE 69
Binding Regions of presenilin 1, alt. transcript (467)
(PS1(467)) and transcription factor CP2 (TFCP2 or LSF)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| PS1(467) | 1 | 91 | TFCP2 | 405 | 502 |
| (GenBank | | | (GenBank | | |
| Accession No. | | | Accession No. | | |
| L42110) | | | U03494) | | |

TABLE 70
Binding Regions of transcription factor CP2
(TFCP2 or LSF) and amyloid A-beta protein precursor,
alt. transcript 1 (695) (APP(695))

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| TFCP2 | 393 | 502 | APP(695) | 1 | 220 |
| (GenBank | | | (GenBank | 1 | 227 |
| Accession No. | | | Accession No. | | |
| U03494) | | | NM 000484) | | |

TABLE 71
Binding Regions of transcription factor CP2 (TFCP2
or LSF) and 4F5 protein, short isoform (4F5S)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| TFCP2 | 393 | 502 | 4F5S | 5 | 63 |
| (GenBank | | | (GenBank | | |
| Accession No. | | | Accession No. | | |
| U03494) | | | AF073518) | | |

### 2.1. Biological Significance

Amyloid beta (Aβ) precursor protein (APP) metabolism is critical to the pathogenesis of Alzheimer's disease (AD), because it leads to the release of either toxic (Aβ) or trophic (sAPP) metabolites (Cummings et al., Neurology 51:S2-S17 (1998); Roch and Puttfarcken, Alz ID Res A1 1:9-16 (1996)). In this respect, it is important to identify proteins involved in the processing and intracellular trafficking of APP. Proteins that interact with the cytosolic C-terminal region of APP play a major role in this process. The interactions of APP with Fe65 (also known as amyloid beta (A4) precursor protein-binding, family B, member 1, isoform E9 (710) or APBB1 (710)), Fe65 L, Mint1 (also known as APBA1), and Mint2 (also known as APBA2), have all been well documented (Russo et al., FEBS Let 434:1-7 (1998)); Sastre et al., J Biol Chem 273:22351-22357 (1998)). We previously described an interaction between APP and HLA-B-associated transcript 3 (BAT3, also known as scythe), and between BAT3 and adaptor-related protein complex 3, delta 1 (δ-adaptin), and we have explained the importance of these interactions in APP trafficking and metabolism (See U.S. patent application Ser. No. 09/466,139 and International Patent Application No. PCT/US99/30396 (WO 00/37483)), filed 21 Dec. 1999). The presenilins (PS1 and PS2) are also involved in AD pathogenesis. Mutations in PS1 and PS2 are known to cause AD (Hardy, Hum Mol Genet 6:1639-1646 (1997); Selkoe, Trends Cell Biol 8:447-453 (1998)), and recently, it was found that the presenilins could be the γ-secretase that cleave APP at the C-terminus of the Aβ peptide (Wolfe et al., Nature 398:513-517 (1999); De Strooper et al., Nature 398:518-522 (1999); Wolfe et al., Biochemistry 38:11223:11230 (1999); Li et al., Proc Natl Acad Sci USA 97:6138-6143 (2000); Li et al., Nature 405:689-694 (2000)). PS1 interacts with δ-catenin (CTNND2) (Zhou et al., Neuroreport 8:2085-2090 (1997); Tanahashi and Tabira, Neuroreport 10:563-568 (1999)) and calcium and integrin binding protein (CIB) interacts with both PS1 and PS2 (Stabler et al., J. Cell Biol 145:1277-1292 (1999)). To extend our understanding of the role of these proteins in APP trafficking and metabolism, we have sought to determine the identities of the proteins with which they interact.

We found that a fragment corresponding to amino acids 271 to 480 of BAT3 interacts with a fragment of glypican 1 (GLYP) corresponding to amino acids 400 to 483. Glypican is one of the several core proteins of heparan sulfate proteoglycan (other core proteins include the various forms of syndecan, perlecan, appican, and others). The glypican cDNA codes for 558 residues, but after removal of the signal peptide (aa 1 to 23) and of the propeptide (aa 531 to 558), the mature form of glypican contains 507 amino acids. Glypican is attached to the membrane through a GPI anchor and was recently shown to be a receptor that mediates ABtoxicity (Schulz et al., Eur J Neurosci 10:2085-2093 (1998)). On the other hand, secreted glypican binds to substrate-bound APP and inhibits neurite extension normally elicited by APP (Williamson et al., J Biol Chem 271:31215-31221 (1996)). The mechanism of inhibition may be a competition of glypican for substrate-bound APP, against other endogenous proteoglycans that are normally required for APP to stimulate neurite outgrowth. In addition, because glypican bears heparan sulfate, and because heparin stimulates β-secretase (Leveugle et al., Neurochem Int 30:643-548 (1997)), glypican could favor release of sAPPβ versus sAPPα from cells, thus reducing the trophic potency of sAPP (sAPPβ is known to have greatly reduced neurite extension (Li et al., J Neurobiol 32:469-480 (1997)) and neuroprotective (Furukawa et al., J Neurochem 67:1882-1896 (1996)) activities compared to sAPPα). Thus, BAT3 interacts with both APP and glypican, which are known to interact with each other and to control phenomena such as neurite extension and neuronal survival. Pharmacological modulation of the BAT3-glypican interaction might influence the neurotrophic effects elicited by APP, as well as the neurotoxic effects mediated by Aβ.

We also found that fragment of BAT3 comprising amino acids 740 to 1040 interacted with a polypeptide comprising amino acids 1 to 304 of low-density lipoprotein receptor-related protein 2 (LRP2). LRP2 (also called glycoprotein 330 and megalin) was previously shown to bind ApoJ (Kounnas et al., J Biol Chem 270:13070-13075 (1995)), as well as ApoE (Orlando et al., Proc Natl Acad Sci USA 94:2368-2373 (1997)). A study of LRP2 function (Zlokovic et al., Proc Natl Acad Sci USA 93:4229-4234 (1996)) suggested that LRP2 is necessary for the transport of ApoJ and ApoJ-AB1-40 complexes across the blood brain barrier, into the brain parenchyma. Another investigation (LaFerla et al., J Clin Invest 100:310-320 (1997)) showed that intracellular accumulation of ApoE is correlated with the presence of intracellular Aβ in the same cytoplasmic granules, suggesting that uptake of lipids may stabilize the hydrophobic Aβ protein within the cell. This work also suggested a role for LRP2 in the ApoE uptake. Thus, LRP2 appears to be involved in the transport and stabilization of the Aβ protein. In this respect, the interactions we have discovered between BAT3 APP, and BAT3 and LRP2, describe a biochemical link between APP and LRP2. We suggest that pharmacological modulation of the BAT3-LRP2 interaction might influence the transport and stabilization of the Aβ protein.

We also found that the same fragment of BAT3 (comprising amino acids 740 to 1040) interacts with a polypeptide comprising amino acids 11 to 361 of low density lipoprotein receptor-related protein-associated protein 1 (LRPAP1). LRPAP1 was first isolated as a 39 kDa component of the alpha 2-macroglobulin (A2M) receptor complex (Striekland et al., J Biol Chem 266:13364-13369 (1991)) and was called A2MRAP (for A2M receptor-associated protein), MRAP, or simply RAP. Subsequent studies (Korenberg et al., Genomics 22:88-93 (1994); Van Leuven et al., Genomics 25:492-500 (1995); Willnow et al., EMBO J 15:2632-2639 (1996); Willnow et al., Proc Natl Acad Sci USA 92:4537-4541 (1995)) showed that the human RAP gene is located on chromosome 4p16.3. RAP, which is predominantly found in the endoplasmic reticulum, binds LRP1 and LRP2 and functions as a chaperone protein that selectively protects endocytic receptors (such as LRPs) by binding to newly synthesized receptor polypeptides, thereby preventing ligand-induced aggregation and subsequent degradation in the ER. In the light of the interaction between BAT3 and LRP2 (described above), it is important to note that A2M (a ligand for LRP1 and LRP2) binds to the Aβ domain of APP (Hughes et al., Proc Natl Acad Sci USA 95:3275-3280 (1998)). Thus, our findings suggest that BAT3 is an adaptor molecule that brings together APP and the components of the LRP-RAP-A2M complexes. A recent study has shown that ligand binding to a receptor of the LDL receptor family triggers not only receptor internalization, but also initiates a signal transduction cascade (Trommsdorff et al., J Biol Chem 273:33556-33560 (1998)). Proteins such as Fe65 (APBB1(710)) and DAB bind to the cytoplasmic tails of LRP, the LDL receptor, and APP, where they can potentially serve as molecular scaffolds for the assembly of cytosolic multiprotein complexes. The interaction pattern of BAT3 (with APP, LRP2, and LRPAP1) suggests a similar role. We suggest that pharmacological modulation of the BAT3-LRP2 and BAT3-LRPAP1 interactions might affect the signal transduction cascade elicited by these receptor molecules, and in turn, control APP trafficking and metabolism.

We also found that the same fragment of BAT3 (comprising amino acids 740 to 1040) interacts with a polypeptide comprising 7 to 148 of transthyretin (prealbumin, amyloidosis type I) (TTR). TTR is responsible for the transport of the thyroid hormone thyroxine from the bloodstream to the brain, is very abundant in the CSF (25% of total CSF protein) and, in the central nervous system, is synthesized exclusively by the epithelial cells of the choroid plexus. The active form of TTR is a homotetramer. Even before the identification of the Aβ protein, TTR was identified as a component of the neuritic plaques, neurofibrillary tangles, and cerebral vessel amyloid deposits (Shirahama et al., Am J Pathol 107:41-50 (1982)). More recent studies have shown that TTR levels are reduced in the CSF of AD patients as compared to age-matched controls (Merched et al., FEBS Lett 425:225-228 (1998)), and TTR binding to Aβ inhibits amyloid fibrils in vitro (Schwarzman et al., Proc Natl Acad Sci USA 91:8368-8372 (1994)). Numerous variants in the transthyretin sequence are associated with various forms of amyloid polyneuropathy. Except for those formed within blood vessels, amyloid deposits are never found in the CNS. The interactions of BAT3 with APP, δ-adaptin (a lysosome targeting protein (See U.S. patent application Ser. No. 09/466,139 and International Patent Application No. PCT/US99/30396 (WO 00/37483)), glypican (a mediator of Aβ toxicity, described above), LRP2 (transport and stabilization of the Aβ protein, described above), and now with TTR, suggest a close involvement of BAT3 in AD pathogenesis. As with the BAT3-LRP2 interaction, we suggest that pharmacological modulation of the BAT3-TTR interaction might influence the transport and stabilization of Aβ.

In three separate experiments we found that a fragment of Fe65 (amyloid beta (Aβ) precursor protein-binding, family B, member 1, isoform E9 (710) or APBB1(710)) comprising amino acids 360 to 552 (the first phosphotyrosine binding domain, PTB), interacted with a novel protein. Three clones encoding this same novel protein were identified during the three separate experiments and all three contain a cDNA having a coding capacity of 289 amino acids and containing stop codons in the other two reading frames. Sequence analysis of the predicted amino acid sequence of the novel protein revealed the presence of a domain with high similarity to phosphatase 2C, from amino acids 78 to 289. Using a variety of methods (RACE, arrayed library screening, plaque lifts), we extended the sequence of the cDNA encoding the novel protein, and ultimately identified a cDNA sequence containing a open reading frame (ORF) coding for 372 amino acids. The nucleotide sequence of the cDNA encoding this novel protein, which we named PN7740, is provided as SEQ ID NO:1, and the amino acid sequence of the encoded protein is provided as SEQ ID NO:2. The putative ATG initiation codon is preceded by a purine (G) residue in position −3, and by several upstream STOP codons, suggesting that it represents the authentic initiation codon. At the end of the 3′ untranslated region (UTR), we found a canonical polyadenylation signal (AATAAA) shortly before the poly A itself. The phosphatase 2C domain of the PN7740 encompasses amino acids 104 through 339. Thus, we have identified a novel phosphatase that binds to the first PTB domain of Fe65/APBB 1 (710). This is notable because the balance of β-secretion versus α-secretion of APP is regulated by phosphorylation (Farber et al., J Neurosci 15:7442-7451 (1995); Caporaso et al., Proc Natl Acad Sci USA 89:3055-3059 (1992); Buxbaum et al., Proc Natl Acad Sci USA 87:6003-6006 (1990); Buxbaum et al., Proc Natl Acad Sci USA 90:9195-9198 (1993); Sabo et al., J Biol Chem 274:7952-7957 (1999)). We suggest that this balance can be modified by the pharmacological modulation of the interaction between Fe65 and the novel phosphatase, PN7740, or by the direct pharmacological modulation of the activity the PN7740 itself. It is also possible that this novel phosphatase modulates the phosphorylation status of proteins involved in APP metabolism, such as PS1, PS2, and nicastrin.

The amyloid beta (AP) precursor protein-binding, family A, member 1 (X11) protein (also called APBA1, Mint1, or adapter protein X11 alpha) is a cytosolic protein that interacts with the C-terminal portion of APP. APBA1 contains a PTB domain and a PDZ domain. Interaction of APBA1 with APP increases the levels of cellular APP and reduces the levels of both α- and β-secreted forms of APP (Borg et al., J Biol Chem 273:14761-14766 (1998)). The mechanism by which APBA1 affects APP metabolism is not clear at this point. In an effort to learn more about this we sought to identify interactors of APBA1. We found that a fragment of APBA1 from amino acids 447 to 758 interacted with a polypeptide comprising amino acids 364 to 589 of KIAA0427. The KDRI (Kazusa DNA Research Institute) database reports that the a full-length clone for KIAA0427 encodes 598 amino acid residues. However, since no well-characterized protein domains have been found among these 598 residues, the function of KIAA0427 is unknown. Consequently, we consider this protein as functionally novel, even though its sequence is not new. Importantly, the mRNA encoding KIAA0427 is found at very high levels in brain, medium levels in lung, kidney, prostate, testis, and ovary, and low levels in all other tissues examined. We suggest that KIAA0427 might mediate the effect of APBA1 on APP metabolism and that pharmacological modulation of the APBA1-KIAA0427 interaction might influence APP secretion.

Additional evidence for the role of APBA1 in APP metabolism comes from its interaction with PS1 which we identified. We found that a fragment of PSI comprising amino acid residuess 1 to 91 interacts with a fragment of of APBA1 comprising amino acids 471 to 822. This portion of APBA1 contains most of the PTB domain (amino acids 457 to 643), which is known to bind the cytoplasmic domain of APP. Thus, PSI and APP might compete for the PTB domain of APBA1 and familial Alzheimer's disease (FAD) associated mutations in PSI are expected to alter its interaction with APBA1. We suggest that pharmacological modulation of the PS1-APBA1 interaction might influence APP metabolism and amyloid production.

We also found that a fragment of APBA1 comprising amino acid residues 739 to 837 interacted with a fragment of glutamate ammonia ligase (GLUL, also called glutamate synthase, GLNS, or GS) comprising amino acid residues 49 to 212. GLUL catalyzes the ATP-dependent conversion of L-glutamate and NH3 to glutamine. In the brain, GLUL is secreted by astrocytes and plays a crucial role in the clearance of excitotoxic glutamate released in synapses. GLUL concentrations are dramatically increased in the CSF of AD patients (Gunnersen and Haley, Proc Natl Acad Sci USA 89:11949-11953 (1992)). This phenomenon could be a defense mechanism against glutamate excitotoxicity, reflecting astrogliosis rather than an Alzheimer specific phenomenon. It is striking that the Aβ peptide interacts with GLUL and inhibits its activity by oxidative modification (Aksenov et al., Free Radic. Res. 27:267-281 (1997)). Thus, the inactivation of GLUL by Aβ could lead to elevated concentration of excitotoxic glutamate. Furthermore, a previous study by the same group (Aksenov et al., J Neurochem 66:2050-2056 (1996)) showed that AB-mediated inactivation of GLUL is accompanied by the loss of immunoreactive GLUL and a concomitant significant increase of Aβ neurotoxicity. The interaction between GLUL and APBA1 suggests that APBA1 may act as an adapter molecule, bringing GLUL into a complex with APP. It is thus possible that APBA1 favors the oxidation of GLUL by AB, with a concomitant elevation in synaptic glutamate concentration. We suggest that pharmacological modulation of the APBA1-GLUL interaction could reduce its oxidation by Aβ and thus keep glutamate concentrations below toxic levels.

CASK is a postsynaptic protein of the MAGUK family, which contains a PDZ domain, an SH3 domain, a guanylate kinase domain, and a calmodulin-binding domain. It interacts with APBA1, with APP, and with the neurexins (Borg et al., J Biol Chem 273:31633-31636 (1998); Borg et al., J Neurosci 19:1307-1316 (1999)). In an effort to better address the potential rold of CASK in AD, we sought to identify its interactors. Using a fragment of CASK comprising amino acid residues 306 to 574 (encompassing the calmodulin-binding domain and PDZ domain), we identified and interacting polypeptide comprising amino acids 909 to 1280 of dystrophin (DMD). Dystrophin is largely known for its involvement in Duchenne muscular dystrophy (Hoffman, Arch Pathol Lab Med 123:1050-1052 (1999)), and was recently localized in post-synaptic densities in rat brain (Kim et al., Proc Natl Acad Sci USA 89:11642-11644 (1992)). Reciprocally, PSD-95 and DLG2 (also known as PSD-93) (Rafael et al., Neuroreport 9:2121-2125 (1998)) as well as APP (Askanas et al., Neurosci Lett 143:96-100 (1992)) are found at neuromuscular junctions, where they participate in the clustering of nicotinic acetylcholine receptors, a phenomenon that is known to require dystrophin (Kong & Anderson, Brain Res 839:298-304 (1999)). The interaction of dystrophin with CASK, together with its localization in post-synaptic densities withing the brain, suggests that this protein (and most probably other proteins from the dystrophin associated complex, like syntrophin) is another component of the synaptic cytoskeletal structure. Interestingly, both APP and dystrophin are found (often with gelsolin) in the pathological features of several neuromuscular diseases (De Bleecker et al., J Neuropathol Exp Neurol 55:563-577 (1996); Nonaka, Rinsho Shinkeigaku 34:1279-1281 (1994)). We suggest that adequate pharmacological modulation of the CASK-dystrophin interaction might help prevent the brain or neuromuscular synaptic degeneration observed in many neuropathological conditions.

Previously, we found that the calcium and integrin-binding protein (CIB) interacts with the PS1 interactor, FKBP25 (See U.S. patent application Ser. No. 09/466,139 and International Patent Application No. PCT/US99/30396 (WO 00/37483)). Based on its sequence similarity with calcineurin B, CIB was proposed to be the regulatory subunit of a yet-to-be-discovered calcium-activated phosphatase (Naik et al., J Biol Chem 272:4651-4654 (1997)). In a previous patent application, we suggested that this novel putative phosphatase might control the activity of the ryanodine receptor, and thus calcium homeostasis. Recently CIB was also found to also interact with PS1 and PS2 (Stabler et al., J Cell Biol 145:1277-1292 (1999)). Because of the causal role of PSI and PS2 mutations in AD, proteins that interact with CIB are likely to play a role in AD pathogenesis. The amyloid beta (A4) precursor protein-binding, family A, member 2 (X11-like) protein (APBA2) (also called Mint2 or X11 beta) is a cytosolic protein that interacts that the cytosolic C-terminus of APP (Tomita et al., Proc Natl Acad Sci USA 94:2025-2030 (1999)). APBA2 contains a PTB domain and two PDZ domains. In addition tointeracting with the cytosolic portion of APP, APBA1 (Mint1) and APBA2 (Mint2) both bind Munc-18, and are involved in the fusion of synaptic vesicles with the presynaptic membrane (Okamoto & Sudhof, J Biol Chem 272:31459-31464 (1997); Okamoto & Sudhof, Eur J Cell Biol 77:161-165 (1998)). Thus, the APBA proteins likely play a role in APP trafficking and synaptic function. Proteins that associate with the APBA proteins are therefore also likely to be involved in AD pathogenesis. Moreover, proteins that associate with CIB and with APBA1 or APBA2 are even more likely to play a central role in AD development. Thus, we sought to identify additional interactors of CIB and the APBA proteins. We found that the site-1 protease (S1P) interacts with both CIB and APBA2.

Site-1 protease (SIP) is a transmembrane protease that catalyzes the first cleavage step of the sterol regulatory element-binding protein (SREBP) processing (Sakai et al., Mol Cell 2:505-514 (1998)). SREBPs are membrane-bound transcription factors that activate the genes for enzymes involved in cholesterol and fatty acids biosynthesis (Brown & Goldstein, Proc Natl Acad Sci USA 96:11041-11048 (1999)). Two sequential cleavage steps are necessary to release the active N-terminal domain of SREBPs from endoplasmic reticulum (ER) membranes, and for the subsequent targeting of this protein domain to the nucleus. The first step in this two-step process is catalyzed by S1P, which cleaves SREBPs in the ER luminal domain, while the second step is catalyzed by Site 2 Protease (S2P), which cleaves the SREBPs in the first transmembrane domain (Rawson et al., Mol Cell 1:47-57 (1997); Ye et al., Proc Natl Acad Sci USA 97:5123-5128 (2000)). The entire process is controlled by the SREBP cleavage-activating protein (SCAP), a large regulatory protein with eight transmembrane domains, that acts as a sterol sensor and is required for the activation of the S1P protease (Nohturfft et al., Proc Natl Acad Sci USA 96:11235-11240 (1999)). This important protein is also known as SKI-1. In addition to SREBPs, S1P/SKI-1 belongs to the subtilisin/kexin family of precursor convertases (Seidah et al., Ann N Y Acad Sci 885:57-74 (1999)), and also cleaves the proBDNF molecule into its active form (Seidah et al., Proc Natl Acad Sci USA 96:1321-1326 (1999). Because CIB interacts with both PSI and PS2, and because APBA2 interacts with APP, S1P might well be involved in APP processing. However, it appears unlikely that S1P is the γ-secretase since it does not cleave in the transmembrane domain but in the luminal domain, and since there is now mounting evidence that PS1 could be the γ-secretase (Wolfe et al., Nature 398:513-517 (1999); Selkoe and Wolfe, Proc Natl Acad Sci USA 97:5690-5692 (2000); Li et al., Proc Natl Acad Sci USA 97:6138-6143 (2000); Li et al., Nature 405:689-694 (2000)), although this is still a matter of controversy (Murphy et al., J Biol Chem 275:26277-26284 (2000); Murphy et al., J Biol Chem 274:11914-11923 (1999)). Recently, two novel enzymes with β-secretase activity have been identified as BACE and BACE-like (Vassar et al., Science 286:735-741 (1999); Hussain et al., Mol Cell Neurosci 14:419-427 (1999); Yan et al., J Biol Chem 274:2145-2156 (1999)). It is thus unlikely that S1P represent yet a third enzyme with β-secretase activity. However, we favor the possibility that S1P might be an α-secretase. Although the exact site of APP α-cleavage is immediately after the Lys16 residue of the Aβ peptide (Anderson et al., Neurosci Lett 128:126-128 (1991)), mutational analyses have shown that a-secretase has poor sequence specificity (substitution of Lys16 by a Gly, Leu, Thr, Arg, or Met residue did not affect cleavage) (Sisodia, Proc Natl Acad Sci USA 89:6075-6079 (1992)) but cleaves at a distance of about 12 to 13 residues away from the membrane. Interestingly, the cleavage of SREBP2 by S1P occurs immediately after the Leu522 residue, which is 12 residues before the second transmembrane domain (Duncan et al., J Biol Chem 272:12778-12785 (1997)). Additionally, it is also remarkable that S1P activity regulates (and is regulated by) cholesterol levels (Brown & Goldstein, Proc Natl Acad Sci USA 96:11041-11048 (1999)), and that raised cholesterol levels reduce the α-secretion of APP (Bodovitz & Klein, J Biol Chem 271:4436-4440 (1996)). Conceivably, high cholesterol levels could lower S1P activity, thus reducing APP α-secretion. In brief, we have identified a transmembrane protease, S1P, which interacts with CIB and APBA2 and might be involved in APP metabolism, and which shows several important features expected from a putative α-secretase. We suggest that adequate pharmacological modulation of S1P activity or modulation of its interaction with CIB or APBA2 might shift the metabolism of APP toward the α-secretase pathway, thereby reducing amyloid production.

There is a growing body of evidence that disruption of energy metabolism is an important factor in neurodegenerative disorders, including AD (Beal, Biochem Biophys Acta 1366:211-223 (1998); Nagy et al., Acta Neuropathol 97:346-354 (1999); Rapoport et al., Neurodegeneration 5:473-476 (1996)). Mitochondrial dysfunction results in low ATP levels and production of free oxiradicals that are extremely toxic to neurons (Simonian & Coyle, Annu Rev Pharmacol Toxicol 36:83-106 (1996); Beal, Curr Opin Neurobiol 6:661-666 (1996)). Mutations in PS1 associated with Familiar Alzheimer's Disease (FAD) have been shown to ultimately trigger neuronal apoptosis through a mechanism involving the disruption of mitochondrial function, energy metabolism, and calcium homeostasis (Guo et al., Nat Med 4:957-962 (1998); Guo et al., J Neurosci Res 56:457-470 (1999); Mattson et al., J Neurosci 20:1358-1364 (2000); Begley et al., J Neurochem 72:1030-1039 (1999)). To gain further insight into the involvement of mitochondrial function and energy metabolism in AD pathogenesis, we sought to find interactors of the presenilins (PS1 and PS2), as well as their common interactor CIB (Stabler et al., J Cell Biol 145:1277-1292 (1999)), which are either mitochondrial proteins, or are involved in energy metabolism. We were encouraged by our previous discovery of the interaction between PS-1 and α-enolase, a glycolytic enzyme that transforms 2-phosphoglycerate into phosphoenol pyruvate, and is thus directly involved in energy production (see U.S. patent application Ser. No. 09/466,139; International Patent Application No. PCT/US99/30396 (WO 00/37483)).

Using a fragment of PS1 comprising amino acid residues 1 through 91, we identified an interacting polypeptide comprising the N-terminus (amino acid residues 1-266) of phosphoglycerate dehydrogenase (PGAD). PGAD is responsible for the oxidation of 3-phosphoglycerate, an intermediate in glycolysis, to 3-phosphohydroxypyruvate, an intermediate of the serine biosynthetic pathway. We also discovered that both PS1 (amino acid residues 1-91) and PS2 (amino acids 1-97) interact with an N-terminal fragment (amino acids 2-190) of glyceraldehyde-3-phosphate dehydrogenase (GAPD). GAPD catalyzes the oxidation of glyceraldehyde-3-phosphate to 1,3-diphosphoglycerate, with the concomitant reduction of NAD+ to NADH. In addition to its role as a glycolytic enzyme, GAPD is directly involved in neuronal apoptosis (Chen et al., Mol Biol Cell 9:3241-3257 (1999)). Its potential involvement in AD pathogenesis is further strengthened by its previously demonstrated interaction with the cytosolic domain of APP (Schulze et al., J Neurochem 60:1915-1922 (1993)). In brief, GAPD is an important enzyme that interacts with all three major Alzheimer proteins (PS1, PS2, and APP), mediates neuronal apoptosis, and is involved in energy metabolism.

We also found that the N-terminal portion (amino acid residues 1-91) of PS1 interacts with a fragment (amino acids 31-242) of the beta subunit of the electron transfer flavoprotein (beta-ETF or EFTB). EFTB is an electron acceptor for several dehydrogenases and transfers electrons to the main respiratory electron transport chain. A disruption of the interaction between PS1 and EFTB (possibly caused by FAD mutations) might alter normal mitochondrial function and energy production, and thus threaten neuronal survival.

Additionally, we also found that fragments comprising amino acid residues 1-191, or 1-137 of CIB interacted with several polypeptides comprising fragments of the beta subunit of ATP synthase, ATPMB. As mentioned above, CIB is a calcium-binding protein that interacts with both PS1 and PS2 (Stabler et al., J Cell Biol 145:1277-1292 (1999)), and with FKBP25, another PS1 interactor that might also be involved in the regulation of calcium homeostasis (see U.S. patent application Ser. No. 09/466,139; International Patent Application No. /US99/30396, (WO 00/37483)). Collectively, the interactions disclosed herein link PS1, PS2, and CIB to several proteins involved in mitochondrial function and energy metabolism—two cellular processes that are severely affected in AD and other neurodegenerative diseases. We suggest that adequate pharmacological modulation of these interactions, or modulation of the enzymatic activities of the identified preys, might prevent, or greatly reduce, the neuronal degeneration observed in AD.

Intracellular calcium is stored mainly inside the endoplasmic reticulum (ER), and is released into the cytosol upon activation of the ryanodine receptor or the inositol-triphosphate (IP3) receptor, both of which are ER transmembrane proteins. The fine regulation of the activities of these two receptors is crucial for the control of calcium homeostasis, and thus for neuronal survival (Mattson & Furukawa, Restor Neurol Neurosci 9:191-205 (1996)). A number of studies suggest that disruption of calcium homeostasis underlies Aβ neurotoxicity (Mattson, Ann NY Acad Sci 747:50-76 (1994)); Joseph & Han, Biochem Biophys Res Commun 184:1441-1447 (1992); Mattson et al., Trends Neurosci 16:409-414 (1993); Guo et al., J Biol Chem 273:12341-12351 (1998)). In addition to their role in the production of AB42, the presenilins are also known to participate in the control of calcium homeostasis through the regulation of calcium release from internal stores (Mattson et al., J Neurochem 70:1-14 (1998); Mattson et al., Ann NY Acad Sci 893:154-175 (1999)). AD-associated mutations in the presenilins have been shown to disrupt this control, leading to neuronal apoptosis (Guo et al., J Biol Chem 273:12341-12351 (1998); Guo et al., Neuroreport 8:379-383 (1996)). PS1 was shown to interact with δ-catenin (Guo et al., J Biol Chem 273:12341-12351 (1998); Guo et al., Neuroreport 8:379-383 (1996)), but the functional significance of this interaction has remained elusive. We have found that 6-catenin interacts with KIAA0443, a protein that contains a lipocalin domain and is thus probably involved in the transport of small lipophilic molecules (See U.S. patent application Ser. No. 09/466,139; International Patent Application No. PCT/U99/30396 (WO 00/37483)). KIAA0433, is now known as G protein-coupled receptor-associated sorting protein, or GASP.

We have now discovered that a polypeptide comprising amino acid residues 901-1200 of GASP, interacts with a polypeptide comprising amino acid residues 567-854 of the enzyme phosphatidylinositol-4 kinase (PI4K). PI4K catalyzes the first commited step in the biosynthesis of IP3, and is reportedly expressed primarily in brain and placenta (Wong et al., Oncogene 9:3057-3061 (1994)). P14K contains several biologically active domains, including an ankyrin repeat domain, a lipid kinase unique domain, a pleckstrin homology domain, a presumed lipid kinase/protein kinase homology domain, a proline-rich region, and an SH3 domain (Nakagawa et al., J Biol Chem 271:12088-12094 (1996)). The interaction of KIAA0443/GASP with PI4K and the presence of a lipocalin domain in KIAA0443/GASP suggest that KIAA0443/GASP might bring a lipid such as phosphatidylninositol in close proximity to the kinase that phosphorylates it. Regulation of this process, which leads to the formation of IP3, is obviously important for the control of calcium homeostasis. Because KIAA0443/GASP interacts with δ-catenin, which is itself a PS1 interactor, it is possible that mutations in PS1 associated with AD disrupt the interaction network that includes PS1, δ-catenin, KIAA0443/GASP, and PI4K. This, in turn, could lead to an alteration of PI4K activity, resulting in abnormal levels of IP3 and the disruption of calcium homeostasis. We suggest that pharmacological modulation of PI4K activity, or modulation of the protein-protein interactions connecting this enzyme with PS1 (via KIAA0443/GASP and δ-catenin), might prevent the disruption of calcium homeostasis and the resulting neuronal apoptosis.

We also found that the same polypeptide fragment of KIAA0443/GASP interacts with a polypeptide comprising amino acid residues 27-132 of the serotonin receptor 2A (5HT-2A). Interestingly, the 5HT-2A and 5HT-2C receptors stimulate APP α-secretion, thus precluding Aβ formation (Nitsch et al., J Biol Chem 271:4188-4194 (1996)). Moreover, the serotonin derivative N-acetylserotonin, as well as melatonin, were shown to improve cognition and protect neurons from Aβ toxicity (Bachurin et al., Ann N Y Acad Sci 890:155-166 (1999)). These findings suggest that 5HT-2A agonists might prevent amyloid formation, as well as protect neurons from the Aβ peptide already present. KIAA0443/GASP appears to link the δ-catenin network (which includes the presenilins) to the serotoninergic system, thus opening a novel and promising therapeutic avenue. We suggest that pharmacological modulation of the 5HT-2A and its interaction with KIAA0443/GASP might prevent amyloid formation and might protect neurons from Aβ toxicity.

We previously reported an interaction between APP and KIAA0351, and we suggested that KIAA0351 might mediate the neurotrophic effects of APP through its pleckstrin homology (PH) domain and a connection to guanine nucleotide exchange factors (GEFS) and cyclic GMP (see U.S. patent application Ser. No. 09/466,139; International Patent Application No. PCT/US99/30396 (WO 00/37483)). We have now found that a portion of KIAA0351 comprising amino acid residues 301-557 interacts with a polypeptide comprising amino acid residues 475-733 of the triple function protein, TRIO. TRIO, which was initially identified as an interactor for LAR, a transmembrane receptor with tyrosine phosphatase activity (Debant et al., Proc Natl Acad Sci USA 93:5466-5471 (1996)), is a large protein (2861 aa) that contains two pleckstrin homology (PH) domains, one SH3 domain, and a protein kinase domain. Interestingly, all of these functional domains are clustered in the C-terminal half of the protein. Additionally, TRIO contains two guanine nucleotide exchange factor (GEF) domains; one rac-specific, and the other rho-specific (Debant et al., Proc Natl Acad Sci USA 93:5466-5471 (1996)). TRIO also contains an Ig-like domain (close to its kinase domain in the C-terminal region), and four spectrin repeats (in the N-terminal region).

Thus, we have found that APP interacts directly with the transmembrane receptor tyrosine phosphatase, PTPZ (see U.S. patent application Ser. No. 09/466,139; International Patent Application No. PCT/US99/30396 (WO 00/37483)), and indirectly (through the KIAA0351 and TRIO connection) with the transmembrane receptor tyrosine phosphatase, LAR. The neurotrophic and neuroprotective effects of sAPP are well documented (Jin and Saitoh, Drugs Aging 6:136-149 (1995); Mattson, Physiol Rev 77:1081-1132 (1997); Saitoh et al., Research Advances in Alzheimer's Disease and Related Disorders (Iqbal et al. eds), pp 693-699, New York: John Wiley & Sons Ltd. (1995); Mattson et al., Ann NY Acad Sci 893:154-175 (1999); Mattson and Duan, J Neurosci Res 58:152-166 (1999)). In this respect, it is important to note that Abl, TRIO, LAR, and other associated proteins are involved in axonal development (Lanier and Gertler, Curr Opin Neurobiol 10:80-87 (2000)). A more recent study also showed that downregulation of LAR activity prevents apoptosis and increases NGF-induced neurite outgrowth (Yeo et al., J Neurosci Res 47:348-360 (1997)). Together with the recent observation that pleiotrophin binding to PTPZ inhibits its activity (Meng et al., Proc Natl Acad Sci USA 97:2603-2608 (2000)), these results suggest that inhibition of receptor tyrosine phosphatase activity is a key element underlying the neurotrophic or neuroprotective effects of secreted factors such as sAPP. We suggest that pharmacological modulation of LAR activity, or modulation of its interaction with TRIO, or modulation of the TRIO interaction with KIAA0351, might potentiate the neuroprotective effect of sAPP.

As described above, CIB is a calcium-binding protein that we found interacts with FKBP25, which is itself a PS1 interactor (see U.S. patent application Ser. No. 09/466,139; International Patent Application No. PCT/US99/30396 (WO 00/37483)). Based upon its sequence similarity with calcineurin B, CIB was proposed to be the regulatory subunit of a yet-to-be-discovered calcium-activated phosphatase (Naik et al., J Biol Chem 272:4651-4654 (1997)). We have suggested that this novel putative phosphatase might control the activity of the ryanodine receptor, and thus calcium homeostasis (see U.S. patent application Ser. No. 09/466,139; International Patent Application No. PCT/US99/30396 (WO 00/37483)). Recently, CIB was also found to interact with PS2 and PS1 (Stabler et al., J Cell Biol 145:1277-1292 (1999)). Because of the causal role of PS1 and PS2 mutations in AD, proteins that interact with CIB are likely to play a major role in AD pathogenesis. We have now found that CIB interacts with a polypeptide comprising amino acid residues 305-549 of the mixed lineage kinase 2 (MLK2), which is now known as mitogen-activated protein kinase kinase kinase 10, alt. transcript (954), or simply MAP3K10.

MAP3K1O/MLK2 was originally cloned from human epithelial tumors and described as protein kinases containing two leucine/isoleucine-zipper domains (Dorow et al., Eur J Biochem 213:701-710 (1993)). In another study, MAP3K10/MLK2 is called MST and described as a kinase of 953 aa, with an SH3 domain, 2 leucine zipper domains, and a proline-rich domain (Katoh et al., Oncogene 10:1447-1451 (1995)). Northern blot data showed that the gene is mostly expressed in brain, skeletal muscle, and testis as a 3.8-kb mRNA. Interestingly, MAP3K1O/MLK2-mediated signaling is activated by polyglutamine-expanded huntingtin, the pathogenic form of the protein found in Huntington's disease (Liu et al., J Biol Chem 275:19035-19040 (2000)). Thus, MAP3K10/MLK2 appears to mediate neuronal toxicity in under certain circumstances. Because MAP3K10/MLK2 interacts with CIB, it is possible that mutations in the presenilins also activate MAP3K10/MLK2, resulting in accelerated neuronal apoptosis, as observed in AD. We suggest that pharmacological modulation of MAP3K10/MLK2 activity, or modulation of its interaction with CIB, might prevent neuronal death.

BCL2-associated X protein, isoform beta (218) (also known as BAX-beta) is a member of the Bcl-2 family that mediates apoptosis. Elevated BAX-beta concentrations in the brains of AD patients suggested that BAX-beta might be responsible, at least in part, for the neuronal death observed in AD (Su et al., J Neuropathol Exp Neurol 56:86-93 (1997)). In order to better understand the role of BAX-beta in AD pathogenesis we set out to identify the proteins with which it interacts. We found that a fragment of BAX-beta comprising amino acid residues 50-107 interacted with a fragment comprising amino acid residues 643-993 of alpha (pore-forming) subunit of the slo (K+ activated) potassium channel (now known as potassium large conductance calcium-activated channel, subfamily M, alpha member 1, or simply KCNMA1). Potassium channels (K channels) are very diverse in structure and function (Jan & Jan, Curr Opin Cell Biol 9:155-160 (1997); Christie, Clin Exp Pharmacol Physiol 22:944-951 (1995)). The slo channel (named for the Drosphila K channel, slowpoke) is a member of the subfamily of large-conductance calcium activated potassium channels (also called Maxi K, BK, or KCa), which belong to the voltage gated K channel (Kv) family. The BK subfamily contains many splice variants, all of which have the typical structure of Kv channels: the alpha subunit is a homotetrameric complex formed by 4 polypeptides, each of which contains 6 transmembrane™ domains and often-large cytosolic N-terminal and C-terminal domains. The channel (pore) region is located between TM5 and TM6, while TM4 acts as a voltage sensor, and calcium-binding sites are found in the C-terminal cytosolic domain. Tetraethyl-ammonium (TEA) blocks the activity of these channels (Jan & Jan, Curr Opin Cell Biol 9:155-160 (1997); Christie, Clin Exp Pharmacol Physiol 22:944-951 (1995)). A dysfunction of a large conductance TEA-sensitive K channel was identified in fibroblast from AD patients (Etcheberrigaray et al., Proc Natl Acad Sci USA 90:8209-8213 (1993)). Recently, the same channels were found to be activated in response to sAPP, resulting in shut down of neuronal activity and protection against a variety of insults, including Aβ toxicity (Furukawa et al., Nature 379:74-78 (1996); Goodman & Mattson, Brain Res 706:328-332 (1996)). Thus, our finding shows that BAX-beta, a mediator of apoptosis, interacts with the slo K channel KCNMA1, which is involved in the neuroprotective effect of sAPP, and whose activity is disrupted in fibroblasts from AD patients. We suggest that pharmacological modulation of the slo K channel activity, or modulation of the interaction between KCNMA1 and BAX-beta, might prevent neuronal apoptosis.

Previously, we reported an interaction between δ-catenin (CTNND2) and the focal adhesion kinase 2 (FAK2), also called proline-rich tyrosine kinase 2 (PYK2) or cell adhesion kinase β (CAKO) (see U.S. patent application Ser. No. 09/466,139; International Patent Application No. PCT/US99/30396 (WO 00/37483) and see below). Focal adhesion kinases (FAKs) form a special subfamily of cytoplasmic protein tyrosine kinases (PTKs). In contrast to other non-receptor PTKs, FAKs do not contain SH2 or SH3 domains, but instead have a carboxy-terminal proline-rich domain, which facilitates protein-protein interactions (Schaller, Soc Gen Physiol Ser 52:241-255 (1997); Schaller & Parsons, Curr Opin Cell Biol 6:705-710 (1994); Parsons et al., J Cell Sci 18:109-113 (1994)). FAK2 is expressed at its highest levels in brain, at medium levels in kidney, lung, and thymus, and at low levels in spleen and lymphocytes (Avraham et al., J Biol Chem 270:27742-27751 (1995)). In the brain, FAK2 is found at its highest levels in the hippocampus and the amygdala (Avraham et al., J Biol Chem 270:27742-27751 (1995)), two areas severely affected in AD. FAK2 is thought to participate in signal transduction mechanisms elicited by cell-to-cell contacts (Sasaki et al., J Biol Chem 270:21206-21219 (1995)). It is involved in the calcium-induced regulation of ion channels, and it is activated by the elevation of intracellular calcium concentration following the activation of G protein-coupled receptors (GPCRs) that signal though Gaq and the phospholipase C (PLC) pathway (Yu et al., J Biol Chem 271:29993-29998 (1996)). Thus, FAK2 is an important intermediate signaling molecule between GPCRs activated by neuropeptides or neurotransmitters and downstream signals that modulate the neuronal activity (channel activation, membrane depolarization). Such a link between intracellular calcium levels, tyrosine phosphorylation, and neuronal activity is clearly important for neuronal survival and synaptic plasticity (Siciliano et al., J Biol Chem 271:28942-28946 (1996)). The previously-reported interaction of FAK2 with δ-catenin, along with its high levels of expression in hippocampus and amygdala suggest that a disruption of FAK2 activity may be related to neuronal death in AD.

To gain more insight into the mechanism by which FAK2 mediates neuronal functions and neuronal survival, we sought to identify the proteins that interact with FAK2. We found that a polypeptide fragment of FAK2 comprising amino acid residues 673-866 interacted with a fragment of the type-1 sulfonylurea receptor, SUR1, comprising amino acid residues 121-270. SUR1 is also known as the ATP-binding cassette transporter, sub-family C (CFTR/MRP), member 8, or simply ABCC8. Two types of sulfonylurea receptors, SUR1 and SUR2, constitute the regulatory unit of ATP-sensitive inward rectifying potassium channels (K_{ATP} channels), while the channel-forming unit belongs to the Kir6.x family (Bryan et al., Biochem Biophys Acta 1461:285-303 (1999); Inagaki I Seino, Jpn J Physiol 48:397:412 (1998)). A major role played by these channels is the linking of the metabolic state of a cell to its membrane potential: K_{ATP} channels close upon binding intracellular ATP to depolarize the cell, and open when ATP concentrations return to resting levels. These channels are involved in events such as insulin secretion from pancreatic b cells, ischemic responses in cardiac and cerebral tissues, and regulation of vascular smooth muscle tone (Inagaki et al., Science 270:1166-1170 (1995)); Ashcroft & Ashcroft, Biochem Biophys Acta 1175:45-49 (1992)). The activity of these channels in pancreatic b cells, where they play a crucial role in the secretion of insulin, has been extensively studied: following an elevation of blood glucose levels, the intracellular concentration of ATP in pancreatic b cells rises, resulting in channel closure and cell depolarization. This allows Ca²⁺ ions to enter the cell through voltage-sensitive Ca²⁺ channels, which triggers the fusion of insulin secretory vesicles with the plasma membrane, and the subsequent release of insulin (Satin, Endocrine 4:191-198 (1996); Ashcroft, Horm Metab Res 28:456-463 (1996)). In neurons the same mechanisms involving K_{ATP} channels (linking the metabolic state of the cell to its membrane potential) control neurotransmitter release.

Previously, we also reported an interaction between acetylcholinesterase and α-endosulfine (ALPEND), which is an endogenous ligand for SUR1/ABCC8 (Virsolvy-Vergine et al., Proc Natl Acad Sci USA 89:6629-6633 (1992)) (see U.S. patent application Ser. No. 09/466,139; International Patent Application No. PCT/US99/30396 (WO 00/37483)). Because of its role in pancreatic beta cells, where is stimulates insulin secretion (Heron et al., Proc Natl Acad Sci USA 95:8387-8391 (1998)), we suggested that in the brain, endosulfine binding to the sulfonylurea receptor would also shut down K_{ATP} channels, leading to depolarization, Ca²⁺ entry, vesicle fusion, and release of the vesicular content into the synaptic cleft. While the activity of K_{ATP} channels is down-regulated by ATP binding to the SUR subunit, phosphorylation of the Kir6.x subunit by PKA stimulates channel activity (Lin et al., EMBO J 19:942-955 (2000)). Interestingly, endosulfine is also a PKA substrate (Virsolvy-Vergine et al., Proc Natl Acad Sci USA 89:6629-6633 (1992); Heron et al., Proc Natl Acad Sci USA 95:8387-8391 (1998); Heron et al., Diabetes 48:1873-1876 (1999)). The interaction of SUR1/ABCC8 with FAK2 suggests that additional phosphorylation events (of any of the channel subunits) might control channel activity. K_{ATP} channels are very amenable to pharmacological modulation and drugs that activate the channels (i.e., K⁺ channels openers (PCOs) such as diazoxide and cromakalim) or inhibit the channels (i.e., K⁺ channels blockers (PCBs) such as the sulfonylureas glibenclamide and tolbutamide) have been identified (Lawson, Pharmacol Ther 70:39-63 (1996); Lawson, Clin Sci 91:651-663 (1996)). The function of K_{ATP} channels in the brain is under intense investigation (Zawar et al., J Physiol 514:327-341 (1999)), and the expression of different K_{ATP} channels in the hippocampus opens a therapeutic opportunity against hippocampal neurodegeneration. In fact, the PCO cromakalim was shown to protect neurons in the hippocampus from glutamate toxicity through a mechanism closely related to the control of calcium homeostasis (Lauritzen et al., J Neurochem 69:1570-1579 (1997)). A more recent study showed that K_{ATP} channels are neuroprotective against the effects cellular stress caused by energy depletion (Lin et al., EMBO J 19:942-955 (2000)). Both calcium homeostasis and energy metabolism are crucial cellular functions that are very affected in neurodegenerative diseases such as AD. We suggest that pharmacological modulation of brain K-channels containing SUR1/ABCC8, or modulation of the interaction between SUR1/ABCC8 and FAK2, might slow or help prevent the neuronal loss observed in the brains of AD patients.

Cyclic GMP (cGMP) is a small molecule involved in a number of cellular functions that relate to neuronal survival or neuronal death. There is evidence that intracellular cGMP mediates some of the neurotrophic effects of sAPP (Barger et al., J Neurochem 64:2087-2096 (1995)), as well as the neuroprotective action of somatostatin (Forloni et al., J Neurochem 68:319-327 (1997)). However, there is also evidence that intracellular cGMP is neurotoxic while extracellular cGMP is neuroprotective (Montoliu et al., Neuropharmacology 38:1883-1891 (1999)). Recently, Chalimoniuk and Strosznajder looked at the effects of aging and the Aβ peptide on nitric oxide (NO) and cGMP signaling in the hippocampus (Chalimoniuk & Strosznajder, Mol Chem Neuropathol 35:77-95 (1998)). They showed that aging coincided with a decrease in the basal level of cGMP as a consequence of a more active degradation of cGMP by a phosphodiesterase in the aged brain as compared to the adult brain. Moreover, a loss of the NMDA receptor-stimulated enhancement of the cGMP level determined in the presence of cGMP-phosphodiesterase inhibitor 3-isobutyl-1-methylxanthine was observed in hippocampus and cerebellum of aged rats. The neurotoxic AB25-35 peptide decreased significantly the NMDA receptor-mediated calcium, and calmodulim-dependent NO synthesis that may then be responsible for disturbances of the NO and cGMP signaling pathway. Chalimoniuk and Strosznajder concluded that cGMP-dependent signal transduction in hippocampus and cerebellum may become insufficient in senescent brain and may have functional consequences in disturbances of learning and memory processes, and that the Aβ peptide may be an important factor in decreasing the NO-dependent signal transduction mediated by NMDA receptors, resulting in decreased cGMP levels. Thus, the effects of cGMP are quite complex and branch into other pathways such as nitric oxide (NO), NMDA receptor, and calcium homeostasis. The growing evidence for a neuroprotective effect of cGMP (Barger et al., J Neurochem 64:2087-2096 (1995); Forloni et al., J Neurochem 68:319-327 (1997); Chalimoniuk & Strosznajder, Mol Chem Neuropathol 35:77-95 (1998)) suggests that inhibition of a cGMP-specific phosphodiesterase such as PDE-9A might prove beneficial. Importantly, we found that an amino-terminal fragment of APBA2, comprising amino acid residues 1-210, interacted with a carboxyl-terminal portion of phosphodiesterase 9A (PDE9A), comprising amino acid residues 269-593. mRNA encoding PDE9A has been found in all tissues examined, with highest levels in spleen, small intestine, and brain (Fisher et al., J Biol Chem 273:15559-15564 (1998)). Because PDE9A interacts directly with a protein from the APP pathway (APBA2), and because cGMP mediates at least some of the neurotrophic effects of sAPP (Barger et al., J Neurochem 64:2087-2096 (1995)), we suggest that pharmacological modulation of PDE9A activity, or modulation of its interaction with APBA2, might potentiate the neurotrophic effects of sAPP and prevent neuronal death observed in the brains of AD patients.

Further investigations of the binding partners of full-length CIB revealed an interaction with a polypeptide comprising amino acid residues 320-359 of the stearoyl-CoenzymeA desaturase 1 (SCD1, also called delta(9)-desaturase). SCD1 is a component of the liver microsomal stearoyl-CoA desaturase system that catalyzes the insertion of a double bond into various fatty acyl-CoA substrates. SCD1 needs iron as a cofactor and is localized in the endoplasmic reticulum. In the peripheral nervous system, SCD2 is involved in lipid biosynthesis associated with myelinogenesis (Garbay et al., J Neurochem 71:1719-1726 (1998)). Its function in the brain is less clear, as its expression pattern through development does not coincide well with that of true myelin genes (Garbay et al., Dev Brain Res 98:197-203 (1997)). Still, SCD1's function in lipid biosynthesis appears to be compatible with a role in myelination. The interaction we discovered between CIB and SCD1 suggests that the metabolic disorder leading to the formation of amyloid plaques and neurofibrilary tangles, neuronal and synaptic loss, could also downregulate SCD activity, and, in turn, result in demyelination, as observed in the brains of AD patients. Thus, we propose that pharmacological modulation of SCD 1 or its interaction with CIB might prevent the myelin loss observed in AD brain and other neurodegenerative conditions.

Previously, we described the interaction between PS1 and rab11, a small GTPase involved in the traffic of intracellular vesicles (see U.S. patent application Ser. No. 09/466,139; International Patent Application No. PCT/US99/30396 (WO 00/37483)). Rab11 is found predominantly in recycling endosomes (Ullrich et al., J Cell Biol 135:913-924 (1996); Sheff et al., J Cell Biol 145:123-139 (1999)). It also plays a role in the transport of vesicles from the trans-Golgi network to the plasma membrane and in secretory mechanisms in PC12 cells (Urbe et al., FEBS Lett 334:175-182 (1993); Chen et al., Mol Biol Cell 9:3241-3257 (1998)). These observations confirm the role of PS1 in vesicular trafficking. In an effort to further define the role of rab11 a in AD pathogenesis, we sought to identify the proteins with which it interacts. We discovered that a rab11a fragment comprising amino acid residues 1-137 interacted with a polypeptide fragments from the carboxyl-terminus of the focal adhesion kinase (FAK). FAK is a tyrosine kinase found at focal adhesion sites, and which mediates the signals elicited by a variety of hormone and neurotransmitter receptors (Schaller & Parsons, Curr Opin Cell Biol 6:705-710 (1994); Parsons et al., J Cell Sci 18:109-113 (1994); Zachary, Int J Biochem Cell Biol (1997); Schlaepfer et al, Prog Biophys Mol Biol 71:435-478 (1999)). These signals are involved in the control of a number of cellular events including cell growth, migration, and survival. In neurons, FAK is also involved in neurite extension (Park et al., J Biol Chem 275:19768-19777 (2000)). In addition to its role in neuronal survival and synaptic stability (Girault et al., Trends Neurosci 22:257-263 (1999); Tamura et al., J Biol Chem 274 (1999)), FAK activity is known to be disrupted by the Aβ protein (Zhang et al., J Biol Chem 269:25247-25250 (1994); Berg et al., J Neurosci Res 50:979-989 (1997)). Thus, we have identified a tyrosine kinase whose activity is important for neuronal survival and function, and which interacts that rab 11 a, a protein involved in vesicular trafficking and which binds to PS1. It is thus possible that mutations in PS1 associated with FAD might alter FAK activity and thus disrupt neuronal function and survival.

To gain additional information about the involvement of FAK in neurodegenerative diseases in general, and AD in particular, we set out to identify the proteins with which FAK interacts. We discovered that a fragment of FAK comprising amino acid residues 724-1052 interacted with a portion of casein kinase II, alpha 2 (CSNK2A2) comprising amino acid residues 264-351. As mentioned above, there is a large body of evidence that phosphorylation cascades are deeply altered in the brains of AD patients (Jin & Saitoh, Drugs Aging 6:136-149 (1995); Saitoh et al., Lab Invest 64:596-616 (1991); and Farlow, Am J Health SystPharm 55 Suppl 2:S5-S1O (1998)). Among the numerous kinases that are affected in AD, CSNK2A2 levels showed a dramatic overall reduction (84%), although CSNK2A2 levels varied considerably between sick (tangle-bearing) neurons and healthy (tangle-free) neurons (limoto et al., Brain Res 507:273-280 (1990)). In addition, although CSNK2A2 is not part of the paired helical filaments (PHF), it is clearly associated with neurofibrillary tangles (Baum et al., Brain Res 573:126-132 (1992)). Since CSNK2A2 alterations were shown to precede tau accumulation and tangle formation (Masliah et al., Am J Pathol 140:263-268 (1992)), it was suggested that CSNK2A2 might play a role in tau hyperphosphorylation, and thus tangle formation. However, the biochemical mechanism whereby CSNK2A2 is activated is still unclear. The observation that CSNK2A2 is activated in cultured cells treated with insulin, IGF-I, and EGF (Krebs et al., Cold Spring Harb Symp Quant Biol 53 Pt 1:77-84 (1988)) (factors that signal through tyrosine kinase receptors) suggests that the aberrant CSNK2A2 cascade observed in AD could reflect an altered tyrosine phosphorylation balance. Recent studies showed that, in turn, CSNK2A2 activity can stimulate the tyrosine phosphorylation cascade elicited by the insulin receptor (Marin et al., Int J Biochem Cell Biol 28:999-1005 (1996)), and that CSNK2A2 itself can have tyrosine kinase activity (Marin et al., J Biol Chem 274:29260-29265 (1999)). Thus, there is clear evidence for a link between CSNK2A2 and tyrosine phosphorylation cascades, and the direct interaction between CSNK2A2 and FAK disclosed herein suggests that their respective activities might be coordinately regulated. We suggest that adequate pharmacological modulation of FAK activity or CSNK2A2 activity, or modulation of the interaction between FAK and rab11, or between FAK and CSNK2A2, might prevent neuronal dysfunction and neuronal death observed in the brain of AD patients and patients suffering from other neurodegenerative conditions.

We also discovered that the same fragment of FAK (amino acid residues 724-1052) interacts with glutathione S-transferase M3 (GTM3), further supporting the involvement of FAK in neurodegeneration and AD. Free radical neurotoxicity orchestrated through the generation of lipid peroxidation products is well documented and was proposed to mediate as least some aspect of Aβ toxicity (Mark et al., Mol Neurobiol 12:211-224 (1996); Butterfield, Chem Res Toxicol 10:495-506 (1997); Whitehouse, Neurology 48 Suppl. 7:S2-S7 (1997)), probably through the generation of 4-hydroxynonenal (HNE) (Keller & Mattson, 1998). There is ample evidence that antioxidant molecules protect neurons, and, in particular, it is clear that glutathione transferase (GST) protects neurons against toxicity induced by HNE (Xie et al., Free Radical Biol Med 25:979-988 (1998)). In this respect, it is interesting that the activity of GST is reduced in the brains and CSF of AD patients, compared to controls (Lovell et al., Neurology 51:1562-1566 (1998)). Thus, this interaction between FAK and GTM3 generates a new link between two independent pathways that are involved in neuron survival and that are altered in the brains of AD patients. We suggest that adequate pharmacological modulation of FAK activity or GTM3 activity, or the modulation of the interaction between FAK and GTM3, might prevent neuronal dysfunction and neuronal death observed in the brain of AD patients and patients suffering from other neurodegenerative conditions.

Previously, we reported an interaction between δ-catenin (CTNND2) and the break point cluster region protein, BCR, and we explained the relevance of this interaction in the context of neurodegeneration and AD (see U.S. patent application Ser. No. 09/466,139; International Patent Application No. PCT/US99/30396 (WO 00/37483)). In subsequent experiments, we have sought to identify the proteins that interact with BCR. We have identified a number of interactions and interactors, reported here, that strengthen our initial assertion that the BCR protein plays an important role in the brain. Using a C-terminal portion of BCR comprising amino acid residues 1206 to 1271, we identified two important interacting proteins: the neuroendocrine protein discs, large (Drosophila) homolog 3 (DLG3) (also known as NE-dlg, or SAP102 for synapse-associated protein 102), and the postsynaptic density protein 95, or PSD95 (also known as SAP90 for synapse-associated protein 90, and DLG4). These two proteins are 67% identical (81% similar) to each other, and both function as synaptic scaffolding proteins that interact with synaptic receptors and associated molecules. PSD95 interacts with the NMDA receptor (Kornau et al., Science 269:1737-1740 (1995)) and this interaction is altered by transient global ischemia (Takagi et al., J Neurochem 74:169-178 (2000)). Nitric oxide synthase (NOS), an enzyme that regulates the activity of the NMDA receptor, also interacts with PSD95, and this interaction can be disrupted by CAPON (Jaffrey et al., Neuron 20:115-124 (1998)). DLG3 also interacts with the NMDA receptor (Lau et al., J Biol Chem 271:21622-21628 (1996); Muller et al., Neuron 17:255-265 (1996)). The well-documented role of the NMDA receptor in long-term potentiation (LTP) in the hippocampus (Muller et al., Synapse 19:37-45 (1995); Sans et al., J Neurosci 20:1260-1271 (2000)) suggests that proteins such as PSD95 and DLG3 play important synaptic functions underlying learning and memory. In addition, PSD95 also interacts with several types of potassium channels (Laube et al., Brain Res Mol Brain Res 42:51-61 (1996); Nehring et al., J Neurosci 20:156-162 (2000)). The activity of those channels is clearly involved in neuronal survival (Holm et al., Proc Natl Acad Sci USA 94:1002-1006 (1997); Mattson, Physiol Rev 77:1081-1132 (1997)), particularly in the hippocampus (Zawar & Neumcke, Pflugers Arch 439:256-262 (2000)). Thus, through its function of clustering potassium channels, PSD95 also plays a role in neuronal survival.

It is also interesting to note that PSD95 interacts with SynGAP (Kim et al., Neuron 20:683-691 (1998)), an activating protein for the GTPase Ras. Thus, PSD95 interacts with at least two proteins that activate GTPases: SynGAP and BCR (Braselmann & McCormick, EMBO J 14:4839-4848 (1995); Diekmann et al., EMBO J 14:5297-5305 (1995)). In attempting to further define a role for BCR in AD we set out to identify any additional proteins with which it interacts, that might play a role in synaptic function. We discovered that several overlapping fragments of BCR were capable of interacting with a portion of the transcription factor HTF4A, comprising amino acid residues 296-494. HTF4A is a protein comprising 682 amino acid residues, from the myc family of basic helix-loop-helix (bHLH) transcription factors. HTF4A activates the transcription of a number of genes by binding to E-box motifs, including the gene for the □1 acetylcholine receptor (AChR) (Neville et al., 1998). HTF4A also stimulates the transcription of the VGF gene (Di Rocco et al., Mol Cell Biol 17:1244-1253 (1997)), a secreted neuropeptide whose expression is induced by several neurotrophins (Snyder et al., Neuroscience 82:7-19 (1998)). Decreased levels of vgf mRNA in the hippocampus have been correlated with age-induced cognitive decline in rats (Sugaya et al., Neurobiol Aging (1998)). Thus, reduced HTF4A-dependent transcriptional activity in the hippocampus could be associated with age-related memory loss. This interation strengthens the finding that BCR and associated proteins play an important role in hippocampal synaptic function.

During our attempts to discover additional BCR interactors we found that a fragment of BCR comprising amino acid residuess 856-1226 interacted with a fragment of a novel human protein. That novel protein, which was 94% identical to mouse semaphorin F (M-sema F), is now known to be semaphorin 4C, alternative transcript (821), or SEMA4C(821). The semaphorins belong to a family of secreted and membrane bound proteins involved in the nervous system development and axonal guidance. Semaphorin F is a transmembrane form (Inagaki et al., Science 270:1166-1170 (1995)). Recently, the cytosolic C-terminal domain of M-sema F was found to interact with GIPC (also called Semcapl) (Wang et al., Mol Cell Biochem (1999)). Thus, semaphorin F/SEMA4C is a common interactor to both BCR and GIPC, as is δ-catenin.

We also discovered that a C-terminal fragment of BCR, comprising amino acid residues 1206-1271, interacted with a fragment of the SNF2-related CBP activator protein (SRCAP), comprising amino acid residues 1916-2088. SRCAP, a CBP interacting protein, was shown to possess ATPase activity and activate the transcription of several genes (Johnston et al., J Biol Chem 274:16370-16376 (1999)). Importantly, CBP (CREB-binding protein), which is a co-activator of a number of transcription factors, interacts with a number of other proteins such as histone acetyltransferases, general transcription factors, and other co-activators. Since BCR interacts with proteins such as δ-catenin, PSD95, SEMA4C(821), and DLG3 (all of which are involved in synaptic function), and because CREB-mediated immediate early transcription is essential for LTP in the hippocampus (Walton et al., J Neurosci Res 58:96-106 (1999)), the interaction between BCR and SRCAP brings together the essential components of hippocampal synaptic modulation. Additionally, because BCR was found as an interactor with δ-catenin (see U.S. patent application Ser. No. 09/466,139; International Patent Application No. PCT/US99/30396 (WO 00/37483)), the interactions reported in the present application (i.e., BCR with PSD95, DLG3, SEMA4C(821), HTF4A, and SRCAP) generate a pathway that links δ-catenin and to synaptic functions and neuronal survival. Consequently, we suggest that adequate pharmacological modulation the interactions between BCR and any of the five BCR interactors described herein might prevent the synaptic dysfunction and neuronal death observed in the brains of AD patients and patients suffering from other neurodegenerative conditions.

In an attempt to further define the role of PSD95 in AD pathogenesis, we sought to indentify additional interactors of the protein. We found that a fragment of PSD95 comprising amino acid residues 149-255 interacted with a fragment of the novel protein PN7740 comprising amino acid residues 27-321. As described above, PSD95 is a member of the MAGUK family (membrane associated guanylate kinase) containing three PDZ domains, one SH3 domain, and one guanylate kinase (GK) domain (Wheal et al., Prog Neurobiol 55:611-640 (1998); Dimitratos et al., Bioessays 21:912-921 (1999)). Also called DLG4 and SAP-90 (Kistner et al., J Biol Chem 268:4580-4583 (1993); Stathakis et al., Genomics 44:71-82 (1997)), PSD95 is found at the post-synaptic density where it interacts with other synaptic scaffolding proteins and with synaptic signalling proteins such as neurotransmitter receptors and channels (e.g. NMDA receptors, potassium channels) (Kornau et al., Science 269:1737-1740 (1995); Nagano et al., J Biochem 124:869-875 (1998); Lau et al., J Biol Chem 271:21622-21628 (1996); Nehring et al., J Neurosci 20:156-162 (2000)). PN7740 is a novel protein that we previously reported interacts with Fe65/APPB1 (see U.S. Pat. No. 6,653,102, issued Nov. 25, 2003). The full-length cDNA for PN7740 (SEQ ID NO:1) contains a open reading frame (ORF) coding for 372 amino acids (SEQ ID NO:2). The putative ATG initiation codon is preceded by a purine (G) residue in position −3, and by several upstream STOP codons, suggesting that it represents the authentic initiation codon. At the end of the 3′ UTR (untranslated region), we found a canonical polyadenylation signal (AATAAA) shortly before the poly A sequence itself. As mentioned above, a phosphatase 2C domain is found from amino acids 104 to 339 of PN7740. Thus, we have identified a novel phosphatase that binds to the first PTB domain of Fe65/APPB1.

Immunosuppressant drugs such as FK506, rapamycin, and cyclosporine A (CsA) act by inhibiting T cell proliferation and bind to a group of proteins collectively called immunophilins. Although most of the studies on immunophilins have focused on lymphocytes, the recent finding that immunophilins are much more abundant in the nervous system than the immune system has opened promising new therapeutic avenues (Snyder et al. Neuroscience 82:7-19 (1998); Steiner et al. Nat Med 3:421-428 (1997); Steiner et al., Proc Natl Acad Sci USA 94:2019-2024 (1997)). In the immune system, CsA and FK506 inhibit the synthesis and secretion of interleukin-2 (IL-2), an early step in the response of T cells to antigen. Rapamycin, on the other hand, blocks the IL-2-induced clonal proliferation of activated T cells by inhibiting signaling through the IL-2 receptor. These findings suggested that CsA and FK506 may act through similar molecular mechanisms, while rapamycin act through a different mechanism (Snyder et al. Neuroscience 82:7-19 (1998)). It is known that CsA binds to an 18 kDa protein called cyclophilin, and FK506 binds to a 12 kDa protein called FKBP12. Curiously, both cyclophilin and FKBP12 show peptide-propyl isomerase (rotamase) activity (Snyder et al., Neuroscience 82:7-19 (1998)). Although the immunophilin ligands inhibit the rotamase activity, several of these ligands lack immunosuppressant activity. This indicated that the rotamase activity is not linked to the immunosuppressant effect. The drug-immunophilin complex was suggested to acquire a gain of function and bind to another protein that neither the drug nor the immunophilin alone would interact with alone. The first drug-immunophilin target was identified as calcineurin, a Ca²⁺-calmodulin activated phosphatase. Calcineurin was found to bind both CsA-cyclophilin A complexes and FK506-FKBP12 complexes (Cameron et al. Cell 83:463-472 (1995)). One of the calcineurin substrates is the phosphorylated form of the transcription nuclear factor of activated t-cells (NF-AT), which is known to activate transcription of many genes in T-cells, including IL-2 and its receptor. Only the non-phosphorylated form of NF-AT can enter the nucleus. Binding of drug-immunophilin complexes to calcineurin inhibits its activity, leading to elevated phosphorylation levels of NF-AT and to reduced transcription of IL-2 and its receptor (as NF-AT is then not able to enter the nucleus). As for rapamycin, it was also shown to bind FKBP12 with very high affinity. However, the rapamycin-FKBP12 complex does not bind to calcineurin but binds to a group of proteins called rapamycin and FKBP12 target 1 (RAFT1), FKBP and rapamycin associated protein (FRAP), and mammalian target of rapamycin (TOR) (Freeman & Livi, Gene 172:143-147 (1996); Lorenz & Heitman, J Biol Chem 270:27531-27537 (1995)). RAFT1 is known to phosphorylate the protein translation regulator 4E-BP1 (Snyder et al., Neuroscience 82:7-19 (1998)).

In the nervous system, immunophilin concentrations are 50-fold higher than in the immune system (Snyder et al. Neuroscience 82:7-19 (1998)). Both cyclophilin and FKBP-12 are almost exclusively neuronal in the brain, with striking regional variations that closely parallel those of calcineurin. Highest levels are found in the granular cells of the cerebellar folia, in the hippocampus, in the striatum, and in the substantia nigra. Two major brain substrates of calcineurin are GAP-43 (mediating neurite outgrowth) and neuronal nitric oxide synthase (nNOS). Nitric oxide is a mediator of glutamate-induced toxicity through NMDA receptors, as nNOS inhibitors, and gene knockouts of nNOS can block this toxic effect. nNOS activity is inhibited when the enzyme is phosphorylated. Therefore, nNOS is expected to be activated by calcineurin, and blocked by calcineurin inhibitors (Snyder et al. Neuroscience 82:7-19 (1998); Steiner et al. Nat Med 3:421-428 (1997); Steiner et al., Proc Natl Acad Sci USA 94:2019-2024 (1997)). Indeed, by inhibiting calcineurin, FK506 was shown to increase the levels of phosphorylated nNOS, thus reducing its catalytic activity, and providing neuroprotection against glutamate. As expected, rapamycin blocked the effect of FK506 (since it binds to FKBP12, but the FK506-FKBP12 complex does not bind to calcineurin). Another effect of FK506 in the brain is the modulation of neurotransmitter release. As nitric oxide is required for neurotransmitter release from PC12 cells and brain synaptosomes stimulated by NMDA, FK506 inhibits neurotransmitter release in these systems, and these effects are blocked by rapamycin. By contrast, neurotransmitter release is stimulated by FK506 in synaptosomes depolarized by K+ channel blockers. This effect is mediated by synapsin I, a synaptic vesicle associated protein, and dynamin I, a GTPase involved in the recycling of synaptic vesicles. The neurotransmitter release activity of both proteins is stimulated by phosphorylation and inhibited by dephosphorylation. Since both synapsin I and dynamin I are substrates for calcineurin, inhibition of the phosphatase activity of calcineurin by FK506 increases the phosphorylation state of synapsin I and dynamin I, thus stimulating neurotransmitter release. Another important effect of the immunophilins in the brain is the modulation of intracellular concentration of Ca²⁺ (i Ca²⁺). FKBP12 binds to the ryanodine receptor and to the IP3 receptor, two proteins involved in the release of Ca²⁺ from intracellular stores. Both receptors are activated when phosphorylated by the protein kinase C (PKC). The binding of FKBP12 to these receptors attracts calcineurin in the complex, which reduces the phosphorylation level of the receptor. In the presence of FK506, the FKBP12-calcineurin complex dissociates from the IP3 receptor, which shows increased activity, resulting in elevated i Ca²⁺ (Snyder et al,. Neuroscience 82:7-19 (1998); Steiner et al., Nat Med 3:421-428 (1997); Steiner et al., Proc Natl Acad Sci USA 94:2019-2024 (1997)).

In addition, FK506 also has neurotrophic activities that were observed in PC12 cells and sensory ganglia at subnanomolar concentration, that are similar to well-characterized neurotrophic factors such as the nerve growth factor (NGF), brain-derived growth factor (BDNF), and neurotrophins NT-3 and NT-4. Recently, FK506 derivatives were synthesized that bind immunophilins (FKBP12) with the same potency as the parent drug, but the drug-immunophilin complexes did not bind calcineurin and had no immunosuppressant activity. However, these new drugs (e.g., GPI1046) retained the full neurotrophic activity of FK506. Stimulation of neurite outgrowth was observed at 1 μM concentrations, with a maximal effect at 1 nM. Furthermore, while the classic neurotrophic proteins (NGF, BDNF, NT-3 and NT-4) each act only in a selected repertoire of neuronal systems, immunophilin ligands (FK506 and derivatives) are active in all the systems examined. However, the neurotrophic actions of the immunophilin ligands are restricted to damaged neurons, but have no effect on normal peripheral or central neurons (while neurotrophic proteins elicit such effect). Thus, immunophilins mediate both calcineurin-dependent and calcineurin-independent neurotrophic activities (Snyder et al., Neuroscience 82:7-19 (1998); Steiner et al., Nat Med 3:421-428 (1997); Steiner et al., Proc Natl Acad Sci USA 94:2019-2024 (1997)).

We have now discovered that the amino-terminal cytosolic region of presenilin-1 comprising amino acid residues 1-91, interacts with a polypeptide corresponding to the carboxyl-terminal region (amino acid residues 166-224) of FKBP25. FKBP25, which is in the same family as FKBP12, is an immunophilin that binds FK506 and rapamycin, and has a rotamase domain in its C-terminal half (Jin et al. J Biol Chem 267:10942-10945 (1992); Galat et al. Biochemistry 31:2427-2434 (1992); Hung, Schreiber, Biochem Biophys Res Commun 184:733-738 (1992); Wiederrecht et al. Biochem Biophys Res Commun 185:298-303 (1992)). It shares about 45% identity with other FKBP proteins (e.g., FKBP12, -13, and -59) in its 97 C-terminal residues, while its amino-terminal region does not share identity or similarity with any known protein. As with the other FKBP proteins, FKBP25 rotamase activity is inhibited by both FK506 and rapamycin, however rapamycin has a much greater potency (IC50=50 nM) than FK506 (IC50=400 nM) (Jin et al. J Biol Chem 267:10942-10945 (1992); Galat et al. Biochemistry 31:2427-2434 (1992); Hung, Schreiber, Biochem Biophys Res Commun 184:733-738 (1992); Wiederrecht et al. Biochem Biophys Res Commun 185:298-303 (1992)).

The cellular and biochemical mechanisms elicited by FKBP25 are at present unknown. Since FKBP12-rapamycin complexes do not act through the calcineurin pathway, and because FKBP25 has a much higher affinity for rapamycin than FK506, it is likely that FKBP25 acts predominantly through calcineurin-independent pathways, and to a lesser extend through calcineurin-dependent pathways. Indeed, FKBP25, which contains a nuclear localization signal in its rotamase domain (that is absent in other FKBPs), was localized in the nucleus, and binds to casein kinase II (CKII) and nucleolin (Jin & Burakoff, Proc Natl Acad Sci USA 90:7769-7773 (1993)). CKII phosphorylates a number of cytosolic and nuclear substrates, and is an important regulator of cell growth. The phosphorylation of nucleolin is a crucial step in ribosome formation. It is possible that the phosphorylation of FKBP25 enhances its translocation to the nucleus, and in turn, the association of CKII with FKBP25 could also facilitate the nuclear translocation of the kinase, which could then phosphorylate nucleolin and other nuclear substrates. Alternatively, the rotamase activity of FKBP25 could inhibit the function of CKII and nucleolin. The high levels of FKBP25 observed in the hippocampus (a severely affected area in the brains of AD patients), and its association with PS-1 and with CKII, suggest that FKBP25 is involved in a brain function that is somehow related to AD pathogenesis. FKBP25 belongs to the immunophilin family, whose neurotrophic actions have been well documented, and it may play a critical role in the survival of hippocampal neurons. In this respect, its association with either wild type or mutant forms of PS-1 could alter its activity. The activity and protein levels of CKII are greatly reduced in AD brains, and this reduction closely matches the regional distribution of the pathological features. One of the targets of CKII is APP, and it is known that APP phosphorylation affects its metabolism. Thus, mutations in PS-1 could alter the function of FKBP25, which, in turn, could change the activity of CKII, and ultimately the phosphorylation state of APP, its metabolism, and the production of AB. Alternatively, the alteration of FKBP25 function (because of an altered interaction with FAD mutant PS-1) could destabilize calcium homeostasis and lead directly to neuronal apoptosis. Thus, the biological effects elicited by FKBP25 may be of great importance for neuron survival and their alteration may be critical in neurodegenerative processes like those observed in AD.

As a first step towards a better understanding of the cellular and biochemical events elicited by FKBP25, we sought to identify the proteins with which it interacts. We found that full-length FKBP25 interacts with full-length calcium and integrin binding protein, CIB. Further characterization of this interaction using shorter FKBP25 fragments showed that the 25 N-terminal amino acid residues of FKBP25 is sufficient for interaction with CIB. This finding suggests that CIB may interact specifically with FKBP25, but with no other FKBPs, sicne the N-terminal region of FKBP25 is not shared with other FKBPs.

CIB is a 191 amino acid protein that was discovered in 1997 in a yeast two-hybrid search using the cytoplasmic domain of integrin allb as bait (Naik et al., J Biol Chem 272:4651-4654 (1997)). CIB contains 2 calcium binding domains (EF hands) and is 58% similar (28% identical) to calcineurin B, the 19 kDa regulatory subunit of calcineurin; and 55% similar (27% identical) to calmodulin. The authors of this study suggest that CIB might be the regulatory subunit of a new, as yet unknown, multi-subunit calcium-dependent phosphatase. Because other FKBPs are known to bind the IP3 and ryanodine receptors, it is also possible that FKBP25, CIB and its associated phosphatase bind to and control the phosphorylation state of the IP3 or the ryanodine receptors. Thus, the PS1-FKBP25-CIB pathway could play a major role in the control of calcium release from internal stores. In support of this hypothesis, PS1 was recently shown to bind the ryanodine receptor directly (Mattson et al., Soc Neurosci Abstr 25:1600 (1999)), and this interaction was shown to control calcium homeostasis. In addition, CIB was recently shown to interact with PS2 and PS1 (Stabler et al., J Cell Biol 145:1277-1292 (1999). The mutations in PS1 and PS2 associated with FAD induce neuronal apoptosis through the disruption of neuronal calcium homeostasis. It is likely that these mutations disrupt the interactions of PS1 and PS2 with other proteins, like FKBP25, CIB, and the ryanodine receptor. Thus, the interaction network generated by our findings provides a direct biochemical link between the presenilins and the control of calcium homeostasis. Pharmacological agents that influence these protein-protein interactions will play a major role in the control of neuronal survival or apoptosis.

As described above, the intracellular trafficking of APP is quite complex. After secretion of the large N-terminal fragment by the α- or β-secretase, the transmembrane C-terminal fragment (which may or may not contain the entire Aβ region) is endocytosed into clathrin-coated pits, and targeted to other intracellular compartments (Selkoe et al., Ann NY Acad Sci 777:57-64 (1996); Selkoe, Annu Rev Cell Biol 10:373-403 (1994)). Some cells have a low secretory activity and also recycle full-length APP back into the intracellular membrane network. Because the final destination of each fragment will determine its eventual fate, the intracellular trafficking of APP metabolites is very important to the production of the Aβ peptide, and its release from cells. APP and its metabolites have been detected in almost all intracellular compartments, like the recycling endosomes (going to the Golgi and endoplasmic reticulum (ER)), and the sorting endosomes (going to the lysosomes or back to the plasma membrane). While the pathways going from the plasma membrane to the Golgi and ER, or to the lysosomes, are responsible for Aβ production or degradation, the recycling route toward the membrane is a crucial step potentially leading to Aβ secretion (Selkoe, Trends Cell Biol 8:447-453 (1998)). Thus, any protein involved in the traffic of intracellular vesicles containing APP metabolites could play a major role in the production and release of AB.

Small GTPases of the rab family play an essential role in the control of intracellular vesicle trafficking (Geppert & Sudhof, Annu Rev Neurosci 21:75-95 (1998)). These proteins are expressed at high levels in the neuro-endocrine system and they represent crucial elements regulating processes like hormone secretion and neurotransmitter release (Deretic, Electrophoresis 18:2537-2541 (1997)). Over 30 different rab proteins have been identified, showing a wide range in levels and locations of expression, from gastric wall to brain, and differential distribution into distinct subcellular compartments. This suggests that different members of the rab family might confer specificity to particular intracellular trafficking pathways. However, the detailed molecular mechanisms of action of the rab proteins are not completely understood. The rab3 protein is involved in the fusion of neurotransmitter-loaded secretory vesicles with the plasma membrane, an event that involves GTP hydrolysis, GDP/GTP exchange with the protein GDI, and an elevation of Ca²⁺ in the synaptic terminus (Park et al., J Biol Chem 272:20857-20865 (1997); Johannes et al., EMBO J 13:2029-2037 (1994); Ahnert-Hilger et al., Eur J Cell Biol 70:1-11 (1996)); Geppert, Sudhof, Annu Rev Neurosci 21:75-95 (1998)). Several isoforms of rab3 have been described, but the specific function of each one of them is not yet known. Nevertheless, it is clear that rab3 is involved in neurotransmitter release. Other rab proteins such as rab4, rab5, rab11, rab17, rab18, and rab20 have all been shown to be involved in a complex endocytotic pathway (Geppert & Sudhof, Annu Rev Neurosci 21:75-95 (1998)), and different rab proteins associate with endosomes targeted to specific subcellular compartments. A number of studies have shown that rab11 associates with recycling endosomes and other post-Golgi membranes such as the trans-Golgi network (TGN) and secretory vesicles. On the other hand, the rab5 protein is associated with sorting endosomes (en route to the lysosomes) and other early factors of the endocytotic traffic. To date, rab11 is the only GTPase known to regulate the intracellular traffic through recycling endosomes (Ullrich et al., J Cell Biol 135:913-924 (1996)).

A number of mutations in PS1 are known to cause AD in certain families. Both in vitro (cell transfection) and in vivo (transgenic mice) studies have demonstrated that these mutations result in an increase of AB42 production and secretion (Duff et al., Nature 383:710-703 (1996); Hutton & Hardy, Hum Mol Genet 6:1639-1646 (1997); Cruts, Van Broeckhoven, Hum Mutat 11:183-190 (1998); Kim & Tanzi, Curr Opin Neurobiol 7:683-688 (1997)); Hardy, Trends Neurosci 20:154-159 (1997); Selkoe, Trends Cell Biol 8:447-453 (1998)), which is evidence for altered APP processing. However, the existence of a direct biochemical link between APP and PS1 is still highly controversial, and it is not at all clear how mutations in PS1 could alter APP metabolism. One study (Wolfe et al., Nature 398:513-517 (1999)) suggested that PS1 could be the γ-secretase itself, although it is equally possible that PS1 is a regulatory protein that modulates the activity of γ-secretase.

Importantly, we have discovered that the amino-terminal cytosolic region of presenilin-1 (amino acid residues 1-91), interacts with the carboxyl-terminal region of rab11a (aa 106 to 216). Additional interactions between these two proteins have also been demonstrated (Dumanchin et al., Hum Mol Genet 8:1263-1269 (1999)). The discovery of direct biochemical interactions between PS1 and rab11a offers an attractive explanation of the mechanism whereby PS1 mutations cause elevated secretion of AB42. As described above, rab11 controls the trafficking of recycling endosomes and targets proteins to the Golgi and ER (Gromov et al., FEBS Lett 429:359-364 (1998); Lai et al., Genomics 22:610-616 (1994); Urbe et al., FEBS Lett 334:175-182 (1993); Sheehan et al., Neuroreport 7:1297-1300 (1996)). The cytoplasmic domain of APP is known to interact with the protein Fe65, which in turn interacts with LSF (Russo et al., FEBS Let 434:1-7 (1998)). As described herein, LSF interacts with both APP and PS1. Thus, the interaction series APP→Fe65→LSF→PS1→rab11a suggests that upon endocytosis, APP can be driven to the Golgi and endoplasmic reticulum through rab11a-containing recycling endosomes. It is expected that mutations in PS1 could alter its interactions with other proteins, including rab11a. This, in turn, could change the ultimate fate of APP-containing vesicles: if the PS1-rab11a interaction is tight, the endocytic vesicles will go to the Golgi and ER compartment. On the other hand, if the PS1-rab11a interaction is loose, the vesicles will become sorting endosomes and either go back to the plasma membrane (a rare event), or to the lysosomes, where APP and its metabolites are completely degraded. This model predicts that the interaction of APP with Fe65 would promote the production of the Aβ peptide, which was recently confirmed (Sabo et al., J Biol Chem 274:7952-7957 (1999). On the other hand, driving APP away from the Golgi-ER compartment and toward lysosomes is expected to reduce Aβ production. This is indeed what was observed (Schrader-Fischer et al., J Neurochem 68:1571-1580 (1997).

In a search for additional interactors of APP, we found that the C-terminal cytoplasmic fragment of APP-695 comprising amino acid residues 639-695 interacted with a polypeptide comprising the carboxyl-terminus (amino acid residues 603-1132) of the HLA-B associated transcript 3 protein, know as BAT3. BAT3 is a protein of unknown function that contains a ubiquitin-like domain in its N-terminal region (amino acid residues 17-77) and two proline-rich domains (amino acid residues 202-207 and 657-670) (Banerji et al., Proc Natl Acad Sci USA 87:2374-2378 (1990); Wang, Liew, Mol Cell Biochem 136:49-57 (1994); Spies et al., Proc Natl Acad Sci USA 86:8955-8958 (1989); Spies et al., Science 243:214-217 (1989)). Thus, the domain of BAT3 that interacts with APP contains the second proline-rich region, but not the ubiquitin-like domain. As mentioned in the background section above, APP is involved in a wide variety of functions throughout the organism. Like APP, BAT3 is expressed in all tissues examined, including brain. Thus, BAT3 might be involved in APP recycling or intracellular trafficking, which, as discussed above, ultimately modulates Aβ production. To find out if and how the interaction of BAT3 with APP could influence APP trafficking, we looked for proteins that interact with BAT3. We found that the N-terminal portion of BAT3, from amino acid residue 1 to 241, interacted with a polypeptide comprising amino acid residues 1062 through 1153 of the adaptor-related protein complex 3, delta 1, which is also known as δ-adaptin, or AP3D1. AP3D1 is the major component of the AP-3 complex (Dell'Angelica et al., Science 280:431-434 (1998)). Transport vesicles are coated by clathrin and by associated protein complexes known as AP-1, AP-2, AP-3, and AP-4 (Hirst & Robinson, Biochem Biophys Acta 1404:173-193 (1998)). Each of these complexes contains a specific set of proteins having extensive sequence similarity with one another. The most notorious of these proteins are called adaptins. Adaptin α and γ are components of the AP-1 and AP-2 complexes, respectively, while d-adaptin is part of the AP-3 complex. Le Borgne and coworkers showed that the AP-3 complex mediates the intracellular transport of transmembrane glycoproteins to lysosomes (Le Borgne et al., J Biol Chem 273:29451-29461 (1998)). Thus, because BAT3 interacts with the cytoplasmic domain of APP, the BAT3-δ-adaptin connection could be a key to the lysosomal targeting of APP. This is of utmost importance because targeting APP to the lysosomal compartment reduces Aβ secretion (Schrader-Fischer et al., J Neurochem 68:1571-1580 (1997)).

In summary, during endocytosis, APP can be targeted to recycling or sorting endosomes. The recycling endosomal vesicles eventually go to the Golgi and the ER, where AB40 and AB42, respectively, are made. On the other hand, sorting endosomes can either go directly back to the plasma membrane (a rare event) or to lysosomes, where APP metabolites are degraded. The rab11 GTPase (a PS1 interactor) is highly enriched in recycling endosomes versus sorting endosomes, and thus may be involved in targeting APP to cell compartments that produce AB. Therefore, a new model of APP trafficking emerges, in which rab11a and PS1 interact with APP (through the Fe65-LSF connection), targeting it to recycling endosomes, while the BAT3-δ-adaptin complex brings APP to sorting endosomes and lysosomes, where no Aβ is produced. Thus, APP trafficking and metabolism may be controlled by a competitive interaction with BAT3 or Fe65. In this respect, pharmacological agents that favor the BAT3-APP interaction are expected to drive APP to the lysosomes, thus reducing Aβ production.

In addition, BAT3 could also be involved in the brain-specific (neurotrophic, synaptotrophic) functions of APP. Using yeast two-hybrid system and co-immunoprecipitation, Hubberstey and colleagues showed that a portion of BAT3 comprising amino acid residues 246 through 360 binds to CAP1, an adenylate cyclase associated protein (Hubberstey et al., J Cell Biochem 61:459-466 (1996)). CAP1 is a 475 amino acid protein with two functionally different domains separated by a proline-rich region. Studies of yeast CAP showed that the N-terminal domain is involved in the activation of adenylate cyclase while the C-terminal domain is involved in nutritional and temperature sensitivity, growth, cell morphology, and budding (Zelicof et al., J Biol Chem 271:18243-18252 (1996)). In this respect, it is interesting that the random budding phenotype, observed in yeast strains that do not express CAP, could be suppressed by over expression of SNC1, a yeast homolog of mammalian synaptobrevin, a protein involved in the fusion of synaptic vesicles with the presynaptic membrane. It is thus possible that in humans, CAP1 and synaptobrevin are involved in similar aspects of synaptic formation and maintenance. As for the activity of the N-terminal fragment of CAP1, the activation of adenylate cyclase results in elevation of intracellular cAMP levels, a phenomenon that has been linked to long-term potentiation (LTP) (Sah & Bekkers, J Neurosci 16:4537-4542 (1996); Kimura et al., J Neurosci 18:8551-8558 (1998); Storm et al., Neuron 20:1199-1210 (1998); Villacres et al., J Neurosci 18:3186-3194 (1998)), considered as the cellular and biochemical substrate for memory (Matzel et al., Rev Neurosci 9:129-167 (1998); Davis, Laroche, C R Acad Sci III 321:97-107 (1998)). Thus, APP (a protein directly involved in AD and with well documented brain functions) interacts with BAT3, a large proline-rich protein. BAT3, in turn, interacts with CAP1, another proline-rich protein containing one domain involved in the regulation of cAMP levels (thus influencing LTP and memory) and another domain that, like synaptobrevin, might participate in synaptic functions. Thus, BAT3 represents a crucial link between APP and CAP1; two proteins with brain specific functions. The BAT3-APP interaction is thus a potential point of intervention in the biochemical and cellular events leading to synaptic formation and LTP (memory), with a direct impact on AD.

Considering the potential effects of BAT3 on both APP metabolism and APP neurotrophic function, as described above, drugs that would favor the BAT3-APP interaction should be useful for protecting against the neurodegeneration observed in AD patients.

The protein tyrosine phosphatase zeta (PTPZ, Swiss-Prot accession number: P23471; GenBank accession number: M93426) is a large type I transmembrane protein of 2314 amino acids, expressed specifically in the central nervous system (Krueger and Saito, Proc Natl Acad Sci USA 89:7417-7421 (1992); Shintani et al., Neurosci Lett 247:135-138 (1998)). It has the typical structure of a cell surface receptor, with a signal peptide from amino acid residues 1 through 24 and a single transmembrane domain from amino acid residues 1636 through 1661. Amino acid residues 25 through 1635 are extracellular, while amino acid residues 1662 through 2314 are cytoplasmic. PTPZ contains two tyrosine phosphatase domains, which comprise amino acid residues 1744 through 1997, and 1998 through 2314. Interestingly, PTPZ expression is increased in response to injury (Li et al., Brain Res Mol Brain Res 60:77-88 (1998)). Additionally, PTPZ is expressed at high levels by neurons and astrocytes during brain development. PTPZ belongs to a large family of phosphatases that play important roles in neuronal functions.

Importantly, we have found that a fragment of APP695 comprising amino acid residues 306-500 interacts with a portion of PTPZ comprising amino acid residues 1052 through 1128. As mentioned above, the secreted form of APP695 (which includes amino acid residues 306 through 500) has well documented neurotrophic activities, and a large body of evidence indicates that these activities are carried out by receptor-mediated mechanisms. Moreover, the balance of tyrosine phosphorylation has been shown to mediate sAPP neurotrophic activity. However, no specific APP receptor protein has yet been described. Thus, the finding that sAPP binds an extracellular portion of PTPZ provides the first biochemical link to the cellular mechanisms that underlie sAPP activity. Since APP metabolism and function, as well as phosphorylation and dephosphorylation reactions are deeply disrupted in the brains of AD patients, and because sAPP activities at the cellular level (neurotrophic, neuroprotective) are reflected by memory enhancement at the behavioral level, it is expected that drugs that alter PTPZ activity will have a tremendous potential for the treatment of neurodegenerative disease in general, and AD, in particular.

In addition to PTPZ, we found that a C-terminal fragments of the hypothetical protein KIAA0351 interacted with the fragment of APP695 comprising amino acid residues 306-500. The nucleotide sequence for KIAA0351 reported in GenBank (AB002349) is 6.3 kb long and contains an ORF encoding 557 residues, with an ATG initiation codon in a reasonably good Kozak environment (A in position −3). Because the encoded KIAA0351 protein is novel, nothing is known about its biological function. Amino acid sequence analysis revealed the presence of a pleckstrin homology (PH) region, between amino acid residues 431 and 480. According to Prosite documentation (PDOC 50003), the PH domain is found in a variety of proteins that are either involved in intracellular signaling, or are components of the cytoskeleton. For example, many proteins with GTPase activity, and GTP exchange factors, contain PH domains. This feature is particularly relevant to the neurotrophic and neuroprotective functions of sAPP, which could be mediated by a membrane-associated guanylate cyclase and by the formation of cGMP (Barger & Mattson, Biochem J 311:45-47 (1995); Barger et al., J Neurochem 64:2087-2096 (1995)). In this respect, KIAA0351 could represent a GTP donor that the guanylate cyclase could use as a substrate to form cGMP, upon activation by sAPP. KIAA0351 shares 48% similarity with GNRP, a guanine nucleotide releasing protein. A PH domain was also found in the Insulin Receptor Substrate 1 (IRS-1), which is important in the light of a study that showed that sAPP neurotrophic activity is mediated by phosphorylation of IRS-1 (Wallace et al., Brain Res Mol Brain Res 52:201-212 (1997). Notably, we have identified an interaction between the neurotrophic region of sAPP and a protein of unknown function—KIAA0351. The presence of a PH domain in KIAA0351 suggests that this protein could mediate the neurotrophic effect of sAPP.

We also found that the same fragment of APP695 (amino acid residues 306-500) that interacted with PTPZ and KIAA0351, also interacted with prostaglandin D synthase (PDG-synt). The interaction between APP695 and PDG-synt is important in light of the well documented inflammatory component of the AD pathology (Yamada et al., Stroke 27:1155-1162 (1996); Kalaria et al., Neurobiol Aging 17:687-693 (1996); Kalaria et al., Neurodegeneration 5:497-503 (1996); Dickson, J Neuropathol Exp Neurol 56:321-339 (1997); Cummings et al., Neurology 51:S2-S17 (1998)). The intricate cross-talk between the amyloid pathway and inflammation pathway make the situation complex. Besides the generation of free radicals, lipid peroxidation, and disruption of calcium homeostasis (Manelli & Puttfarcken, Brain Res Bull 38:569-576 (1995); Weiss et al., J Neurochem 62:372-375 (1994); Mark et al., J Neurochem 68:255-264 (1997); Mark et al., J Neurosci 15:6239-6249 (1995); Mattson, Alz Dis Review 2:1-14 (1997), there is evidence that Aβ toxicity can be mediated in part by some inflammatory factors (Fagarasan & Aisen, Brain Res 723:231-234 (1996); McRae et al., Gerontology 43:95-108 (1997)), including components of the complement cascade (Pasinetti, Neurobiol Aging 17:707-716 (1996)). Furthermore, cyclo-oxygenase 1 and 2 (COX1 and COX2) activities are elevated in the brains of AD patients, and prostaglandins are known neurotoxins (Prasad et al., Proc Soc Exp Biol Med 219:120-125 (1998); Pasinetti & Aisen, Neuroscience 87:319-324 (1998); Lee et al.1999; Kitamura et al. 1999). Reciprocally, factors released by activated microglial cells appear to accelerate the transition of diffuse plaques into the mature neuritic plaques observed in the brains of AD patients (Sheng et al., Acta Neuropathol (Ber1) 94:1-5 (1997)). The secreted form of APP (sAPP) has well documented survival, neurotrophic, and neuroprotective activities (Roch et al., Ann N Y Acad Sci 695:149-157 (1993); Saitoh & Roch, DN&P 8:206-215 (1995); Roch & Puttfarcken, Alz ID Res A1 1:9-16 (1996); Goodman & Mattson, Exp Neurol 128:1-12 (1994); Mattson et al., Neuron 10:243-254 (1993); Mattson, Physiol Rev 77:1081-1132 (1997)). These effects at the cellular levels are reflected by memory enhancement at the behavioral levels (Roch et al., Proc Natl Acad Sci USA 91:7450-7454 (1994); Meziane et al., Proc Natl Acad Sci USA 95:12683-12688 (1998); Huber et al., Neuroscience 80:313-320 (1997); Roch & Puttfarcken, Alz ID Res A1 1:9-16 (1996); Huber et al., Brain Res 603:348-352 (1993)). The domain involved in these activities was localized between amino acid residues Ala319 and Met335 of APP695 (Roch et al., Ann N Y Acad Sci 695:149-157 (1993); Saitoh & Roch, DN&P 8:206-215 (1995); Roch & Puttfarcken, Alz ID Res A1 1:9-16 (1996)), which overlaps with the fragment that we found interacts with prostaglandin D synthase. The sAPP interaction with prostaglandin D synthase is believed to control prostaglandin D synthesis. Since prostaglandins can be neurotoxic, drugs that modulate the activity of prostaglandin D synthase, or modulate its interaction with APP, could be used to reduce the levels of prostaglandin D in the brain, and alleviate the prostaglandin-mediated neurotoxicity. Additionally, the preferential localization of prostaglandin D in brain makes it an attractive drug target.

The calcium-activated neutral proteinase (CANP) calpain, an enzyme involved in intracellular signaling, is a heterodimer of a large (80 kDa) catalytic and a small (30 kDa) regulatory subunit (Suzuki et al., Biol Chem Hoppe Seyler 376:523-529 (1995)). The catalytic subunit exists in 2 variants, μ- and m-, activated by micromolar and millimolar calcium concentrations, respectively. The physiological function of calpain is quite complex and has not yet been fully elucidated. Unlike many proteases involved in protein degradation, calpain activity triggers a number of cellular modifications such as enzyme modulation (e.g., phospholipase C, calcineurin, PKC), and the conformational change of structural proteins (e.g., microtubule-associated proteins, lens proteins), membrane-associated proteins (e.g., receptors, ion channels, adhesion molecules), transcription factors (e.g., Fos, Jun), and more (Suzuki et al., Biol Chem Hoppe Seyler 376:523-529 (1995)). From the perspective of AD pathogenesis, it is of particular interest that APP itself was initially identified as a calpain substrate in activated platelets (Li et al., J Biol Chem 270:14140-14147 (1995). Moreover, calpain was found to be activated in the brains of AD patients, compared to controls, and that this activation was more pronounced in the regions of the brain most affected by the disease (Nixon et al., Ann N Y Acad Sci 747:77-91 (1994); Saito et al., Proc Natl Acad Sci USA 90:2628-2632 (1993)).

We have now discovered that a polypeptide fragment of acetylcholine esterase (AChE) comprising amino acid residues 31-137 interacts with the small (regulatory) subunit of calpain 4 (CAPN4). This finding is especially intriguing because cholinergic neurons are particularly affected in AD, and because the interaction between a calcium-activated protease and a cholinergic-specific enzyme allows the elaboration of the following model: a change in APP metabolism (due, for instance, to mutations in APP or the presenilins) results in a disruption of calcium homeostasis that alters calpain activity and triggers additional downstream modifications. These modifications can include further alterations of APP metabolism, as well as abnormal activation of AChE. Eventually, this cascade of events could result in amyloid accumulation and acetylcholine depletion. It is also important to note that calpain is essential for LTP (long term potentiation, the biochemical substrate of memory) in the hippocampus, the part of the brain most severely affected in AD (Denny et al., Brain Res 534:317-320 (1990); Muller et al., Synapse 19:37-45 (1995)). Thus, an interaction loop between APP and calpain (through calcium homeostasis) could independently affect the cholinergic system (interaction with AChE) and memory (modulation of LTP). This is not surprising, since memory is believed to be mediated in large part by hippocampal cholinergic neurons. Finally, the involvement of calpain in AD is supported by recent reports of interactions between calpain and the presenilins (Steiner et al., J Biol Chem 273:32322-32331 (1998); Shinozaki et al., Int J Mol Med 1:797-799 (1998)). In summary, calpain is a protease that plays a crucial role in normal neuronal and synaptic function, and interacts with major proteins involved in Alzheimer's disease (i.e., AChE, APP, the presenilins). Calpain levels and activity show profound alterations in the brains of AD patients. Therefore, modulation of calpain activity and/or modulation of its interaction pattern with other proteins is a promising new avenue for the development of new therapeutic agents for the treatment of AD, or its symptoms.

In an effort to better define the role of AcheE in AD pathogenesis, we sought to find additional proteins with which it interacts. To our surprise, we found that two non-overlapping domains of AChE, comprising amino acid residues 31 to 136, and 266 to 354, interacted with several polypeptides that correspond in sequence to carboxyl-terminal fragments of the novel hypothetical protein KIAA0436. The GenBank entry for KIAA0436 (AB007896) indicates that the sequence is likely incomplete, probably because no stop codon was found upstream of the putative ATG initiation codon. However, our data suggest that the first ATG in the GenBank sequence may indeed be the correct initiation codon, for several reasons. First, our Northern data show that the KIAA0436 protein is encoded by a 4.6 kilobase messenger RNA, which is approximately the same length as the GenBank entry. Thus, the GenBank sequence must be close to complete. Second, our 5′ RACE experiments conducted on KIAA0436 identified only about 50 nucleotides upstream of the GenBank sequence, and a few of these sequences contained an in-frame stop codon upstream of the first ATG. Finally, the putative ATG initiation codon is in a good Kozak environment, with an A in position −3 and a G in position +4. Therefore, since this ATG is the first initiation codon that occurs within the sequence, and since it is in a good Kozak environment, we consider it as the authentic initiation codon for the KIAA0436 protein. The encoded protein is thus a total of 638 amino acid residues long (and not 689, as reported in GenBank). The region of KIAA0436 that interacts with both fragments of AChE contains a domain similar to prolyl-oligopeptidase from amino acid residues 397 through 475. Consequently, we believe that the KIAA0436 protein is a novel protease that interacts with AChE. The mRNA transcript encoding KIAA0436 is found in highest levels in the brain, medium levels in heart, low levels in kidney and pancreas, and is undetectable in placenta, lungs, liver, and skeletal muscle. In summary, we have identified a novel protease that is expressed preferentially in brain, and interacts with AChE. As proteolytic events are known to be severely altered in the brains of AD patients, KIAA0436 may represent a promising new target candidate for drug discovery.

We also found that a fragment of AChE comprising amino acid residues 31 through 136 interacted with a polypeptide comprising amino acid residues 24-121 of the small α-endosulfine protein (ALPEND).ALPEND is 76% identical and 84% similar to the cAMP-regulated phosphoprotein 19 (Virsolvy-Vergine et al., Diabetologia 39:135-141 (1996)). The cAMP-regulated phosphoprotein 19 is a protein kinase A (PKA) substrate (Horiuchi et al., J Biol Chem 265:9476-9484 (1990); Girault et al., J Neurosci 10: 1124-1133 (1990)), as is endosulfine itself (Roch et al., Soc Neurosci A bstr 23:855 (1997)). Endosulfine is an endogenous ligand for SUR1, the type-1 sulfonylurea receptor. SUR1 is the regulatory subunit of ATP-sensitive inward rectifying potassium channels (K_{ATP} channels), while the channel-forming unit belongs to the Kir6.x family (Inagaki et al., FEBS Lett 409:232-236 (1997)). A major function of these channels is to link the metabolic state of the cell to its membrane potential: K_{ATP} channels close upon binding intracellular ATP to depolarize the cell and open when ATP concentrations return to resting levels (Ashcroft, Annu Rev Neurosci 11:97-118 (1988); Aguilar-Bryan et al., Science 268:423-426 (1995); Inagaki et al., Science 270:1166-1170 (1995)); Freedman & Lin, Neuroscientist 2:145-152 (1996)). These channels are involved in events such as insulin secretion from pancreatic β cells, ischemic responses in cardiac and cerebral tissues, and regulation of vascular smooth muscle tone. The activity of these channels in pancreatic β cells, where they play a crucial role in the secretion of insulin (Bryan & Aguilar-Bryan Curr Opin Cell Biol 9:553-559 (1997)), has been extensively studied: following an elevation of blood glucose levels, the intracellular concentration of ATP in pancreatic β cells rise, resulting in channel closure and cell depolarization. This allows Ca²⁺ ions to enter the cell through voltage-sensitive Ca²⁺ channels, which will trigger the fusion of insulin secretory vesicles with the plasma membrane and the subsequent release of insulin. In neurons, the same mechanisms involving K_{ATP} channels (linking the metabolic state of the cell to its membrane potential) control neurotransmitter release. It was shown in the pancreas that when endosulfine binds SUR1, the channel shuts down, thus stimulating insulin release. It is therefore believed that in the brain, endosulfine binding to SUR1 would also shut down K_{ATP} channels, leading to depolarization, Ca²⁺ entry, vesicle fusion, and release of the vesicular content into the synaptic cleft. In brief, α-endosulfine/ALPEND is a small protein regulating processes like neurotransmitter release and secretion of other factors from polarize cells. Its interaction with AChE suggests that α-endosulfine/ALPEND may be expressed in cholinergic neurons, and may control the release of acetylcholine and/or AChE from synaptic terminals.

We also discovered that AChE interacts directly with δ-catenin (CTNND2). Because δ-catenin interacts with PS1 (Zhou et al., Neuroreport 8:1489-1494 (1997)); Tanahashi & Tabira, Neuroreport 10:563-568 (1999); Kosik, Science 279:463-465 (1998)) and because of the involvement of the cholinergic nervous system in AD (Gooch & Stennett, Am J Health Syst Pharm 53:1545-1557 (1996); Alvarez et al., J Neurosci 18:3213-3223 (1998); Inestrosa, Alarcon, J Physiol Paris 92:341-344 (1998)), this novel interaction puts δ-catenin and AChE interactors at the heart of AD pathology.

We also found that the RGS-GAIP interacting protein GIPC interacts with both AchE and δ-catenin. GIPC is reported to contain a PDZ domain (De Vries et al., Proc Natl Acad Sci USA 95:12340-12345 (1998)), while the C-terminus of δ-catenin (comprising amino acid residues 1006-1225) appears to contain a PDZ-binding domain. GIPC is also reported, in this same study, to interact with the C-terminus of a protein called RGS-GAIP, which is a GTPase activating protein for Gαi heterotrimeric G-proteins (De Vries et al., Proc Natl Acad Sci USA 95:12340-12345 (1998)). GAIP was recently shown to be located on clathrin-coated vesicles (De Vries et al., Mol Biol Cell 9:1123-1134 (1998)). Therefore, when considering the interactions between PS1 and δ-catenin (Zhou et al., Neuroreport 8:1489-1494 (1997); Tanahashi & Tabira, Neuroreport 10:563-568) (1999); Kosik, Science 279:463-465 (1998)) and between PS1 and rab11a, as described above, the pieces of a complex puzzle come together: the GAIP-GIPC complex (involved in GTPase activation) could be brought into the proximity of a potential GTPase target like rab11a through interactions of GIPC with δ-catenin, δ-catenin with PS1, and PS1 with rab11a. It is also remarkable that both GAIP and PS1 have been found in clathrin-coated vesicles (De Vries et al., Mol Biol Cell 9:1123-1134(1998); Efthimiopoulos et al., J Neurochem 71:2365-2372(1998)), and that we found δ-catenin to interact with clathrin. When PS1 was first discovered (and first named S182), its physiological function was unknown, although it was thought to be involved in protein trafficking (Hardy, Trends Neuroscience 20:154-159 (1997)). The pattern of interactions that is now taking shape around PS1 fully supports this original speculation. The interactions of PS1 and δ-catenin with rab11a, GIPC, and clathrin suggest a crucial role in the control of intracellular vesicle trafficking. Since APP is also found in rab11-positive clathrin-coated vesicles, the control of vesicle trafficking is likely important in determining the ultimate fate of the APP molecules; leading to Aβ release or secretion of neurotrophic/protective sAPP. It should also be noted that a mouse homolog of GIPC was cloned and described in GenBank. In the first GenBank entry, the mouse GIPC was named synactin (accession number AF104358), a protein that interacts with syndecan, a cell surface heparin-sulfate proteoglycan that links the cytoskeleton to the extracellular matrix. In another GenBank entry, mouse GIPC is called Semcapl (accession number AF061263), which stands for “semaphorin F cytoplasmic domain associated protein 1.” Thus, GIPC is also thought to interact with semaphorin F, and therefore, is possibly involved in axonal outgrowth and guidance.

The interaction pattern of GIPC places it at the heart of vesicle trafficking and membrane fusion control, with direct consequences on the metabolism of proteins such as APP, PSI, δ-catenin, and AChE.

APP metabolism is critical to the pathogenesis of AD, because it leads to the release of either toxic (AB) or trophic (sAPP) metabolites (Cummings et al., Neurology 51:S2-S17 (1998); Roch & Puttfarcken, Alz ID Res Al 1:9-16 (1996)). In this respect, it is very important to identify proteins involved in the intracellular trafficking of APP. Genetic evidence suggest that PS1 and PS2 participate in this process, which may be perturbed by AD-associated mutations in APP or the presenilins (Hardy, Trends Neurosci 20:154-159 (1997); Selkoe, Trends Cell Biol 8:447-453 (1998)). The finding that PS1 interacts with rab11a (see provisional patent application Ser. No. 60/113,534, filed 22 Dec. 1998, incorporated herein by reference) also supports a role for PS1 in the control of APP trafficking.

The family of proteins containing armadillo domains includes the members plakophilin 1 and 2, neural-specific plakophilin (also known as δ-catenin), and α-, β-, and γ-catenin. These proteins combine their structural roles, as cell-contact and cytoskeleton-associated proteins, with signaling functions, by generating and transducing signals affecting gene expression (Hatzfeld, Int Rev Cytol 186:179-224 (1999)). Recently, PS1 was found to interact with several members of the armadillo family, including β-, δ-, and γ-catenin (Zhou et al., Neuroreport 8:1489-1494 (1997); Yu et al., J Biol Chem 273:16470-16475 (1998); Murayama et al., FEBS Lett 433:73-77 (1998); Zhou et al., Neuroreport 8:2085-2090 (1997); Tanahashi & Tabira, Neuroreport 10:563-568 (1999); Kosik, Science 279:463-465 (1998)). While the significance of the interaction with δ-catenin (CTNND2) is not clear, it was suggested that the interaction between PS1 and δ-catenin is important for neuronal survival (Zhang et al., Nature 395:698-702 (1998)). To date, the interaction between PS1 and δ-catenin has not yielded many clues to AD pathogenesis, however the brain-specific expression pattern of δ-catenin suggests that it has an important function in neuronal cells, which could readily be disrupted by mutations in the presenilins. In addition, as described above, we identified an interaction between AChE and δ-catenin; between overlapping AChE fragments comprising amino acid residues 63 to 534, 355 to 614, and 355 to 517 (the smallest fragment, which still includes the δ-catenin binding domain). Because δ-catenin interacts with PS1 (Zhou et al., Neuroreport 8:1489-1494 (1997); Tanahashi & Tabira, Neuroreport 10:563-568 (1999); Kosik, Science 279:463-465 (1998)) and because of the involvement of the cholinergic is system in AD pathogenesis (Gooch & Stennett, Am J Health SystPharm 53:1545-1557(1996); Alvarez et al., J Neurosci 18:3213-3223 (1998); Inestrosa & Alarcon, J Physiol Paris 92:341-344 (1998)), this novel interaction puts δ-catenin and AChE interactors at the heart of AD pathology. In other words, all δ-catenin interactors are potentially involved in the pathoaetiology of AD. Intriguingly, a structural role for δ-catenin is suggested by our discovery that a portion of δ-catenin comprising amino acid residues 516 to 833 interacts with a polypeptide comprising amino acid residues 1311-1676 of the heavy chain of clathrin (also known as KIAA0034 or CLTC). The C-terminal fragment of APP contains the YENPTY consensus sequence of proteins that are recycled from the plasma membrane into clathrin-coated pits, and from clathrin-coated pits to endosomes (McLoughlin & Miller, FEBS Lett 397:197-200 (1996); Zambrano et al., J Biol Chem 273:20128-20133 (1997); Russo et al., FEBS Lett 434:1-7 (1998)). Moreover, a recent study showed that C- and N-terminal proteolytic fragments of PS1 are enriched in clathrin-coated vesicles of the somato-dendritic neuronal compartment (Efthimiopoulos et al., J Neurochem 71:2365-2372 (1998)). The authors concluded “PS1 proteolytic fragments are targeted to specific populations of neuronal vesicles where they may regulate vesicular function.” Thus, the interaction pattern that has emerged from our studies suggests that the δ-catenin—PS1 complex may play a central role in the intracellular trafficking of APP, through interactions with clathrin and rab 1I a. This concept is further supported by the discovery of additional interactions involving δ-catenin, described below.

Proper cell-cell adhesion is critical during development, tissue morphogenesis, and for the regulation of cell migration and proliferation—all crucial events in brain development and function. Desmosomes are adhesive intercellular junctions that anchor the intermediate filament network to the plasma membrane. By functioning both as an adhesive complex and as a cell-surface attachment site for intermediate filaments, desmosomes link the intermediate filament cytoskeletons between cells and play an important role in maintaining tissue integrity. We have discovered that a portion of δ-catenin (CTNND2) comprising amino acid residues 516 to 833 interacts with a polypeptide comprising amino acid residues 649-817 of plakophilin 2, alternative transcript b (881) (PKP2(881)). Like δ-catenin, plakophilin 2 is a member of the armadillo family. Specifically, plakophilin 2 has been found both in desmosomes and in the nucleus (Mertens et al., J Cell Biol 135:1009-1025 (1996)), suggesting a dual cellular role. The interaction between δ-catenin (a brain-specific armadillo protein) and plakophilin 2 suggests that δ-catenin and its interactors (including PS1) are involved in functions such as cell adhesion and control of gene expression. In this respect, it is worth noting that APP can not only mediate cell adhesion (Breen et al., J Neurosci Res 28:90-100 (1991)), but has also been found to be associated with nuclear proteins and transcription factors (Russo et al., FEBS Lett 434:1-7 (1998)). Hence, APP has a potential role in transcriptional regulation.

NAC (Non-AP Component of amyloid plaques) is a peptide of 35 residues originally isolated from amyloid material in brain cortex from an AD patient (Ueda et al., Proc Natl Acad Sci USA 90:11282-11286 (1993)). Cloning of a cDNA encoding NAC revealed that NAC is generated by proteolytic cleavage of a larger protein, the NAC precursor, or NACP (Ueda et al., Proc Natl Acad Sci USA 90:11282-11286 (1993)). Recently, we discovered that δ-catenin interacts directly with NACP (see International Patent Application No. PCT/US99/30396 (WO 00/37483)). It is interesting that the two major components of the senile plaques (Aβ and NAC) are both generated by cleavage of a precursor protein (APP and NACP, respectively). Subsequent studies have demonstrated that the NAC peptide is itself amyloidogenic (i.e., it self-aggregates into amyloid material) and that it binds Aβ and stimulates its aggregation (Yoshimoto et al., Proc Natl Acad Sci USA 92:9141-9145 (1995); Iwai et al., Biochemistry 34:10139-10145 (1995)). In addition, NACP was identified as a presynaptic protein in the central nervous system, suggesting that it plays a role in synaptic function (Iwai et al., Biochemistry 34:10139-10145 (1995)). Thus, cleavage o f NACP into NAC results in the release of an amyloidogenic fragment and may independently impair synaptic function. The similarity of these features with APP/Aβ is again striking. Indeed, another study suggested that there is a connection between the metabolism of presynaptic proteins and amyloid formation (Masliah et al., Am J Pathol 148:201-210 (1996)). In this respect, it should also be noted that ApoE4 binding to NAC is twice as strong as that of ApoE3 (Olesen et al., Brain Res Mol Brain Res 44:105-112 (1997)), and the presence of the E4 allele has been identified as a risk factor for AD (Hardy, Am J Med Genet 60:456-460 (1995); Strittmatter & Roses, Proc Natl Acad Sci USA 92:4725-4727 (1995); Falduto & LaDu, Alzheimer's Disease (Brioni J D, Decker M W, eds.), New York: Wiley Press (1996)). Recently, mutations in NACP have been found to co-segregate with early-onset familial Parkinson's disease (Polymeropoulos et al., Science 276:2045-2047 (1997)). Furthermore, these mutations were shown to disrupt NACP binding to brain vesicles involved in fast axonal transport (Jensen et al., J Biol Chem 273:26292-26294 (1998)). Since APP is known to undergo fast axonal transport (Koo et al., Proc Natl Acad Sci USA 87:1561-1565 (1990)), the δ-catenin—NACP connection again brings δ-catenin right into the process of intracellular trafficking of APP, at the very heart of AD pathogenesis.

The mechanism of Aβ toxicity has always been controversial (Iversen et al., Biochem J 311:1-16 (1995); Manelli & Puttfarcken, Brain Res Bull 38:569-576 (1995); Gillardon et al., Brain Res 706:169-172 (1996); Behl et al., Biochem Biophys Res Commun 186:944-950 (1992); Weiss et al., J Neurochem 62:372-375 (1994); Octave, Rev Neurosci 6:287-316 (1995); Furukawa et al., J Neurochem 67:1882-1896 (1996); Schubert, Eur J Neurosci 9:770-777 (1997)). Reports of neuronal apoptosis have been contradicted by studies showing that necrosis was the cause of cell death (Loo et al., Proc Natl Acad Sci USA 90:7951-7955 (1993); Behl et al., Brain Res 645:253-264 (1994); Bancher et al., J Neural Transm 104:Suppl. 50:141-152 (1997); Schubert, Eur J Neurosci 9:770-777 (1997)). In any case, it is clear that events such as generation of free radicals, lipid peroxidation, and disruption of calcium homeostasis play major roles in Aβ toxicity (Weiss et al., J Neurochem 62:372-375 (1994); Abe & Kimura, J Neurochem 67:2074-2078 (1996); Mark et al., J Neurochem 68:255-264 (1997); Kruman et al., J Neurosci 17:5089-5100 (1997)). To elucidate this phenomenon, investigators used the yeast two-hybrid system to look for proteins that interact with the Aβ peptide and which could mediate its toxicity. A novel protein named ERAB was identified (Yan et al., J Biol Chem 274:2145-2156 (1997)), which later turned out to be identical to a 3-hydroxyacyl-CoA dehydrogenase (He et al., J Biol Chem 273:10741-10746 (1998)). The original report also claimed that ERAB mediates Aβ toxicity (Yan et al., J Biol Chem 274:2145-2156 (1997)), and a recent study showed that it does so by generating toxic adlehydes from alcohol (Yan et al., J Biol Chem 274:2145-2156 (1999)). To gain more information about ERAB, we sought to identify addition interactors of the full-length protein. Intriguingly, we discovered that ERAB interacts with a polypeptide comprising amino acid residues 257-792 of δ-catenin (CTNND2). This interaction, like the δ-catenin—NACP interaction described above, brings δ-catenin to the heart of APP metabolism. Also, the interactions between ERAB and Aβ (a proteolytic product of APP), between ERAB and δ-catenin, and between δ-catenin and PS-1 generate a possible biochemical link between PS-1 and APP, which could explain how the mutations in PS1 associated with FAD could alter APP metabolism.

Thus, the five novel protein-protein interactions we have identified so far that involve δ-catenin (with AChE, ERAB, NACP, clathrin, and plakophilin 2) put δ-catenin at the crossroads of biochemical and cellular events involved in AD pathogenesis. Although δ-catenin alone may prove not be a suitable drug target, compounds that alter its interaction pattern with its many binding partners could be of therapeutic value for the treatment of AD or its symptoms. Likewise, the proteins that interact with δ-catenin could themselves prove to be attractive drug targets, precisely because of the intimate connection between δ-catenin and AD pathoaetiology.

The product of the bcl-2 proto-oncogene is a mitochondrial protein that was shown to block neuronal apoptosis (Hockenbery et al., Nature 348:334-336 (1990)). The anti-apoptotic activity of bcl-2 is quite relevant to AD in the light of two recent studies that showed that bcl-2 blocks neuronal death induced by Aβ in transgenic mice (Cribbs et al., Soc Neurosci Abstr 20:604 (1994)), or by FAD-associated PS1 mutations in transfected cells (Guo et al., J Neurosci 17:4212-4222 (1997)). However, a direct biochemical link between bcl-2 and AD-related protein has not yet been shown. In an effort to better define the role of bcl-2-alpha, we sought to identify more of the proteins with which it interacts. We initially discovered that a portion of bcl-2-alpha comprising amino acid residues 1 through 75 interacted with a fragment of δ-catenin comprising amino acids 690 to 1225. We have since discovered that a smaller fragment of δ-catenin, comprising only amino acid residues 257-792, still interacts with the N-terminal region of bcl-2-alpha. This interaction effectively generates a link between PS1 and bcl-2-alpha and might explain the anti-apoptotic activity of wild-type PS1, as well as why FAD associated mutations in PS1 activate neuronal apoptosis (Guo et al., J Neurosci 17:4212-4222 (1997); Kim & Tanzi, Curr Opin Neurobiol 7:683-688 (1997); Kovacs & Tanzi, Cell Mol Life Sci 54:902-909 (1998); Tesco et al., J Biol Chem 273:33909 (1998)). Given this link, drugs that modulate the interaction between δ-catenin and PS1, and between δ-catenin and bcl-2-alpha, might help prevent neuronal apoptosis as observed in the brains of AD patients.

Investigating further the interactions made by δ-catenin we discovered that polypeptides comprising amino acid residues 516 through 833, and 1006 through 1158, interacted with the break point cluster protein (BCR) amino acid residues 1100-1227, and essentially full-length 14-3-3 3 protein (14-3-3-b(246)), respectively. Interestingly, these two proteins were previously known to interact with each other (Braselmann & McCormick, EMBO J 14:4839-4848 (1995)). Of these two proteins, BCR is a GTP-binding protein that activates GTPases of the Ras family (Diekmann et al., EMBO J 14:5297-5305 (1995)), which participates in the chromosomal translocation with the c-Abl oncogene to generate the hybrid Bcr-Abl oncogene that is responsible for several forms of leukemia (Warmuth et al., Ann Hemotol 78:49-64 (1999)). Curiously, the two individual proteins comprising the hybrid oncogene, BCR and c-Abl, were also shown to interact directly with each other (Pendergast et al., Cell 66:161-171 (1991)). The GTPase activating function of BCR is interesting in the light of the PS1-rab11a interaction we observed previously (see provisional patent application Ser. No. 60/113,534, filed 22 Dec. 1998, incorporated herein by reference). The rab11a protein is also a GTPase, involved in intracellular vesicle trafficking and membrane fusion, and expressed in the CNS (Ullrich et al., J Cell Biol 135:913-924 (1996); Sheehan et al., Neuroreport 7:1297-1300 (1996); Chen et al., Mol Biol Cell 9:3241-3257 (1998)). Thus, the δ-catenin-BCR complex could modulate vesicle trafficking though interactions with PS1 and rab11a. Mutations in PS1 associated with FAD could disrupt these interaction and alter the normal trafficking machinery, leading to the production of toxic metabolites like Aβ. The 14-3-3β protein (14-3-3-b(246)) is a well known modulator of protein kinase C (PKC), and is expressed at high levels in the CNS (Skoulakis & Davis, Mol Neurobiol 16:269-284 (1998); Aitken et al., Mol Cell Biochem 149-150:41-49 (1995)). PKC activity is a critical factor regulating the α-secretion of APP (Govoni et al., Ann NY Acad Sci 777:332-337 (1996); Rossner et al., Prog Neurobiol 56:541-569 (1998); Jin & Saitoh, Drugs Aging 6:136-149 (1995)). Thus, as PS1 interacts with δ-catenin, and δ-catenin interacts with BCR and 14-3-3β (which also interact with each other), mutations in PS-1 associated with FAD could influence the stability of the complex formed by δ-catenin, BCR, and 14-3-3β, which, in turn, could affect PKC activity and the α-secretion of APP. A similar model has recently been proposed for the effect of FAD-associated PS1 mutations that could destabilize a β-catenin complex and trigger neuronal apoptosis (Zhang et al., Nature 395:698-702 (1998)). Therefore, drugs that modulate the interactions of δ-catenin with BCR and/or 14-3-3β could potentially control a-secretase activity and the eventual generation of the trophic secreted form of APP, or the toxic Aβ peptide. Finally, another important connection can be made between the δ-catenin—14-3-3β pathway and the PS1—FKBP25 pathway. As described above, FKBP25 is a protein from the immunophilin family that is involved in the neurotrophic effects of immunosuppressant drugs such as FK506 and rapamycin (Snyder et al., Trends Pharmacol Sci 19:21-26 (1998); Steiner et al., Nat Med 3:421-428 (1997); Steiner et al., Proc Natl Acad Sci USA 94:2019-2024 (1997)). While the FK506 effects are mediated by the calcium-activated phosphatase calcineurin (Snyder et al., Trends Pharmacol Sci 19:21-26 (1998)), rapamycin effects are transduced by the TOR kinase (Chiu et al., Proc Natl Acad Sci USA 91:12574-12578 (1994); Lorenz & Heitman, J Biol Chem 270:27531-27537 (1995)). Although FKBP25 binds FK506, it has a much higher affinity for rapamycin (Galat et al., Biochemistry (1992)), suggesting that FKBP25 signals through the TOR kinase system. Recently, it was shown that the rapamycin signaling pathway makes use of 14-3-30. (Bertram et al., Curr Biol 8:1259-1267 (1998)). Thus, the neurotrophic effects elicited by FKBP25 (a PS1 interactor) are likely mediated by 14-3-3β (a δ-catenin interactor). Again, it is possible that FAD-associated mutations in PS1 could serve to disrupt its interaction with δ-catenin, and thus impair the 14-3-3β-mediated neurotrophic effect of FKBP25.

In the same search for δ-catenin interactors, we discovered that the same fragment of δ-catenin (comprising amino acid residues 1006 to 1158) interacted with the 14-3-3ζ protein (14-3-3z). 14-3-3ζ is also a PKC modulator (Aitken et al., Mol Cell Biochem 149-150:41-49 (1995)) and is 87% identical (93% similar) to 14-3-3β. Despite this high level of similarity, it is not known whether 14-3-3ζ interacts with BCR, as 14-3-3β does. In any case, the PKC modulating activity of 14-3-3ζ, and its interaction with δ-catenin also make possible for the PS1:δ-catenin complex to control α-secretase activity and thereby control the production of the trophic secreted form of APP, or the toxic Aβ peptide.

In the same search for δ-catenin interactors, we discovered that the same fragment of δ-catenin (comprising amino acid residues 1006 to 1158) interacted with a polypeptide comprising amino acid residues 625-1009 of the focal adhesion kinase 2 (FAK2). As mentioned above, FAK2, also called proline-rich tyrosine kinase 2 (PYK2) or cell adhesion kinase β (CAKβ), is the second member of the focal adhesion kinase family. Focal adhesion kinases (FAKs) form a special subfamily of cytoplasmic protein tyrosine kinases (PTKs). In contrast to other non-receptor PTKs, FAKs do not contain SH2 or SH3 domains, but, instead, have a carboxyl-terminal proline-rich domain, which is important for protein-protein interactions (Schaller, Soc Gen Physiol Ser 52:241-255 (1997); Schaller & Parsons, Curr Opin Cell Biol 6:705-710 (1994); Parsons et al., J Cell Sci 18:109-113 (1994)). FAK2 is expressed at its highest levels in brain, at medium levels in kidney, lung, and thymus, and at low levels in spleen and lymphocytes (Avraham et al., J Biol Chem 270:27742-27751 (1995)). In brain, FAK2 is found at highest levels in the hippocampus and amygdala (Avraham et al., J Biol Chem 270:27742-27751 (1995)), two areas severely affected in AD. FAK2 is thought to participate in signal transduction mechanisms elicited by cell-to-cell contacts (Sasaki et al., J Biol Chem 270:21206-21219 (1995)). It is involved in the calcium-induced regulation of ion channels, and it is activated by the elevation of intracellular calcium concentration following the activation of G protein-coupled receptors (GPCRs) that signal though Gaq and the phospholipase C (PLC) pathway (Yu et al., J Biol Chem 271:29993-29998 (1996)). Thus, FAK2 is an important intermediate signaling molecule between GPCRs activated by neuropeptides or neurotransmitters and downstream signals that modulate the neuronal activity (e.g., channel activation, membrane depolarization). Such a link between intracellular calcium levels, tyrosine phosphorylation, and neuronal activity is clearly important for neuronal survival and synaptic plasticity (Siciliano et al., J Biol Chem 271:28942-28946 (1996)). The interaction of FAK2 with δ-catenin, and its high levels of expression in hippocampus and amygdala, suggest that a disruption of its activity may be related to neuronal death in AD. Compounds that modulate the activity of FAK2, or modulate its interaction with δ-catenin may thus prove beneficial therapeutic agents for the treatment of AD.

Continuing our search for for δ-catenin interactors, we discovered that a fragment comprised of amino acid residues 516 to 833 of δ-catenin (CTNND2) interacted with a polypeptide comprised of amino acid residues 343-822 of the epidermal growth factor receptor kinase substrate 8 (EPS8). EPS8, a protein of 822 amino acids, is an intracellular substrate for a several different receptors with tyrosine kinase activity, as well as a non-receptor kinase. Upon binding to the EGF receptor, EPS8 enhances mitogenic signals mediated by EGF (Fazioli et al., EMBO J 12:3799-3808 (1993); Wong et al., Oncogene 9:3057-3061 (1994)). EPS8 is thought to play an essential function in cell growth regulation and in the reorganization of the cytoskeleton, perhaps by acting as a docking site for other signaling molecules (Provenzano et al., Exp Cell Res 242:186-200 (1998)). In this respect, δ-catenin could serve as a bridge between EPS8 and FAK2 or another tyrosine kinase. Since EPS8 is associated with cell division, abnormal signaling through EPS8 leading to mitosis could trigger apoptosis in post-mitotic cells such as neurons. Thus, compounds that modulate EPS8 phosphorylation, or its interactions with other proteins, could enhance neuronal survival and serve as therapeutic agents for the treatment of AD or its symptoms.

Using fragments of δ-catenin (CTNND2) comprising amino acid residues 1006 through 1158 and 1006 through 1225 we discovered and confirmed an interaction with polypeptide fragments of the KIAA0443 protein, comprising amino acid residues 1161-1245 and 1161-1395, respectively.) At the time of our discovery, KIAA0443 was a novel protein for which a cDNA was randomly cloned out of a human brain library (Ishikawa et al., DNA Res 4:307-313 (1997)). Searching for motifs and patterns in the KIAA0443 amino acid sequence we noted the presence of an ATP/GTP binding domain. Therefore, we postulated that it was possible that KIAA0443 was a GTP or ATP exchange factor that functions together with another δ-catenin interactor such as Bcr or FAK2, or with a PS1 interactor such as rab11. We also identified several lipocalin signature domains in KIAA0443, which suggest that this protein may be involved in the transport of small hydrophobic molecules. Although the biological functions of KIAA0443 were not initially clear from its structure, its interaction with δ-catenin, a brain-specific protein, suggested that it was involved in some kind of brain-specific function. Hence, we suggested that compounds that modulate the δ-catenin-KIAA0443 interaction could thus influence neuronal and synaptic functions. More recently, Whistler and colleagues identified KIAA0443 as a protein that binds preferentially to the cytoplasmic tail of the delta opioid receptor (DOR), and serves as a candidate heterotrimeric GTP-binding protein (G protein)-coupled receptor-associated sorting protein. Consequently, they named the protein GASP. They also showed that disruption of the DOR-GASP interaction through receptor mutation, or overexpression of a dominant negative fragment of GASP, inhibited receptor trafficking to lysosomes and promoted recycling. They noted that the GASP family of proteins may modulate lysosomal sorting and functional down-regulation of a variety of G protein-coupled receptors (Whistler et al., Science 297:615-620 (2002)).

As was noted above, there is a growing body of evidence that disruption of energy metabolism is an important factor in neurodegenerative disorders, including AD (Beal, Biochem Biophys Acta 1366:211-223 (1998); Nagy et al., Acta Neuropathol 97:346-354 (1999); Rapoport et al., Neurodegeneration 5:473-476 (1996)). Mitochondrial dysfunctions result in low ATP levels and production of free oxiradicals that are extremely toxic to neurons (Simonian & Coyle, Annu Rev Pharmacol Toxicol 36:83-106 (1996); Beal, Curr Opinion Neurobiol 6:661-666 (1996)). To gain insight into the involvement of the mitochondrial machinery in AD pathogenesis, we sought to identify interactors of Alzheimer related proteins that are either mitochondrial proteins, or somehow involved in energy metabolism.

We first discovered an interaction between the 91 N-terminal amino acid residues of PS-1 and amino acid residues 135-433 of α-enolase (ENOA), a glycolytic enzyme that transforms 2-phosphoglycerate into phosphoenol pyruvate, and is thus directly involved in energy production. Next we found that the enzymes citrate synthase (cit-synt) and aldolase C (ALDOC) interacted with amino acid residues 301-600 of axin. Aldolase is active as a homotetramer, involved in glycolysis—it cleaves fructose bi-phosphate into dihydroxyacetone phosphate and glyceraldehyde 3-phosphate. The 3 isoforms of aldolase, A, B, and C, are found in muscle, liver, and brain, respectively. Citrate synthase catalyzes the first step of the Krebs cycle—the condensation of oxaloacetate and acetyl-CoA into citrate, with the concomitant release of CoA and energy (7.7 kcal/mol). Unlike aldolase and α-enolase (cytosolic), citrate synthase is located in the mitochondrial matrix. We also found an interaction between amino acid residues 1-300 of axin and creatine kinase B (CKB). CKB is a well-characterized cytosolic enzyme involved in energy metabolism, which is likely to be very important for an organ like brain where the demand for energy fluctuates rapidly and over a large range. Creatine kinase exists in two cytosolic isoforms called M and B, plus two mitochondrial isoforms. The cytosolic enzyme is active either as a homo- or heterodimer. The MM enzyme is found in heart and skeletal muscle, the MB enzyme mostly in heart, and the BB enzyme in many other tissues, especially brain.

In addition, we identified an interaction between amino acid residues 301-600 of axin and neurogranin (NRGN). NRGN is a small (78 amino acid residue) protein that belongs to the calpacitin family, along with GAP-43 and PEP-19. While GAP-43 is found in the axonal compartment, neurogranin is associated with post-synaptic membranes (Gerendasy & Sutcliffe, Mol Neurobiol 15:131-163 (1997)). It is involved in the development of dendritic spines, LTP, LTD, learning and memory (Gerendasy & Sutcliffe, Mol Neurobiol 15:131-163 (1997)). Although its exact function is not yet clear, available models suggest that neurogranin regulates the availability of calmodulin, and, in turn, calmodulin regulates neurogranin's ability to amplify the mobilization of calcium in response to stimulation of metabotropic glutamate receptor. Neurogranin and GAP-43 release calmodulin rapidly in response to a large calcium influx, and slowly in response to a small influx. Therefore, these proteins act like a “calcium capacitor” (hence the name calpacitin). The amount of calcium that the system can handle (capacitance) is regulated by PKC phosphorylation of neurogranin (and GAP-43), which blocks its binding to calmodulin (Gerendasy & Sutcliffe, Mol Neurobiol 15:131-163 (1997)). Therefore, the ratio of phosphorylated to non-phosphorylated neurogranin could control the LTP/LTD sliding threshold (together with calcium-calmodulin dependent kinase II). Most importantly, neurogranin has been reported to be associated with mitochondria, in order to couple energy production with dendritic spine formation and synaptic plasticity (Gerendasy & Sutcliffe, Mol Neurobiol 15:131-163 (1997)).

Additionally, we found that a variety of fragments of axin from the region encompassing amino acid residues 301-750, interacted with a thioredoxin-dependent peroxide reductase, otherwise known as anti-oxidant mitochondrial protein (AOP-1), or peroxiredoxin 3 (PRDX3). The anti-oxidant properties of PRDX3 suggest that is might protect neurons role against oxidative insults, as does the anti-oxidant vitamin (Behl et al., Biochem Biophys Res Commun 186:944-950 (1992)).

Remarkably, we have now identified six important interacting proteins, or interactors, of the two neuronal proteins axin and PS1. Four of these interactors are enzymes involved in energy production (α-enolase, aldolase C, citrate synthase, and creatine kinase B), while one is a protein involved in the formation of dendritic spines, LTP, and memory, and the other is a known anti-oxidant protein. In light of the well documented association of certain mitochondrial disorders with particular neurodegenerative conditions (Beal, Biochem Biophys Acta 1366:211-223 (1998); Nagy et al., Acta Neuropathol 97:346-354 (1999)), often involving the production of toxic oxiradical species (Busciglio & Yankner, Nature 378:776-779 (1995); Richardson et al., Ann NY Acad Sci 777:362-367 (1996); Simonian & Coyle, Annu Rev Pharmacol Toxicol 36:83-106 (1996); Beal, Curr Opin Neurobiol 6:661-666 (1996)), these newly identified interactions open new promising therapeutic and diagnostic avenues.

We also found that a polypeptide comprising amino acid residues 301-600 of axin interacted with polypeptides representing the N-terminal half of the small GTPase rab3A. Like rab11, rab3A is involved in intracellular vesicle trafficking. Specifically, rab3A plays a major role in the trafficking of synaptic vesicles (Geppert & Sudhof, Annu Rev Neurosci 21:75-95 (1998)) and thus, may regulate neurotransmitter release. Rab3A expression is reported to be brain specific, and essential for LTP of mossy fiber synapses in the hippocampus (Castillo et al., Nature 388:590-593 (1997)), the most severely affected area in the brains of AD patients. This observation is crucial because LTP is known to be impaired in the hippocampus of mice transgenic for the carboxy-terminal region of APP (Nalbantoglu et al., Nature 387:500-505 (1997)).

We also discovered that a portion of axin comprising amino acid residues 301-600 interacted with two proteins involved in RNA metabolism—the splicing factors SRp30c (also known as splicing factor, arginine/serine-rich 9, or SFRS9) and SMN1 (for survival for motor neurons 1). These two proteins are composed of 221 and 294 amino acid residues, respectively, and are part of the spliceosome complex (Screaton et al., EMBO J 14:4336-4349 (1995); Pellizzoni et al., Cell 95:615-624 (1998); Talbot et al., Hum Mol Genet 6:497-500 (1997)). The relevance of these interactions from an AD disease perspective is that mutations in SMN1 cause a variety of autosomal recessive neurodegenerative disorders, including SMA (spinal muscular atrophy), that are distinguished by the age of onset and the severity of the clinical features, and are characterized by the degeneration of lower motor neurons, resulting paralysis (Lefebvre et al., Hum Mol Genet 7:1531-1536 (1998); Lefebvre et al., Cell 80:155-165 (1995)). Sadly, SMA is often fatal. SMN1 is expressed in many regions of the central nervous system, including the spinal cord, brainstem, cerebellum, thalamus, cortex (especially the layer V, which is most affected in AD patients) and hippocampus (also deeply affected in AD patients) (Bechade et al., Eur J Neurosci 11:293-304 (1999)). A role for SMN1 in nucleo-cytoplasmic and dendritic transport has also been proposed (Bechade et al., 1999). In addition, the role of SMN1 in neuron survival is thought to be mediated by the anti-apoptotic protein bcl-2 (Lefebvre et al., Hum Mol Genet 7:1531-1536 (1998), which we found to interact with δ-catenin (see above). Thus, axin interacts with two proteins involved in RNA splicing, one of which is directly linked to neuron survival and is expressed in regions of the brain that are severely affected in AD.

Transcription factor CP2 (TFCP2), which is also known as LSF, is a transcription factor that was reported to interact with Fe65 (also known as APPB1), a well known APP interactor described above (Zambrano et al., J Biol Chem 273:20128-20133 (1998)). The relevance of this interaction remains obscure, although it has been proposed that the LSF/TFCP2:Fe65 complex could control APP trafficking and metabolism (Russo et al., FEBS Lett 434:1-7 (1998)). Our own data reveal two important novel interactions that address the potential role of LSF/TFCP2 in AD pathogenesis: First, we have found that amino acid residues 1-91 of PS1 interact with a polypeptide comprising amino acid residues 405-502 of LSF/TFCP2. Second, we have discovered that a polypeptide comprising amino acid residues 393-502 of LSF/TFCP2 interacts directly with an N-terminal fragment of APP (comprising amino acid residues 1-220). Thus, LSF/TFCP2 interacts directly with Fe65, APP, and PS1. This finding puts LSF/TFCP2 and its interactors at the center of AD pathogenesis. Finally, we have also found that the same polypeptide comprising amino acid residues 393-502 of LSF/TFCP2 interacts with a small protein (71 amino acids) called 4F5s. Although the function of this protein is not known, it was reported to be a potential genetic modifier of SMN1 (Scharf et al., Nat Genet 20:83-86 (1998)). At this juncture, however, it is unknown whether SMN1 and 4F5s directly interact.

In conclusion, we have identified a series of interactions (axin with SRp30c and SMN1, LSF/TFCP2 with PS1, APP and 4F5s), which generate a protein-interaction network that brings the splicing factors SRp30c and SMN1, and the small protein 4F5s, into the center of AD pathoaetiology. Two of these proteins are directly involved in neuron survival, and the expression pattern of one of them matches the regions of the brain most severely affected in AD patients. Thus, these newly identified interactions open promising new therapeutic and diagnostic avenues for use in the treatment AD and its symptoms.

In view of the above description new pathways involving the major Alzheimer proteins can be elucidated. APP is the metabolic precursor of the Aβ peptide found in the core of neuritic amyloid plaques, and which is directly toxic to neurons. One APP processing pathway leads to the release of βsAPP, which shows a weak activity of neuronal survival, neurite outgrowth, synaptic maintenance and enhanced memory. However, another metabolic pathway (which is non-amyloidogenic) releases βsAPP, whose neurotrophic activity is much stronger than that of βsAPP. Mutations in PS1 are known to influence APP metabolism and result in the production of more Aβ42—the most toxic form of the Aβ peptide. Axin was found to interact with peroxiredoxin 3, a mitochondrial enzyme that protects neurons against oxidative insults by free radicals. Axin was also found to interact with citrate synthase, aldolase C, and creatine kinase B, while PS1 was found to interact with a-enolase. These four enzyme interactors are all involved in energy metabolism, the disruption of which is a known cause of neurodegeneration (Beal, Biochem Biophys Acta 1366:211-223 (1998); Nagy et al., Acta Neuropathol 97:346-354 (1999); Rapoport et al., Neurodegeneration 5:473-476 (1996)). Axin was also found to interacts with rab3a and neurogranin, two proteins involved in the development of dendritic spines (a process that requires large amount of energy), which are essential for LTP in the hippocampus.

APP and PS1 were both found to interact with LSF/TFCP2, which also interacts with Fe65, which in turn interacts with APP. PS1 was found to interact with δ-catenin, which, in turn, interacts with ERAB, an APP interactor. Thus, LSF/TFCP2, 6-catenin, and their interacting protein partners are at the center of AD pathoaetiology. Axin was also found to interact with SMN1 and SRp30c, two proteins involved in RNA metabolism. In addition, SMN1 is involved in neuronal survival, an activity that is mediated by bcl2, a δ-catenin interactor. Finally, the protein 4F5s, which is an apparant genetic modifier of SMN1, was also found to interact with LSF/TFCP2.

The proteins disclosed in the present invention were found to interact with PSI, APP or other proteins involved in AD pathogenesis, in the yeast two-hybrid system. Because of the involvement of these proteins in AD, the interacting proteins disclosed herein likely also participate in the pathoaetiology of AD. Therefore, the present invention provides a list of interacting proteins heretofore unknown to be involved in AD pathogenesis, and discloses uses of those proteins, and DNA encoding those proteins, for the development of diagnostic and therapeutic tools for the study, early diagnosis, and possible treatment of AD and its symptoms. This list includes, but is not limited to, the following examples.

### 2.2. Protein Complexes

Accordingly, the present invention provides protein complexes formed by interacting pairs of proteins described in the tables above. The present invention also provides protein complexes in which one or more of the interacting protein members are native proteins or homologues, derivatives or fragments of native proteins.

Thus, for example, one interacting partner in a protein complex can be a complete native δ-catenin, a δ-catenin homologue capable of interacting with, e.g., FAK2, a δ-catenin derivative, a derivative of the δ-catenin homologue, a δ-catenin fragment capable of interacting with FAK2 (e.g., δ-catenin fragment(s) containing the coordinates shown in Tables 53 and 54), a homologue or derivative of the δ-catenin fragment, or a fusion protein containing (1) complete native δ-catenin, (2) a δ-catenin homologue capable of interacting with FAK2 or (3) a δ-catenin fragment capable of interacting with FAK2. Besides native FAK2, useful interacting partners for δ-catenin, or a homologue or derivative or fragment thereof, also include homologues of FAK2 capable of interacting with δ-catenin, derivatives of the native or homologue FAK2 capable of interacting with δ-catenin, fragments of the FAK2 capable of interacting with δ-catenin (e.g., a fragment containing the identified interacting regions shown in Tables 53 and 54), derivatives of the FAK2 fragments, or fusion proteins containing (1) a complete FAK2, (2) a FAK2 homologue capable of interacting with δ-catenin, or (3) a FAK2 fragment capable of interacting with δ-catenin.

FAK2 fragments capable of interacting with δ-catenin can be identified by the combination of molecular engineering of a FAK2-encoding nucleic acid and a method for testing protein-protein interaction. For example, the coordinates in Tables 53 and 54 can be used as starting points and various FAK2 fragments falling within the coordinates can be generated by deletions from either or both ends of the coordinates. The resulting fragments can be tested for their ability to interact with δ-catenin using any methods known in the art for detecting protein-protein interactions (e.g., yeast two-hybrid methods). Alternatively, various FAK2 fragments can be made by chemical synthesis. The chemically-synthesized FAK2 fragments can then be tested for their ability to interact with δ-catenin using any method known in the art for detecting protein-protein interactions. Examples of such methods include protein affinity chromatography, affinity blotting, in vitro binding assays, yeast two-hybrid assays, and the like. Likewise, δ-catenin fragments capable of interacting with FAK2 can also be identified in a similar manner.

Other protein complexes can be formed in a similar manner based on any of the other interacting pairs of proteins provided in the tables above.

In a specific embodiment of the protein complex of the present invention, two or more interacting partners are directly fused together, or covalently linked through a peptide linker, forming a hybrid protein having a single unbranched polypeptide chain. Thus, the protein complex may be formed by “intramolecular” interactions between two portions of the hybrid protein. Again, one or both of the fused or linked interacting partners in this protein complex may be a native protein or a homologue, derivative or fragment of a native protein.

The protein complexes of the present invention can also be in a modified form. For example, an antibody selectively immunoreactive with the protein complex can be bound to the protein complex. In another example, a non-antibody modulator capable of enhancing the interaction between the interacting partners in the protein complex may be included. Alternatively, the protein members in the protein complex may be cross-linked for purposes of stabilization. Various crosslinking reagents and methods may be used. For example, a bifunctional reagent in the form of R—S—S—R′ may be used in which the R and R′ groups can react with certain amino acid side chains in the protein complex forming covalent linkages. See e.g., Traut et al., in Creighton ed., Protein Function: A Practical Approach, IRL Press, Oxford, 1989; Baird et al., J Biol Chem, 251:6953-6962 (1976). Other useful crosslinking agents include, e.g., Denny-Jaffee reagent, a heterbiofunctional photoactivable moiety cleavable through an azo linkage (See Denny et al., Proc Nat Acad Sci USA, 81:5286-5290 (1984)), and 1251-{S-[N-(3-iodo-4-azidosalicyl) cysteaminyl]-2-thiopyridine}, a cysteine-specific photocrosslinking reagent (see Chen et al., Science, 265:90-92 (1994)). The above-described protein complexes may further include any additional components, e.g., other proteins, nucleic acids, lipid molecules, monosaccharides or polysaccharides, ions, etc.

The present invention also provides isolated nucleic acid molecules. The nucleic acid molecules can be in the form of DNA, RNA, or a chimera or hybrid thereof, and can be in any physical structures including single-stranded or double-stranded molecules, or in the form of a triple helix. In one embodiment, the isolated nucleic acid molecule has a sequence of SEQ ID NO: 1 or the complement thereof.

In addition, nucleic acid molecules are also contemplated, which are capable of specifically hybridizing, under stringent hybridization conditions, to a nucleic acid molecule having the sequence of SEQ ID NO: 1 or the coding sequence or complement thereof. Preferably, such nucleic acid molecules encode a polypeptide having the sequence of SEQ ID NO:2, or a fragment thereof.

In another embodiment, an isolated nucleic acid molecule is provided, which has a sequence that is at least 50%, preferably at least 60%, more preferably at least 75%, 80%, 82%, 85%, even more preferably at least 90%, 91%, 92%, 93%, 94%, 95%, 96%, 97%, 98%, or 99% identical to the sequence of SEQ ID NO:1 or the coding sequence or complement thereof. Preferably, such nucleic acid molecules encode a polypeptide having the sequence of SEQ ID NO:2.

As is apparent to skilled artisans, homologous nucleic acids or nucleic acids capable of hybridizing with a nucleic acid of the sequence of SEQ ID NO: 1, or the coding sequence thereof, can be prepared by manipulating a nucleic acid molecule having a sequence of SEQ ID NO:1. For example, various nucleotide substitutions, deletions or insertions can be incorporated into the nucleic acid molecule by standard molecular biology techniques. As will be apparent to skilled artisans, such nucleic acids are useful irrespective of whether they encode a functional protein. For example, they can be used as probes for isolating and/or detecting nucleic acids. Nevertheless, preferably the homologous nucleic acids or the nucleic acids capable of hybridizing with a nucleic acid of the sequence of SEQ ID NO: 1 encode a polypeptide having one or more activities of the polypeptides encoded by SEQ ID NO: 1.

In addition, nucleic acid molecules that encode the proteins having an amino acid sequence of SEQ ID NO:2 are also intended to fall within the scope of the present invention. As will be immediately apparent to a skilled artisan, due to genetic code degeneracy, such nucleic acid molecules can be designed conveniently by nucleotide substitutions in the wild-type nucleotide sequence of SEQ ID NO:1.

The present invention further encompasses nucleic acid molecules encoding a protein that has a sequence that is at least 75%, preferably at least 85%, 90%, 91%, 92%, 93%, or 94%, and more preferably at least 95%, 96%, 97%, 98%, or 99% identical to the sequence of SEQ ID NO:2. The various nucleic acid molecules may be produced by chemical synthesis and/or recombinant techniques based on an isolated nucleic acid molecule having a sequence of SEQ ID NO:1, or the complement thereof.

In another embodiment of the present invention, oligonucleotides are provided having a contiguous span of at least 10, 12, 15, 16, 17, 18, 19, 20, 21, 22, 23, 24, 25, 26, 27, 28, 29, 30, 35, 50, 75, 100, 200, 300, 400, 500, 600, 700, 800, 900, 1000, 1100, 1200, 1300, 1400, 1500, 1600, 1800, 2000, 2200, 2400, 2600 or 2760 nucleotides of the sequence of SEQ ID NO: 1, or the complement thereof. Preferably, the oligonucleotides are less than the full length of the sequence of SEQ ID NO: 1, more preferably no greater than 1200, 800, 600, 400, 200, 100, or 50 nucleotides in length. In a preferred embodiment, the oligonucleotides have a length of about 12-18, 19-25, 26-34, 35-50, or 51-100 nucleotides. In a specific embodiment, the oligonucleotide is a sequence encoding a contiguous span of at least 4, 5, 6, 7, 8, 9, 10, 11, 12, 13, 14, 15, 17, 20, 22, 25, 30, 35, 50, 100, 150, 200, 300, 400, or 500 amino acids of SEQ ID NO:1. In another specific embodiment, the oligonucleotide is an antisense oligo as described in Section 6.2.3, below. In another specific embodiment, the oligonucleotide is a ribozyme molecule as described in Section 6.2.4, below. In yet another specific embodiment, the oligonucleotide can serve as a primer for nucleic acid amplification reactions, such as the Polymerase Chain Reaction (PCR). In yet another specific embodiment, the oligonucleotides of the present invention are used to prepare siRNAs capable of reducing the expression of specific, gene products by inducing RNA interference of RNA transcripts of corresponding sequence, as described in Section 6.2.2, below.

The present invention further encompasses oligonucleotides that have a length of at least 10, 12, 15, 17, 18, 19, 20, 21, 22, 23, 24, 25, 30, 50, 75, 100, 200, 300, 400, 500, or 600 nucleotides and preferably no greater than 1500, 1300, 1100, 800, 600, 400, 200 or 100 nucleotides, and are at least 85%, 90%, 92% or 94%, and more preferably at least 95%, 96%, 97%, 98%, or 99% identical to a contiguous span of nucleotides of the sequence of SEQ ID NO:1, or the complement thereof. The oligonucleotides can have a length of about 12-18, 19-25, 26-34, 35-50, 51-100, 101-250, 251-500, 501-1000, 1000-1500, 1500-2000, 2000-2500, or 2500-2740 nucleotides. In a preferred embodiment, the oligonucleotides have a length of about 12-100, 15-75, 17-50, 21-50, or preferably 25-50 nucleotides. Preferably, the oligonucleotide is a sequence encoding a contiguous span of at least 4, 5, 6, 7, 8, 9, 10, 11, 12, 13, 14, 15, 17, 20, 22, 25, 30, 35, 50, 100, 150, 200, 250, 300, 350, or 372 amino acids of SEQ ID NO:2.

In addition, oligonucleotides are also contemplated having a length of at least 10, 12, 15, 17, 18, 19, 20, 21, 22, 23, 24, 25, 30, 50, 75, 100, 200, 300, 400, 500, or 600 nucleotides, and preferably no greater than 1500, 1300, 1100, 800, 600, 400, 200 or 100 nucleotides, and capable of hybridizing to the nucleotide sequence of SEQ ID NO:1, or the complement thereof, under stringent hybridization conditions. In a preferred embodiment, the oligonucleotides have a length of about 12-100, 15-75, 17-50, 21-50, or preferably 25-50 nucleotides. In another preferred embodiment, the oligonucleotides capable of hybridizing to the nucleotide sequence of SEQ ID NO:1 or the complement thereof encode a contiguous span of at least 7, 8, 9, 10, 11, 12, 13, 14, 15, 16, 17, 18, 19, 20, 25, 30, 50, 100, 150, 200, 250, 300, 350, or 372 amino acids of SEQ ID NO:2.

As will be apparent to skilled artisans, the various oligonucleotides of the present invention are useful as probes for detecting nucleic acids in cells and tissues. They can also be used as primers for procedures including the amplification of nucleic acids or homologues thereof, sequencing nucleic acids, and the detection of mutations in nucleic acids or homologues thereof. In addition, the oligonucleotides may be used to encode a fragment, epitope or domain of proteins or a homologue thereof, which is useful in a variety of applications including use as antigenic epitopes for preparing antibodies against proteins.

It should be understood that the nucleic acid molecules of the present invention may be in standard forms with conventional nucleotide bases and backbones, but can also be in various modified forms, e.g., having therein modified nucleotide bases or backbones. Examples of modified nucleotide bases or backbones are described in Section 6.2.3 in the context of modified antisense compounds, and such modified nucleic acids should be equally applicable in this respect.

The present invention also provides isolated polypeptides. The present invention also encompasses a polypeptide having an amino acid sequence that is at least 50%, preferably at least 60%, more preferably at least 75%, 80%, 85%, 90%, 91%, 92%, 93%, 94%, and even more preferably at least 95%, 96%, 97%, 98%, or 99% identical to the amino acid sequence of SEQ ID NO:2. In a specific embodiment, the homologous polypeptide is a naturally occurring variant of a protein identified in a human population. Such a variant may be identified by assaying the nucleic acids or protein in a population, as is generally known in the art. In another embodiment, the present invention also provides an isolated polypeptide that is encoded by an isolated nucleic acid molecule that specifically hybridizes fully to the isolated nucleic acid molecule of SEQ ID NO:1, or the complement thereof, under moderate or high stringency conditions.

The present invention further encompasses protein fragments having a contiguous span of at least 6, 7, 8, 9, 10, 11, 12, 13, 14, 15, 16, 17, 18, 19, 20, 25, 50, 100, 150, 200, 250, 300, 350, or at least 370 amino acids of the sequence of SEQ ID NO:2, but less than the full length of the sequence of SEQ ID NO:2. For example, such fragments can be generated as a result of the deletion of a contiguous span of a certain number of amino acids from either or both of the amino and carboxyl termini of the protein having the sequence of SEQ ID NO:2. In specific embodiments, a polypeptide is provided including a contiguous span of at least 4, 5, 6, 7, 8, 9, 10, 11, 12, 13, 14, 15, 16, 17, 18, 19, 20, 25, 50, 100, 150, 200, 250, 300, 350, or at least 370 amino acids of the sequence of SEQ ID NO:2. In other specific embodiments, the polypeptide fragments contain immunogenic or antigenic epitopes. Such epitopes can be readily determined by computer programs such as MacVector® from International Biotechnologies, Inc. and Protean® from DNAStar, Inc. In addition, epitopes can also be selected experimentally by any methods known in the art, e.g., the methods provided in U.S. Pat. Nos. 4,833,092 and 5,194,392, both of which are incorporated herein by reference.

In addition, the present invention is also directed to polypeptides that are homologous to the foregoing polypeptide fragments. Such a homologous polypeptide may have the same length as one of the foregoing polypeptide fragments of the present invention (e.g., from 5 to 50, from 5 to 30, or from 7 to 25, or preferably 8 to 20 amino acids) but has an amino acid sequence that is at least 75%, 80%, 85%, 90%, preferably at least 95%, 96%, 97%, 98%, or, more preferably, at least 99% identical to the amino acid sequence of the corresponding polypeptide fragment.

Additionally, the present invention further relates to a hybrid polypeptide having any one of the foregoing polypeptides of the present invention covalently linked to another polypeptide. Such other polypeptides can also be one of the foregoing polypeptides of the present invention. Alternatively, such other polypeptides are not one of the foregoing polypeptides of the present invention. The covalent linkage in the hybrid polypeptide of the present invention can be merely a covalent bond between the two components of the hybrid polypeptide. Alternatively, any linker molecules may be used to connect the polypeptides. For example, a peptide or a non-peptidic organic molecule may be used as a linker molecule.

### 2.3. Methods of Preparing Protein Complexes

The protein complexes of the present invention can be prepared by a variety of methods. Specifically, a protein complex can be isolated directly from an animal tissue sample, preferably a human tissue sample containing the protein complex. Alternatively, a protein complex can be purified from host cells that recombinantly express the members of the protein complex. As will be apparent to a skilled artisan, a protein complex can be prepared from a tissue sample or recombinant host cells by coimmunoprecipitation using an antibody immunoreactive with an interacting protein partner, or preferably an antibody selectively immunoreactive with the protein complex, as will be discussed in detail below.

The antibodies can be monoclonal or polyclonal. Coimmunoprecipitation is a commonly used method in the art for isolating or detecting bound proteins. In this procedure, generally a serum sample or tissue or cell lysate is admixed with a suitable antibody. The protein complex bound to the antibody is precipitated and washed. The bound protein complexes are then eluted and the constituent proteins are identified by some method (e.g., Western blotting).

Alternatively, immunoaffinity chromatography and immunoblotting techniques may also be used in isolating the protein complexes from native tissue samples or recombinant host cells using an antibody immunoreactive with an interacting protein partner, or preferably an antibody selectively immunoreactive with the protein complex. For example, in protein immunoaffinity chromatography, the antibody is covalently or non-covalently coupled to a matrix (e.g., Sepharose), which is then packed into a column. Extract from a tissue sample, or lysate from recombinant cells is passed through the column where it contacts the antibodies attached to the matrix. The column is then washed with a low-salt solution to wash away the unbound or loosely (non-specifically) bound components. The protein complexes that are retained in the column can be then eluted from the column using a high-salt solution, a competitive antigen of the antibody, a chaotropic solvent, or a denaturant or detergent such as sodium dodecyl sulfate (SDS), or the like. In immunoblotting, crude proteins samples from a tissue sample extract or recombinant host cell lysate are fractionated by polyacrylamide gel electrophoresis (PAGE) and then transferred to a membrane, e.g., nitrocellulose. Components of the protein complex can then be located on the membrane and identified by a variety of techniques, e.g., probing with specific antibodies.

In another embodiment, individual interacting protein partners may be isolated or purified independently from tissue samples or recombinant host cells using similar methods as described above. The individual interacting protein partners are then combined under conditions conducive to their interaction thereby forming a protein complex of the present invention. It is noted that different protein-protein interactions may require different conditions. As a starting point, for example, a buffer having 20 mM Tris-HCl, pH 7.0 and 500 mM NaCl may be used. Several different parameters may be varied, including temperature, pH, salt concentration, reducing agent, and the like. Some minor degree of experimentation may be required to determine the optimum incubation condition, this being well within the capability of one skilled in the art and apprised of the present disclosure.

In yet another embodiment, the protein complex of the present invention may be prepared from tissue samples or recombinant host cells or other suitable sources by protein affinity chromatography or affinity blotting. That is, one of the interacting protein partners is used to isolate the other interacting protein partner(s) by binding affinity that leads to the formation of protein complexes. Thus, an interacting protein partner prepared by purification from tissue samples or by recombinant expression or chemical synthesis may be bound covalently or non-covalently to a matrix, e.g., Sepharose, which is then packed into a chromatography column. The tissue sample extract or cell lysate from the recombinant cells can then be contacted with the bound protein on the matrix. A low-salt solution is used to wash off the unbound or loosely bound components, and a high-salt solution is then employed to elute the bound protein complexes in the column. In affinity blotting, crude protein samples from a tissue sample or recombinant host cell lysate can be fractionated by PAGE and then transferred to a membrane, e.g., nitrocellulose. The purified interacting protein member is then bound to its interacting protein partner(s) on the membrane, thereby forming protein complexes, which are then isolated from the membrane.

It will be apparent to skilled artisans that any recombinant expression methods may be used in the present invention for purposes of expressing the protein complexes or individual interacting proteins. Generally, a nucleic acid encoding an interacting protein member can be introduced into a suitable host cell. For purposes of forming a recombinant protein complex within a host cell, nucleic acids encoding two or more interacting protein members should be introduced into the host cell.

Typically, the nucleic acids, preferably in the form of DNA, are incorporated into a vector to form expression vectors capable of directing the production of the interacting protein member(s) once introduced into a host cell. Many types of vectors can be used for the present invention. Methods for the construction of an expression vector for purposes of this invention should be apparent to skilled artisans apprised of the present disclosure. See generally, Current Protocols in Molecular Biology, Vol. 2, Ed. Ausubel, et al., Greene Publish. Assoc. & Wiley Interscience, Ch. 13, 1988; Glover, DNA Cloning, Vol. II, IRL Press, Wash., D.C., Ch. 3, 1986; Bitter, et al., in Methods in Enzymology 153:516-544 (1987); The Molecular Biology of the Yeast Saccharomyces, Eds. Strathern et al., Cold Spring Harbor Press, Vols. I and II, 1982; and Sambrook et al., Molecular Cloning: A Laboratory Manual, Cold Spring Harbor Press, 1989.

Generally, the expression vectors include an expression cassette having a promoter operably linked to a DNA encoding an interacting protein member. The promoter can be a native promoter, i.e., the promoter found in naturally occurring cells to be responsible for the expression of the interacting protein member in the cells. Alternatively, the expression cassette can be a chimeric one, i.e., having a heterologous promoter that is not the native promoter responsible for the expression of the interacting protein member in naturally occurring cells. The expression vector may further include an origin of DNA replication for the replication of the vectors in host cells. Preferably, the expression vectors also include a replication origin for the amplification of the vectors in, e.g., E. coli, and selection marker(s) for selecting and maintaining only those host cells harboring the expression vectors. Additionally, the expression cassettes preferably also contain inducible elements, which function to control the transcription from the DNA encoding the interacting protein member. Other regulatory sequences such as transcriptional enhancer sequences and translation regulation sequences (e.g., Shine-Dalgarno sequence) can also be operably included in the expression cassettes. Termination sequences such as the polyadenylation signals from bovine growth hormone, SV40, lacZ and AcMNPV polyhedral protein genes may also be operably linked to the DNA encoding an interacting protein member in the expression cassettes. A nucleotide sequence encoding an epitope tag can also be operably linked to the DNA encoding an interacting protein member such that a fusion protein is expressed. Such epitope tags are chosen and added to simplify the detection and/or purification of the fusion protein. Examples of useful epitope tags include, but are not limited to, influenza virus hemagglutinin (HA), Simian Virus 5 (V5), polyhistidine (6xHis), c-myc, lacZ, GST, and the like. Proteins with polyhistidine tags can be easily detected and/or purified with Ni²⁺ affinity columns, while specific antibodies immunoreactive with many epitope tags are generally commercially available. The expression vectors may also contain components that direct the expressed protein extracellularly, or to a particular intracellular compartment. Signal peptides, nuclear localization sequences, endoplasmic reticulum retention signals, mitochondrial localization sequences, myristoylation signals, palmitoylation signals, and transmembrane sequences are examples of optional vector components that can determine the destination of expressed proteins. When it is desirable to express two or more interacting protein members in a single host cell, the DNA fragments encoding the interacting protein members may be incorporated into a single vector or different vectors.

The resulting expression vectors can be introduced into host cells by any techniques known in the art, e.g., by direct DNA transformation, microinjection, electroporation, viral infection, lipofection, biolystics, and the like. The expression of the interacting protein members may be transient or stable. The expression vectors can be maintained in host cells in an extrachromosomal state, i.e., as self-replicating plasmids or viruses. Alternatively, the expression vectors can be integrated into chromosomes of the host cells by conventional techniques such as site-specific recombination and selection of stable cell lines. In stable cell lines, at least the expression cassette portion of the expression vector is integrated into a chromosome of the host cells.

The vector construct can be designed to be suitable for protein expression in various host cells, including but not limited to bacterial cells, yeast cells, plant cells, insect cells, and mammalian and human cells. Methods for preparing expression vectors for expression in different host cells should be apparent to a skilled artisan.

Homologues and fragments of the native interacting protein members can also be expressed using the recombinant methods described above. For example, to express a protein fragment, the DNA fragment incorporated into the expression vector can be selected such that it only encodes the protein fragment. Likewise, a specific hybrid protein can be expressed using a recombinant DNA encoding the hybrid protein. Similarly, a homologue protein may be expressed from a DNA sequence encoding the homologue protein. A homologue-encoding DNA sequence may be obtained by manipulating the native protein-encoding sequence using recombinant DNA techniques. For this purpose, random or site-directed mutagenesis can be conducted using techniques generally known in the art. To make protein derivatives, for example, the amino acid sequence of a native interacting protein member may be changed in predetermined manners by site-directed DNA mutagenesis to create or remove consensus sequences for, e.g., phosphorylation by protein kinases, glycosylation, ribosylation, myristolation, palmytoylation, ubiquitination, and the like. Alternatively, non-natural amino acids can be incorporated into an interacting protein member during the synthesis of the protein in recombinant host cells. For example, photoreactive lysine derivatives can be incorporated into an interacting protein member during translation by using a modified lysyl-tRNA. See, e.g., Wiedmann et al., Nature, 328:830-833 (1989); Musch et al., Cell, 69:343-352 (1992). Other photoreactive amino acid derivatives can also be incorporated in a similar manner. See, e.g., High et al., J Biol Chem, 368:28745-28751 (1993). Indeed, the photoreactive amino acid derivatives thus incorporated into an interacting protein member can act to cross-link the protein to its interacting protein partner in a protein complex under predetermined conditions.

In addition, derivatives of the native interacting protein members of the present invention can also be prepared by chemically linking certain moieties to amino acid side chains of the native proteins.

If desired, the homologues and derivatives thus generated can be tested to determine whether they are capable of interacting with their intended partners to form protein complexes. Testing can be conducted by e.g., the yeast two-hybrid system or other methods known in the art for detecting protein-protein interaction.

A hybrid protein as described above having any interacting pair of the proteins described in the tables, or a homologue, derivative, or fragment thereof covalently linked together by a peptide bond or a peptide linker can be expressed recombinantly from a chimeric nucleic acid, e.g., a DNA or mRNA fragment encoding the fusion protein. Accordingly, the present invention also provides a nucleic acid encoding the hybrid protein of the present invention. In addition, an expression vector having incorporated therein a nucleic acid encoding the hybrid protein of the present invention is also provided. The methods for making such chimeric nucleic acids and expression vectors containing them will be apparent to skilled artisans apprised of the present disclosure.

### 2.4. Protein Microchip

In accordance with another embodiment of the present invention, a protein microchip or microarray is provided having one or more of the protein complexes and/or antibodies selectively immunoreactive with the protein complexes of the present invention. Protein microarrays are becoming increasingly important in both proteomics research and protein-based detection and diagnosis of diseases. The protein microarrays in accordance with this embodiment of the present invention will be useful in a variety of applications including, e.g., large-scale or high-throughput screening for compounds capable of binding to the protein complexes or modulating the interactions between the interacting protein members in the protein complexes.

The protein microarray of the present invention can be prepared in a number of methods known in the art. An example of a suitable method is that disclosed in MacBeath & Schreiber, Science, 289:1760-1763 (2000). Essentially, glass microscope slides are treated with an aldehyde-containing silane reagent (SuperAldehyde Substrates purchased from TeleChem International, Cupertino, Calif.). Nanoliter volumes of protein samples in a phosphate-buffered saline with 40% glycerol are then spotted onto the treated slides using a high-precision contact-printing robot. After incubation, the slides are immersed in a bovine serum albumin (BSA)-containing buffer to quench the unreacted aldehydes and to form a BSA layer that functions to prevent non-specific protein binding in subsequent applications of the microchip. Alternatively, as disclosed in MacBeath and Schreiber, proteins or protein complexes of the present invention can be attached to a BSA-NHS slide by covalent linkages. BSA-NHS slides are fabricated by first attaching a molecular layer of BSA to the surface of glass slides and then activating the BSA with N,N′-disuccinimidyl carbonate. As a result, the amino groups of the lysine, aspartate, and glutamate residues on the BSA are activated and can form covalent urea or amide linkages with protein samples spotted on the slides. See MacBeath and Schreiber, Science, 289:1760-1763 (2000).

Another example of a useful method for preparing the protein microchip of the present invention is that disclosed in PCT Publication Nos. WO 00/4389A2 and WO 00/04382, both of which are assigned to Zyomyx and are incorporated herein by reference. First, a substrate or chip base is covered with one or more layers of thin organic film to eliminate any surface defects, insulate proteins from the base materials, and to ensure uniform protein array. Next, a plurality of protein-capturing agents (e.g., antibodies, peptides, etc.) are arrayed and attached to the base that is covered with the thin film. Proteins or protein complexes can then be bound to the capturing agents forming a protein microarray. The protein microchips are kept in flow chambers with an aqueous solution.

The protein microarray of the present invention can also be made by the method disclosed in PCT Publication No. WO 99/36576 assigned to Packard Bioscience Company, which is incorporated herein by reference. For example, a three-dimensional hydrophilic polymer matrix, i.e., a gel, is first dispensed on a solid substrate such as a glass slide. The polymer matrix gel is capable of expanding or contracting and contains a coupling reagent that reacts with amine groups. Thus, proteins and protein complexes can be contacted with the matrix gel in an expanded aqueous and porous state to allow reactions between the amine groups on the protein or protein complexes with the coupling reagents thus immobilizing the proteins and protein complexes on the substrate. Thereafter, the gel is contracted to embed the attached proteins and protein complexes in the matrix gel.

Alternatively, the proteins and protein complexes of the present invention can be incorporated into a commercially available protein microchip, e.g., the ProteinChip System from Ciphergen Biosystems Inc., Palo Alto, Calif. The ProteinChip System comprises metal chips having a treated surface, which interact with proteins. Basically, a metal chip surface is coated with a silicon dioxide film. The molecules of interest such as proteins and protein complexes can then be attached covalently to the chip surface via a silane coupling agent.

The protein microchips of the present invention can also be prepared with other methods known in the art, e.g., those disclosed in U.S. Pat. Nos. 6,087,102, 6,139,831, 6,087,103; PCT Publication Nos. WO 99/60156, WO 99/39210, WO 00/54046, WO 00/53625, WO 99/51773, WO 99/35289, WO 97/42507, WO 01/01142, WO 00/63694, WO 00/61806, WO 99/61148, WO 99/40434, all of which are incorporated herein by reference.

### 3. Antibodies

In accordance with another aspect of the present invention, an antibody immunoreactive against a protein complex of the present invention is provided. In one embodiment, the antibody is selectively immunoreactive with a protein complex of the present invention. Specifically, the phrase “selectively immunoreactive with a protein complex” as used herein means that the immunoreactivity of the antibody of the present invention with the protein complex is substantially higher than that with the individual interacting members of the protein complex so that the binding of the antibody to the protein complex is readily distinguishable from the binding of the antibody to the individual interacting member proteins based on the strength of the binding affinities. Preferably, the binding constants differ by a magnitude of at least 2 fold, more preferably at least 5 fold, even more preferably at least 10 fold, and most preferably at least 100 fold. In a specific embodiment, the antibody is not substantially immunoreactive with the interacting protein members of the protein complex.

The antibodies of the present invention can be readily prepared using procedures generally known in the art. See, e.g., Harlow and Lane, Antibodies: A Laboratory Manual, Cold Spring Harbor Press, 1988. Typically, the protein complex against which an immunoreactive antibody is desired is used as the antigen for producing an immune response in a host animal. In one embodiment, the protein complex used consists of the native proteins. Preferably, the protein complex includes only protein fragments containing interacting regions provided in the tables. As a result, a greater portion of the total antibodies may be selectively immunoreactive with the protein complexes. The interaction domains can be selected from, e.g., those regions summarized in the tables above. In addition, various techniques known in the art for predicting epitopes may also be employed to design antigenic peptides based on the interacting protein members in a protein complex of the present invention to increase the possibility of producing an antibody selectively immunoreactive with the protein complex. Suitable epitope-prediction computer programs include, e.g., MacVector® from International Biotechnologies, Inc. and Protean® from DNAStar, Inc.

In a specific embodiment, a hybrid protein as described above in Section 2.2 is used as an antigen which has a first protein that is any one of the proteins described in the tables, or a homologue, derivative, or fragment thereof covalently linked by a peptide bond or a peptide linker to a second protein which is the interacting partner of the first protein, or a homologue, derivative, or fragment of the second protein. In a preferred embodiment, the hybrid protein consists of two interacting domains selected from the regions identified in a table above, or homologues or derivatives thereof, covalently linked together by a peptide bond or a linker molecule.

The antibody of the present invention can be a polyclonal antibody to a protein complex of the present invention. To produce the polyclonal antibody, various animal hosts can be employed, including, e.g., mice, rats, rabbits, goats, guinea pigs, hamsters, etc. A suitable antigen which is a protein complex of the present invention or a derivative thereof as described above can be administered directly to a host animal to illicit immune reactions. Alternatively, it can be administered together with a carrier such as keyhole limpet hemocyanin (KLH), bovine serum albumin (BSA), ovalbumin, and Tetanus toxoid. Optionally, the antigen is conjugated to a carrier by a coupling agent such as carbodiimide, glutaraldehyde, and MBS. Any conventional adjuvants may be used to boost the immune response of the host animal to the protein complex antigen. Suitable adjuvants known in the art include but are not limited to Complete Freund's Adjuvant (which contains killed mycobacterial cells and mineral oil), incomplete Freund's Adjuvant (which lacks the cellular components), aluminum salts, MF59 from Chiron (Emeryville, Calif.), monophospholipid, synthetic trehalose dicorynomycolate (TDM) and cell wall skeleton (CWS) both from Corixa Corp. (Seattle, Wash.), non-ionic surfactant vesicles (NISV) from Proteus International PLC (Cheshire, U.K.), and saponins. The antigen preparation can be administered to a host animal by subcutaneous, intramuscular, intravenous, intradermal, or intraperitoneal injection, or by injection into a lymphoid organ.

The antibodies of the present invention may also be monoclonal. Such monoclonal antibodies may be developed using any conventional techniques known in the art. For example, the popular hybridoma method disclosed in Kohler and Milstein, Nature, 256:495-497 (1975) is now a well-developed technique that can be used in the present invention. See U.S. Pat. No. 4,376,110, which is incorporated herein by reference. Essentially, B-lymphocytes producing a polyclonal antibody against a protein complex of the present invention can be fused with myeloma cells to generate a library of hybridoma clones. The hybridoma population is then screened for antigen binding specificity and also for immunoglobulin class (isotype). In this manner, pure hybridoma clones producing specific homogenous antibodies can be selected. See generally, Harlow and Lane, Antibodies: A Laboratory Manual, Cold Spring Harbor Press, 1988. Alternatively, other techniques known in the art may also be used to prepare monoclonal antibodies, which include but are not limited to the EBV hybridoma technique, the human N-cell hybridoma technique, and the trioma technique.

In addition, antibodies selectively immunoreactive with a protein complex of the present invention may also be recombinantly produced. For example, cDNAs prepared by PCR amplification from activated B-lymphocytes or hybridomas may be cloned into an expression vector to form a cDNA library, which is then introduced into a host cell for recombinant expression. The cDNA encoding a specific desired protein may then be isolated from the library. The isolated cDNA can be introduced into a suitable host cell for the expression of the protein. Thus, recombinant techniques can be used to produce specific native antibodies, hybrid antibodies capable of simultaneous reaction with more than one antigen, chimeric antibodies (e.g., the constant and variable regions are derived from different sources), univalent antibodies that comprise one heavy and light chain pair coupled with the Fc region of a third (heavy) chain, Fab proteins, and the like. See U.S. Pat. No. 4,816,567; European Patent Publication No. 0088994; Munro, Nature, 312:597 (1984); Morrison, Science, 229:1202 (1985); Oi et al., BioTechniques, 4:214 (1986); and Wood et al., Nature, 314:446-449 (1985), all of which are incorporated herein by reference. Antibody fragments such as Fv fragments, single-chain Fv fragments (scFv), Fab′ fragments, and F(ab′)₂ fragments can also be recombinantly produced by methods disclosed in, e.g., U.S. Pat. No. 4,946,778; Skerra & Plückthun, Science, 240:1038-1041(1988); Better et al., Science, 240:1041-1043 (1988); and Bird, et al., Science, 242:423-426 (1988), all of which are incorporated herein by reference.

In a preferred embodiment, the antibodies provided in accordance with the present invention are partially or fully humanized antibodies. For this purpose, any methods known in the art may be used. For example, partially humanized chimeric antibodies having V regions derived from the tumor-specific mouse monoclonal antibody, but human C regions are disclosed in Morrison and Oi, Adv Immunol, 44:65-92 (1989). In addition, fully humanized antibodies can be made using transgenic non-human animals. For example, transgenic non-human animals such as transgenic mice can be produced in which endogenous immunoglobulin genes are suppressed or deleted, while heterologous antibodies are encoded entirely by exogenous immunoglobulin genes, preferably human immunoglobulin genes, recombinantly introduced into the genome. See e.g., U.S. Pat. Nos. 5,530,101; 5,545,806; 6,075,181; PCT Publication No. WO 94/02602; Green et. al., Nat Genetics, 7: 13-21 (1994); and Lonberg et al., Nature 368: 856-859 (1994), all of which are incorporated herein by reference. The transgenic non-human host animal may be immunized with suitable antigens such as a protein complex of the present invention or one or more of the interacting protein members thereof to illicit specific immune response thus producing humanized antibodies. In addition, cell lines producing specific humanized antibodies can also be derived from the immunized transgenic non-human animals. For example, mature B-lymphocytes obtained from a transgenic animal producing humanized antibodies can be fused to myeloma cells and the resulting hybridoma clones may be selected for specific humanized antibodies with desired binding specificities. Alternatively, cDNAs may be extracted from mature B-lymphocytes and used in establishing a library that is subsequently screened for clones encoding humanized antibodies with desired binding specificities.

In yet another embodiment, a bifunctional antibody is provided that has two different antigen binding sites, each being specific to a different interacting protein member in a protein complex of the present invention. The bifunctional antibody may be produced using a variety of methods known in the art. For example, two different monoclonal antibody-producing hybridomas can be fused together. One of the two hybridomas may produce a monoclonal antibody specific against an interacting protein member of a protein complex of the present invention, while the other hybridoma generates a monoclonal antibody immunoreactive with another interacting protein member of the protein complex. The thus formed new hybridoma produces different antibodies including a desired bifunctional antibody, i.e., an antibody immunoreactive with both of the interacting protein members. The bifunctional antibody can be readily purified. See Milstein and Cuello, Nature, 305:537-540 (1983).

Alternatively, a bifunctional antibody may also be produced using heterobifunctional crosslinkers to chemically link two different monoclonal antibodies, each being immunoreactive with a different interacting protein member of a protein complex. Therefore, the aggregate will bind to two interacting protein members of the protein complex. See Staerz et al., Nature, 314:628-631(1985); Perez et al., Nature, 316:354-356 (1985).

In addition, bifunctional antibodies can also be produced by recombinantly expressing light and heavy chain genes in a hybridoma that itself produces a monoclonal antibody. As a result, a mixture of antibodies including a bifunctional antibody is produced. See DeMonte et al., Proc Natl Acad Sci USA, 87:2941-2945 (1990); Lenz and Weidle, Gene, 87:213-218 (1990).

Preferably, a bifunctional antibody in accordance with the present invention is produced by the method disclosed in U.S. Pat. No. 5,582,996, which is incorporated herein by reference. For example, two different Fabs can be provided and mixed together. The first Fab can bind to an interacting protein member of a protein complex, and has a heavy chain constant region having a first complementary domain not naturally present in the Fab but capable of binding a second complementary domain. The second Fab is capable of binding another interacting protein member of the protein complex, and has a heavy chain constant region comprising a second complementary domain not naturally present in the Fab but capable of binding to the first complementary domain. Each of the two complementary domains is capable of stably binding to the other but not to itself. For example, the leucine zipper regions of c-fos and c-jun oncogenes may be used as the first and second complementary domains. As a result, the first and second complementary domains interact with each other to form a leucine zipper thus associating the two different Fabs into a single antibody construct capable of binding to two antigenic sites.

Other suitable methods known in the art for producing bifunctional antibodies may also be used, which include those disclosed in Holliger et al., Proc Natl Acad Sci USA, 90:6444-6448 (1993); de Kruif et al., J Biol Chem, 271:7630-7634 (1996); Coloma & Morrison, Nat. Biotechnol., 15:159-163 (1997); Muller et al., FEBS Lett, 422:259-264 (1998); and Muller et al., FEBS Lett, 432:45-49 (1998), all of which are incorporated herein by reference.

### 4. Methods of Detecting Protein Complexes

Another aspect of the present invention relates to methods for detecting the protein complexes of the present invention, particularly for determining the concentration of a specific protein complex in a sample from a patient.

In one embodiment, the concentration of a protein complex of the present invention is determined in cells, tissue, or an organ of a patient. For example, the protein complex can be isolated or purified from a patient sample obtained from cells, tissue, or an organ of the patient and the amount thereof is determined. As described above, the protein complex can be prepared from cells, tissue or organ samples by coimmuno-precipitation using an antibody immunoreactive with an interacting protein member, a bifunctional antibody that is immunoreactive with two or more interacting protein members of the protein complex, or preferably an antibody selectively immunoreactive with the protein complex. When bifunctional antibodies or antibodies immunoreactive with only free interacting protein members are used, individual interacting protein members not complexed with other proteins may also be isolated along with the protein complex containing such individual proteins. However, they can be readily separated from the protein complex using methods known in the art, e.g., size-based separation methods such as gel filtration, or by subtracting the protein complex from the mixture using an antibody specific against another individual interacting protein member. Additionally, proteins in a sample can be separated in a gel such as polyacrylamide gel and subsequently immunoblotted using an antibody immunoreactive with the protein complex.

Alternatively, the concentration of the protein complex can be determined in a sample without separation, isolation or purification. For this purpose, it is preferred that an antibody selectively immunoreactive with the specific protein complex is used in an immunoassay. For example, immunocytochemical methods can be used. Other well known antibody-based techniques can also be used including, e.g., enzyme-linked immunosorbent assay (ELISA), radioimmunoassay (RIA), immunoradiometric assays (IRMA), fluorescent immunoassays, protein A immunoassays, and immunoenzymatic assays (IEMA). See e.g., U.S. Pat. Nos. 4,376,110 and 4,486,530, both of which are incorporated herein by reference.

In addition, since a specific protein complex is formed from its interacting protein members, if one of the interacting protein members is at a relatively low concentration in a patient, it may be reasonably expected that the concentration of the protein complex in the patient may also be low. Therefore, the concentration of an individual interacting protein member of a specific protein complex can be determined in a patient sample which can then be used as a reasonably accurate indicator of the concentration of the protein complex in the sample. For this purpose, antibodies against an individual interacting protein member of a specific complex can be used in any one of the methods described above. In a preferred embodiment, the concentration of each of the interacting protein members of a protein complex is determined in a patient sample and the relative concentration of the protein complex is then deduced.

In addition, the relative protein complex concentration in a patient can also be determined by determining the concentration of the mRNA encoding an interacting protein member of the protein complex. Preferably, each interacting protein member's mRNA concentration in a patient sample is determined. For this purpose, methods for determining mRNA concentration generally known in the art may all be used. Examples of such methods include, e.g., Northern blot assay, dot blot assay, PCR assay (preferably quantitative PCR assay), in situ hybridization assay, and the like.

As discussed above, each interaction between members of an interacting protein pair of the present invention suggests that the proteins and/or the protein complexes formed by such proteins may be involved in common biological processes and disease pathways. In addition, the interactions under physiological conditions may lead to the formation of protein complexes in vivo. The protein complexes are expected to mediate the functions and biological activities of the interacting members of the protein complexes. Thus, aberrations in the protein complexes or the individual proteins and the degree of the aberration may be indicators for the diseases or disorders. These aberrations may be used as parameters for classifying and/or staging one of the above-described diseases. In addition, they may also be indicators for patients' response to a drug therapy.

Association between a physiological state (e.g., physiological disorder, predisposition to the disorder, a disease state, response to a drug therapy, or other physiological phenomena or phenotypes) and a specific aberration in a protein complex of the present invention or an individual interacting member thereof can be readily determined by comparative analysis of the protein complex and/or the interacting members thereof in a normal population and an abnormal or affected population. Thus, for example, one can study the concentration, localization and distribution of a particular protein complex, mutations in the interacting protein members of the protein complex, and/or the binding affinity between the interacting protein members in both a normal population and a population affected with a particular physiological disorder described above. The study results can be compared and analyzed by statistical means. Any detected statistically significant difference in the two populations would indicate an association. For example, if the concentration of the protein complex is statistically significantly higher in the affected population than in the normal population, then it can be reasonably concluded that higher concentration of the protein complex is associated with the physiological disorder.

Thus, once an association is established between a particular type of aberration in a particular protein complex of the present invention or in an interacting protein member thereof and a physiological disorder or disease or predisposition to the physiological disorder or disease, then the particular physiological disorder or disease or predisposition to the physiological disorder or disease can be diagnosed or detected by determining whether a patient has the particular aberration.

Accordingly, the present invention also provides a method for diagnosing in a patient a disease or physiological disorder, or a predisposition to the disease or disorder, including neurological disorders, ailments and diseases, including mild cognitive impairment, depression, schizophrenia, obsessive-compulsive disorder, bipolar disorder, and neurodegenerative diseases and disorders and motor neuron diseases and disorders such as Alzheimer's disease, Parkinson's disease, dementia with Lewy bodies, amyotrophic lateral sclerosis or Lou Gehrig's disease, Alpers' disease, Leigh's disease, Pelizaeus-Merzbacher disease, Olivopontocerebellar atrophy, Friedreich's ataxia, leukodystrophies, Rett syndrome, Ramsay Hunt syndrome type II, and Down's syndrome, by determining whether there is any aberration in the patient with respect to a protein complex identified according to the present invention. The same protein complex is analyzed in a normal individual, or a collection of normal individuals, and is compared with the results obtained in the patient. In this manner, any protein complex aberration in the patient can be detected. As used herein, the term “aberration” when used in the context of protein complexes of the present invention means any alterations of a protein complex including increased or decreased concentration of the protein complex in a particular cell or tissue or organ or the total body, altered localization of the protein complex in cellular compartments or in locations of a tissue or organ, changes in binding affinity of an interacting protein member of the protein complex, mutations in an interacting protein member or the gene encoding the protein, and the like. As will be apparent to a skilled artisan, the term “aberration” is used in a relative sense. That is, an aberration is relative to a normal condition.

As used herein, the term “diagnosis” means detecting a disease or disorder or determining the stage or degree of a disease or disorder. The term “diagnosis” also encompasses detecting a predisposition to a disease or disorder, determining the therapeutic effect of a drug therapy, or predicting the pattern of response to a drug therapy or xenobiotics. The diagnosis or diagnostic methods of the present invention may be used independently, or in combination with other diagnosing and/or staging methods known in the medical art for a particular disease or disorder.

Thus, in one embodiment, the method of diagnosis is conducted by detecting, in a patient, the concentrations of one or more protein complexes of the present invention using any one of the methods described above, and determining whether the patient has an aberrant concentration of the protein complexes.

The diagnosis may also be based on the determination of the concentrations of one or more interacting protein members (at the protein, cDNA or mRNA level) of a protein complex of the present invention. An aberrant concentration of an interacting protein member may indicate a physiological disorder or a predisposition to a physiological disorder.

In another embodiment, the method of diagnosis comprises determining, in a patient, the cellular localization, or tissue or organ distribution of a protein complex of the present invention and determining whether the patient has an aberrant localization or distribution of the protein complex. For example, immunocytochemical or immunohistochemical assays can be performed on a cell, tissue or organ sample from a patient using an antibody selectively immunoreactive with a protein complex of the present invention. Antibodies immunoreactive with both an individual interacting protein member and a protein complex containing the protein member may also be used, in which case it is preferred that antibodies immunoreactive with other interacting protein members are also used in the assay. In addition, nucleic acid probes may also be used in in situ hybridization assays to detect the localization or distribution of the mRNAs encoding the interacting protein members of a protein complex. Preferably, the mRNA encoding each interacting protein member of a protein complex is detected concurrently.

In yet another embodiment, the method of diagnosis of the present invention comprises detecting any mutations in one or more interacting protein members of a protein complex of the present invention. In particular, it is desirable to determine whether the interacting protein members have any mutations that will lead to, or are associated with, changes in the functional activity of the proteins or changes in their binding affinity to other interacting protein members in forming a protein complex of the present invention. Examples of such mutations include but are not limited to, e.g., deletions, insertions and rearrangements in the genes encoding the protein members, and nucleotide or amino acid substitutions and the like. In a preferred embodiment, the domains of the interacting protein members that are responsible for the protein-protein interactions, and lead to protein complex formation, are screened to detect any mutations therein. For example, genomic DNA or cDNA encoding an interacting protein member can be prepared from a patient sample, and sequenced. The thus obtained sequence may be compared with known wild-type sequences to identify any mutations. Alternatively, an interacting protein member may be purified from a patient sample and analyzed by protein sequencing or mass spectrometry to detect any amino acid sequence changes. Any methods known in the art for detecting mutations may be used, as will be apparent to skilled artisans apprised of the present disclosure.

In another embodiment, the method of diagnosis includes determining the binding constant of the interacting protein members of one or more protein complexes. For example, the interacting protein members can be obtained from a patient by direct purification or by recombinant expression from genomic DNAs or cDNAs prepared from a patient sample encoding the interacting protein members. Binding constants represent the strength of the protein-protein interaction between the interacting protein members in a protein complex. Thus, by measuring binding constants, subtle aberrations in binding affinity may be detected.

A number of methods known in the art for estimating and determining binding constants in protein-protein interactions are reviewed in Phizicky and Fields, et al., Microbiol Rev, 59:94-123 (1995), which is incorporated herein by reference. For example, protein affinity chromatography may be used. First, columns are prepared with different concentrations of an interacting protein member, which is covalently bound to the columns. Then a preparation of an interacting protein partner is run through the column and washed with buffer. The interacting protein partner bound to the interacting protein member linked to the column is then eluted. A binding constant is then estimated based on the concentrations of the bound protein and the eluted protein. Alternatively, the method of sedimentation through gradients monitors the rate of sedimentation of a mixture of proteins through gradients of glycerol or sucrose. At concentrations above the binding constant, proteins can sediment as a protein complex. Thus, binding constant can be calculated based on the concentrations. Other suitable methods known in the art for estimating binding constant include but are not limited to gel filtration column such as nonequilibrium “small-zone” gel filtration columns (See e.g., Gill et al., J Mol Biol, 220:307-324 (1991)), the Hummel-Dreyer method of equilibrium gel filtration (See e.g., Hummel & Dreyer, Biochim Biophys Acta, 63:530-532 (1962)) and large-zone equilibrium gel filtration (See e.g., Gilbert & Kellett, J Biol Chem, 246:6079-6086 (1971)), sedimentation equilibrium (See e.g., Rivas & Minton, Trends Biochem., 18:284-287 (1993)), fluorescence methods such as fluorescence spectrum (See e.g., Otto-Bruc et al., Biochemistry, 32:8632-8645 (1993)) and fluorescence polarization or anisotropy with tagged molecules (See e.g., Weiel & Hershey, Biochemistry, 20:5859-5865 (1981)), solution equilibrium measured with immobilized binding protein (See e.g., Nelson & Long, Biochemistry, 30:2384-2390 (1991)), and surface plasmon resonance (See e.g., Panayotou et al., Mol Cell Biol, 13:3567-3576 (1993)).

In another embodiment, the diagnosis or diagnostic method of the present invention comprises detecting protein-protein interactions in functional assay systems such as the yeast two-hybrid system. Accordingly, to determine the protein-protein interaction between two interacting protein members that normally form a protein complex in normal individuals, cDNAs encoding the interacting protein members can be isolated from a patient to be diagnosed. The thus cloned cDNAs or fragments thereof can be subcloned into vectors for use in yeast two-hybrid systems. Preferably a reverse yeast two-hybrid system is used such that failure of interaction between the proteins may be positively detected. The use of yeast two-hybrid systems or other systems for detecting protein-protein interactions is known in the art and is described below in Section 5.3.1.

A kit may be used for conducting the diagnosis or diagnostic methods of the present invention. Typically, the kit should contain, in a carrier or compartmentalized container, reagents useful in any of the above-described embodiments of the diagnosis method. The carrier can be a container or support, in the form of, e.g., bag, box, tube, rack, and is optionally compartmentalized. The carrier may define an enclosed confinement for safety purposes during shipment and storage. In one embodiment, the kit includes an antibody selectively immunoreactive with a protein complex of the present invention. In addition, antibodies against individual interacting protein members of the protein complexes may also be included. The antibodies may be labeled with a detectable marker such as radioactive isotopes, or enzymatic or fluorescence markers. Alternatively secondary antibodies such as labeled anti-IgG and the like may be included for detection purposes. Optionally, the kit can include one or more of the protein complexes of the present invention prepared or purified from a normal individual or an individual afflicted with a physiological disorder associated with an aberration in the protein complexes or an interacting protein member thereof. In addition, the kit may further include one or more of the interacting protein members of the protein complexes of the present invention prepared or purified from a normal individual or an individual afflicted with a physiological disorder associated with an aberration in the protein complexes or an interacting protein member thereof. Suitable oligonucleotide primers useful in the amplification of the genes or cDNAs for the interacting protein members may also be provided in the kit. In particular, in a preferred embodiment, the kit includes a first oligonucleotide selectively hybridizable to the mRNA or cDNA encoding one member of an interacting pair of proteins and a second oligonucleotide selectively hybridizable to the mRNA or cDNA encoding the other of the interacting pair. Additional oligonucleotides hybridizing to a region of the genes encoding an interacting pair of proteins may also be included. Such oligonucleotides may be used as PCR primers for, e.g., quantitative PCR amplification of mRNAs encoding the interacting proteins, or as hybridizing probes for detecting the mRNAs. The oligonucleotides may have a length of from about 8 nucleotides to about 100 nucleotides, preferably from about 12 to about 50 nucleotides, and more preferably from about 15 to about 30 nucleotides. In addition, the kit may also contain oligonucleotides that can be used as hybridization probes for detecting the cDNAs or mRNAs encoding the interacting protein members. Preferably, instructions for using the kit or reagents contained therein are also included in the kit.

### 5. Use of Protein Complexes or Interacting Protein Members Thereof in Screening Assays for Modulators

The protein complexes of the present invention and the interacting members thereof can also be used in screening assays to identify modulators of the protein complexes, and/or modulators of the interacting proteins. In addition, homologues, derivatives or fragments of the interacting proteins provided in this invention may also be used in such screening assays. As used herein, the term “modulator” encompasses any compounds that can cause any form of alteration of the biological activities or functions of the proteins or protein complexes, including, e.g., enhancing or reducing their biological activities, increasing or decreasing their stability, altering their affinity or specificity to certain other biological molecules, etc. In addition, the term “modulator” as used herein also includes any compounds that simply bind any of the proteins described in the tables, and/or the proteins complexes of the present invention. For example, a modulator can be an “interaction antagonist” capable of interfering with or disrupting or dissociating protein-protein interaction between an interacting pair of proteins identified in the tables, or homologues, fragments or derivatives thereof. A modulator can also be an “interaction agonist” that initiates or strengthens the interaction between the protein members of a protein complex of the present invention, or homologues, fragments or derivatives thereof.

In addition, the discovery of protein ligands of the present invention allows the use of screening assays to identify modulators of individual proteins of the protein complexes. Typical high-throughput screening assays involve measuring the modulation of the enzymatic activity of a protein. However, typical high-throughput screening assays are not applicable to proteins that exhibit little or no measurable enzymatic activity. The present discovery of novel ligands of proteins allows a screen to be set up that does not require measurement of enzymatic activity. Consequently, the present invention enables a non-enzymatic high-throughput assay to be performed for modulators of individual proteins and/or protein complexes described in the tables above.

Accordingly, the present invention provides screening methods for selecting modulators of any of the proteins described in the tables above, or a mutant form thereof, or a protein-protein interaction between an interacting pair of proteins provided in the present invention, or homologues, fragments or derivatives thereof.

The selected compounds can be tested for their ability to modulate (interfere with or strengthen) the interaction between the interacting partners of the protein complexes of the present invention. In addition, the compounds can also be further tested for their ability to modulate (inhibit or enhance) cellular functions such as Aβ42 production or secretion, neuronal apoptosis, neuronal survival or protection, neurotransmission, axonal guidance or nervous system development, signal transduction, calcium homeostasis, mitochondrial function, or intermediary metabolism in cells, as well as their effectiveness in treating neurological disorders, ailments and diseases, including mild cognitive impairment, depression, schizophrenia, obsessive-compulsive disorder, bipolar disorder, and neurodegenerative diseases and disorders and motor neuron diseases and disorders such as Alzheimer's disease, Parkinson's disease, dementia with Lewy bodies, amyotrophic lateral sclerosis or Lou Gehrig's disease, Alpers' disease, Leigh's disease, Pelizaeus-Merzbacher disease, Olivopontocerebellar atrophy, Friedreich's ataxia, leukodystrophies, Rett syndrome, Ramsay Hunt syndrome type II, and Down's syndrome.

The modulators selected in accordance with the screening methods of the present invention can be effective in modulating the functions or activities of individual interacting proteins, or the protein complexes of the present invention. For example, compounds capable of binding to the protein complexes may be capable of modulating the functions of the protein complexes. Additionally, compounds that interfere with, weaken, dissociate or disrupt, or alternatively, initiate, facilitate or stabilize the protein-protein interaction between the interacting protein members of the protein complexes can also be effective in modulating the functions or activities of the protein complexes. Thus, the compounds identified in the screening methods of the present invention can be made into therapeutically or prophylactically effective drugs for preventing or ameliorating diseases, disorders or symptoms caused by or associated with a protein complex or an interacting member thereof. Alternatively, they may be used as leads to aid the design and identification of therapeutically or prophylactically effective compounds for diseases, disorders or symptoms caused by or associated with the protein complex or interacting protein members thereof. The protein complexes and/or interacting protein members thereof in accordance with the present invention can be used in any of a variety of drug screening techniques. Drug screening can be performed as described herein or using well-known techniques, such as those described in U.S. Pat. Nos. 5,800,998 and 5,891,628, both of which are incorporated herein by reference.

### 5.1. Test Compounds

Any test compounds may be screened in the screening assays of the present invention to select modulators of the protein complexes or interacting members thereof. The terms “selecting” or “to select” compounds are intended to encompass both (a) choosing compounds from a group previously unknown to be modulators of a protein complex or interacting protein members thereof, and (b) testing compounds that are known to be capable of binding, or modulating the functions and activities of, a protein complex or interacting protein members thereof. Both types of compounds are generally referred to herein as “test compounds.” The test compounds may include, by way of example, proteins (e.g., antibodies, small peptides, artificial or natural proteins), nucleic acids, and derivatives, mimetics and analogs thereof, and small organic molecules having a molecular weight of no greater than 10,000 daltons, more preferably less than 5,000 daltons. Preferably, the test compounds are provided in library formats known in the art, e.g., in chemically synthesized libraries, recombinantly expressed libraries (e.g., phage display libraries), and in vitro translation-based libraries (e.g., ribosome display libraries).

For example, the screening assays of the present invention can be used in the antibody production processes described in Section 3 to select antibodies with desirable specificities. Various forms of antibodies or derivatives thereof may be screened, including but not limited to, polyclonal antibodies, monoclonal antibodies, bifunctional antibodies, chimeric antibodies, single chain antibodies, antibody fragments such as Fv fragments, single-chain Fv fragments (scFv), Fab′ fragments, and F(ab′)₂ fragments, and various modified forms of antibodies such as catalytic antibodies, and antibodies conjugated to toxins or drugs, and the like. The antibodies can be of any types such as IgG, IgE, IgA, or IgM. Humanized antibodies are particularly preferred. Preferably, the various antibodies and antibody fragments may be provided in libraries to allow large-scale high throughput screening. For example, expression libraries expressing antibodies or antibody fragments may be constructed by a method disclosed, e.g., in Huse et al., Science, 246:1275-1281 (1989), which is incorporated herein by reference. Single-chain Fv (scFv) antibodies are of particular interest in diagnostic and therapeutic applications. Methods for providing antibody libraries are also provided in U.S. Pat. Nos. 6,096,551; 5,844,093; 5,837,460; 5,789,208; and 5,667,988, all of which are incorporated herein by reference.

Peptidic test compounds may be peptides having L-amino acids and/or D-amino acids, phosphopeptides, and other types of peptides. The screened peptides can be of any size, but preferably have less than about 50 amino acids. Smaller peptides are easier to deliver into a patient's body. Various forms of modified peptides may also be screened. Like antibodies, peptides can also be provided in, e.g., combinatorial libraries. See generally, Gallop et al., J Med Chem, 37:1233-1251 (1994). Methods for making random peptide libraries are disclosed in, e.g., Devlin et al., Science, 249:404-406 (1990). Other suitable methods for constructing peptide libraries and screening peptides therefrom are disclosed in, e.g., Scott & Smith, Science, 249:386-390 (1990); Moran et al., J Am Chem Soc, 117:10787-10788 (1995) (a library of electronically tagged synthetic peptides); Stachelhaus et al., Science, 269:69-72 (1995); U.S. Pat. Nos. 6,156,511; 6,107,059; 6,015,561; 5,750,344; 5,834,318; 5,750,344, all of which are incorporated herein by reference. For example, random-sequence peptide phage display libraries may be generated by cloning synthetic oligonucleotides into the gene III or gene VIII of an E. coli filamentous phage. The thus generated phage can propagate in E. coli. and express peptides encoded by the oligonucleotides as fusion proteins on the surface of the phage. Scott & Smith, Science, 249:368-390 (1990). Alternatively, the “peptides on plasmids” method may also be used to form peptide libraries. In this method, random peptides may be fused to the C-terminus of the E. coli. Lac repressor by recombinant technologies and expressed from a plasmid that also contains Lac repressor-binding sites. As a result, the peptide fusions bind to the same plasmid that encodes them.

Small organic or inorganic non-peptide non-nucleotide compounds are preferred test compounds for the screening assays of the present invention. They too can be provided in a library format. See generally, Gordan et al. J Med Chem, 37:1385-1401 (1994). For example, benzodiazepine libraries are provided in Bunin & Ellman, J Am Chem Soc,114:10997-10998 (1992), which is incorporated herein by reference. Methods for constructing and screening peptoid libraries are disclosed in Simon et al., Proc Natl Acad Sci USA, 89:9367-9371 (1992). Methods for the biosynthesis of novel polyketides in a library format are described in McDaniel et al., Science, 262:1546-1550 (1993) and Kao et al., Science, 265:509-512 (1994). Various libraries of small organic molecules and methods of construction thereof are disclosed in U.S. Pat. No. 6,162,926 (multiply-substituted fullerene derivatives); U.S. Pat. No. 6,093,798 (hydroxamic acid derivatives); U.S. Pat. No. 5,962,337 (combinatorial 1,4-benzodiazepin-2, 5-dione library); U.S. Pat. No. 5,877,278 (Synthesis of N-substituted oligomers); U.S. Pat. No. 5,866,341 (compositions and methods for screening drug libraries); U.S. Pat. No. 5,792,821 (polymerizable cyclodextrin derivatives); U.S. Pat. No. 5,766,963 (hydroxypropylamine library); and U.S. Pat. No. 5,698,685 (morpholino-subunit combinatorial library), all of which are incorporated herein by reference.

Other compounds such as oligonucleotides and peptide nucleic acids (PNA), and analogs and derivatives thereof may also be screened to identify clinically useful compounds. Combinatorial libraries of oligonucleotides are also known in the art. See Gold et al., J Biol Chem, 270:13581-13584 (1995).

### 5.2. In Vitro Screening Assays

The test compounds may be screened in an in vitro assay to identify compounds capable of binding the protein complexes or interacting protein members thereof in accordance with the present invention. For this purpose, a test compound is contacted with a protein complex or an interacting protein member thereof under conditions and for a time sufficient to allow specific interaction between the test compound and the target components to occur, thereby resulting in the binding of the compound to the target, and the formation of a complex. Subsequently, the binding event is detected.

Various screening techniques known in the art may be used in the present invention. The protein complexes and the interacting protein members thereof may be prepared by any suitable methods, e.g., by recombinant expression and purification. The protein complexes and/or interacting protein members thereof (both are referred to as “target” hereinafter in this section) may be free in solution. A test compound may be mixed with a target forming a liquid mixture. The compound may be labeled with a detectable marker. Upon mixing under suitable conditions, the binding complex having the compound and the target may be co-immunoprecipitated and washed. The compound in the precipitated complex may be detected based on the marker on the compound.

In a preferred embodiment, the target is immobilized on a solid support or on a cell surface. Preferably, the target can be arrayed into a protein microchip in a method described in Section 2.4. For example, a target may be immobilized directly onto a microchip substrate such as glass slides or onto multi-well plates using non-neutralizing antibodies, i.e., antibodies capable of binding to the target but do not substantially affect its biological activities. To affect the screening, test compounds can be contacted with the immobilized target to allow binding to occur to form complexes under standard binding assay conditions. Either the targets or test compounds are labeled with a detectable marker using well-known labeling techniques. For example, U.S. Pat. No. 5,741,713 discloses combinatorial libraries of biochemical compounds labeled with NMR active isotopes. To identify binding compounds, one may measure the formation of the target-test compound complexes or kinetics for the formation thereof. When combinatorial libraries of organic non-peptide non-nucleic acid compounds are screened, it is preferred that labeled or encoded (or “tagged”) combinatorial libraries are used to allow rapid decoding of lead structures. This is especially important because, unlike biological libraries, individual compounds found in chemical libraries cannot be amplified by self-amplification. Tagged combinatorial libraries are provided in, e.g., Borchardt & Still, J Am Chem Soc, 116:373-374 (1994) and Moran et al., J Am Chem Soc, 117:10787-10788 (1995), both of which are incorporated herein by reference.

Alternatively, the test compounds can be immobilized on a solid support, e.g., forming a microarray of test compounds. The target protein or protein complex is then contacted with the test compounds. The target may be labeled with any suitable detection marker. For example, the target may be labeled with radioactive isotopes or fluorescence marker before binding reaction occurs. Alternatively, after the binding reactions, antibodies that are immunoreactive with the target and are labeled with radioactive materials, fluorescence markers, enzymes, or labeled secondary anti-Ig antibodies may be used to detect any bound target thus identifying the binding compound. One example of this embodiment is the protein probing method. That is, the target provided in accordance with the present invention is used as a probe to screen expression libraries of proteins or random peptides. The expression libraries can be phage display libraries, in vitro translation-based libraries, or ordinary expression cDNA libraries. The libraries may be immobilized on a solid support such as nitrocellulose filters. See e.g., Sikela & Hahn, Proc Natl Acad Sci USA, 84:3038-3042 (1987). The probe may be labeled with a radioactive isotope or a fluorescence marker. Alternatively, the probe can be biotinylated and detected with a streptavidin-alkaline phosphatase conjugate. More conveniently, the bound probe may be detected with an antibody.

In one embodiment, the proteins identified in the tables are used as targets in an assay to select modulators of the interacting proteins in the tables. In a specific embodiment, a screening assay for modulators of δ-catenin is performed using FAK2 as a ligand for δ-catenin. For example, in this screen, δ-catenin can be immobilized on a solid support and is contacted with test compounds. FAK2 can be labeled with a detectable marker such as radioactive materials or fluorescence markers using labeling techniques known in the art. The labeled FAK2 is allowed to contact the immobilized δ-catenin and levels of APP(695):FAK2 protein complex formed are detected by washing away unbound FAK2. The ability of the test compounds to modulate δ-catenin is determined by comparing the level of δ-catenin:FAK2 complex formed when δ-catenin is contacted with test compounds, to the level formed in the absence of test compounds. Alternatively, as will be apparent to skilled artisans, the FAK2 protein can be detected with labeled antibody against FAK2, or by an antibody specific to a polypeptide that is fused to FAK2.

In yet another embodiment, the protein complexes identified in the tables are used as a target in the assay. In a specific embodiment, a protein complex used in the screening assay includes a hybrid protein as described in Section 2.2, which is formed by fusion of two interacting protein members or fragments or interaction domains thereof. The hybrid protein may also be designed such that it contains a detectable epitope tag fused thereto. Suitable examples of such epitope tags include sequences derived from, e.g., influenza virus hemagglutinin (HA), Simian Virus 5 (V5), polyhistidine (6xHis), c-myc, lacZ, GST, and the like.

In addition, a known ligand capable of binding to the target can be used in competitive binding assays. Complexes between the known ligand and the target can be formed and then contacted with test compounds. The ability of a test compound to interfere with the interaction between the target and the known ligand is measured. One exemplary ligand is an antibody capable of specifically binding the target. Particularly, such an antibody is especially useful for identifying peptides that share one or more antigenic determinants of the target protein complex or interacting protein members thereof.

In a specific preferred embodiment, the target is one member of an interacting pair of proteins disclosed according the present invention, or a homologue, derivative or fragment thereof, and the competitive ligand is the other member of the interacting pair of proteins, or a homologue, derivative or fragment thereof. Preferably, either the target or the ligand or both are labeled with or detectable marker. Alternatively, either the target or the ligand or both are fusion proteins that contain a detectable epitope tag having one or more sequences derived from, e.g., influenza virus hemagglutinin (HA), Simian Virus 5 (V5), polyhistidine (6xHis), c-myc, lacZ, GST, and the like.

Thus, for example, the target can be immobilized to a solid support. The ligand can be a fusion protein having a fragment of an interactor of the target protein fused to an epitope tag, e.g., c-myc. The ligand can be contacted with the target in the presence or absence of one or more test compounds. Both ligand molecules associated with the immobilized target and ligand molecules not associated with the target can be detected with, e.g., an antibody against the c-myc tag. As a result, test compounds capable of binding the target or ligand, or disrupting the protein-protein interaction between the target and ligand can be identified or selected.

Test compounds may also be screened in an in vitro assay to identify compounds capable of dissociating the protein complexes identified in the tables above. Thus, for example, any one of the interacting pairs of proteins described in the tables above can be contacted with a test compound and the integrity of the protein complex can be assessed. Conversely, test compounds may also be screened to identify compounds capable of enhancing the interactions between the constituent members of the protein complexes formed by the interactions described in the tables. The assays can be conducted in a manner similar to the binding assays described above. For example, the presence or absence of a particular pair of interacting proteins can be detected by an antibody that is selectively immunoreactive with the protein complex formed by those two proteins. Thus, after incubation of the protein complex with a test compound, an immuno-precipitation assay can be conducted with the antibody. If the test compound disrupts the protein complex, then the amount of immunoprecipitated protein complex in this assay will be significantly less than that in a control assay in which the same protein complex is not contacted with the test compound. Similarly, two proteins—the interaction between which is to be enhanced—may be incubated together with a test compound. Thereafter, a protein complex formed by the two interacting proteins can be detected by the selectively immunoreactive antibody. The amount of protein complex may be compared to that formed in the absence of the test compound. Various other detection methods may be suitable in the dissociation assay, as will be apparent to a skilled artisan apprised of the present disclosure.

In another embodiment, fluorescent resonance energy transfer (FRET) is used to screen for modulators of interacting proteins of the protein complexes of the present invention. FRET assays measure the energy transfer of one fluorescent label to another fluorescent label. Fluorescent labels absorb light preferentially at one wavelength and emit light preferentially at a second wavelength. FRET assays utilize this characteristic by selecting a fluorescent label, called a donor fluorophore, that emits light preferentially at the wavelength a second label, called the acceptor fluorophore, preferentially absorbs light. The proximity of the donor and acceptor fluorophore can be determined by measuring the energy transfer from the donor fluorophore to the acceptor fluorophore. Measuring the energy transfer is performed by shining light on a solution containing acceptor and donor fluorophores at the wavelength the donor fluorophore absorbs light and measuring fluorescence at the wavelength the acceptor fluorophore emits light. The amount of fluorescence of the acceptor fluorophore indicates the proximity of the donor and acceptor fluorophores.

For example, FRET assays can be used to screen for modulators of δ-catenin by labeling δ-catenin or an antibody to δ-catenin with an acceptor fluorophore and labeling a δ-catenin substrate or interactor (e.g., FAK2) or an antibody to a δ-catenin substrate/interactor with an acceptor fluorophore. If the test compound is a δ-catenin modulator it will decrease the fluorescence of the acceptor fluorophore because the acceptor and donor fluorphore will not be as close to each other.

In a specific embodiment of a FRET assay, TP³⁺ is attached to an antibody to δ-catenin, and BODIPY-TMR is attached to an antibody to an interactor (e.g., FAK2). The fluorescently labeled antibodies, δ-catenin, and δ-catenin substrates are put in solution together. Light at the wavelength that TP³⁺ preferentially absorbs light is shined on the solution and the fluorescence of the solution is measured at the wavelength that BODIPY-TMR preferentially emits light. A test compound is then added to the solution and and light at the wavelength that TP³⁺ preferentially absorbs light is shined on the solution and the fluorescence of the solution is measured at the wavelength that BODIPY-TMR preferentially emits light. If the fluorescence of the solution with the test compound decreases compared to the fluorescence of the solution without the test compound then the test compound is a δ-catenin modulator.

Similarly, but in another, perhaps preferred embodiment, fluorescence polarization (FP) is used to screen for potential modulators that bind the interacting proteins of the protein complexes of the present invention. FP takes advantage of the fact that a fluorescent molecule, when excited with plane polarized light, reemits light in the same plane, provided the molecule remains stationary during the time between excitation and emission (e.g., about 4 nanoseconds for fluorescein). If, however, the fluorescent molecule rotates out of its initial orientation during this time, the light emitted will be emitted in a different plane from that of the excitation light. Consequently, the movement (rotation and tumbling) of fluorescently labeled molecules in solution can be monitored by measuring the amount of light emitted in the original excitation plane, compared to the amount of light emitted in a perpendicular plane. For example, if fluorescently labeled molecules (e.g., test compounds with a fluorescent tag attached) are excited in with light of the appropriate excitation energy, which is polarized in the verticle plane, monitoring the amount of light emitted at the appropriate emission energy in the vertical and horizontal planes will provide information that can be used to determine the degree of movement of the fluorescently labeled molecule relative to its original position. If a molecule is very large, or is bound to a large binding partner, little movement occurs between the moments of excitation and emission, and the emitted light remains highly polarized, and in the same plane as the excitiation light. If the molecule is small, or is not bound to a large binding partner, it will rotate and tumble away from its orientation at the moment of excitation more readily and rapidly, and the light it emits will be more depolarized relative to the excitation plane. As a result, FP is a particularly useful technique for studying the interaction between relatively small, fluorescently labeled test compounds, and relatively large biomolecules (proteins or protein complexes), giving a nearly instantaneous measure of the bound/free ratio of the fluorescently labeled test compound. FP experiments are done in solution and in real time, and when preferred fluorescent tags are employed they allow true equlibrium analysis down to the low picomolar range. Furthermore, FP measurements do not spoil samples, so the sample can be reanalyzed after adjustments in pH, temperature, and salt concentrations, or after addition of a competitive inhibitor or interacting protein partner.

Advantageously, for those pairs of interacting proteins disclosed in the tables above where one or both interacting proteins exhibits a measurable enzymatic or other biological activity, in vitro screening assays can be designed to detect alterations in that measurable activity. For example, as described above, we have discovered that FAK2 interacts with δ-catenin, and both this interaction and high levels of expression of FAK2 in hippocampus and amygdala suggest that an alteration in FAK2s activity may be related to neuronal death in AD. Hence, compounds that modulate the activity of FAK2, or modulate its interaction with δ-catenin may thus prove beneficial therapeutic agents for the treatment of AD. Additionally, FAK2 (which is also known as Pyk2/RAFTK) is known to phosporylate tyrosine residue 125 of alpha-Synuclein (Nakamura et al., FEBS Lett 521:190-194 (2002)). Consequently, in a specific example, in vitro screening assays for test compounds that modulate this phosphorylation activity of FAK2 in the presence and/or absence of its interactor, δ-catenin, can be designed using cell lysates or purified components and procedures widely known in the art (i.e., phosphorylation or kinase assays; see Nakamura et al., FEBS Lett 521:190-194 (2002)). Thus, the instant invention provides for designing and conducting assays using the kinase activity of FAK2 as a means for screening for compounds useful in the treatment of neurological disorders, ailments and diseases, including mild cognitive impairment, depression, schizophrenia, obsessive-compulsive disorder, bipolar disorder, and neurodegenerative diseases and disorders and motor neuron diseases and disorders such as Alzheimer's disease, Parkinson's disease, dementia with Lewy bodies, amyotrophic lateral sclerosis or Lou Gehrig's disease, Alpers' disease, Leigh's disease, Pelizaeus-Merzbacher disease, Olivopontocerebellar atrophy, Friedreich's ataxia, leukodystrophies, Rett syndrome, Ramsay Hunt syndrome type II, and Down's syndrome.

The instant invention also provides for in-vitro screening assays for compounds capable of modulating the interaction between FAK2 and δ-catenin and/or the activity of the FAK2:δ-catenin complex, as well as interactions formed between FAK2 and its other binding partners.

### 5.3. In Vivo Screening Assays

Test compounds can also be screened in a wide variety of in vivo assays designed to select modulators of the protein complexes or interacting protein members thereof in accordance with the present invention. For example, any in vivo assays known in the art to be useful in identifying compounds capable of strengthening or interfering with the stability of the protein complexes of the present invention may be used.

In a specific example, a screening assay for modulators of a δ-catenin is performed by using FAK2 as a ligand for δ-catenin. In this screen, δ-catenin is contacted with test compounds in the presence of FAK2and the levels of δ-catenin FAK2 protein complex formed when δ-catenin is contacted with the test compound in the presence of FAK2 is detected. The ability of the test compounds to modulate δ-catenin is determined by comparing the level of δ-catenin:FAK2 complex formed when δ-catenin is contacted with test compounds to the level formed in the absence of test compounds. If the level of δ-catenin:FAK2protein complex formed when δ-catenin is contacted with the test compound then the test compound is a modulator of δ-catenin.

To screen peptidic compounds for modulators of δ-catenin, the two-hybrid systems described in Section 4 may be used in the screening assays in which the δ-catenin protein is expressed in, e.g., a bait fusion protein and the peptidic test compounds are expressed in, e.g., prey fusion proteins. Screening peptidic compounds for modulators of the proteins identified in the tables can also be performed using the two-hybrid systems described in Section 4 by expressing the proteins identified in the tables in, e.g., a bait fusion protein and expressing the peptidic test compounds in e.g., prey fusion proteins.

To screen for modulators of the protein-protein interaction between δ-catenin and a δ-catenin interacting protein, the methods of the present invention typically comprise contacting the δ-catenin protein with the δ-catenin interacting protein in the presence of a test compound, and determining the interaction between the δ-catenin protein and the δ-catenin interacting protein. In a preferred embodiment, a two-hybrid system, e.g., a yeast two-hybrid system as described in detail in Section 4 is employed.

Importantly, it has been discovered using the cell-based Aβ secretion assay disclosed in Example 8, below, that modulation of levels of expression of several of the interacting proteins of the instant invention affects the processing of APP, the production of Aβ in general, and the secretion of the neurotoxic Aβ42 peptide, in particular. Specifically, it has been discovered that overexpression of FAK2, SCD, CIB, and BAT3 results in increased secretion of Aβ42 in the cell based assay of Example 8. Consequently, it is expected that inhibition of FAK2, SCD, CIB, and BAT3, or a reduction in their expression, will result in decreased Aβ42 secretion. (This hypothesis has been tested and confirmed in the case of SCD.) Additionally, using the schemes described above, inhibitors of FAK2, SCD, CIB, and BAT3 can be selected and these selected inhibitors can be further tested for their ability to reduce Aβ42 secretion in the cell-based assay described in Example 8. The inhibitors so discovered can be subjected to iterative rounds of SAR, as described above, and the modified compounds can be further tested for their ability to more effectively reduce Aβ42 secretion. The compounds with the most desirable features can then be tested in subsequent pre-clinical trials in animals, before ultimately being tested in clinical trials in human patients in need of such treatment.

### 5.3.1. Two-Hybrid Assays

In a specific embodiment, one of the yeast two-hybrid systems or their analogous or derivative forms is used. Examples of suitable two-hybrid systems known in the art include, but are not limited to, those disclosed in U.S. Pat. Nos. 5,283,173; 5,525,490; 5,585,245; 5,637,463; 5,695,941; 5,733,726; 5,776,689; 5,885,779; 5,905,025; 6,037,136; 6,057,101; 6,114,111; and Bartel and Fields, eds., The Yeast Two-Hybrid System, Oxford University Press, New York, N.Y., 1997, all of which are incorporated herein by reference.

Typically, in a classic transcription-based two-hybrid assay, two chimeric genes are prepared encoding two fusion proteins: one contains a transcription activation domain fused to an interacting protein member of a protein complex of the present invention or an interaction domain or fragment of the interacting protein member, while the other fusion protein includes a DNA binding domain fused to another interacting protein member of the protein complex or a fragment or interaction domain thereof. For the purpose of convenience, the two interacting protein members, fragments or interaction domains thereof are referred to as “bait fusion protein” and “prey fusion protein,” respectively. The chimeric genes encoding the fusion proteins are termed “bait chimeric gene” and “prey chimeric gene,” respectively. Typically, a “bait vector” and a “prey vector” are provided for the expression of a bait chimeric gene and a prey chimeric gene, respectively.

### 5.3.1.1. Vectors

Many types of vectors can be used in a transcription-based two-hybrid assay. Methods for the construction of bait vectors and prey vectors should be apparent to skilled artisans in the art apprised of the present disclosure. See generally, Current Protocols in Molecular Biology, Vol. 2, Ed. Ausubel, et al., Greene Publish. Assoc. & Wiley Interscience, Ch. 13, 1988; Glover, DNA Cloning, Vol. II, IRL Press, Wash., D.C., Ch. 3, 1986; Bitter, et al., in Methods in Enzymology 153:516-544 (1987); The Molecular Biology of the Yeast Saccharomyces, Eds. Strathern et al., Cold Spring Harbor Press, Vols. I and II, 1982; and Rothstein in DNA Cloning: A Practical Approach, Vol. 11, Ed. D M Glover, IRL Press, Wash., D.C., 1986.

Generally, the bait and prey vectors include an expression cassette having a promoter operably linked to a chimeric gene for the transcription of the chimeric gene. The vectors may also include an origin of DNA replication for the replication of the vectors in host cells and a replication origin for the amplification of the vectors in, e.g., E. coli, and selection marker(s) for selecting and maintaining only those host cells harboring the vectors. Additionally, the expression cassette preferably also contains inducible elements, which function to control the expression of a chimeric gene. Making the expression of the chimeric genes inducible and controllable is especially important in the event that the fusion proteins or components thereof are toxic to the host cells. Other regulatory sequences such as transcriptional enhancer sequences and translation regulation sequences (e.g., Shine-Dalgarno sequence) can also be included in the expression cassette. Termination sequences such as the bovine growth hormone, SV40, lacZ and AcMNPV polyhedral polyadenylation signals may also be operably linked to a chimeric gene in the expression cassette. An epitope tag coding sequence for detection and/or purification of the fusion proteins can also be operably linked to the chimeric gene in the expression cassette. Examples of useful epitope tags include, but are not limited to, influenza virus hemagglutinin (HA), Simian Virus 5 (V5), polyhistidine (6xHis), c-myc, lacZ, GST, and the like. Proteins with polyhistidine tags can be easily detected and/or purified with Ni affinity columns, while specific antibodies to many epitope tags are generally commercially available. The vectors can be introduced into the host cells by any techniques known in the art, e.g., by direct DNA transformation, microinjection, electroporation, viral infection, lipofection, gene gun, and the like. The bait and prey vectors can be maintained in host cells in an extrachromosomal state, i.e., as self-replicating plasmids or viruses. Alternatively, one or both vectors can be integrated into chromosomes of the host cells by conventional techniques such as selection of stable cell lines or site-specific recombination.

The in vivo assays of the present invention can be conducted in many different host cells, including but not limited to bacteria, yeast cells, plant cells, insect cells, and mammalian cells. A skilled artisan will recognize that the designs of the vectors can vary with the host cells used. In one embodiment, the assay is conducted in prokaryotic cells such as Escherichia coli, Salmonella, Klebsiella, Pseudomonas, Caulobacter, and Rhizobium. Suitable origins of replication for the expression vectors useful in this embodiment of the present invention include, e.g., the Co1E1, pSC101, and M13 origins of replication. Examples of suitable promoters include, for example, the T7 promoter, the lacZ promoter, and the like. In addition, inducible promoters are also useful in modulating the expression of the chimeric genes. For example, the lac operon from bacteriophage lambda plac5 is well known in the art and is inducible by the addition of IPTG to the growth medium. Other known inducible promoters useful in a bacteria expression system include pL of bacteriophage λ, the trp promoter, and hybrid promoters such as the tac promoter, and the like.

In addition, selection marker sequences for selecting and maintaining only those prokaryotic cells expressing the desirable fusion proteins should also be incorporated into the expression vectors. Numerous selection markers including auxotrophic markers and antibiotic resistance markers are known in the art and can all be useful for purposes of this invention. For example, the bla gene, which confers ampicillin resistance, is the most commonly used selection marker in prokaryotic expression vectors. Other suitable markers include genes that confer neomycin, kanamycin, or hygromycin resistance to the host cells. In fact, many vectors are commercially available from vendors such as Invitrogen Corp. of Carlsbad, Calif., Clontech Corp. of Palo Alto, Calif., and Stratagene Corp. of La Jolla, Calif., and Promega Corp. of Madison, Wis. These commercially available vectors, e.g., pBR322, pSPORT, pBluescriptIISK, pcDNAI, and pcDNAII all have a multiple cloning site into which the chimeric genes of the present invention can be conveniently inserted using conventional recombinant techniques. The constructed expression vectors can be introduced into host cells by various transformation or transfection techniques generally known in the art.

In another embodiment, mammalian cells are used as host cells for the expression of the fusion proteins and detection of protein-protein interactions. For this purpose, virtually any mammalian cells can be used including normal tissue cells, stable cell lines, and transformed tumor cells. Conveniently, mammalian cell lines such as CHO cells, Jurkat T cells, NIH 3T3 cells, HEK-293 cells, CV-1 cells, COS-1 cells, HeLa cells, VERO cells, MDCK cells, W138 cells, and the like are used. Mammalian expression vectors are well known in the art and many are commercially available. Examples of suitable promoters for the transcription of the chimeric genes in mammalian cells include viral transcription promoters derived from adenovirus, simian virus 40 (SV40) (e.g., the early and late promoters of SV40), Rous sarcoma virus (RSV), and cytomegalovirus (CMV) (e.g., CMV immediate-early promoter), human immunodeficiency virus (HIV) (e.g., long terminal repeat (LTR)), vaccinia virus (e.g., 7.5K promoter), and herpes simplex virus (HSV) (e.g., thymidine kinase promoter). Inducible promoters can also be used. Suitable inducible promoters include, for example, the tetracycline responsive element (TRE) (See Gossen et al., Proc Natl Acad Sci USA, 89:5547-5551 (1992)), metallothionein IIA promoter, ecdysone-responsive promoter, and heat shock promoters. Suitable origins of replication for the replication and maintenance of the expression vectors in mammalian cells include, e.g., the Epstein Barr origin of replication in the presence of the Epstein Barr nuclear antigen (see Sugden et al., Mol Cell Biol, 5:410-413 (1985)) and the SV40 origin of replication in the presence of the SV40 T antigen (which is present in COS-1 and COS-7 cells) (see Margolskee et al., Mol Cell Biol, 8:2837 (1988)). Suitable selection markers include, but are not limited to, genes conferring resistance to neomycin, hygromycin, zeocin, and the like. Many commercially available mammalian expression vectors may be useful for the present invention, including, e.g., pCEP4, pcDNAI, pIND, pSecTag2, pVAX1, pcDNA3.1, and pBI-EGFP, and pDisplay. The vectors can be introduced into mammalian cells using any known techniques such as calcium phosphate precipitation, lipofection, electroporation, and the like. The bait vector and prey vector can be co-transformed into the same cell or, alternatively, introduced into two different cells which are subsequently fused together by cell fusion or other suitable techniques.

Viral expression vectors, which permit introduction of recombinant genes into cells by viral infection, can also be used for the expression of the fusion proteins. Viral expression vectors generally known in the art include viral vectors based on adenovirus, bovine papilloma virus, murine stem cell virus (MSCV), MFG virus, and retrovirus. See Sarver, et al., Mol Cell Biol, 1: 486 (1981); Logan & Shenk, Proc Natl Acad Sci USA, 81:3655-3659 (1984); Mackett, et al., Proc Natl Acad Sci USA, 79:7415-7419 (1982); Mackett, et al., J Virol, 49:857-864 (1984); Panicali, et al., Proc Natl Acad Sci USA, 79:4927-4931 (1982); Cone & Mulligan, Proc Natl Acad Sci USA, 81:6349-6353 (1984); Mann et al., Cell, 33:153-159 (1993); Pear et al., Proc Natl Acad Sci USA, 90:8392-8396 (1993); Kitamura et al., Proc Natl Acad Sci USA, 92:9146-9150 (1995); Kinsella et al., Human Gene Therapy, 7:1405-1413 (1996); Hofmann et al., Proc Natl Acad Sci USA, 93:5185-5190 (1996); Choate et al., Human Gene Therapy, 7:2247 (1996); WO 94/19478; Hawley et al., Gene Therapy, 1:136 (1994) and Rivere et al., Genetics, 92:6733 (1995), all of which are incorporated by reference.

Generally, to construct a viral vector, a chimeric gene according to the present invention can be operably linked to a suitable promoter. The promoter-chimeric gene construct is then inserted into a non-essential region of the viral vector, typically a modified viral genome. This results in a viable recombinant virus capable of expressing the fusion protein encoded by the chimeric gene in infected host cells. Once in the host cell, the recombinant virus typically is integrated into the genome of the host cell. However, recombinant bovine papilloma viruses typically replicate and remain as extrachromosomal elements.

In another embodiment, the detection assays of the present invention are conducted in plant cell systems. Methods for expressing exogenous proteins in plant cells are well known in the art. See generally, Weissbach & Weissbach, Methods for Plant Molecular Biology, Academic Press, NY, 1988; Grierson & Corey, Plant Molecular Biology, 2nd Ed., Blackie, London, 1988. Recombinant virus expression vectors based on, e.g., cauliflower mosaic virus (CaMV) or tobacco mosaic virus (TMV) can all be used. Alternatively, recombinant plasmid expression vectors such as Ti plasmid vectors and Ri plasmid vectors are also useful. The chimeric genes encoding the fusion proteins of the present invention can be conveniently cloned into the expression vectors and placed under control of a viral promoter such as the 35S RNA and 19S RNA promoters of CaMV or the coat protein promoter of TMV, or of a plant promoter, e.g., the promoter of the small subunit of RUBISCO and heat shock promoters (e.g., soybean hsp17.5-E or hsp17.3-B promoters).

In addition, the in vivo assay of the present invention can also be conducted in insect cells, e.g., Spodoptera frugiperda cells, using a baculovirus expression system. Expression vectors and host cells useful in this system are well known in the art and are generally available from various commercial vendors. For example, the chimeric genes of the present invention can be conveniently cloned into a non-essential region (e.g., the polyhedrin gene) of an Autographa californica nuclear polyhedrosis virus (AcNPV) vector and placed under control of an AcNPV promoter (e.g., the polyhedrin promoter). The non-occluded recombinant viruses thus generated can be used to infect host cells such as Spodoptera frugiperda cells in which the chimeric genes are expressed. See U.S. Pat. No. 4,215,051.

In a preferred embodiment of the present invention, the fusion proteins are expressed in a yeast expression system using yeasts such as Saccharomyces cerevisiae, Hansenula polymorpha, Pichia pastoris, and Schizosaccharomyces pombe as host cells. The expression of recombinant proteins in yeasts is a well-developed field, and the techniques useful in this respect are disclosed in detail in The Molecular Biology of the Yeast Saccharomyces, Eds. Strathern et al., Vols. I and II, Cold Spring Harbor Press, 1982; Ausubel et al., Current Protocols in Molecular Biology, New York, Wiley, 1994; and Guthrie and Fink, Guide to Yeast Genetics and Molecular Biology, in Methods in Enzymology, Vol. 194, 1991, all of which are incorporated herein by reference. Sudbery, Curr Opin Biotech, 7:517-524 (1996) reviews the successes in the art of expressing recombinant proteins in various yeast species; the entire content and references cited therein are incorporated herein by reference. In addition, Bartel and Fields, eds., The Yeast Two-Hybrid System, Oxford University Press, New York, N.Y., 1997 contains extensive discussions of recombinant expression of fusion proteins in yeasts in the context of various yeast two-hybrid systems, and cites numerous relevant references. These and other methods known in the art can all be used for purposes of the present invention. The application of such methods to the present invention should be apparent to a skilled artisan apprised of the present disclosure.

Generally, each of the two chimeric genes is included in a separate expression vector (bait vector and prey vector). Both vectors can be co-transformed into a single yeast host cell. As will be apparent to a skilled artisan, it is also possible to express both chimeric genes from a single vector. In a preferred embodiment, the bait vector and prey vector are introduced into two haploid yeast cells of opposite mating types, e.g., α-type and a-type, respectively. The two haploid cells can be mated at a desired time to form a diploid cell expressing both chimeric genes.

Generally, the bait and prey vectors for recombinant expression in yeast include a yeast replication origin such as the 2μ origin or the ARSH4 sequence for the replication and maintenance of the vectors in yeast cells. Preferably, the vectors also have a bacteria origin of replication (e.g., Co1E1) and a bacteria selection marker (e.g., ampR marker, i.e., bla gene). Optionally, the CEN6 centromeric sequence is included to control the replication of the vectors in yeast cells. Any constitutive or inducible promoters capable of driving gene transcription in yeast cells may be employed to control the expression of the chimeric genes. Such promoters are operably linked to the chimeric genes. Examples of suitable constitutive promoters include but are not limited to the yeast ADH1, PGK1, TEF2 , GPD1, HIS3, and CYC1 promoters. Examples of suitable inducible promoters include but are not limited to the yeast GAL1 (inducible by galactose), CUP1 (inducible by Cu⁺⁺), and FUS1 (inducible by pheromone) promoters; the AOX/MOX promoter from H. polymorpha and P. pastoris (repressed by glucose or ethanol and induced by methanol); chimeric promoters such as those that contain LexA operators (inducible by LexA-containing transcription factors); and the like. Inducible promoters are preferred when the fusion proteins encoded by the chimeric genes are toxic to the host cells. If it is desirable, certain transcription repressing sequences such as the upstream repressing sequence (URS) from SPO13 promoter can be operably linked to the promoter sequence, e.g., to the 5′ end of the promoter region. Such upstream repressing sequences function to fine-tune the expression level of the chimeric genes.

Preferably, a transcriptional termination signal is operably linked to the chimeric genes in the vectors. Generally, transcriptional termination signal sequences derived from, e.g., the CYC1 and ADH1 genes can be used.

Additionally, it is preferred that the bait vector and prey vector contain one or more selectable markers for the selection and maintenance of only those yeast cells that harbor one or both chimeric genes. Any selectable markers known in the art can be used for purposes of this invention so long as yeast cells expressing the chimeric gene(s) can be positively identified or negatively selected. Examples of markers that can be positively identified are those based on color assays, including the lacZ gene (which encodes 0-galactosidase), the firefly luciferase gene, secreted alkaline phosphatase, horseradish peroxidase, the blue fluorescent protein (BFP), and the green fluorescent protein (GFP) gene (see Cubitt et al., Trends Biochem Sci, 20:448-455 (1995)). Other markers allowing detection by fluorescence, chemiluminescence, UV absorption, infrared radiation, and the like can also be used. Among the markers that can be selected are auxotrophic markers including, but not limited to, URA3, HIS3, TRP1, LEU2, LYS2, ADE2, and the like. Typically, for purposes of auxotrophic selection, the yeast host cells transformed with bait vector and/or prey vector are cultured in a medium lacking a particular nutrient. Other selectable markers are not based on auxotrophies, but rather on resistance or sensitivity to an antibiotic or other xenobiotic. Examples of such markers include but are not limited to chloramphenicol acetyl transferase (CAT) gene, which confers resistance to chloramphenicol; CAN1 gene, which encodes an arginine permease and thereby renders cells sensitive to canavanine (see Sikorski et al., Meth Enzymol, 194:302-318 (1991)); the bacterial kanamycin resistance gene (kan^{R}), which renders eukaryotic cells resistant to the aminoglycoside G418 (see Wach et al., Yeast, 10:1793-1808 (1994)); and CYH2 gene, which confers sensitivity to cycloheximide (see Sikorski et al., Meth Enzymol, 194:302-318 (1991)). In addition, the CUP1 gene, which encodes metallothionein and thereby confers resistance to copper, is also a suitable selection marker. Each of the above selection markers may be used alone or in combination. One or more selection markers can be included in a particular bait or prey vector. The bait vector and prey vector may have the same or different selection markers. In addition, the selection pressure can be placed on the transformed host cells either before or after mating the haploid yeast cells.

As will be apparent, the selection markers used should complement the host strains in which the bait and/or prey vectors are expressed. In other words, when a gene is used as a selection marker gene, a yeast strain lacking the selection marker gene (or having mutation in the corresponding gene) should be used as host cells. Numerous yeast strains or derivative strains corresponding to various selection markers are known in the art. Many of them have been developed specifically for certain yeast two-hybrid systems. The application and optional modification of such strains with respect to the present invention will be apparent to a skilled artisan apprised of the present disclosure. Methods for genetically manipulating yeast strains using genetic crossing or recombinant mutagenesis are well known in the art. See e.g., Rothstein, Meth Enzymol, 101:202-211 (1983). By way of example, the following yeast strains are well known in the art, and can be used in the present invention upon necessary modifications and adjustment:

L40 strain which has the genotype MATa his3Δ200 trp1-901 leu2-3,112 ade2 L YS2:: (lexAop) 4-HIS3 URA3:: (lexAop) 8-lacZ;

EGY48 strain which has the genotype MATa trp1 his3 ura3 6ops-LEU2; and

MaV103 strain which has the genotype MATa ura3-52 leu2-3,112 trp1-901 his3Δ200 ade2-101 gal4Δ gal80Δ SPAL10::URA3 GAL1::HIS3::lys2 (see Kumar et al., J Biol Chem 272:13548-13554(1997); Vidal et al., Proc Natl Acad Sci USA, 93:10315-10320 (1996)). Such strains are generally available in the research community, and can also be obtained by simple yeast genetic manipulation. See, e.g., The Yeast Two-Hybrid System, Bartel and Fields, eds., pages 173-182, Oxford University Press, New York, N.Y., 1997.

In addition, the following yeast strains are commercially available:

Y190 strain which is available from Clontech, Palo Alto, Calif. and has the genotype MATa gal4 gal80 his3×200 trp1-901 ade2-101 ura3-52 leu2-3, 112 URA3::GAL1-lacZLYS2::GAL1-HIS3 cyhr; and

YRG-2 Strain which is available from Stratagene, La Jolla, Calif. and has the genotype MATa ura3-52 his3-200 ade2-101 lys2-801 trp1-901 leu2-3, 112 gal4-542 gal80-538 LYS2::GAL1-HIS3 URA3::GAL1/CYC1-lacZ.

In fact, different versions of vectors and host strains specially designed for yeast two-hybrid system analysis are available in kits from commercial vendors such as Clontech, Palo Alto, Calif. and Stratagene, La Jolla, Calif., all of which can be modified for use in the present invention.

### 5.3.1.2. Reporters

Generally, in a transcription-based two-hybrid assay, the interaction between a bait fusion protein and a prey fusion protein brings the DNA-binding domain and the transcription-activation domain into proximity forming a functional transcriptional factor that acts on a specific promoter to drive the expression of a reporter protein. The transcription activation domain and the DNA-binding domain may be selected from various known transcriptional activators, e.g., GAL4, GCN4, ARD1, the human estrogen receptor, E. coli LexA protein, herpes simplex virus VP16 (Triezenberg et al., Genes Dev 2:718-729 (1988)), the E. coli B42 protein (acid blob, see Gyuris et al., Cell, 75:791-803 (1993)), NF-kB p65, and the like. The reporter gene and the promoter driving its transcription typically are incorporated into a separate reporter vector. Alternatively, the host cells are engineered to contain such a promoter-reporter gene sequence in their chromosomes. Thus, the interaction or lack of interaction between two interacting protein members of a protein complex can be determined by detecting or measuring changes in the assay system's reporter. Although the reporters and selection markers can be of similar types and used in a similar manner in the present invention, the reporters and selection markers should be carefully selected in a particular detection assay such that they are distinguishable from each other and do not interfere with each other's function.

Many different types of reporters are useful in the screening assays. For example, a reporter protein may be a fusion protein having an epitope tag fused to a protein. Commonly used and commercially available epitope tags include sequences derived from, e.g., influenza virus hemagglutinin (HA), Simian Virus 5 (V5), polyhistidine (6xHis), c-myc, lacZ, GST, and the like. Antibodies specific to these epitope tags are generally commercially available. Thus, the expressed reporter can be detected using an epitope-specific antibody in an immunoassay.

In another embodiment, the reporter is selected such that it can be detected by a color-based assay. Examples of such reporters include, e.g., the lacZ protein (0-galactosidase), the green fluorescent protein (GFP), which can be detected by fluorescence assay and sorted by flow-activated cell sorting (FACS) (See Cubitt et al., Trends Biochem Sci, 20:448-455 (1995)), secreted alkaline phosphatase, horseradish peroxidase, the blue fluorescent protein (BFP), and luciferase photoproteins such as aequorin, obelin, mnemiopsin, and berovin (See U.S. Pat. No. 6,087,476, which is incorporated herein by reference).

Alternatively, an auxotrophic factor is used as a reporter in a host strain deficient in the auxotrophic factor. Thus, suitable auxotrophic reporter genes include, but are not limited to, URA3, HIS3, TRP1, LEU2, LYS2, ADE2, and the like. For example, yeast cells containing a mutant URA3 gene can be used as host cells (Ura⁻ phenotype). Such cells lack URA3-encoded functional orotidine-5′-phosphate decarboxylase, an enzyme required by yeast cells for the biosynthesis of uracil. As a result, the cells are unable to grow on a medium lacking uracil. However, wild-type orotidine-5′-phosphate decarboxylase catalyzes the conversion of a non-toxic compound 5-fluoroorotic acid (5-FOA) to a toxic product, 5-fluorouracil. Thus, yeast cells containing a wild-type URA3 gene are sensitive to 5-FOA and cannot grow on a medium containing 5-FOA. Therefore, when the interaction between the interacting protein members in the fusion proteins results in the expression of active orotidine-5′-phosphate decarboxylase, the Ura⁻ (FoaR) yeast cells will be able to grow on a uracil deficient medium (SC-Ura plates). However, such cells will not survive on a medium containing 5-FOA. Thus, protein-protein interactions can be detected based on cell growth.

Additionally, antibiotic resistance reporters can also be employed in a similar manner. In this respect, host cells sensitive to a particular antibiotic are used. Antibiotic resistance reporters include, for example, the chloramphenicol acetyl transferase (CAT) gene and the kan^{R} gene, which confer resistance to G418 in eukaryotes, and kanamycin in prokaryotes, respectively.

### 5.3.1.3. Screening Assays for Interaction Antagonists

The screening assays of the present invention are useful for identifying compounds capable of interfering with, disrupting, or dissociating the protein-protein interactions formed between members of the interacting protein pairs disclosed in the tables above, or between mutant and wild type, or mutant and mutant forms of these proteins. Since the protein complexes of the present invention are associated with neurological disorders, ailments and diseases, including mild cognitive impairment, depression, schizophrenia, obsessive-compulsive disorder, bipolar disorder, and neurodegenerative diseases and disorders and motor neuron diseases and disorders such as Alzheimer's disease, Parkinson's disease, dementia with Lewy bodies, amyotrophic lateral sclerosis or Lou Gehrig's disease, Alpers' disease, Leigh's disease, Pelizaeus-Merzbacher disease, Olivopontocerebellar atrophy, Friedreich's ataxia, leukodystrophies, Rett syndrome, Ramsay Hunt syndrome type II, and Down's syndrome (either directly through their known cellular roles or functions or through the association of mutant forms of these proteins with the disease, or indirectly—through their interactions with other proteins known to be linked to neurological disorders, ailments and diseases, including mild cognitive impairment, depression, schizophrenia, obsessive-compulsive disorder, bipolar disorder, and neurodegenerative diseases and disorders and motor neuron diseases and disorders such as Alzheimer's disease, Parkinson's disease, dementia with Lewy bodies, amyotrophic lateral sclerosis or Lou Gehrig's disease, Alpers' disease, Leigh's disease, Pelizaeus-Merzbacher disease, Olivopontocerebellar atrophy, Friedreich's ataxia, leukodystrophies, Rett syndrome, Ramsay Hunt syndrome type II, and Down's syndrome), disruption or dissociation of particular protein-protein interactions may be desirable to ameliorate the disease condition, or to alleviate disease symptoms. Alternatively, if the disease or disorder is associated with increased expression of any of the proteins presented in the tables, or with expression of a mutant form, or forms, of these proteins, then the disease or disorder may be ameliorated, or symptoms reduced, by weakening or dissociating the interaction between the interacting proteins in patients. Also, if a disease or disorder is associated with a mutant form of an interacting protein that form stronger protein-protein interactions with its protein partner than its wild type counterpart, then the disease or disorder may be treated with a compound that weakens, disrupts or interferes with the interaction between the mutant protein and its interacting partner.

In a screening assay for an interaction antagonist, a first protein, which is a protein selected from any of the protein pairs described in the tables (or a homologue, fragment or derivative thereof), or a mutant form of the first protein (or a homologue, fragment or derivative thereof), and a second protein, which is the interacting partner of the first protein identified in the tables above (or a homologue, fragment or derivative thereof), or a mutant form of the second protein (or a homologue, fragment or derivative thereof), are used as test proteins expressed in the form of fusion proteins as described above for purposes of a two-hybrid assay. The fusion proteins are expressed in a host cell and allowed to interact with each other in the presence of one or more test compounds.

In a preferred embodiment, a counterselectable marker is used as a reporter such that a detectable signal (e.g., appearance of color or fluorescence, or cell survival) is present only when the test compound is capable of interfering with the interaction between the two test proteins. In this respect, the reporters used in various “reverse two-hybrid systems” known in the art may be employed. Reverse two-hybrid systems are disclosed in, e.g., U.S. Pat. Nos. 5,525,490; 5,733,726; 5,885,779; Vidal et al., Proc Natl Acad Sci USA, 93:10315-10320 (1996); and Vidal et al., Proc Natl Acad Sci USA, 93:10321-10326 (1996), all of which are incorporated herein by reference.

Examples of suitable counterselectable reporters useful in a yeast system include the URA3 gene (encoding orotidine-5′-decarboxylase, which converts 5-fluroorotic acid (5-FOA) to the toxic metabolite 5-fluorouracil), the CAN1 gene (encoding arginine permease, which transports the toxic arginine analog canavanine into yeast cells), the GAL1 gene (encoding galactokinase, which catalyzes the conversion of 2-deoxygalactose to toxic 2-deoxygalactose-1-phosphate), the LYS2 gene (encoding a-aminoadipate reductase, which renders yeast cells unable to grow on a medium containing a-aminoadipate as the sole nitrogen source), the MET15 gene (encoding O-acetylhomoserine sulfhydrylase, which confers on yeast cells sensitivity to methyl mercury), and the CYH2 gene (encoding L29 ribosomal protein, which confers sensitivity to cycloheximide). In addition, any known cytotoxic agents including cytotoxic proteins such as the diphtheria toxin (DTA) catalytic domain can also be used as counterselectable reporters. See U.S. Pat. No. 5,733,726. DTA causes the ADP-ribosylation of elongation factor-2 and thus inhibits protein synthesis and causes cell death. Other examples of cytotoxic agents include ricin, Shiga toxin, and exotoxin A of Pseudomonas aeruginosa.

For example, when the URA3 gene is used as a counterselectable reporter gene, yeast cells containing a mutant URA3 gene can be used as host cells (Ura-FoaR phenotype) for the in vivo assay. Such cells lack URA3-encoded functional orotidine-5′-phosphate decarboxylase, an enzyme required for the biosynthesis of uracil. As a result, the cells are unable to grow on media lacking uracil. However, because of the absence of a wild-type orotidine-5′-phosphate decarboxylase, the yeast cells cannot convert non-toxic 5-fluoroorotic acid (5-FOA) to a toxic product, 5-fluorouracil. Thus, such yeast cells are resistant to 5-FOA and can grow on a medium containing 5-FOA. Therefore, for example, to screen for a compound capable of disrupting interactions between δ-catenin (or a homologue, fragment or derivative thereof), or a mutant form of δ-catenin (or a homologue, fragment or derivative thereof), and FAK2 (or a homologue, fragment or derivative thereof), or a mutant form of FAK2 (or a homologue, fragment or derivative thereof), δ-catenin (or a homologue, fragment or derivative thereof) is expressed as a fusion protein with a DNA-binding domain of a suitable transcription activator while FAK2 (or a homologue, fragment or derivative thereof) is expressed as a fusion protein with a transcription activation domain of a suitable transcription activator. In the host strain, the reporter URA3 gene may be operably linked to a promoter specifically responsive to the association of the transcription activation domain and the DNA-binding domain. After the fusion proteins are expressed in the Ura⁻ FoaR yeast cells, an in vivo screening assay can be conducted in the presence of a test compound with the yeast cells being cultured on a medium containing uracil and 5-FOA. If the test compound does not disrupt the interaction between δ-catenin and FAK2, active URA3 gene product, i.e., orotidine-5′-decarboxylase, which converts 5-FOA to toxic 5-fluorouracil, is expressed. As a result, the yeast cells cannot grow. On the other hand, when the test compound disrupts the interaction between δ-catenin and FAK2, no active orotidine-5′-decarboxylase is produced in the host yeast cells. Consequently, the yeast cells will survive and grow on the 5-FOA-containing medium. Therefore, compounds capable of interfering with or dissociating the interaction between δ-catenin and FAK2 can thus be identified based on colony formation.

As will be apparent, the screening assay of the present invention can be applied in a format appropriate for large-scale screening. For example, combinatorial technologies can be employed to construct combinatorial libraries of small organic molecules or small peptides. See generally, e.g., Kenan et al., Trends Biochem Sci, 19:57-64 (1994); Gallop et al., J Med Chem, 37:1233-1251 (1994); Gordon et al., J Med Chem, 37:1385-1401 (1994); Ecker et al., Biotechnology, 13:351-360 (1995). Such combinatorial libraries of compounds can be applied to the screening assay of the present invention to isolate specific modulators of particular protein-protein interactions. In the case of random peptide libraries, the random peptides can be co-expressed with the fusion proteins of the present invention in host cells and assayed in vivo. See e.g., Yang et al., Nucl Acids Res, 23:1152-1156 (1995). Alternatively, they can be added to the culture medium for uptake by the host cells.

Conveniently, yeast mating is used in an in vivo screening assay. For example, haploid cells of α-mating type expressing one fusion protein as described above are mated with haploid cells of α-mating type expressing the other fusion protein. Upon mating, the diploid cells are spread on a suitable medium to form a lawn. Drops of test compounds can be deposited onto different areas of the lawn. After culturing the lawn for an appropriate period of time, drops containing a compound capable of modulating the interaction between the particular test proteins in the fusion proteins can be identified by stimulation or inhibition of growth in the vicinity of the drops.

The screening assays of the present invention for identifying compounds capable of modulating protein-protein interactions can also be fine-tuned by various techniques to adjust the thresholds or sensitivity of the positive and negative selections. Mutations can be introduced into the reporter proteins to adjust their activities. The uptake of test compounds by the host cells can also be adjusted. For example, yeast high uptake mutants such as the erg6 mutant strains can facilitate yeast uptake of the test compounds. See Gaber et al., Mol Cell Biol, 9:3447-3456 (1989). Likewise, the uptake of the selection compounds such as 5-FOA, 2-deoxygalactose, cycloheximide, α-aminoadipate, and the like can also be fine-tuned.

Generally, a control assay is performed in which the above screening assay is conducted in the absence of the test compound. The result of this assay is then compared with that obtained in the presence of the test compound.

### 5.3.1.4. Screening Assays for Interaction Agonists

The screening assays of the present invention can also be used to identify compounds that trigger or initiate, enhance or stabilize the protein-protein interactions formed between members of the interacting protein pairs disclosed in the tables above, or between combinations of mutant and wild type forms of such proteins, or pairs of mutant proteins. For example, if a disease or disorder is associated with the decreased expression of any one of the individual proteins, or one of the protein pairs selected from the tables, then the disease or disorder may be treated by strengthening or stabilizing the interactions between the interacting partner proteins in patients. Alternatively, if a disease or disorder is associated with a mutant form, or forms, of the interacting proteins that exhibit weakened or abolished interactions with their binding partner(s), then the disease or disorder may be treated with a compound that initiates or stabilizes the interaction between the mutant form, or forms, of the interacting proteins.

Thus, a screening assay can be performed in the same manner as described above, except that a positively selectable marker is used. For example, a first protein, which is any protein selected from the proteins described in the tables (or a homologue, fragment, or derivative thereof), or a mutant form of the first protein (or a homologue, fragment, or derivative thereof), and a second protein, which is an interacting partner of the first protein (or a homologue, fragment, or derivative thereof), or a mutant form of the second protein (or a homologue, fragment, or derivative thereof), are used as test proteins expressed in the form of fusion proteins as described above for purposes of a two-hybrid assay. The fusion proteins are expressed in host cells and are allowed to interact with each other in the presence of one or more test compounds.

A gene encoding a positively selectable marker such as β-galatosidase may be used as a reporter gene such that when a test compound enables, enhances or strengthens the interaction between a first protein, (or a homologue, fragment, or derivative thereof), or a mutant form of the first protein (or a homologue, fragment, or derivative thereof), and a second protein (or a homologue, fragment, or derivative thereof), or a mutant form of the second (or a homologue, fragment, or derivative thereof), β-galatosidase is expressed. As a result, the compound may be identified based on the appearance of a blue color when the host cells are cultured in a medium containing X-Gal.

Generally, a control assay is performed in which the above screening assay is conducted in the absence of the test compound. The result of this assay is then compared with that obtained in the presence of the test compound.

### 5.4. Optimization of the Identified Compounds

Once test compounds are selected that are capable of modulating the interaction between the interacting protein pairs of proteins described in the tables, or modulating the activity or intracellular levels of their constituent proteins, a secondary assay can be performed to confirm the specificity and effect of the compounds selected in the primary screens. These secondary assays can be designed to test whether a selected compound has a particularly desired effect at the cellular level, or in an animal model of a particular disease or disorder. Exemplary secondary assays include both cell-based assays and animal-based assays.

For example, in the case of compounds selected for further investigation as potential therapeutic agents for the treatment of Alzheimer's disease, or its symptoms, compounds can be tested for their ability to reduce Aβ42 production or secretion in cell-based systems and animal models of Alzheimer's disease, such as those described in the Examples section below. The same compounds can also be tested in neuroprotection assays, such as the assay presented in Example 10, below. Alternatively, compounds selected for further investigation as potential therapeutic agents for the treatment of Alzheimer's disease, or its symptoms, compounds can be tested for their ability to reduce cognitive decline in a mouse model of Alzheimer's disease, as described in Example 11.

In addition, once test compounds are selected that are capable of modulating the proteins in the tables or the interaction between the interacting pairs of proteins described in the tables, or modulating the activity or intracellular levels of their constituent proteins, or reducing Aβ42 production or secretion, or promoting neuroprotection, or reducing cognitive decline, a data set including data defining the identity or characteristics of the test compounds can be generated. The data set may include information relating to the properties of a selected test compound, e.g., chemical structure, chirality, molecular weight, melting point, etc. Alternatively, the data set may simply include assigned identification numbers understood by the researchers conducting the screening assay and/or researchers receiving the data set as representing specific test compounds. The data or information can be cast in a transmittable form that can be communicated or transmitted to other researchers, particularly researchers in a different country. Such a transmittable form can vary and can be tangible or intangible. For example, the data set defining one or more selected test compounds can be embodied in texts, tables, diagrams, molecular structures, photographs, charts, images or any other visual forms. The data or information can be recorded on a tangible media such as paper or embodied in computer-readable forms (e.g., electronic, electromagnetic, optical or other signals). The data in a computer-readable form can be stored in a computer usable storage medium (e.g., floppy disks, magnetic tapes, optical disks, and the like) or transmitted directly through a communication infrastructure. In particular, the data embodied in electronic signals can be transmitted in the form of email or posted on a website on the Internet or Intranet. In addition, the information or data on a selected test compound can also be recorded in an audio form and transmitted through any suitable media, e.g., analog or digital cable lines, fiber optic cables, etc., via telephone, facsimile, wireless mobile phone, internet phone and the like.

Thus, the information and data on a test compound selected in a screening assay described above, or by virtual screening as discussed below, can be produced anywhere in the world and transmitted to a different location. For example, when a screening assay is conducted offshore, the information and data on a selected test compound can be generated and cast in a transmittable form as described above. The data and information in a transmittable form thus can be imported into the U.S. or transmitted to any other country, where the data and information may be used in further testing of the selected test compound and/or in modifying and optimizing the selected test compound to develop lead compounds for testing in clinical trials.

Compounds can also be selected based on structural models of the target protein or protein complex and/or test compounds. In addition, once an effective compound is identified, structural analogs or mimetics thereof can be produced based on rational drug design with the aim of improving drug efficacy and stability, and reducing side effects. Methods known in the art for rational drug design can be used in the present invention. See, e.g., Hodgson et al., Bio/Technology, 9:19-21 (1991); U.S. Pat. Nos. 5,800,998 and 5,891,628, all of which are incorporated herein by reference. An example of rational drug design is the development of HIV protease inhibitors. See Erickson et al., Science, 249:527-533 (1990).

In this respect, structural information on the target protein or protein complex is obtained. Preferably, atomic coordinates defining a three-dimensional structure of the target protein or protein complex can be obtained. For example, each of the interacting pairs can be expressed and purified. The purified interacting protein pairs are then allowed to interact with each other in vitro under appropriate conditions. Optionally, the interacting protein complex can be stabilized by crosslinking or other techniques. The interacting complex can be studied using various biophysical techniques including, e.g., X-ray crystallography, NMR, computer modeling, mass spectrometry, and the like. Likewise, structural information can also be obtained from protein complexes formed by interacting proteins and a compound that initiates or stabilizes the interaction of the proteins. Methods for obtaining such atomic coordinates by X-ray crystallography, NMR, and the like are known in the art and the application thereof to the target protein or protein complex of the present invention should be apparent to skilled persons in the art of structural biology. See Smyth & Martin, Mol Pathol, 53:8-14 (2000); Oakley & Wilce, Clin Exp Pharmacol Physiol, 27(3):145-151 (2000); Ferentz & Wagner, Q Rev Biophys, 33:29-65 (2000); Hicks, Curr Med Chem, 8(6):627-650 (2001); and Roberts, Curr Opin Biotechnol, 10:42-47 (1999).

In addition, understanding of the interaction between the proteins of interest in the presence or absence of a modulator can also be derived by mutagenic analysis using a yeast two-hybrid system or other methods for detecting protein-protein interactions. In this respect, various mutations can be introduced into the interacting proteins and the effect of the mutations on protein-protein interaction examined by a suitable method such as the yeast two-hybrid system.

Various mutations including amino acid substitutions, deletions and insertions can be introduced into a protein sequence using conventional recombinant DNA technologies. Generally, it is particularly desirable to decipher the protein binding sites. Thus, it is important that the mutations introduced only affect protein-protein interactions and cause minimal structural disturbances. Mutations are preferably designed based on knowledge of the three-dimensional structure of the interacting proteins. Preferably, mutations are introduced to alter charged amino acids or hydrophobic amino acids exposed on the surface of the proteins, since ionic interactions and hydrophobic interactions are often involved in protein-protein interactions. Alternatively, the “alanine scanning mutagenesis” technique is used. See Wells, et al., Meth Enzymol, 202:301-306 (1991); Bass et al., Proc Natl Acad Sci USA, 88:4498-4502 (1991); Bennet et al., J Biol Chem, 266:5191-5201 (1991); Diamond et al., J Virol, 68:863-876 (1994). Using this technique, charged or hydrophobic amino acid residues of the interacting proteins are replaced by alanine, and the effect on the interaction between the proteins is analyzed using, e.g., the yeast two-hybrid system. For example, the entire protein sequence can be scanned in a window of five amino acids. When two or more charged or hydrophobic amino acids appear in a window, the charged or hydrophobic amino acids are changed to alanine using standard recombinant DNA techniques. The alanine substitution mutant proteins are the used as “test proteins” in the above-described two-hybrid assays to examine the effect of the mutations on protein-protein interaction. Preferably, the mutational analyses are conducted both in the presence and in the absence of an identified modulator compound. In this manner, the domains or residues of the proteins important to protein-protein interaction and/or the interaction between the modulator compound and the interacting proteins can be identified.

Based on the information obtained, structural relationships between the interacting proteins, as well as between the identified modulators and the interacting proteins are elucidated. For example, for the identified modulators (i.e., lead compounds), the three-dimensional structure and chemical moieties critical to their modulating effect on the interacting proteins are revealed. Using this information and various techniques known in the art of molecular modeling (i.e., simulated annealing), medicinal chemists can then design analog compounds that might be more effective modulators of the protein-protein interactions of the present invention. For example, the analog compounds might show more specific or tighter binding to their targets, and thereby might exhibit fewer side effects, or might have more desirable pharmacological characteristics (e.g., greater solubility).

In addition, if the lead compound is a peptide, it can also be analyzed by the alanine scanning technique and/or the two-hybrid assay to determine the domains or residues of the peptide important to its modulating effect on particular protein-protein interactions. The peptide compound can be used as a lead molecule for rational design of small organic molecules or peptide mimetics. See Huber et al., Curr Med Chem, 1:13-34 (1994).

The domains, residues or moieties critical to the modulating effect of the identified compound constitute the active region of the compound known as its “pharmacophore.” Once the pharmacophore has been elucidated, a structural model can be established by a modeling process that may incorporate data from NMR analysis, X-ray diffraction data, alanine scanning, spectroscopic techniques and the like. Various techniques including computational analysis (e.g., molecular modeling and simulated annealing), similarity mapping and the like can all be used in this modeling process. See e.g., Perry et al., in OSAR: Quantitative Structure-Activity Relationships in Drug Design, pp.189-193, Alan R. Liss, Inc., 1989; Rotivinen et al., Acta Pharm Fennica, 97:159-166 (1988); Lewis et al., Proc R Soc Lond, 236:125-140 (1989); McKinaly et al., Annu Rev Pharmacol Toxiciol, 29:111-122 (1989). Commercial molecular modeling systems available from Polygen Corporation, Waltham, Mass., include the CHARMm program, which performs energy minimization and molecular dynamics functions, and QUANTA program, which performs construction, graphic modeling and analysis of molecular structure. Such programs allow interactive construction, modification, and visualization of molecules. Other computer modeling programs are also available from BioDesign, Inc. (Pasadena, Calif.), Hypercube, Inc. (Cambridge, Ontario), and Allelix, Inc. (Mississauga, Ontario, Canada).

A template can be formed based on the established model. Various compounds can then be designed by linking various chemical groups or moieties to the template. Various moieties of the template can also be replaced. In addition, in the case of a peptide lead compound, the peptide or mimetics thereof can be cyclized, e.g., by linking the N-terminus and C-terminus together, to increase its stability. These rationally designed compounds are further tested. In this manner, pharmacologically acceptable and stable compounds with improved efficacy and reduced side effects can be developed. The compounds identified in accordance with the present invention can be incorporated into a pharmaceutical formulation suitable for administration to an individual.

In addition, the structural models or atomic coordinates defining a three-dimensional structure of the target protein or protein complex can also be used in virtual screen to select compounds capable of modulating the target protein or protein complex. Various methods of computer-based virtual screen using atomic coordinates are generally known in the art. For example, U.S. Pat. No. 5,798,247 (which is incorporated herein by reference) discloses a method of identifying a compound (specifically, an interleukin converting enzyme inhibitor) by determining binding interactions between an organic compound and binding sites of a binding cavity within the target protein. The binding sites are defined by atomic coordinates.

The compounds designed or selected based on rational drug design or virtual screen can be tested for their ability to modulate (interfere with or strengthen) the interaction between the interacting partners within the protein complexes of the present invention. In addition, the compounds can also be further tested for their ability to modulate (inhibit or enhance) cellular functions such as Aβ production in, or secretion from, cells, neuroprotection, neuronal cell survival,as well as their effectiveness in treating neurological disorders, ailments and diseases, including mild cognitive impairment, depression, schizophrenia, obsessive-compulsive disorder, bipolar disorder, and neurodegenerative diseases and disorders and motor neuron diseases and disorders such as Alzheimer's disease, Parkinson's disease, dementia with Lewy bodies, amyotrophic lateral sclerosis or Lou Gehrig's disease, Alpers' disease, Leigh's disease, Pelizaeus-Merzbacher disease, Olivopontocerebellar atrophy, Friedreich's ataxia, leukodystrophies, Rett syndrome, Ramsay Hunt syndrome type II, and Down's syndrome.

Following the selection of desirable compounds according to the methods disclosed above, the methods of the present invention further provide for the manufacture of the selected compounds. Compounds found to desirably modulate the interaction between the interacting pairs of proteins of the present invention, or to desirably modulate the activity or intracellular levels of their constituent proteins, can be manufactured for further experimental studies, or for therapeutic use.

### 6. Therapeutic Applications

As described above, the interactions between the interacting pairs of proteins of the present invention suggest that these proteins and/or the protein complexes formed by them may be involved in common biological processes and disease pathways. The protein complexes may mediate the functions of the individual proteins of each interacting protein pair, or of the interacting pairs themselves, in the biological processes or disease pathways. Thus, one may modulate such biological processes or treat diseases by modulating the functions and activities of any of the individual proteins described in the tables, and/or a protein complex comprising some combination of these proteins. As used herein, modulating a protein selected from the tables, or a protein complex comprising some combination of these proteins means altering (enhancing or reducing) the intracellular concentrations or activities of the proteins or protein complexes, e.g., increasing the concentrations of a particular protein described in the tables, or a protein complex comprising some combination of these proteins, enhancing or reducing their biological activities, increasing or decreasing their stability, altering their affinity or specificity to certain other biological molecules, etc. For example, a pair of interacting proteins listed in the tables may be involved in Aβ production or secretion, neuronal apoptosis, neuronal survival or protection, neurotransmission, axonal guidance or nervous system development, signal transduction, calcium homeostasis, mitochondrial function or intermediary metabolism. Thus, assays such as those described in Section 5 may be used in determining the effect of an aberration in a particular protein complex or an interacting member thereof on Aβ production or secretion, neuronal apoptosis, neuronal survival or protection, neurotransmission, axonal guidance or nervous system development, signal transduction, calcium homeostasis, mitochondrial function or intermediary metabolism. In addition, it is also possible to determine, using the same assay methods, the presence or absence of an association between a protein complex of the present invention or an interacting member thereof and a physiological disorder or disease including neurological disorders, ailments and diseases, including mild cognitive impairment, depression, schizophrenia, obsessive-compulsive disorder, bipolar disorder, and neurodegenerative diseases and disorders and motor neuron diseases and disorders such as Alzheimer's disease, Parkinson's disease, dementia with Lewy bodies, amyotrophic lateral sclerosis or Lou Gehrig's disease, Alpers' disease, Leigh's disease, Pelizaeus-Merzbacher disease, Olivopontocerebellar atrophy, Friedreich's ataxia, leukodystrophies, Rett syndrome, Ramsay Hunt syndrome type II, and Down's syndrome, or predisposition to one of these physiological disorders or diseases.

Once such associations are established, the diagnostic methods as described in Section 4 can be used in diagnosing the disease or disorder, or a patient's predisposition to it. In addition, various in vitro and in vivo assays may be employed to test the therapeutic or prophylactic efficacies of the various therapeutic approaches described in Sections 6.2 and 6.3 that are aimed at modulating the functions and activities of a particular protein complex of the present invention, or an interacting member thereof. Similar assays can also be used to test whether the therapeutic approaches described in Sections 6.2 and 6.3 result in the modulation of Aβ production or secretion, neuronal apoptosis, neuronal survival or protection, neurotransmission, axonal guidance or nervous system development, signal transduction, calcium homeostasis, mitochondrial function or intermediary metabolism. The cell model or transgenic animal model described in Section 7 may be employed in the in vitro and in vivo assays.

In accordance with this aspect of the present invention, methods are provided for modulating (promoting or inhibiting) a protein complex of the present invention formed by the interactions described in the tables. The human cells can be in in vitro cell or tissue cultures. The methods are also applicable to human cells in a patient.

In one embodiment, the concentration of a protein complex formed by the interactions described in the tables is reduced in the cells. Various methods can be employed to reduce the concentration of the protein complex. For example, the concentration of the protein complex can be reduced by interfering with the interactions between the interacting protein partners. Hence, compounds capable of interfering with interactions between interacting pairs of proteins identified in the tables can be administered to the cells in vitro or in vivo in a patient. Such compounds can be compounds capable of binding specific proteins listed in the tables. They can also be antibodies immunoreactive with specific proteins identified in the tables. Also, the compounds can be small peptides derived from a first interacting protein of the present invention, or a mimetic thereof, that are capable of binding a second protein of the present invention, the second protein being a binding partner of the first protein as shown in the tables above.

In another embodiment, the method of modulating the protein complex includes inhibiting the expression of any of the individual proteins described in the tables. The inhibition can be at the transcriptional, translational, or post-translational level. For example, antisense compounds and ribozyme compounds can be administered to human cells in cultures or in human bodies. In addition, RNA interference technologies may also be employed to administer to cells double-stranded RNA or RNA hairpins capable of “knocking down” the expression of any of the interacting proteins of the present invention.

In the various embodiments described above, preferably the concentrations or activities of both partners in an interacting pair of proteins of the present invention are reduced or inhibited, or the concentration or activitie of a single constituent protein of a protein complex formed by the interactions described in the tables is reduced or inhibited.

In yet another embodiment, an antibody selectively immunoreactive with a pair of interacting proteins identified in the tables is administered to cells in vitro or in human bodies to inhibit the protein complex activities and/or reduce the concentration of the protein complex in the cells or patient.

Further provided by the present invention is a method of treatment of a disease or disorder comprising identifying a patient that has a particular disease or disorder, shows symptoms of having a particular disease or disorder, is predisposed to, or at risk of developing a particular disease or disorder, and treating the disease or disorder by modulating a protein or protein-protein interaction according to the present invention.

The present invention also provides for the treatment of neurological disorders, ailments and diseases, including mild cognitive impairment, depression, schizophrenia, obsessive-compulsive disorder, bipolar disorder, and neurodegenerative diseases and disorders and motor neuron diseases and disorders such as Alzheimer's disease, Parkinson's disease, dementia with Lewy bodies, amyotrophic lateral sclerosis or Lou Gehrig's disease, Alpers' disease, Leigh's disease, Pelizaeus-Merzbacher disease, Olivopontocerebellar atrophy, Friedreich's ataxia, leukodystrophies, Rett syndrome, Ramsay Hunt syndrome type II, and Down's syndrome through the modulation of the concentration and/or activity of particular proteins interactors of the present invention, including, e.g., BAT3, CIB, FAK2, FKBP25, and SCD.

In highly preferred embodiments of the present invention, the treatment of Alzheimer's disease or its symptoms is achieved through the modulation of the concentration and/or activity of particular proteins interactors of the present invention, including, e.g., BAT3, CIB, FAK2, FKBP25, and SCD. In specific embodiments, the prevention or treatment of Alzheimer's disease or its symptoms is achieved through the reduction in the secretion of Aβ42 in a human patient by the inhibition of the activity, or reduction in expression of BAT3, CIB, FAK2 and/or SCD.

As used herein, the phrase “reducing Aβ42 levels,” with respect to human patients, means reducing the amount of Aβ42 that can be detected in a biological sample, e.g., plasma or cerebrospinal fluid. When used in reference to animal models, however, the phrase “reducing Aβ42 levels” can also mean reducing the amount of Aβ42 that can be detected in the brain, or within specific parts of the brain.

For the purposes of the following discussion the phrase “inhibiting FAK2” means reducing the FAK2 activity, preferably by 20, 30, or 40 percent, more preferably by 50, 60, or 70 percent, and even more preferably 80, 90, 95 percent, or more. In addition, the phrase “selectively inhibiting FAK2” means preferentially reducing FAK2 tyrosine kinase activity over, at least, the activities of other types of kinases; preferably preferentially reducing FAK2 tyrosine kinase activity over the activities of other types of tyrosine kinases; and even more preferably, preferentially reducing FAK2 tyrosine kinase activity over FAK1 and FAK3 tyrosine kinases.

Additionally the word “treatment” when used in the context of diseases and disorders is meant to encompass all aspects of therapeutic benefit, including delaying the onset and slowing the progression of the disease or disorder. The phrase “delaying the onset,” when used in the context of neurological diseases and disorders, such as Alzheimer's disease, means delaying the onset of specific symptoms of Alzheimer's disease by at least 6 months from when they might appear otherwise. For example, as further described in Example 12, one particular symptom that can be used to determine the delay of onset can be a score obtained on the Mini-Mental State Examination (MMSE, Mohs et al., Int Psychogeriatr 8:195-203 (1996)), or a score obtained on the Geriatric Depression Scale (GDS), or some combination thereof.

### 6.1. Applicable Diseases

The methods for modulating the functions and activities of a protein complex of the present invention, or an interacting member thereof, may be employed to modulate Aβ production or secretion, neuronal apoptosis, neuronal survival or protection, neurotransmission, axonal guidance or nervous system development, signal transduction, calcium homeostasis, mitochondrial function or intermediary metabolism. In addition, the methods may also be used in the treatment or prevention of diseases and disorders associated with such cellular functions and activities, particularly neurological disorders, ailments and diseases, including mild cognitive impairment, depression, schizophrenia, obsessive-compulsive disorder, bipolar disorder, and neurodegenerative diseases and disorders and motor neuron diseases and disorders such as Alzheimer's disease, Parkinson's disease, dementia with Lewy bodies, amyotrophic lateral sclerosis or Lou Gehrig's disease, Alpers' disease, Leigh's disease, Pelizaeus-Merzbacher disease, Olivopontocerebellar atrophy, Friedreich's ataxia, leukodystrophies, Rett syndrome, Ramsay Hunt syndrome type II, and Down's syndrome. The methods may also be useful for treating or preventing other diseases such as dislipidemia, diabetes, obesity, cardiovascular diseases such as atherosclerosis, and coronary heart disease.

### 6.2. Inhibiting Protein Complex or Interacting Protein Members Thereof

In one aspect of the present invention, methods are provided for reducing in cells or tissue the concentration and/or activity of a protein complex identified in accordance with the present invention that comprises one or more of the interacting pairs of proteins described in the tables above. In addition, methods are also provided for reducing in cells or tissue the concentration and/or activity of any of the individual proteins identified in the tables. By reducing the concentration of a protein complex and/or one or more of the protein constituents of the protein complex and/or inhibiting the functional activities of the protein complex and/or one or more of the protein constituents of the protein complex, the diseases involving such a protein complex or protein constituents of the protein complex may be treated or prevented. The present invention also provides for the treatment of neurological disorders, ailments and diseases, including mild cognitive impairment, depression, schizophrenia, obsessive-compulsive disorder, bipolar disorder, and neurodegenerative diseases and disorders and motor neuron diseases and disorders such as Alzheimer's disease, Parkinson's disease, dementia with Lewy bodies, amyotrophic lateral sclerosis or Lou Gehrig's disease, Alpers' disease, Leigh's disease, Pelizaeus-Merzbacher disease, Olivopontocerebellar atrophy, Friedreich's ataxia, leukodystrophies, Rett syndrome, Ramsay Hunt syndrome type II, and Down's syndrome through the modulation of the concentration and/or activity of the protein complexes of the present invention, or of particular interacting proteins, including, e.g., BAT3, CIB, FAK2, FKBP25, and SCD. Modulation of the concentration and/or activity of the protein complexes of the present invention, or of particular interacting proteins, including, e.g., BAT3, CIB, FAK2, FKBP25, and SCD, can be achieved by any method known in the art, including antibody therapy, siRNA therapy, antisense therapy, ribozyme therapy, or treatment with peptides or small organic molecules that bind to the protein complexes of the present invention, or the constituent interacting proteins thereof, producing a desired effect, as specifically disclosed in the following Sections.

### 6.2.1. Antibody Therapy

In one embodiment, an antibody may be administered to cells or tissue in vitro or to patients. The antibody administered may be immunoreactive with any of the individual proteins described in the tables, or with one of the protein complexes of the present invention. Suitable antibodies may be monoclonal or polyclonal that fall within any antibody class, e.g., IgG, IgM, IgA, IgE, etc. The antibody suitable for this invention may also take a form of various antibody fragments including, but not limited to, Fab and F(ab′)₂, single-chain fragments (scFv), and the like. In another embodiment, an antibody selectively immunoreactive with the protein complex formed from at least one of the interacting pairs of proteins described in the tables, is administered to cells or tissue in vitro or in to patient. In yet another embodiment, an antibody specific to an individual protein selected from any of the tables is administered to cells or tissue in vitro or in a patient. Methods for making the antibodies of the present invention should be apparent to a person of skill in the art, especially in view of the discussions in Section 3 above. The antibodies can be administered in any suitable form via any suitable route as described in Section 8 below. Preferably, the antibodies are administered in a pharmaceutical composition together with a pharmaceutically acceptable carrier.

Alternatively, the antibodies may be delivered by a gene-therapy approach. That is, nucleic acids encoding the antibodies, particularly single-chain fragments (scFv), may be introduced into cells or tissue in vitro or in a patient such that desirable antibodies may be produced recombinantly in vivo from the nucleic acids. For this purpose, the nucleic acids with appropriate transcriptional and translation regulatory sequences can be directly administered into the patient. Alternatively, the nucleic acids can be incorporated into a suitable vector as described in Sections 2.3 and 5.3.1.1, above, and delivered into cells or tissue in vitro or in a patient along with the vector. The expression vector containing the nucleic acids can be administered directly to cells or tissue in vitro or in a patient. It can also be introduced into cells, preferably cells derived from a patient to be treated, and subsequently delivered into the patient by cell transplantation. See Section 6.3.2 below.

### 6.2.2. siRNA Therapy

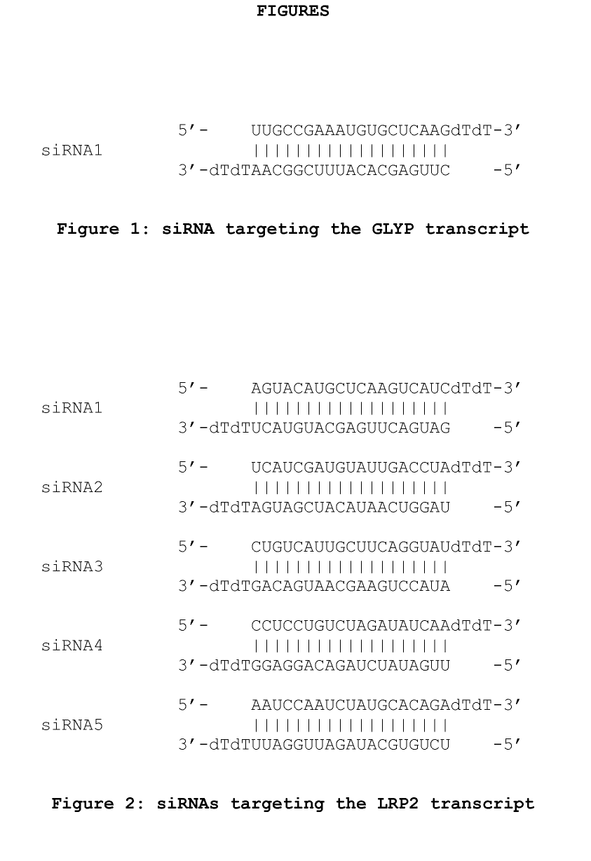

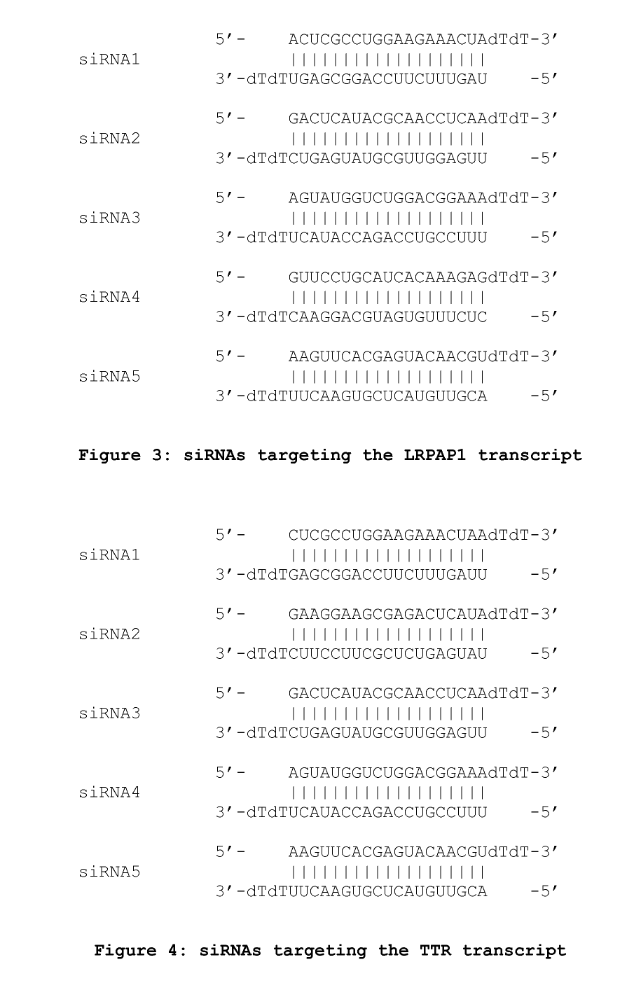

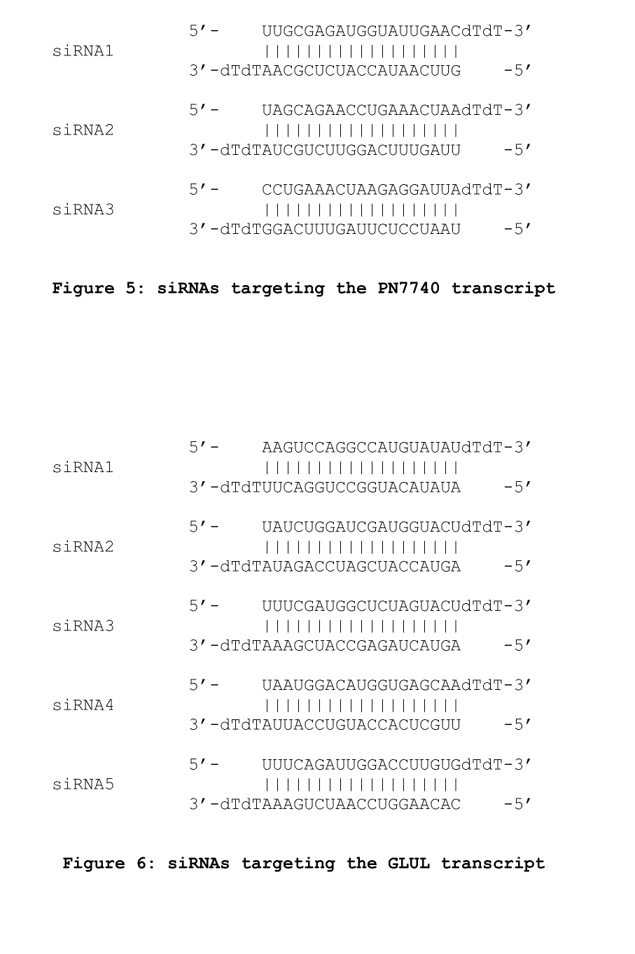

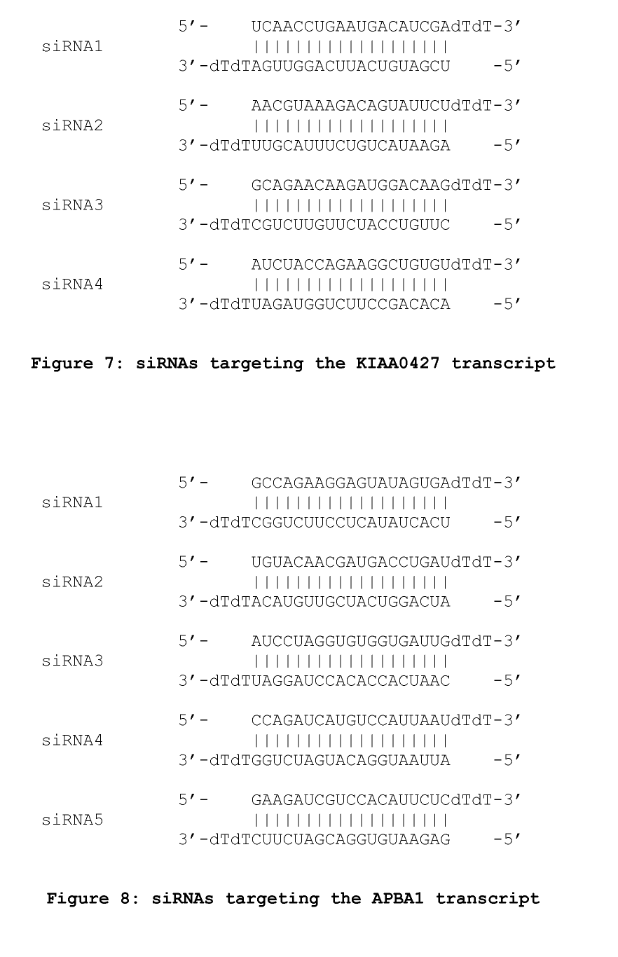

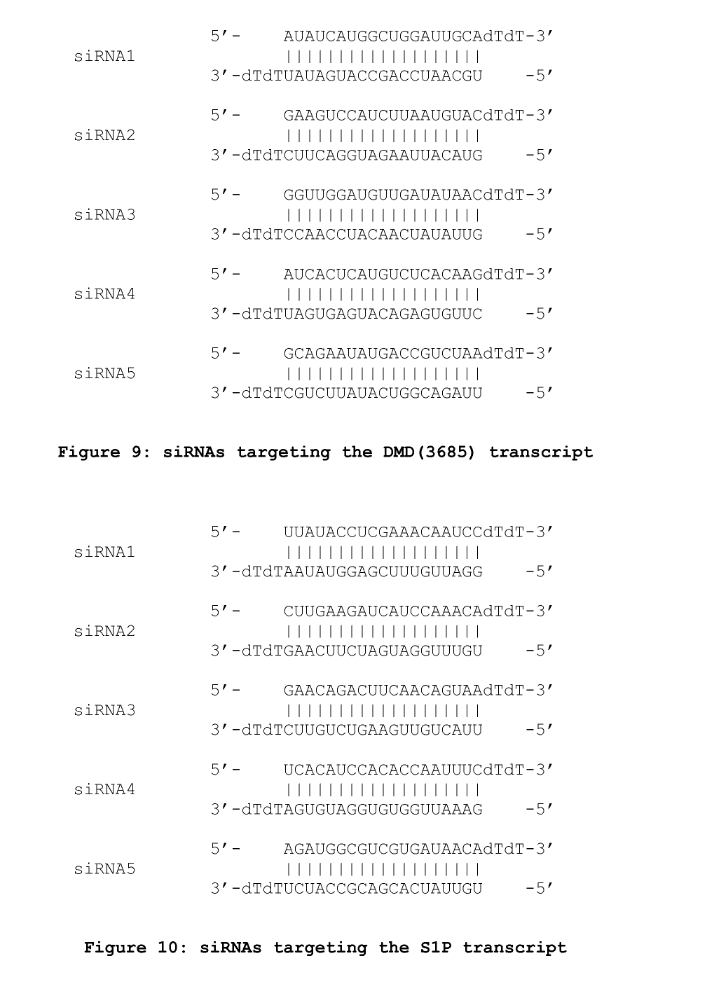

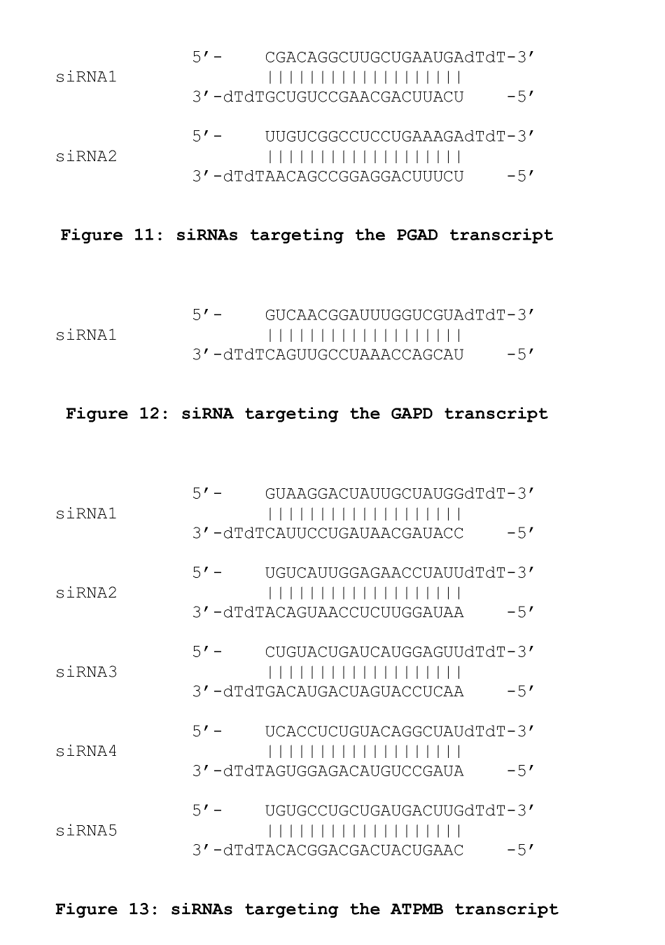

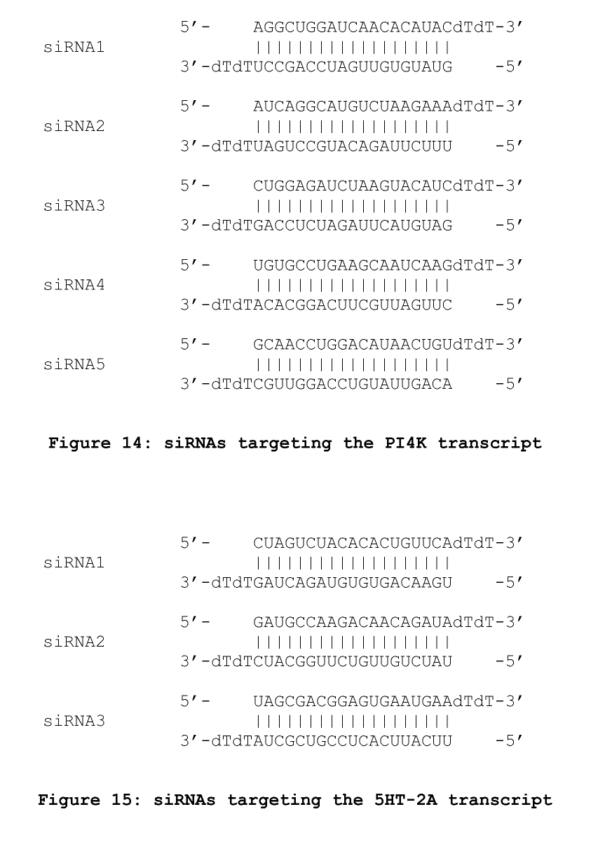

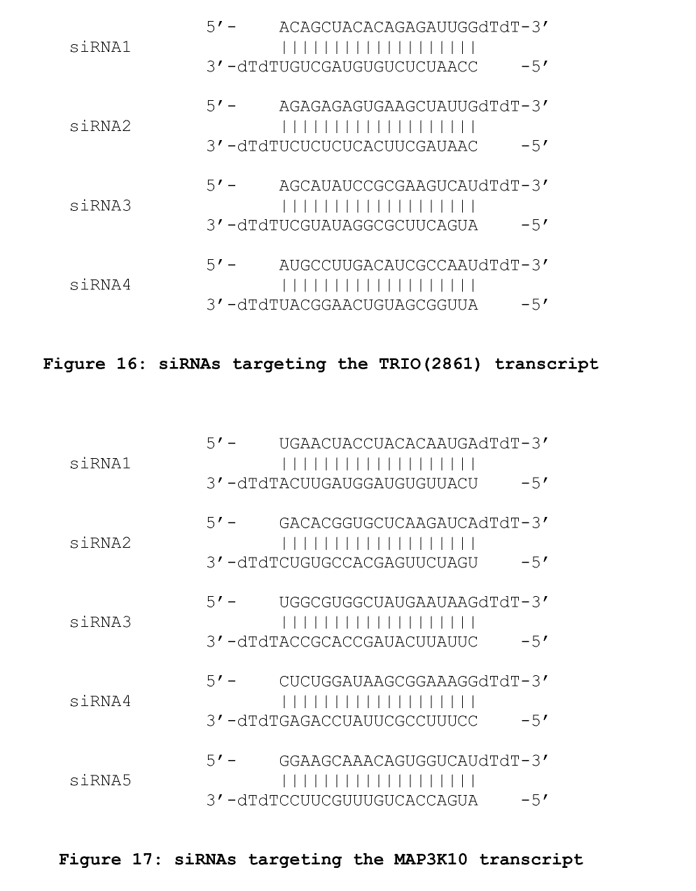

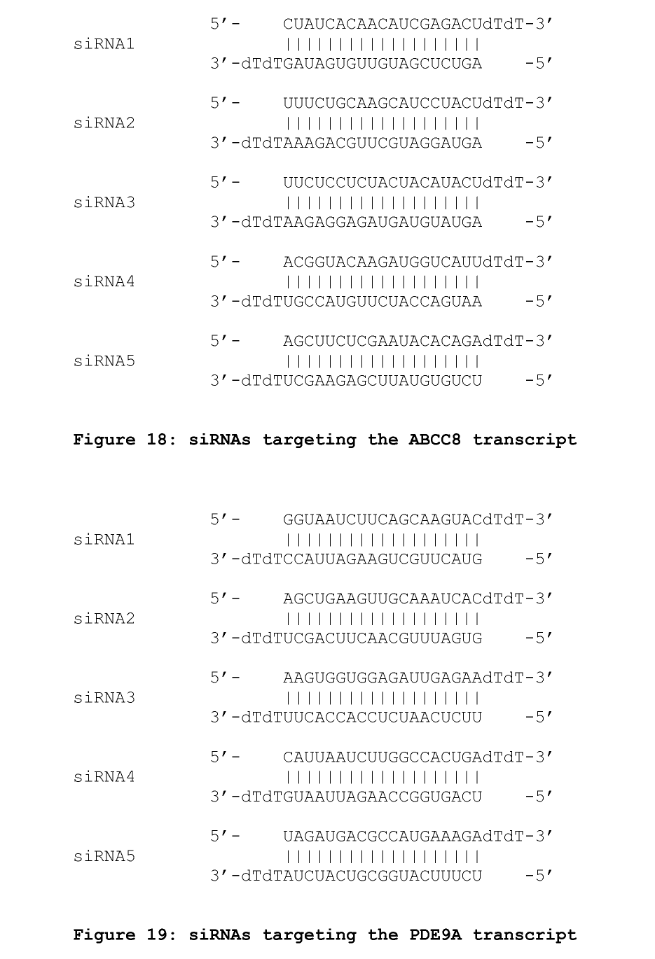

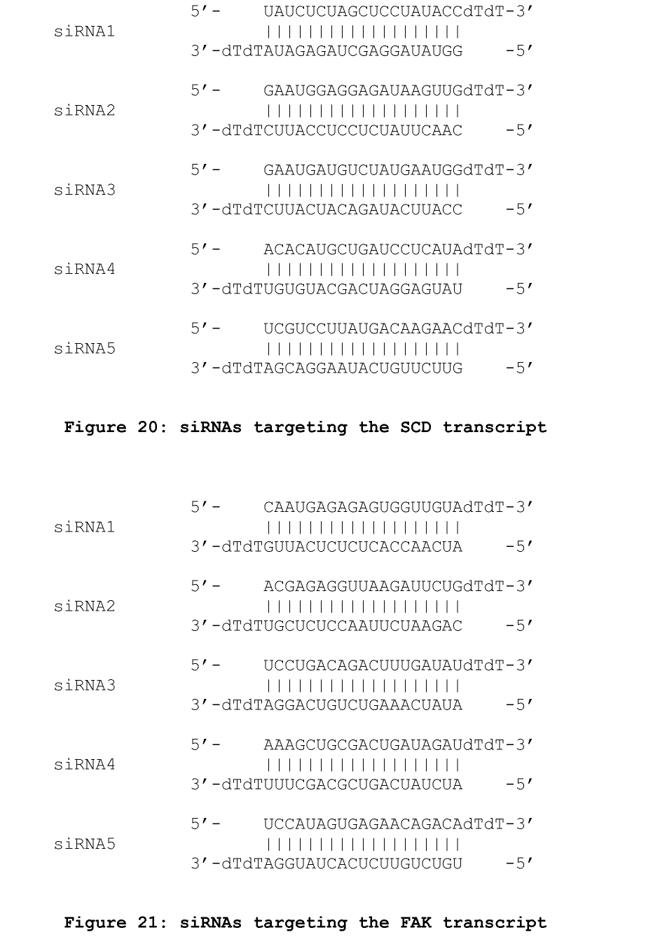

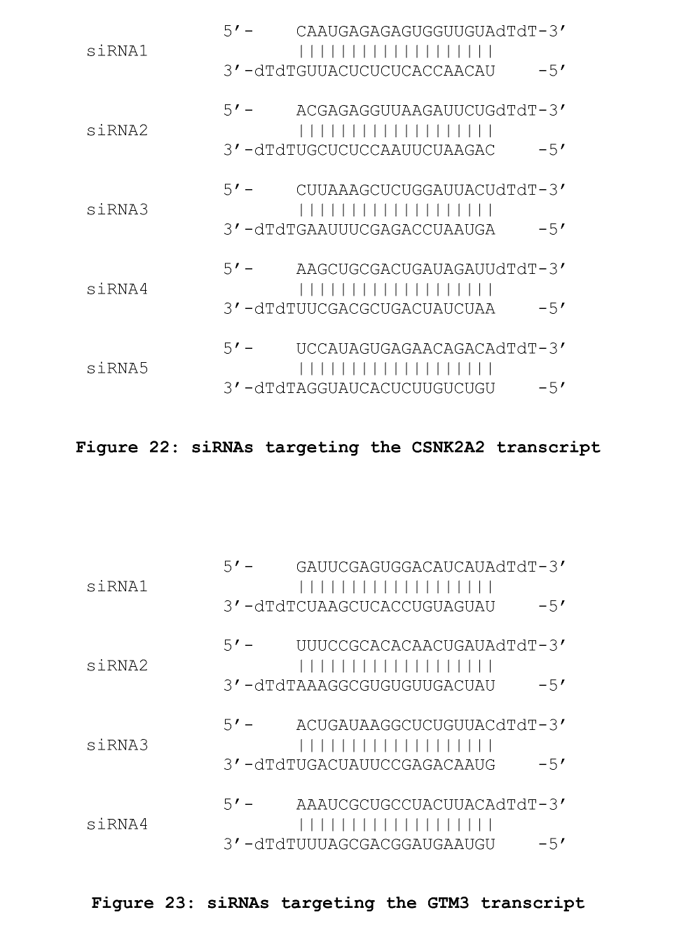

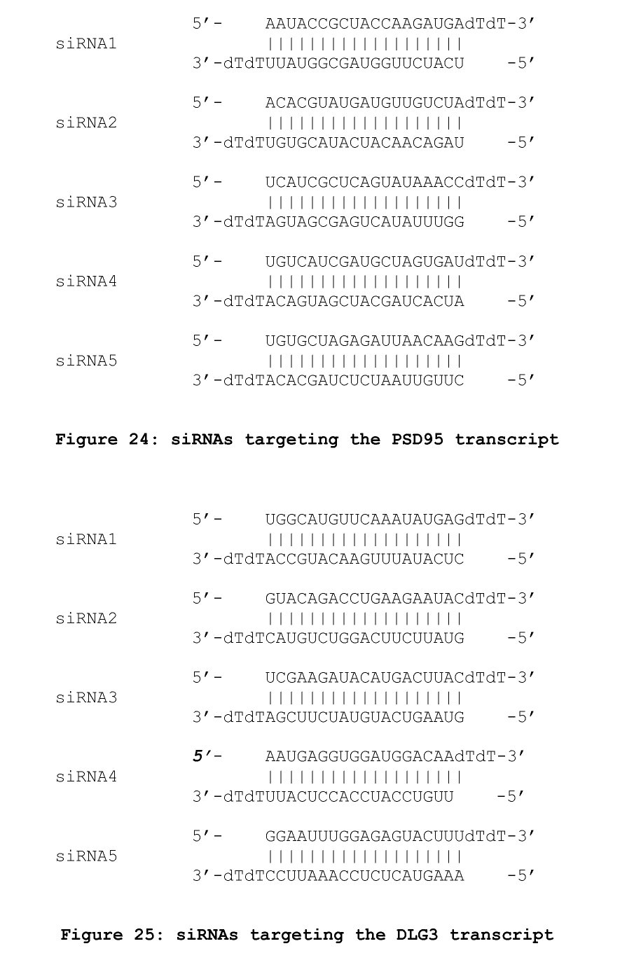

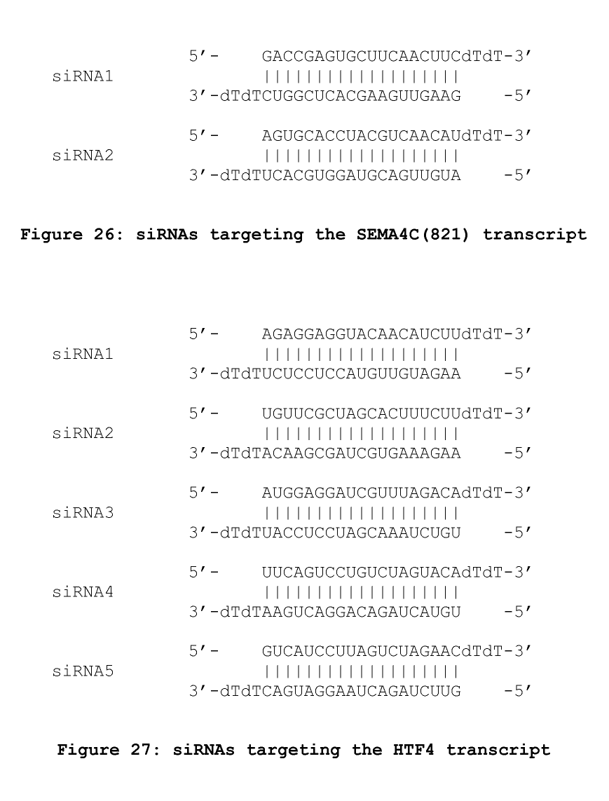

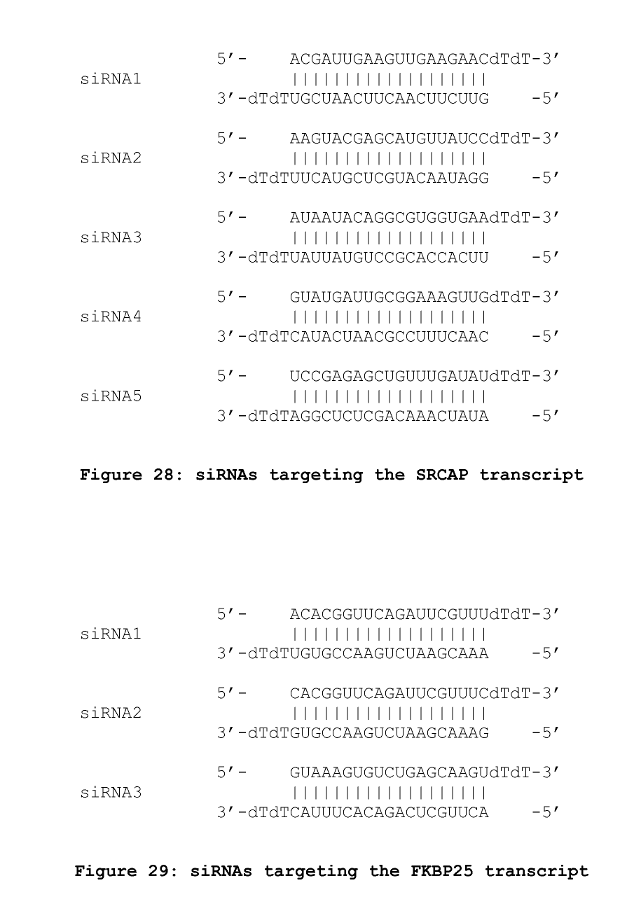

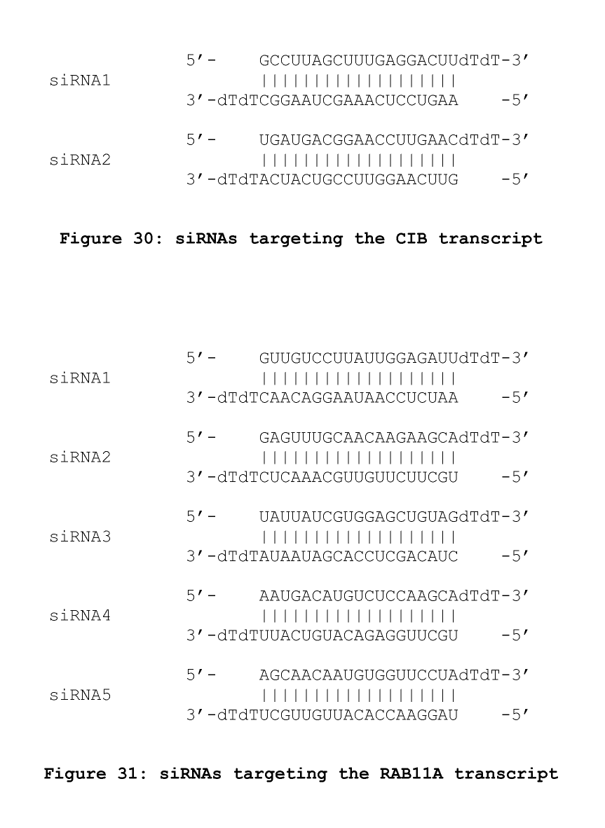

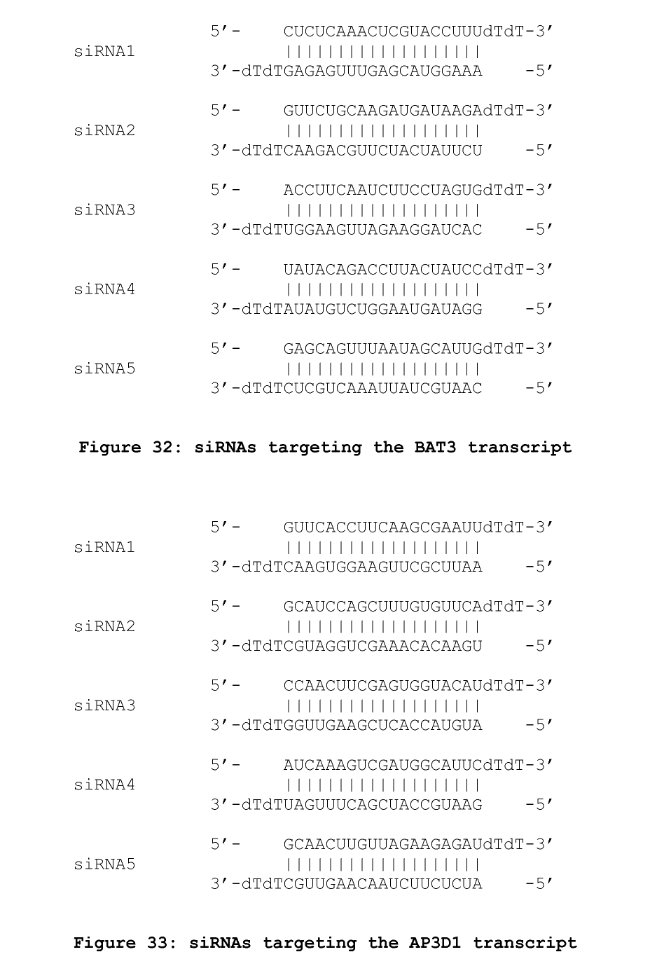

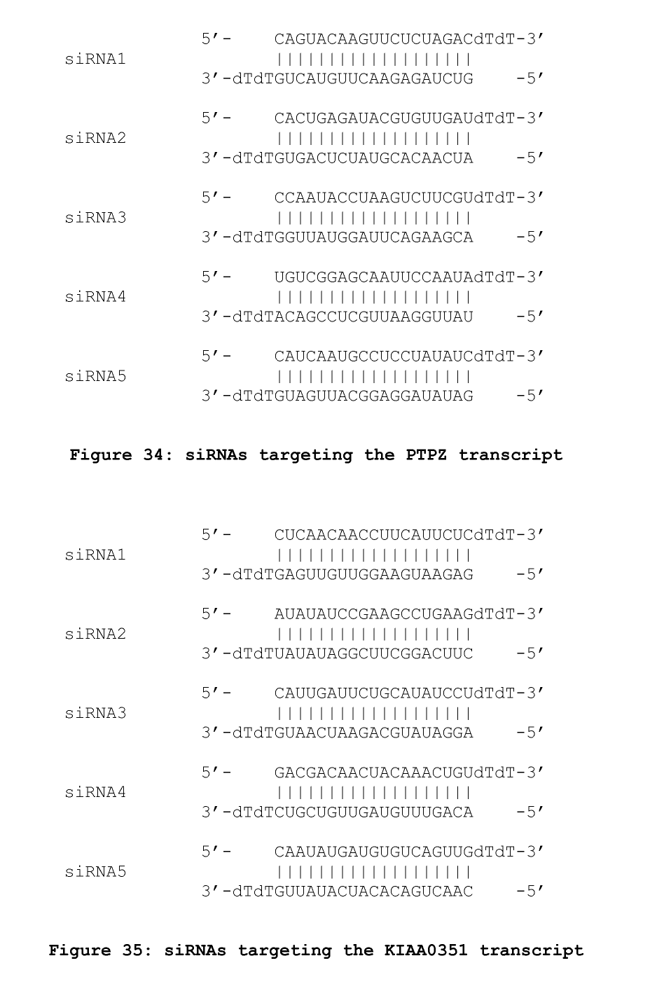

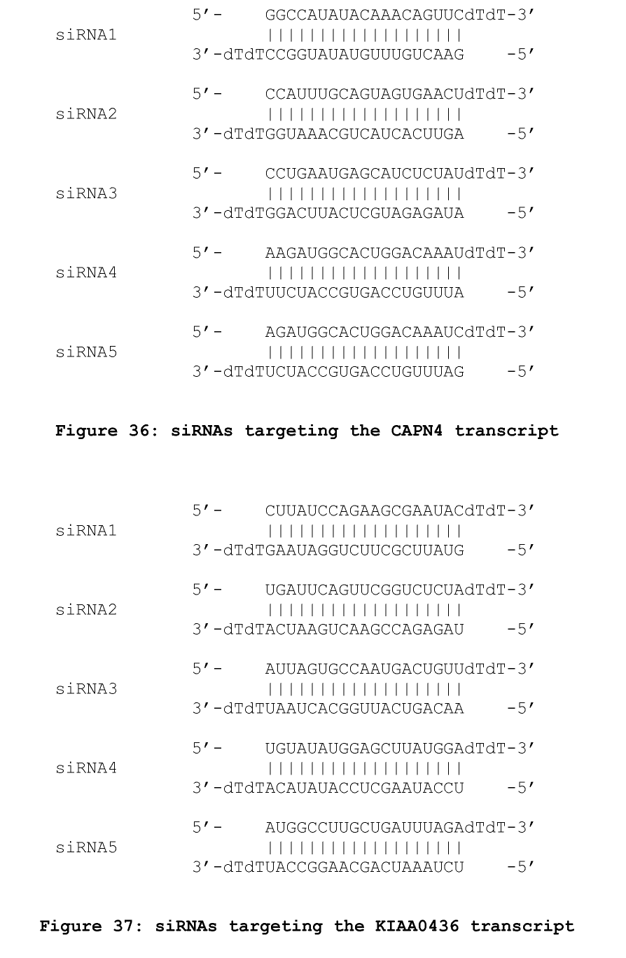

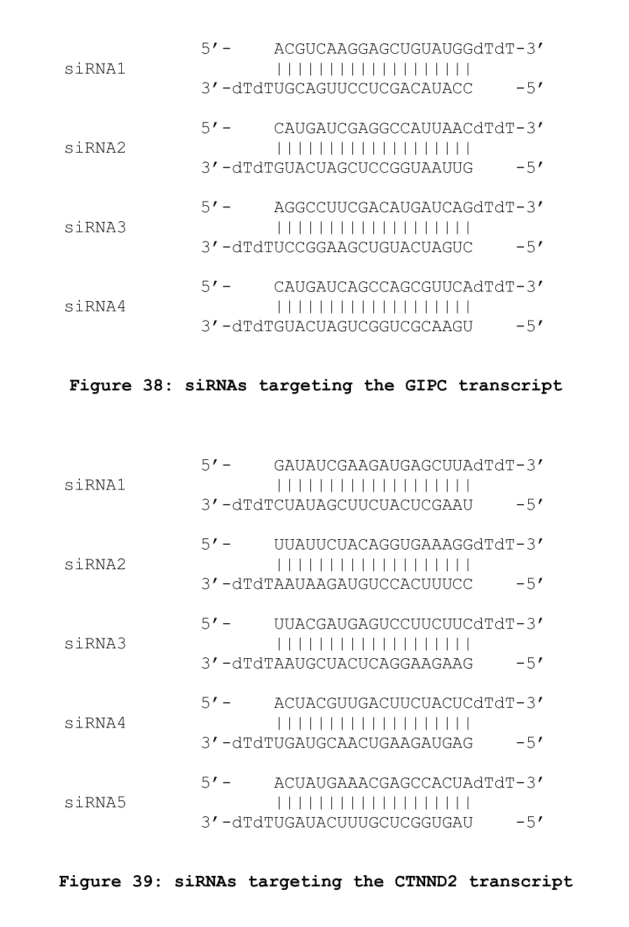

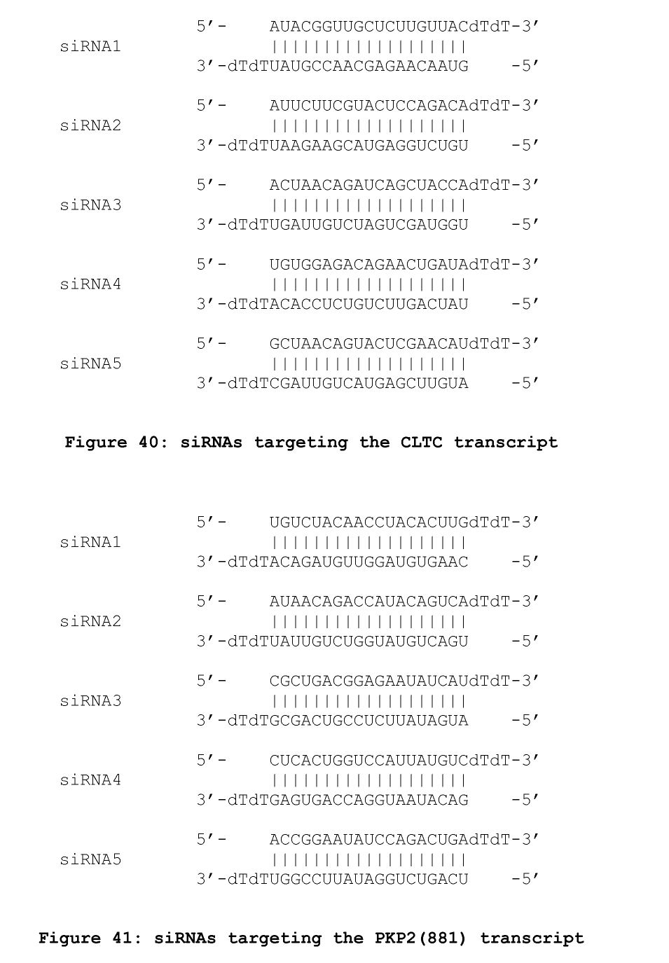

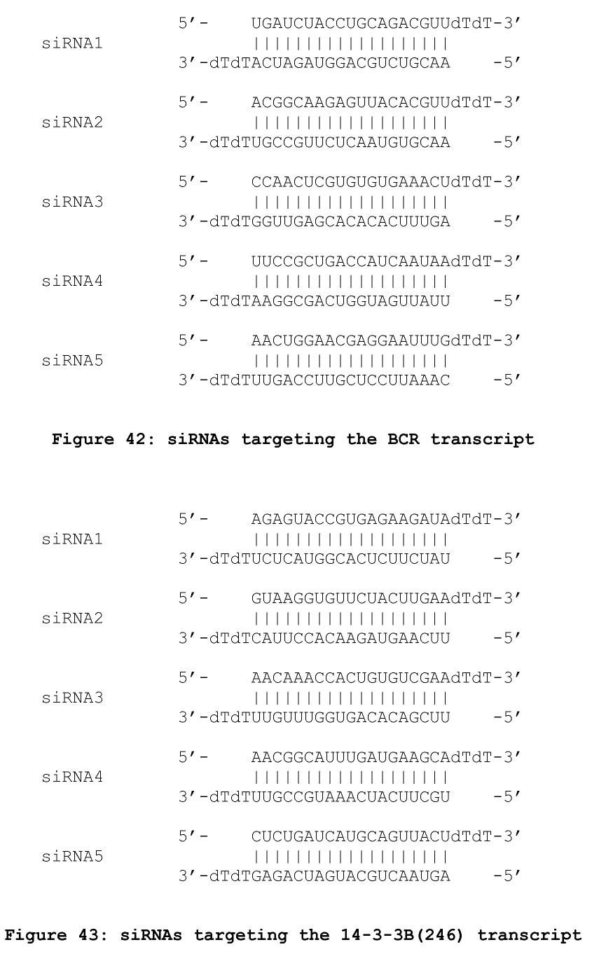

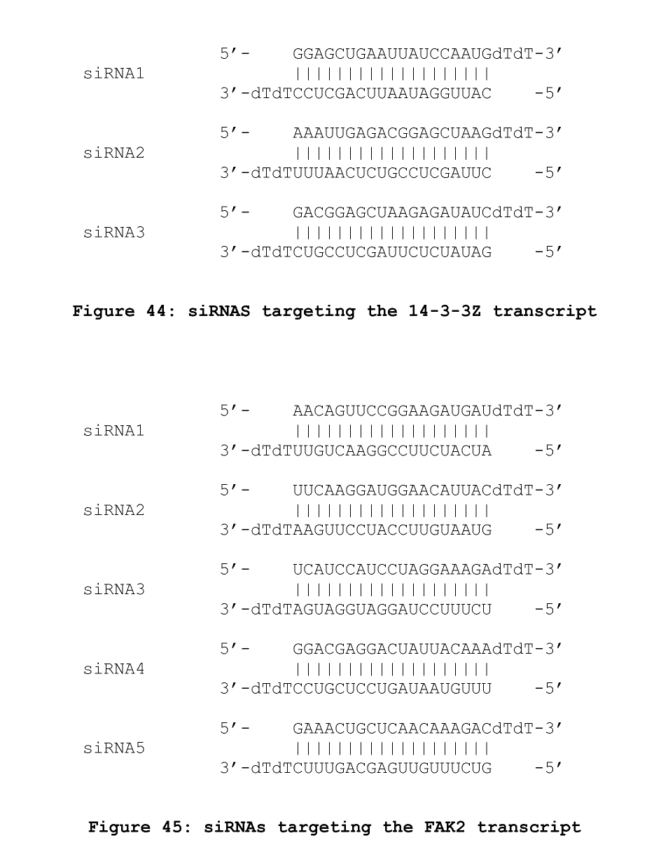

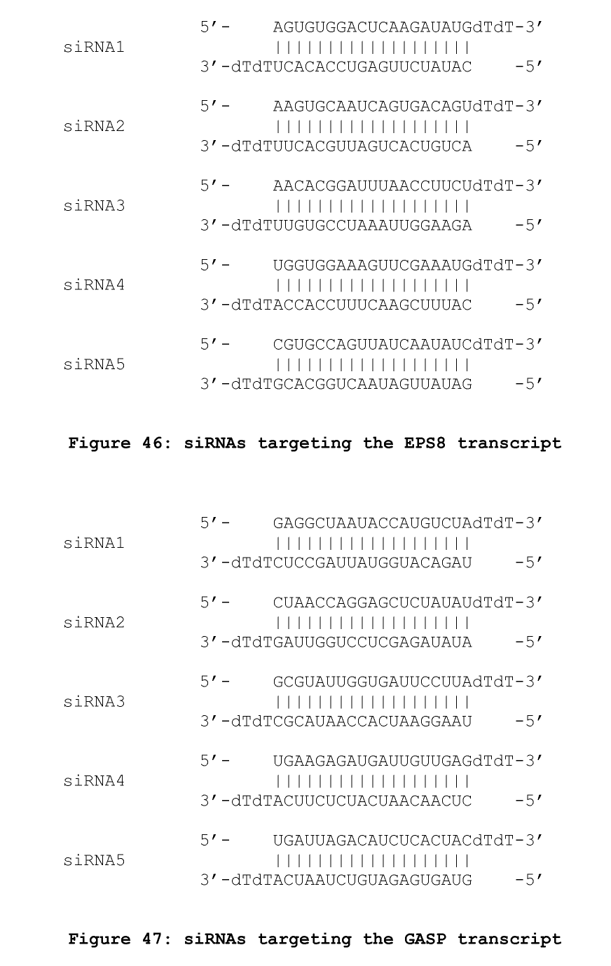

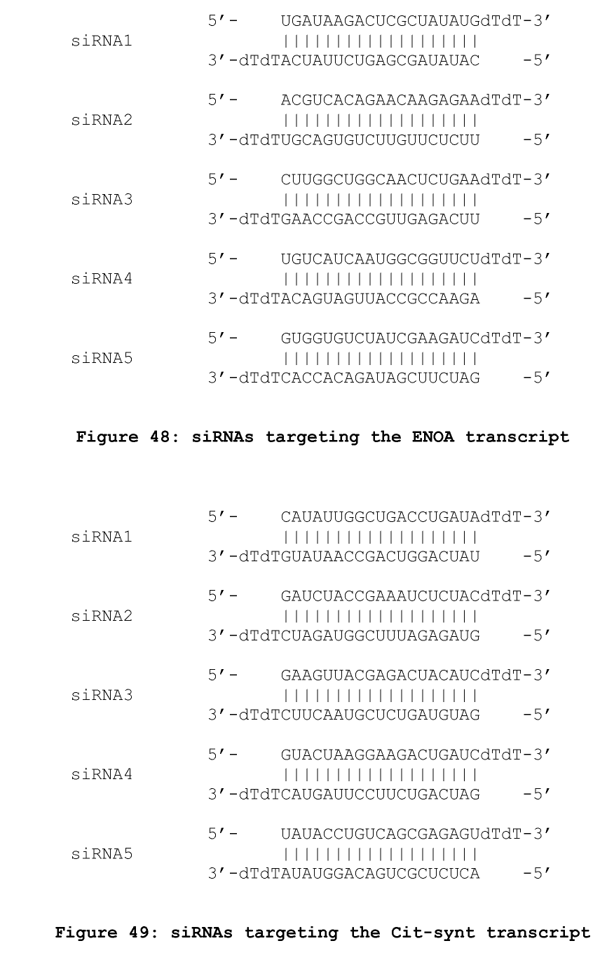

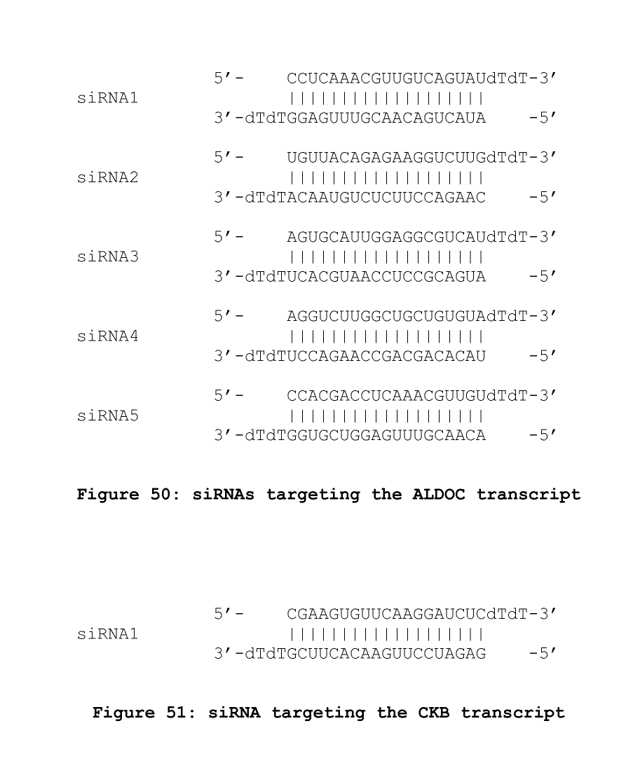

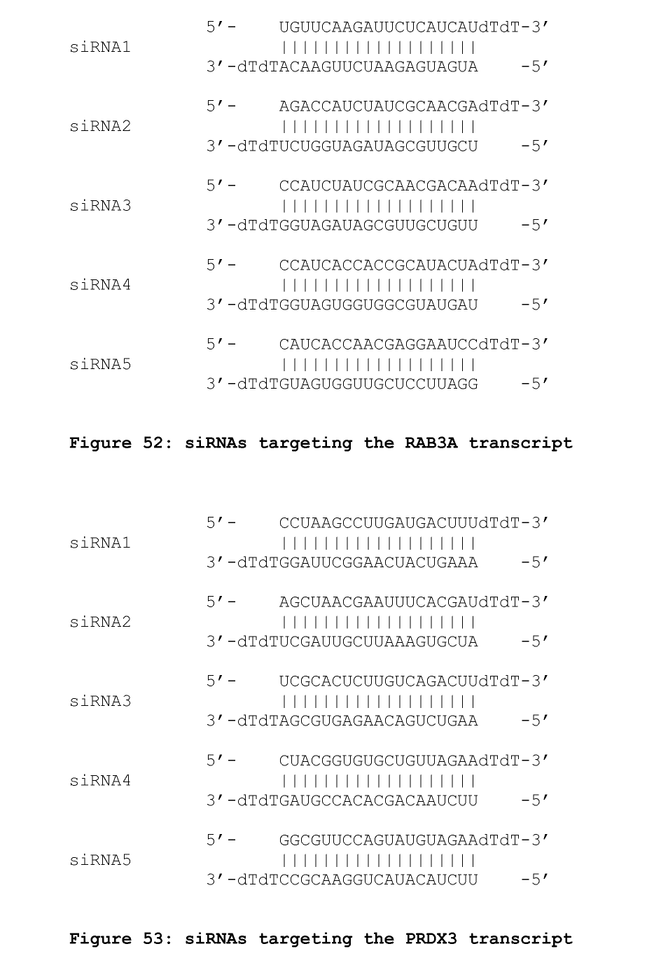

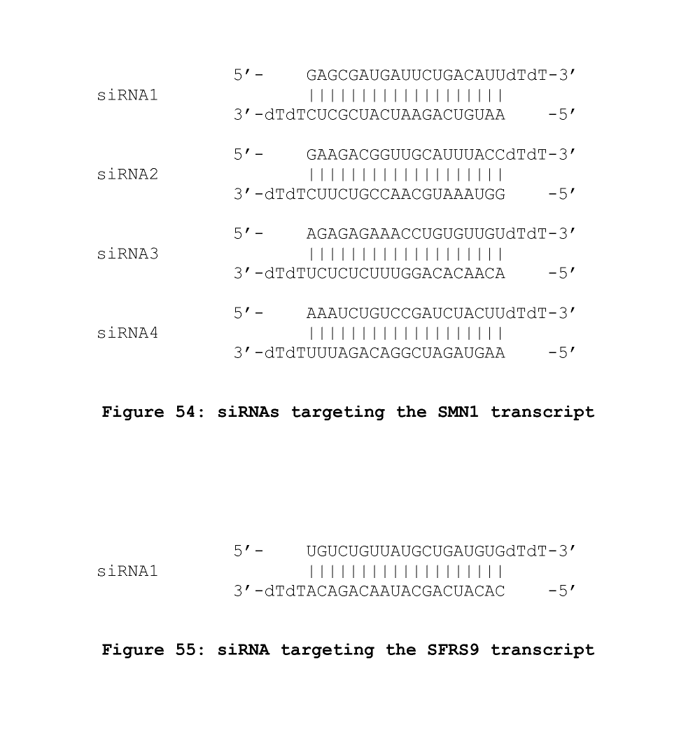

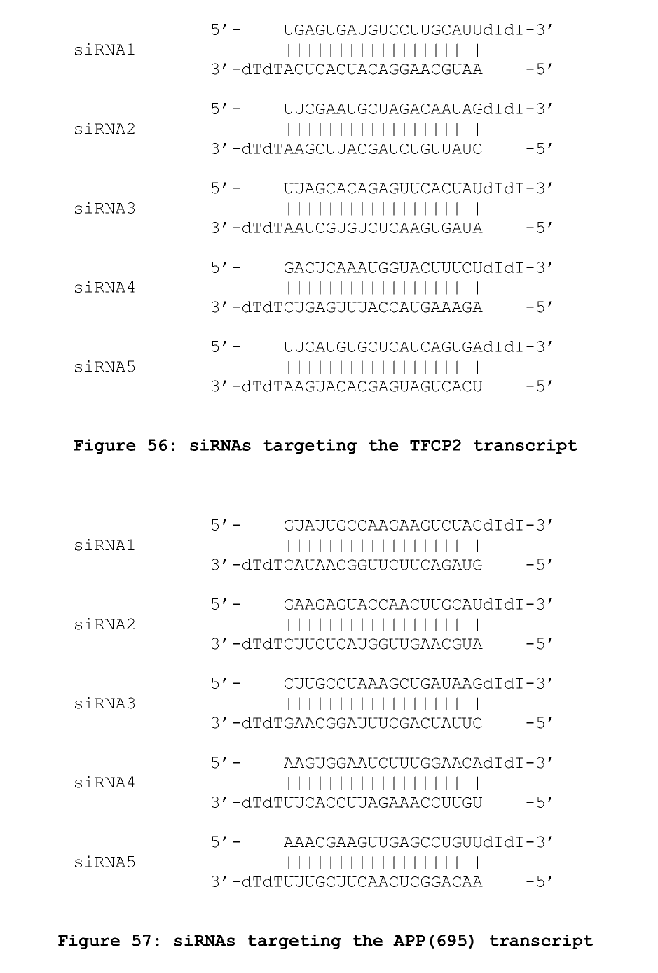

In another embodiment, double-stranded small interfering RNA (siRNA) compounds specific to nucleic acids encoding one or more interacting protein members of a protein complex identified in the present invention are administered to cells or tissue in vitro or in a patient to be therapeutically or prophylactically treated. FIGS. 1-59 depict the structures of siRNA compounds designed to reduce the expression of specific proteins that comprise the protein complexes of the present invention.

As is generally known in the art, siRNA compounds are RNA duplexes comprising two complementary single-stranded RNAs of 21 nucleotides that form 19 base pairs and possess 3′ overhangs of two nucleotides. See Elbashir et al., Nature 411:494-498 (2001); and PCT Publication Nos. WO 00/44895; WO 01/36646; WO 99/32619; WO 00/01846; WO 01/29058; WO 99/07409; and WO 00/44914. When appropriately targeted via its nucleotide sequence to a specific mRNA in cells, an siRNA can specifically suppress gene expression through a process known as RNA interference (RNAi). See e.g., Zamore & Aronin, Nature Medicine, 9:266-267 (2003). siRNAs can reduce the cellular level of specific mRNAs, and decrease the level of proteins coded by such mRNAs. siRNAs utilize sequence complementarity to target an mRNA for destruction, and are sequence-specific. Thus, they can be highly target-specific, and in mammals have been shown to target mRNAs encoded by different alleles of the same gene. Because of this precision, side effects typically associated with traditional drugs can be reduced or eliminated. In addition, they are relatively stable, and like antisense and ribozyme molecules, they can also be modified to achieve improved pharmaceutical characteristics, such as increased stability, deliverability, and ease of manufacture. Moreover, because siRNA molecules take advantage of a natural cellular pathway, i.e., RNA interference, they are highly efficient in destroying targeted mRNA molecules. As a result, it is relatively easy to achieve a therapeutically effective concentration of an siRNA compound in patients. Thus, siRNAs are a promising new class of drugs being actively developed by pharmaceutical companies.

Indeed, in vivo inhibition of specific gene expression by RNAi has been achieved in various organisms including mammals. For example, Song et al., Nature Medicine, 9:347-351 (2003) discloses that intravenous injection of Fas siRNA compounds into laboratory mice with autoimmune hepatitis specifically reduced Fas mRNA levels and expression of Fas protein in mouse liver cells. The gene silencing effect persisted without diminution for 10 days after the intravenous injection. The injected siRNA was effective in protecting the mice from liver failure and fibrosis. Song et al., Nature Medicine, 9:347-351 (2003). Several other approaches for delivery of siRNA into animals have also proved to be successful. See e.g., McCaffery et al., Nature, 418:38-39 (2002); Lewis et al., Nature Genetics, 32:107-108 (2002); and Xia et al., Nature Biotech., 20:1006-1010 (2002).

The siRNA compounds provided according to the present invention can be synthesized using conventional RNA synthesis methods. For example, they can be chemically synthesized using appropriately protected ribonucleoside phosphoramidites and a conventional DNA/RNA synthesizer. Various applicable methods for RNA synthesis are disclosed in, e.g., Usman et al., J Am Chem Soc, 109:7845-7854 (1987) and Scaringe et al., Nucleic Acids Res, 18:5433-5441 (1990). Custom siRNA synthesis services are available from commercial vendors such as Ambion (Austin, Tex., USA), Dharmacon Research (Lafayette, Colo., USA), Pierce Chemical (Rockford, Ill., USA), ChemGenes (Ashland, Mass., USA), Proligo (Hamburg, Germany), and Cruachem (Glasgow, UK).

The siRNA compounds can also be various modified equivalents of the siRNA structures. As used herein, “modified equivalent” means a modified form of a particular siRNA compound having the same target-specificity (i.e., recognizing the same mRNA molecules that complement the unmodified particular siRNA compound). Thus, a modified equivalent of an unmodified siRNA compound can have modified ribonucleotides, that is, ribonucleotides that contain a modification in the chemical structure of an unmodified nucleotide base, sugar and/or phosphate (or phospodiester linkage). As is known in the art, an “unmodified ribonucleotide” has one of the bases adenine, cytosine, guanine, and uracil joined to the 1′ carbon of beta-D-ribo-furanose.

Preferably, modified siRNA compounds contain modified backbones or non-natural internucleoside linkages, e.g., modified phosphorous-containing backbones and non-phosphorous backbones such as morpholino backbones; siloxane, sulfide, sulfoxide, sulfone, sulfonate, sulfonamide, and sulfamate backbones; formacetyl and thioformacetyl backbones; alkene-containing backbones; methyleneimino and methylenehydrazino backbones; amide backbones, and the like.

Examples of modified phosphorous-containing backbones include, but are not limited to phosphorothioates, phosphorodithioates, chiral phosphorothioates, phosphotriesters, aminoalkylphosphotriesters, alkyl phosphonates, thionoalkylphosphonates, phosphinates, phosphoramidates, thionophosphoramidates, thionoalkylphosphotriesters, and boranophosphates and various salt forms thereof. See e.g., U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference.

Examples of the non-phosphorous containing backbones described above are disclosed in, e.g., U.S. Pat. Nos. 5,034,506; 5,185,444; 5,214,134; 5,216,141; 5,235,033; 5,264,562; 5,264,564; 5,405,938; 5,434,257; 5,470,967; 5,489,677; 5,541,307; 5,561,225; 5,596,086; 5,610,289; 5,602,240; 5,608,046; 5,610,289; 5,618,704; 5,623,070; 5,663,312; 5,677,437; and 5,677,439, each of which is herein incorporated by reference.

Modified forms of siRNA compounds can also contain modified nucleosides (nucleoside analogs), i.e., modified purine or pyrimidine bases, e.g., 5-substituted pyrimidines, 6-azapyrimidines, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), 2-thiouridine, 4-thiouridine, 5-(carboxyhydroxymethyl)uridine, 5′-carboxymethylaminomethyl-2-thiouridine, 5-carboxymethylaminomethyluridine, 5-methoxyaminomethyl-2-thiouridine, 5-methylaminomethyluridine, 5-methylcarbonylmethyluridine, 5-methyloxyuridine, 5-methyl-2-thiouridine, 4-acetylcytidine, 3-methylcytidine, propyne, quesosine, wybutosine, wybutoxosine, beta-D-galactosylqueosine, N-2, N-6 and O-substituted purines, inosine, 1-methyladenosine, 1-methylinosine, 2,2-dimethylguanosine, 2-methyladenosine, 2-methylguanosine, N6-methyladenosine, 7-methylguanosine, 2-methylthio-N6-isopentenyladenosine, beta-D-mannosylqueosine, uridine-5-oxyacetic acid, 2-thiocytidine, threonine derivatives, and the like. See e.g., U.S. Pat. Nos. 3,687,808; 4,845,205; 5,130,302; 5,175,273; 5,367,066; 5,432,272; 5,459,255; 5,484,908; 5,502,177; 5,525,711; 5,587,469; 5,594,121; 5,596,091; 5,681,941; and 5,750,692, PCT Publication No. WO 92/07065; PCT Publication No. WO 93/15187; and Limbach et al., Nucleic Acids Res, 22:2183 (1994), each of which is incorporated herein by reference in its entirety.

In addition, modified siRNA compounds can also have substituted or modified sugar moieties, e.g., 2′-O-methoxyethyl sugar moieties. See e.g., U.S. Pat. Nos. 4,981,957; 5,118,800; 5,319,080; 5,393,878; 5,446,137; 5,466,786; 5,514,785; 5,567,811; 5,576,427; 5,591,722; 5,610,300; 5,627,0531 5,639,873; 5,646,265; 5,658,873; 5,670,633; and 5,700,920, each of which is herein incorporated by reference.

Modified siRNA compounds may be synthesized by the methods disclosed in, e.g., U.S. Pat. No. 5,652,094; International Publication Nos. WO 91/03162; WO 92/07065 and WO 93/15187; European Patent Application No. 92110298.4; Perrault et al., Nature, 344:565 (1990); Pieken et al., Science, 253:314 (1991); and Usman & Cedergren, Trends Biochem Sci, 17:334 (1992).

Preferably, the 3′ overhangs of the siRNAs of the present invention are modified to provide resistance to cellular nucleases. In one embodiment the 3′ overhangs comprise 2′-deoxyribonucleotides. In preferred embodiments (depicted in FIGS. 1 -72) these 3′ overhangs comprise a dinucleotide made of two 2′-deoxythymine residues (i.e., dTdT) linked by a 5′-3′ phosphodiester linkage.

siRNA compounds may be administered to mammals by various methods through different routes. For example, they can be administered by intravenous injection. See Song et al., Nature Medicine, 9:347-351 (2003). They can also be delivered directly to a particular organ or tissue by any suitable localized administration methods. Several other approaches for delivery of siRNA into animals have also proved to be successful. See e.g., McCaffery et al., Nature, 418:38-39 (2002); Lewis et al., Nature Genetics, 32:107-108 (2002); and Xia et al., Nature Biotech., 20:1006-1010 (2002). Alternatively, they may be delivered encapsulated in liposomes, by iontophoresis, or by incorporation into other vehicles such as hydrogels, cyclodextrins, biodegradable nanocapsules, and bioadhesive microspheres.

In addition, they may also be delivered by a gene therapy approach, using a DNA vector from which siRNA compounds in, e.g., small hairpin form (shRNA), can be transcribed directly. Recent studies have demonstrated that while double-stranded siRNAs are very effective at mediating RNAi, short, single-stranded, hairpin-shaped RNAs can also mediate RNAi, presumably because they fold into intramolecular duplexes that are processed into double-stranded siRNAs by cellular enzymes. Sui et al., Proc Natl Acad Sci USA, 99:5515-5520 (2002); Yu et al., Proc Natl Acad Sci USA, 99:6047-6052 (2002); and Paul et al., Nature Biotech., 20:505-508 (2002)). This discovery has significant and far-reaching implications, since the production of such shRNAs can be readily achieved in vivo by transfecting cells or tissues with DNA vectors bearing short inverted repeats separated by a small number of (e.g., 3 to 9) nucleotides that direct the transcription of such small hairpin RNAs. Additionally, if mechanisms are included to direct the integration of the transcription cassette into the host cell genome, or to ensure the stability of the transcription vector, the RNAi caused by the encoded shRNAs, can be made stable and heritable. Not only have such techniques been used to “knock down” the expression of specific genes in mammalian cells, but they have now been successfully employed to knock down the expression of exogenously expressed transgenes, as well as endogenous genes in the brain and liver of living mice. See generally Hannon, Nature. 418:244-251 (2002) and Shi, Trends Genet, 19:9-12 (2003); see also Xia et al., Nature Biotech., 20:1006-1010 (2002).

Additional siRNA compounds targeted at different sites of the nucleic acids encoding one or more interacting protein members of a protein complex identified in the present invention may also be designed and synthesized according to general guidelines provided herein and generally known to skilled artisans. See e.g., Elbashir, et al. (Nature 411: 494-498 (2001). For example, guidelines have been compiled into “The siRNA User Guide” which is available at the website of The Rockefeller University, New York, N.Y.

Additionally, to assist in the design of siRNAs for the efficient RNAi-mediated silencing of any target gene, several siRNA supply companies maintain web-based design tools that utilize these general guidelines for “picking” siRNAs when presented with the mRNA or coding DNA sequence of the target gene. Examples of such tools can be found at the web sites of Dharmacon, Inc. (Lafayette, Colo.), Ambion, Inc. (Austin, Tex.), and Qiagen, Inc. (Valencia, Calif.), among others. Generally speaking, when provided with an mRNA or coding DNA sequence, these design tools scan the sequence for potential siRNA targets, using several distinct criteria. For example, the design tools may scan for an open reading frame and limit further scanning to that region of sequence. They may then scan for a particular dinucleotide, the most desirable of which being AA, or alternatively CA, GA or TA. Upon finding one of these dinucleotides, they will then examine the dinucleotide and the 19 nucleotides immediately 3′ of it for G/C content, nucleotide triplets (esp. GGG & CCC), and, using a BLAST algorithm search, for whether or not the 19 nucleotide sequence is unique to a specific target gene in the human genome. The features that make for an “ideal” target sequence are: (1) a 5′-most dinucleotide sequence of AA, or, less preferably, CA, GA or TA; (2) a G/C content of approximately 30-50%; (3) lack of trinucleotide repeats, especially GGG and CCC, and (4) being unique to the target gene (i.e., sequences that share no significant homology with genes other than the one being targeted), so that other genes are not inadvertently targeted by the same siRNA designed for this particular target sequence. Another criteria to be considered is whether or not the target sequence includes a known polymorphic site. If so, siRNAs designed to target one particular allele may not effectively target another allele, since single base mismatches between the target sequence and its complementary strand in a given siRNA can greatly reduce the effectiveness of RNAi induced by that siRNA. Given that target sequence and such design tools and design criteria, an ordinarily skilled artisan apprised of the present disclosure should be able to design and synthesized additional siRNA compounds useful in reducing the mRNA level and therefore protein level of one or more interacting protein members of a protein complex identified in the present invention.

### 6.2.3. Antisense Therapy

In another embodiment, antisense compounds specific to nucleic acids encoding one or more interacting protein members of a protein complex identified in the present invention are administered to cells or tissue in vitro or in a patient to be therapeutically or prophylactically treated. The antisense compounds should specifically inhibit the expression of the one or more interacting protein members. Examples of antisense compounds specific to nucleic acids encoding individual proteins in the tables above are provided in SEQ ID NOs:3-224.

As is known in the art, antisense drugs generally act by hybridizing to a particular target nucleic acid thus blocking gene expression. Methods for designing antisense compounds and using such compounds in treating diseases are well known and well developed in the art. For example, the antisense drug Vitravene® (fomivirsen), a 21-base long oligonucleotide, has been successfully developed and marketed by Isis Pharmaceuticals, Inc. for treating cytomegalovirus (CMV)-induced retinitis.

Any methods for designing and making antisense compounds may be used for the purpose of the present invention. See generally, Sanghvi et al., eds., Antisense Research and Applications, CRC Press, Boca Raton, 1993. Typically, antisense compounds are oligonucleotides designed based on the nucleotide sequence of the mRNA or gene of one or more target proteins, e.g., the interacting protein members of a particular protein complex of the present invention. In particular, antisense compounds can be designed to specifically hybridize to a particular region of the gene sequence or mRNA of one or more of the interacting protein members to modulate (increase or decrease) replication, transcription, or translation. As used herein, the term “specifically hybridize” or paraphrases thereof means a sufficient degree of complementarity or pairing between an antisense oligo and a target DNA or mRNA such that stable and specific binding occurs therebetween. In particular, 100% complementary or pairing is not required. Specific hybridization takes place when sufficient hybridization occurs between the antisense compound and its intended target nucleic acids in the substantial absence of non-specific binding of the antisense compound to non-target sequences under predetermined conditions, e.g., for purposes of in vivo treatment, preferably under physiological conditions. Preferably, specific hybridization results in the interference with normal expression of the target DNA or mRNA.

For example, antisense oligonucleotides can be designed to specifically hybridize to target genes, in regions critical for regulation of transcription; to pre-mRNAs, in regions critical for correct splicing of nascent transcripts; and to mature mRNAs, in regions critical for translation initiation or mRNA stability and localization.

As is generally known in the art, commonly used oligonucleotides are oligomers or polymers of ribonucleotides or deoxyribonucleotides, that are composed of a naturally-occurring nitrogenous base, a sugar (ribose or deoxyribose) and a phosphate group. In nature, the nucleotides are linked together by phosphodiester bonds between the 3′ and 5′ positions of neighboring sugar moieties. However, it is noted that the term “oligonucleotides” also encompasses various non-naturally occurring mimetics and derivatives, i.e., modified forms, of naturally occurring oligonucleotides as described below. Typically an antisense compound of the present invention is an oligonucleotide having from about 6 to about 200, and preferably from about 8 to about 30 nucleoside bases.

The antisense compounds preferably contain modified backbones or non-natural internucleoside linkages, including but not limited to, modified phosphorous-containing backbones and non-phosphorous backbones such as morpholino backbones; siloxane, sulfide, sulfoxide, sulfone, sulfonate, sulfonamide, and sulfamate backbones; formacetyl and thioformacetyl backbones; alkene-containing backbones; methyleneimino and methylenehydrazino backbones; amide backbones, and the like.

Examples of modified phosphorous-containing backbones include, but are not limited to phosphorothioates, phosphorodithioates, chiral phosphorothioates, phosphotriesters, aminoalkylphosphotriesters, alkyl phosphonates, thionoalkylphosphonates, phosphinates, phosphoramidates, thionophosphoramidates, thionoalkylphosphotriesters, and boranophosphates and various salt forms thereof. See e.g., U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference.

Examples of the non-phosphorous containing backbones described above are disclosed in, e.g., U.S. Pat. Nos. 5,034,506; 5,185,444; 5,214,134; 5,216,141; 5,235,033; 5,264,562; 5,264,564; 5,405,938; 5,434,257; 5,470,967; 5,489,677; 5,541,307; 5,561,225; 5,596,086; 5,610,289; 5,602,240; 5,608,046; 5,610,289; 5,618,704; 5,623,070; 5,663,312; 5,677,437; and 5,677,439, each of which is herein incorporated by reference.

Another useful modified oligonucleotide is peptide nucleic acid (PNA), in which the sugar-backbone of an oligonucleotide is replaced with an amide containing backbone, e.g., an aminoethylglycine backbone. See U.S. Pat. Nos. 5,539,082 and 5,714,331; and Nielsen et al., Science, 254, 1497-1500 (1991), all of which are incorporated herein by reference. PNA antisense compounds are resistant to RNase H digestion and thus exhibit longer half-life. In addition, various modifications may be made in PNA backbones to impart desirable drug profiles such as better stability, increased drug uptake, higher affinity to target nucleic acid, etc.

Alternatively, the antisense compounds are oligonucleotides containing modified nucleosides, i.e., modified purine or pyrimidine bases, e.g., 5-substituted pyrimidines, 6-azapyrimidines, and N-2, N-6 and O-substituted purines, and the like. See e.g., U.S. Pat. Nos. 3,687,808; 4,845,205; 5,130,302; 5,175,273; 5,367,066; 5,432,272; 5,459,255; 5,484,908; 5,502,177; 5,525,711; 5,587,469; 5,594,121; 5,596,091; 5,681,941; and 5,750,692, each of which is incorporated herein by reference in its entirety.

In addition, oligonucleotides with substituted or modified sugar moieties may also be used. For example, an antisense compound may have one or more 2′-O-methoxyethyl sugar moieties. See e.g., U.S. Pat. Nos. 4,981,957; 5,118,800; 5,319,080; 5,393,878; 5,446,137; 5,466,786; 5,514,785; 5,567,811; 5,576,427; 5,591,722; 5,610,300; 5,627,0531 5,639,873; 5,646,265; 5,658,873; 5,670,633; and 5,700,920, each of which is herein incorporated by reference.

Other types of oligonucleotide modifications are also useful including linking an oligonucleotide to a lipid, phospholipid or cholesterol moiety, cholic acid, thioether, aliphatic chain, polyamine, polyethylene glycol (PEG), or a protein or peptide. The modified oligonucleotides may exhibit increased uptake into cells, and improved stability, i.e., resistance to nuclease digestion and other biodegradations. See e.g., U.S. Pat. No. 4,522,811; Burnham, Am J Hosp Pharm, 15:210-218 (1994).

Antisense compounds can be synthesized using any suitable methods known in the art. In fact, antisense compounds may be custom made by commercial suppliers. Alternatively, antisense compounds may be prepared using DNA synthesizers available commercially from various vendors, e.g., Applied Biosystems Group of Norwalk, Conn.

The antisense compounds can be formulated into a pharmaceutical composition with suitable carriers and administered into cells or tissue in vitro or in a patient using any suitable route of administration. Alternatively, the antisense compounds may also be used in a “gene-therapy” approach. That is, the oligonucleotide is subcloned into a suitable vector and transformed into human cells. The antisense oligonucleotide is then produced in vivo through transcription. Methods for gene therapy are disclosed in Section 6.3.2 below.

In a specific embodiment of the instant invention, antisense oligonucleotides are used to decrease the expression of Stearoyl-CoA desaturase (SCD) in a patient in need of such treatment, and thereby prevent, delay the onset, treat the symptoms, or slow the progression of AD. In particular, the methods of Crooke and Graham, as described in U.S. Patent Application No. 2003/0083282 (published May 1, 2003, and hereby incorporated by reference in its entirety) are used to achieve the antisense modulation of SCD expression and thereby prevent, delay the onset, treat the symptoms, or slow the progression of AD.

In another specific embodiment of the instant invention, antisense oligonucleotides are used to decrease the expression of PSD-95/SAP in a patient in need of such treatment, and thereby prevent, delay the onset, treat the symptoms, or slow the progression of AD. In particular, the methods of Johns and Tao, as described in PCT Patent Application No. PCT/USO1/15372 (Publicaiton No. WO 01/87285, published 22 November 2001, and hereby incorporated by reference in its entirety) are used to achieve the antisense modulation of PSD-95/SAP expression and thereby prevent, delay the onset, treat the symptoms, or slow the progression of AD.

Similarly, in other specific embodiments of the instant invention, antisense oligonucleotides are used to decrease the expression of FAK2, PN7740, BAT3 and/or FKBP25, in a patient in need of such treatment, and thereby prevent, delay the onset, treat the symptoms, or slow the progression of AD. In each of these cases, antisense oligonucleotides can be test for efficacy in decreasing the expression of their targets in cell-based model systems, before being tested in experimental animals. The efficacy of a particular antisense oligonucleotide can be readily assessed by a quantitative analysis of mRNA transcript or protein levels using any of a variety of techniques commonly practiced in the art.

### 6.2.4. Ribozyme Therapy

In another embodiment, an enzymatic RNA or ribozyme is designed to target the nucleic acids encoding one or more of the interacting protein members of the protein complexes of the present invention. Ribozymes are RNA molecules possessing enzymatic activity. One class of ribozymes is capable of repeatedly cleaving other separate RNA molecules into two or more pieces in a nucleotide base sequence specific manner. See Kim et al., Proc Natl Acad Sci USA, 84:8788 (1987); Haseloff & Gerlach, Nature, 334:585 (1988); and Jefferies et al., Nucleic Acid Res, 17:1371 (1989). Such ribozymes typically have two functional domains: a catalytic domain and a binding sequence that guides the binding of ribozymes to a target RNA through complementary base-pairing. Once a specifically-designed ribozyme is bound to a target mRNA, it enzymatically cleaves the target mRNA, typically reducing its stability and destroying its ability to direct translation of an encoded protein. After a ribozyme has cleaved its RNA target, it is released from that target RNA and thereafter can bind and cleave another target. That is, a single ribozyme molecule can repeatedly bind and cleave new targets. Therefore, one advantage of ribozyme treatment is that a lower amount of exogenous RNA is required as compared to conventional antisense therapies. In addition, ribozymes exhibit less affinity to mRNA targets than DNA-based antisense oligonucleotides, and therefore are less prone to bind to unintended targets.

In accordance with the present invention, a ribozyme may target any portion of the mRNA encoding one or more interacting protein members of the protein complexes formed by the interactions described in the tables. Methods for selecting a ribozyme target sequence and designing and making ribozymes are generally known in the art. See e.g., U.S. Pat. Nos. 4,987,071; 5,496,698; 5,525,468; 5,631,359; 5,646,020; 5,672,511; and 6,140,491, each of which is incorporated herein by reference in its entirety. For example, suitable ribozymes may be designed in various configurations such as hammerhead motifs, hairpin motifs, hepatitis delta virus motifs, group I intron motifs, or RNase P RNA motifs. See e.g., U.S. Pat. Nos. 4,987,071; 5,496,698; 5,525,468; 5,631,359; 5,646,020; 5,672,511; and 6,140,491; Rossi et al., AIDS Res Human Retroviruses 8:183 (1992); Hampel & Tritz, Biochemistry 28:4929 (1989); Hampel et al., Nucleic Acids Res, 18:299 (1990); Perrotta & Been, Biochemistry 31:16 (1992); and Guerrier-Takada et al., Cell, 35:849 (1983).

Ribozymes can be synthesized by the same methods used for normal RNA synthesis. For example, such methods are disclosed in Usman et al., J Am Chem Soc, 109:7845-7854 (1987) and Scaringe et al., Nucleic Acids Res, 18:5433-5441 (1990). Modified ribozymes may be synthesized by the methods disclosed in, e.g., U.S. Pat. No. 5,652,094; International Publication Nos. WO 91/03162; WO 92/07065 and WO 93/15187; European Patent Application No. 92110298.4; Perrault et al., Nature, 344:565 (1990); Pieken et al., Science, 253:314 (1991); and Usman & Cedergren, Trends Biochem Sci, 17:334 (1992).

Ribozymes of the present invention may be administered to cells by any known methods, e.g., disclosed in International Publication No. WO 94/02595. For example, they can be administered directly to cells or tissue in vitro or in a patient through any suitable route, e.g., intravenous injection. Alternatively, they may be delivered encapsulated in liposomes, by iontophoresis, or by incorporation into other vehicles such as hydrogels, cyclodextrins, biodegradable nanocapsules, and bioadhesive microspheres. In addition, they may also be delivered by a gene therapy approach, using a DNA vector from which the ribozyme RNA can be transcribed directly. Gene therapy methods are disclosed in detail below in Section 6.3.2.

### 6.2.5. Other Methods

The in-patient concentrations and activities of the protein complexes and interacting proteins of the present invention may also be altered by other methods. For example, compounds identified in accordance with the methods described in Section 5 that are capable of interfering with or dissociating protein-protein interactions between the interacting protein members of a protein complex may be administered to cells or tissue in vitro or in a patient. Compounds identified in in vitro binding assays described in Section 5.2 that bind to the protein complexes of the present invention, or the interacting members thereof, may also be used in the treatment. Compounds identified in in vivo binding assays described in Section 5.3 that bind to the protein complexes of the present invention, or the interacting members thereof, or alter their functions and activities in a desirable way, may also be used in the treatment.

In addition, potentially useful agents also include incomplete proteins, i.e., fragments of the interacting protein members that are capable of binding to their respective binding partners in a protein complex but are defective with respect to their normal cellular functions. For example, binding domains of the interacting member proteins of a protein complex may be used as competitive inhibitors of the activities of the protein complex. As will be apparent to skilled artisans, derivatives or homologues of the binding domains may also be used. Binding domains can be easily identified using molecular biology techniques, e.g., mutagenesis in combination with yeast two-hybrid assays. Preferably, the protein fragment used is a fragment of an interacting protein member having a length of less than 90%, 80%, more preferably less than 75%, 65%, 50%, or less than 40% of the full length of the protein member. Examples of protein fragments of the proteins in the tables above that are potentially useful agents are provided by SEQ ID NOs:225-688.

Additionally and advantageously, several of the interacting proteins disclosed in the tables above are enzymes that act upon protein susbstrates. In these cases, fragments of the protein substrates can often be used to inhibit or interfere with the activity of the enzymes acting upon them. For example, as mentioned above, PN7740 is a novel protein containing a protein phosphatase 2C domain, which likely acts to dephosphorylate specific phospho-serine or phospho-threonine residues on particular protein substrates. Recently, using the yeast two-hybrid technique, Flajolet and coworkers discovered that the 50 amino acid residue carboxyl terminal, and therefore cytoplasmic, tail of the meabotropic glutamate receptor 3 (mGluR3) interacts specifically with protein phosphatase 2Cα (PP2Cα) (Flajolet et al., Proc Natl Acad Sci USA 100:16006-16011 (2003)). Using subsequent GST pull-down experiments and coimmunoprecipitation assays, they further demonstrated the C-terminal tail of mGluR3 interacts not only with PP2Cα, but also with the β, γ, and δ, PP2C isoforms. Flajolet and colleagues further characterized the interaction of mGluR3 and PP2Cα by conducting GST pull-down experiments with various increasing truncated GST-mGluR3 peptides. They found that a minimal region of only 20 amino acid residues of mGluR3, residues 836-855, was required for the interaction with PP2Cα. Flajolet and coworkers identified a particular serine residue in this 20-mer peptide that was phosphorylated by PKA and dephosphorylated by the various isoforms of PP2C. In additional experiments designed characterize the interaction of the dephospho- and phospho-forms of the mGluR3 cytoplasmic tail with PP2Cα , they discovered that a synthetic peptide comprising amino acid residues 830-979 of mGluR3 effectively inhibitied the dephosphorylation of a synthetic phospho-casein substrate by the protein phosphatase.

Given the findings of Flajolet and coworkers described above (see Flajolet et al., Proc Natl Acad Sci USA 100: 16006-16011 (2003)) we propose that peptide fragments of mGluR3 (provided as SEQ ID NOs: 689-695)—or other protein substrate dephosphorylated by PP2Cs—can be used to effectively inhibit the phosphatase activity of PN7740. Consequently, such peptides represent potentially useful therapeutics agents for the treatment of neurodegenerative disorders or diseases, such as Alzheimer's disease, or for the delay of onset, or treatment of their symptoms.

In one embodiment, a fragment of a protein identified in the tables above is administered. In a specific embodiment, one or more of the interaction domains of a protein identified in the tables, within the regions listed in the tables, is administered to cells or tissue in vitro, or are administered to a patient in need of such treatment. For example, suitable protein fragments can include polypeptides having a contiguous span of 4, 5, 6, 7, 8, 9, 10, 11, 12, 13, 14, 15, 18, 20 or 25, preferably from 4 to 30, 40 or 50 amino acids or more of the sequence of a first protein identified in the tables, that are capable of interacting with a second protein described in the tables. Also, suitable protein fragments can include peptides capable of binding one or more of the proteins described in the tables, and having an amino acid sequence of from 4 to 30 amino acids that is at least 75%, 80%, 82%, 85%, 87%, 90%, 95% or more identical to a contiguous span of amino acids of a protein described in the tables. Alternatively, a polypeptide capable of interacting with a first protein of an interacting pair of proteins of the present invention, and having a contiguous span of 4, 5, 6, 7, 8, 9, 10, 11, 12, 13, 14, 15, 18, 20 or 25, preferably from 4 to 30, 40 or 50 or more amino acids of the amino acid sequence of a second protein of the same interacting pair of proteins, may be administered. Also, other examples of suitable compounds include a peptide capable of binding a first interacting partner of a pair of interacting proteins of the present invention and having an amino acid sequence of from 4 to 30, 40, 50 or more amino acids that is at least 75%, 80%, 82%, 85%, 87%, 90%, 92%, 95% or more identical to a contiguous span of amino acids from a second interacting partner of a pair of interacting proteins of the present invention. In addition, the administered compounds can be an antibody or antibody fragment, preferably a single-chain antibody immunoreactive with any of the proteins listed in the tables, or a protein complex of the present invention.

The protein fragments suitable as competitive inhibitors can be delivered into cells by direct cell internalization, receptor mediated endocytosis, or via a “transporter.” It is noted that when the target proteins or protein complexes to be modulated reside inside cells, the compound administered to cells in vitro or in vivo in the method of the present invention preferably is delivered into the cells in order to achieve optimal results. Thus, preferably, the compound to be delivered is associated with a transporter capable of increasing the uptake of the compound by cells harboring the target protein or protein complex. As used herein, the term “transporter” refers to an entity (e.g., a compound or a composition or a physical structure formed from multiple copies of a compound or multiple different compounds) that is capable of facilitating the uptake of a compound of the present invention by animal cells, particularly human cells. That is, the cell uptake of a compound of the present invention in the presence of a “transporter” is at least 50% higher than the cell uptake of the compound in the absence of the “transporter.” Preferably, a “transporter” is selected such that the cell uptake of a compound of the present invention in the presence of a “transporter” is at least 75% higher, preferably at least 100% or 200% higher, and more preferably at least 300%, 400% or 500% higher than the cell uptake of the compound in the absence of the “transporter.” Methods of assaying cell uptake of a compound should be apparent to skilled artisans. For example, the compound to be delivered can be labeled with a radioactive isotope or another detectable marker (e.g., a fluorescence marker), and added to cultured cells in the presence or absence of a transporter, and incubated for a time period sufficient to allow maximal uptake. Cells can then be separated from the culture medium and the detectable signal (e.g., radioactivity) caused by the compound inside the cells can be measured. The result obtained in the presence of a transporter can be compared to that obtained in the absence of a transporter.

Many molecules and structures known in the art can be used as “transporters.” In one embodiment, a penetratin is used as a transporter. For example, the homeodomain of Antennapedia, a Drosophila transcription factor, can be used as a transporter to deliver a compound of the present invention. Indeed, any suitable member of the penetratin class of peptides can be used to carry a compound of the present invention into cells. Penetratins are disclosed in, e.g., Derossi et al., Trends Cell Biol, 8:84-87 (1998), which is incorporated herein by reference. Penetratins transport molecules attached thereto across cytoplasmic membranes or nuclear membranes efficiently, in a receptor-independent, energy-independent, and cell type-independent manner. Methods for using a penetratin as a carrier to deliver oligonucleotides and polypeptides are also disclosed in U.S. Pat. No. 6,080,724; Pooga et al., Nature Biotech, 16:857 (1998); and Schutze et al., J Immunol, 157:650 (1996), all of which are incorporated herein by reference. U.S. Pat. No. 6,080,724 defines the minimal requirements for a penetratin peptide as a peptide of 16 amino acids with 6 to 10 of which being hydrophobic. The amino acid at position 6 counting from either the N- or C-terminus is tryptophan, while the amino acids at positions 3 and 5 counting from either the N- or C-terminus are not both valine. Preferably, the helix 3 of the homeodomain of Drosophila Antennapedia is used as a transporter. More preferably, a peptide having a sequence of amino acid residues 43-58 of the homeodomain Antp is employed as a transporter. In addition, other naturally occurring homologs of the helix 3 of the homeodomain of Drosophila Antennapedia can be used. For example, homeodomains of Fushi-tarazu and Engrailed have been shown to be capable of transporting peptides into cells. See Han et al., Mol Cell, 10:728-32 (2000). As used herein, the term “penetratin” also encompasses peptoid analogs of the penetratin peptides. Typically, the penetratin peptides and peptoid analogs thereof are covalently linked to a compound to be delivered into cells thus increasing the cellular uptake of the compound.

In another embodiment, the HIV-1 tat protein or a derivative thereof is used as a “transporter” covalently linked to a compound according to the present invention. The use of HIV-1 tat protein and derivatives thereof to deliver macromolecules into cells has been known in the art. See Green & Loewenstein, Cell, 55:1179 (1988); Frankel & Pabo, Cell, 55:1189 (1988); Vives et al., J Biol Chem, 272:16010-16017 (1997); Schwarze et al., Science, 285:1569-1572 (1999). It is known that the sequence responsible for cellular uptake consists of the highly basic region, amino acid residues 49-57. See e.g., Vives et al., J Biol Chem, 272:16010-16017 (1997); Wender et al., Proc Natl Acad Sci USA, 97:13003-13008 (2000). The basic domain is believed to target the lipid bilayer component of cell membranes. It causes a covalently linked protein or nucleic acid to cross cell membrane rapidly in a cell type-independent manner. Proteins ranging in size from 15 to 120 kD have been delivered with this technology into a variety of cell types both in vitro and in vivo. See Schwarze et al., Science, 285:1569-1572 (1999). Any HIV tat-derived peptides or peptoid analogs thereof capable of transporting macromolecules such as peptides can be used for purposes of the present invention. For example, any native tat peptides having the highly basic region, amino acid residues 49-57 can be used as a transporter by covalently linking it to the compound to be delivered. In addition, various analogs of the tat peptide of amino acid residues 49-57 can also be useful transporters for purposes of this invention. Examples of various such analogs are disclosed in Wender et al., Proc Natl Acad Sci USA, 97:13003-13008 (2000) (which is incorporated herein by reference) including, e.g., d-Tat₄₉₋₅₇, retro-inverso isomers of l- or d-Tat₄₉₋₅₇ (i.e., l-Tat₅₇₋₄₉ and d-Tat₅₇₋₄₉), L-arginine oligomers, D-arginine oligomers, L-lysine oligomers, D-lysine oligomers, L-histine oligomers, D-histine oligomers, L-ornithine oligomers, D-ornithine oligomers, and various homologues, derivatives (e.g., modified forms with conjugates linked to the small peptides) and peptoid analogs thereof. Preferably, arginine oligomers are preferred to the other oligomers, since arginine oligomers are much more efficient in promoting cellular uptake. As used herein, the term “oligomer” means a molecule that includes a covalently linked chain of amino acid residues of the same amino acids having a large enough number of such amino acid residues to confer transporter activities on the molecule. Typically, an oligomer contains at least 6, preferably at least 7, 8, or 9 such amino acid residues. In one embodiment, the transporter is a peptide that includes at least six contiguous amino acid residues that are a combination of two or more of L-arginine, D-arginine, L-lysine, D-lysine, L-histidine, D-histine, L-ornithine, and D-ornithine.

Other useful transporters known in the art include, but are not limited to, short peptide sequences derived from fibroblast growth factor (See Lin et al., J Biol Chem, 270:14255-14258 (1998)), Galparan (See Pooga et al., FASEB J 12:67-77 (1998)), and HSV-1 structural protein VP22 (See Elliott & O'Hare, Cell, 88:223-233 (1997)).

As the above-described various transporters are generally peptides, fusion proteins can be conveniently made by recombinant expression to contain a transporter peptide covalently linked by a peptide bond to a competitive protein fragment. Alternatively, conventional methods can be used to chemically synthesize a transporter peptide or a peptide of the present invention or both.

The hybrid peptide can be administered to cells or tissue in vitro or to a patient in a suitable pharmaceutical composition as provided in Section 8.

In addition to peptide-based transporters, various other types of transporters can also be used, including but not limited to cationic liposomes (see Rui et al., J Am Chem Soc, 120:11213-11218 (1998)), dendrimers (Kono et al., Bioconjugate Chem, 10:1115-1121 (1999)), siderophores (Ghosh et al., Chem Biol, 3:1011-1019 (1996)), etc. In a specific embodiment, the compound according to the present invention is encapsulated into liposomes for delivery into cells.

Additionally, when a compound according to the present invention is a peptide, it can be administered to cells by a gene therapy method. That is, a nucleic acid encoding the peptide can be administered to in vitro cells or to cells in vivo in a human or animal body. Any suitable gene therapy methods may be used for purposes of the present invention. Various gene therapy methods are well known in the art and are described in Section 6.3.2. below. Successes in gene therapy have been reported recently. See e.g., Kay et al., Nature Genet, 24:257-61 (2000); Cavazzana-Calvo et al., Science, 288:669 (2000); and Blaese et al., Science, 270: 475 (1995); Kantoff, et al., J Exp Med, 166:219 (1987).

In yet another embodiment, the gene therapy methods discussed in Section 6.3.2 below are used to “knock out” the gene encoding an interacting protein member of a protein complex, or to reduce the gene expression level. For example, the gene may be replaced with a different gene sequence or a non-functional sequence or simply deleted by homologous recombination. In another gene therapy embodiment, the method disclosed in U.S. Pat. No. 5,641,670, which is incorporated herein by reference, may be used to reduce the expression of the genes for the interacting protein members. Essentially, an exogenous DNA having at least a regulatory sequence, an exon and a splice donor site can be introduced into an endogenous gene encoding an interacting protein member by homologous recombination such that the regulatory sequence, the exon and the splice donor site present in the DNA construct become operatively linked to the endogenous gene. As a result, the expression of the endogenous gene is controlled by the newly introduced exogenous regulatory sequence. Therefore, when the exogenous regulatory sequence is a strong gene expression repressor, the expression of the endogenous gene encoding the interacting protein member is reduced or blocked. See U.S. Pat. No. 5,641,670.

### 6.3. Activation of Protein Complex or Interacting Protein Members Thereof

The present invention also provides methods for increasing in cells or tissue in vitro or in a patient the concentration and/or activity of a protein complex, or of an individual protein member thereof, identified in accordance with the present invention. Such methods can be particularly useful in instances where a reduced concentration and/or activity of a protein complex, or a protein member thereof, is associated with a particular disease or disorder to be treated, or where an increased concentration and/or activity of a protein complex, or a protein member thereof, would be beneficial to the improvement of a cellular function or disease state. By increasing the concentration of the protein complex, or a protein member thereof, and/or stimulating the functional activities of the protein complex or a protein member thereof, the disease or disorder may be treated or prevented.

### 6.3.1. Administration of Protein Complex or Protein Members Thereof

Where the concentration or activity of a particular protein complex of the present invention, or any individual protein constituent of a protein complex in cells or tissue in vitro or in a patient is determined to be low or is desired to be increased, the protein complex, or an individual constituent protein of the protein complex may be administered directly to the patient to increase the concentration and/or activity of the protein complex, or the individual constituent protein. For this purpose, protein complexes prepared by any one of the methods described in Section 2.3 may be administered to the patient, preferably in a pharmaceutical composition as described below. Alternatively, one or more individual interacting protein members of the protein complex may also be administered to the patient in need of treatment. For example, one or more of the individual proteins or the interacting pairs of proteins described in the tables may be given to cells or tissue in vitro or to a patient. Proteins isolated or purified from normal individuals or recombinantly produced can all be used in this respect. Preferably, two or more interacting protein members of a protein complex are administered. The proteins or protein complexes may be administered to a patient needing treatment using any of the methods described in Section 8.

### 6.3.2. Gene Therapy

In another embodiment, the concentration and/or activity of a particular protein complex comprising one or more of the interacting pairs of proteins described in the tables or an individual constituent protein of a protein complex of the present invention is increased or restored in patients, tissue or cells by a gene therapy approach. For example, nucleic acids encoding one or more protein members of a protein complex of the present invention, or portions or fragments thereof are introduced into patients, tissue, or cells such that the protein(s) are expressed from the introduced nucleic acids. For these purposes, nucleic acids encoding one or more of the proteins described in the tables, or fragments, homologues or derivatives thereof can be used in the gene therapy in accordance with the present invention. For example, if a disease-causing mutation exists in one of the protein members in cells or tissue in vitro or in a patient, then a nucleic acid encoding a wild-type protein can be introduced into tissue cells of the patient. The exogenous nucleic acid can be used to replace the corresponding endogenous defective gene by, e.g., homologous recombination. See U.S. Pat. No. 6,010,908, which is incorporated herein by reference. Alternatively, if the disease-causing mutation is a recessive mutation, the exogenous nucleic acid is simply used to express a wild-type protein in addition to the endogenous mutant protein. In another approach, the method disclosed in U.S. Pat. No. 6,077,705 may be employed in gene therapy. That is, the patient is administered both a nucleic acid construct encoding a ribozyme and a nucleic acid construct comprising a ribozyme resistant gene encoding a wild type form of the gene product. As a result, undesirable expression of the endogenous gene is inhibited and a desirable wild-type exogenous gene is introduced. In yet another embodiment, if the endogenous gene is of wild-type and the level of expression of the protein encoded thereby is desired to be increased, additional copies of wild-type exogenous genes may be introduced into the patient by gene therapy, or alternatively, a gene activation method such as that disclosed in U.S. Pat. No. 5,641,670 may be used.

Various gene therapy methods are well known in the art. Successes in gene therapy have been reported recently. See e.g., Kay et al., Nature Genet, 24:257-61 (2000); Cavazzana-Calvo et al., Science, 288:669 (2000); and Blaese et al., Science, 270: 475 (1995); Kantoff, et al., J Exp Med 166:219 (1987).

Any suitable gene therapy methods may be used for the purposes of the present invention. Generally, a nucleic acid encoding a desirable protein (e.g., one selected from any of the tables) is incorporated into a suitable expression vector and is operably linked to a promoter in the vector. Suitable promoters include but are not limited to viral transcription promoters derived from adenovirus, simian virus 40 (SV40) (e.g., the early and late promoters of SV40), Rous sarcoma virus (RSV), and cytomegalovirus (CMV) (e.g., CMV immediate-early promoter), human immunodeficiency virus (HIV) (e.g., long terminal repeat (LTR)), vaccinia virus (e.g., 7.5K promoter), and herpes simplex virus (HSV) (e.g., thymidine kinase promoter). Where tissue-specific expression of the exogenous gene is desirable, tissue-specific promoters may be operably linked to the exogenous gene. In addition, selection markers may also be included in the vector for purposes of selecting, in vitro, those cells that contain the exogenous gene. Various selection markers known in the art may be used including, but not limited to, e.g., genes conferring resistance to neomycin, hygromycin, zeocin, and the like.

In one embodiment, the exogenous nucleic acid (gene) is incorporated into a plasmid DNA vector. Many commercially available expression vectors may be useful for the present invention, including, e.g., pCEP4, pcDNAI, pIND, pSecTag2, pVAX1, pcDNA3.1, and pB1-EGFP, and pDisplay.

Various viral vectors may also be used. Typically, in a viral vector, the viral genome is engineered to eliminate the disease-causing capability of the virus, e.g., the ability to replicate in the host cells. The exogenous nucleic acid to be introduced into cells or tissue in vitro or in a patient may be incorporated into the engineered viral genome, e.g., by inserting it into a viral gene that is non-essential to the viral infectivity. Viral vectors are convenient to use as they can be easily introduced into cells, tissues and patients by way of infection. Once in the host cell, the recombinant virus typically is integrated into the genome of the host cell. In rare instances, the recombinant virus may also replicate and remain as extrachromosomal elements.

A large number of retroviral vectors have been developed for gene therapy. These include vectors derived from oncoretroviruses (e.g., MLV), lentiviruses (e.g., HIV and SIV) and other retroviruses. For example, gene therapy vectors have been developed based on murine leukemia virus (See, Cepko, et al., Cell, 37:1053-1062 (1984), Cone & Mulligan, Proc Natl Acad Sci USA, 81:6349-6353 (1984)), mouse mammary tumor virus (See, Salmons et al., Biochem Biophys Res Commun,159:1191-1198 (1984)), gibbon ape leukemia virus (See, Miller et al., J Virology, 65:2220-2224 (1991)), HIV, (See Shimada et al., J Clin Invest, 88:1043-1047 (1991)), and avian retroviruses (See Cosset et al., J Virology, 64:1070-1078 (1990)). In addition, various retroviral vectors are also described in U.S. Pat. Nos. 6,168,916; 6,140,111; 6,096,534; 5,985,655; 5,911,983; 4,980,286; and 4,868,116, all of which are incorporated herein by reference.

Adeno-associated virus (AAV) vectors have been successfully tested in clinical trials. See e.g., Kay et al., Nature Genet 24:257-61 (2000). AAV is a naturally occurring defective virus that requires other viruses such as adenoviruses or herpes viruses as helper viruses. See Muzyczka, Curr Top Microbiol Immun, 158:97 (1992). A recombinant AAV virus useful as a gene therapy vector is disclosed in U.S. Pat. No. 6,153,436, which is incorporated herein by reference.

Adenoviral vectors can also be useful for purposes of gene therapy in accordance with the present invention. For example, U.S. Pat. No. 6,001,816 discloses an adenoviral vector, which is used to deliver a leptin gene intravenously to a mammal to treat obesity. Other recombinant adenoviral vectors may also be used, which include those disclosed in U.S. Pat. Nos. 6,171,855; 6,140,087; 6,063,622; 6,033,908; and 5,932,210, and Rosenfeld et al., Science, 252:431-434 (1991); and Rosenfeld et al., Cell, 68:143-155 (1992).

Other useful viral vectors include recombinant hepatitis viral vectors (See, e.g., U.S. Pat. No. 5,981,274), and recombinant entomopox vectors (See, e.g., U.S. Pat. Nos. 5,721,352 and 5,753,258).

Other non-traditional vectors may also be used for purposes of this invention. For example, International Publication No. WO 94/18834 discloses a method of delivering DNA into mammalian cells by conjugating the DNA to be delivered with a polyelectrolyte to form a complex. The complex may be microinjected into or taken up by cells.

The exogenous gene fragment or plasmid DNA vector containing the exogenous gene may also be introduced into cells by way of receptor-mediated endocytosis. See e.g., U.S. Pat. No. 6,090,619; Wu & Wu, J Biol Chem, 263:14621 (1988); Curiel et al., Proc Natl Acad Sci USA, 88:8850 (1991). For example, U.S. Pat. No. 6,083,741 discloses introducing an exogenous nucleic acid into mammalian cells by associating the nucleic acid to a polycation moiety (e.g., poly-L-lysine having 3-100 lysine residues), which is itself coupled to an integrin receptor binding moiety (e.g., a cyclic peptide having the sequence Arg-Gly-Asp).

Alternatively, the exogenous nucleic acid or vectors containing it can also be delivered into cells via amphiphiles. See e.g., U.S. Pat. No. 6,071,890. Typically, the exogenous nucleic acid or a vector containing the nucleic acid forms a complex with the cationic amphiphile. Mammalian cells contacted with the complex can readily take it up.

The exogenous gene can be introduced into cells or tissue in vitro or in a patient for purposes of gene therapy by various methods known in the art. For example, the exogenous gene sequences alone or in a conjugated or complex form described above, or incorporated into viral or DNA vectors, may be administered directly by injection into an appropriate tissue or organ of a patient. Alternatively, catheters or like devices may be used to deliver exogenous gene sequences, complexes, or vectors into a target organ or tissue. Suitable catheters are disclosed in, e.g., U.S. Pat. Nos. 4,186,745; 5,397,307; 5,547,472; 5,674,192; and 6,129,705, all of which are incorporated herein by reference.

In addition, the exogenous gene or vectors containing the gene can be introduced into isolated cells using any known techniques such as calcium phosphate precipitation, microinjection, lipofection, electroporation, biolystics, receptor-mediated endocytosis, and the like. Cells expressing the exogenous gene may be selected and redelivered back to the patient by, e.g., injection or cell transplantation. The appropriate amount of cells delivered to a patient will vary with patient conditions, and desired effect, which can be determined by a skilled artisan. See e.g., U.S. Pat. Nos. 6,054,288; 6,048,524; and 6,048,729. Preferably, the cells used are autologous, i.e., cells obtained from the patient being treated.

In specific embodiments of the present invention, gene therapy techniques are used to increase the concentration and/or activity of the protein complexes, or individual interacting proteins of the protein complexes disclosed above, in cells, tissues or organs, and especially in the brains of AD patients. In particular, gene therapy techniques are used to restore the concentration or activity of alpha-endosulfine (ALPEND/ENSA) and peroxiredoxin 3 (PRDX3), which were found to interact with acetylcholinesterase (ACHE(614)) and axin, respectively. Concentrations of ALPEND/ENSA have been found to be extremely decreased in the brains of AD patients, which could result in the continuous opening of K(ATP) channels with subsequent derease of neurotransmitter release and change of potassium fluxes (Kim & Lubec, Neurosci Lett 310:77-80 (2001)). Restoration of ALPEND/ENSA concentration through the use of gene therapy techniques would restore protein-protein complexes formed by the interaction of ALPEND/ENSA with acetylcholinesterase. Concentrations of PRDX3 are significantly decreased in the brains of patients with AD and Down syndrome (Kim et al., J Neural Transm Suppl 61:223-235 (2001)). Hence, restoration of PRDX3 concentration through the use of gene therapy techniques would restore protein-protein complexes formed by the interaction of PRDX3 with axin. Restoration of these protein complexes of the present invention can restore biological functions that would otherwise be reduced or lost in the brains of AD patients.

### 6.4. Small Organic Compounds

Diseases or disorders in cells or tissue in vitro, or in a patient, associated with the decreased concentration or activity of a protein complex of the present invention, or an individual protein constituent of a protein complex identified in accordance with the present invention, can also be ameliorated by administering to the patient a compound identified by the methods described in Sections 5.1-5.3, and capable of modulating the functions or intracellular levels of the protein complex or a constituent protein member thereof, e.g., by triggering or initiating, enhancing or stabilizing protein-protein interaction between the interacting protein members of the protein complex, or the mutant forms of such interacting protein members found in the patient. Such compounds can therefore be used for the treatment of neurological disorders, ailments and diseases, including mild cognitive impairment, depression, schizophrenia, obsessive-compulsive disorder, bipolar disorder, and neurodegenerative diseases and disorders and motor neuron diseases and disorders such as Alzheimer's disease, Parkinson's disease, dementia with Lewy bodies, amyotrophic lateral sclerosis or Lou Gehrig's disease, Alpers' disease, Leigh's disease, Pelizaeus-Merzbacher disease, Olivopontocerebellar atrophy, Friedreich's ataxia, leukodystrophies, Rett syndrome, Ramsay Hunt syndrome type II, and Down's syndrome. In specific situations, small organic compounds that modulate the functions of specific proteins of the interacting pairs described above have already been identified, and, in some cases, have been proposed for use as therapeutics for other diseases such as diabetes and obesity. In such cases, the previously identified compounds can be used for the treatment of neurological disorders, ailments and diseases, including mild cognitive impairment, depression, schizophrenia, obsessive-compulsive disorder, bipolar disorder, and neurodegenerative diseases and disorders and motor neuron diseases and disorders such as Alzheimer's disease, Parkinson's disease, dementia with Lewy bodies, amyotrophic lateral sclerosis or Lou Gehrig's disease, Alpers' disease, Leigh's disease, Pelizaeus-Merzbacher disease, Olivopontocerebellar atrophy, Friedreich's ataxia, leukodystrophies, Rett syndrome, Ramsay Hunt syndrome type II, and Down's syndrome.

Importantly, it has been discovered that modulation of expression levels of several of the interacting proteins of the instant invention affects the processing of APP, and ultimately the production of Aβ42. This has been demonstrated using the Aβ secretion assay described in Example 8, below. In particular, it has been discovered that, under certain conditions, overexpression of FAK2, SCD, BAT3 and CIB results in an increase in Aβ secretion. Consequently, it is expected that either inhibiting the activities of these proteins, or reducing their expression, can lead to decreased Aβ secretion. This hypothesis has been tested and confirmed in the case of SCD. Thus, in another aspect of the present invention, small molecule inhibitors of SCD and other proteins are provided.

6.4.1. Small Molecule Inhibitors of FAK2:

In one embodiment, preferred compounds for use in the instant invention are those that inhibit the kinase FAK2 (see, e.g., GenBank accession no. U33284; Lev et al. Nature 376:737-745 (1995)). Without wishing to be bound by theory, it is believed that by inhibiting human protein tyrosine kinase FAK2, with compounds such as 4-amino-5-(4-chlorophenyl)-7-(t-butyl)pyrazolo[3,4-d]pyrimidine (as disclosed in Wang & Reiser J Neurochem 84:1349-1357 (2003), which is herein incorporated by reference in its entirety), or with tyrphostin A9 (as disclosed in Loeser et al., J Biol Chem 278:24577-24585 (2003), which is herein incorporated by reference in its entirety), or with thapsigargin and/or GF109203X (as disclosed in Pace et al., J Biol Chem 278:19008-19016 (2003), which is herein incorporated by reference in its entirety), one can modulate amyloid precursor protein processing, and thus treat and/or prevent Alzheimer's disease according to the methods of the invention. Similarly, without wishing to be bound by any theory, it is also believed that inhibiting human protein tyrosine kinase FAK2 with any of the compounds disclosed in U.S. Pat. No. 6,593,326 (issued Jul. 15, 2003 and incorporated by reference herein in its entirety) one can modulate amyloid precursor protein processing, and thus treat and/or prevent Alzheimer's disease according to the methods of the invention. Specifically, it is believed that Alzheimer's disease can be treated or prevented by administering a therapeutically or prophylactically effective dose of a compound of the following Formula I: 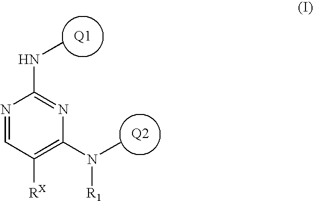
wherein:

R¹ is selected from hydrogen, C₁₋₆ alkyl [optionally substituted by one or two substituents independently selected from halo, amino, C₁₋₄ alkylamino, di-(C₁₋₄ alkyl)amino, hydroxy, cyano, C₁₋₄ alkoxy, C₁₋₄ alkoxycarbonyl, carbamoyl, —NHCOC₁₋₄ alkyl, trifluoromethyl, phenylthio, phenoxy, pyridyl, morpholino], benzyl, 2-phenylethyl, C₃₋₅ alkenyl [optionally substituted by up to three halo substituents, or by one trifluoromethyl substituent, or one phenyl substituent], N-phthalimido-C₁₋₄ alkyl, C₃₋₅ alkynyl [optionally substituted by one phenyl substituent] and C₃₋₆ cycloalkyl-C₁₋₆ alkyl; wherein any phenyl or benzyl group in R¹ is optionally substituted by up to three substituents independently selected from halo, hydroxy, nitro, amino, C₁₋₃ alkylamino, di-(C₁₋₃ alkyl)amino, cyano, trifluoromethyl, C₁₋₃ alkyl [optionally substituted by 1 or 2 substituents independently selected from halo, cyano, amino, C₁₋₃ alkylamino, di-(C₁₋₃ alkyl)amino, hydroxy and trifluoromethyl], C₃₋₅ alkenyl [optionally substituted by up to three halo substituents, or by one trifluoromethyl substituent], C₃₋₅ alkynyl, C₁₋₃ alkoxy, mercapto, C₁₋₃ alkylthio, carboxy, C₁₋₃ alkoxycarbonyl;

R^{x} is selected from halo, hydroxy, nitro, amino, cyano, mercapto, carboxy, sulphamoyl, formamido, ureido or carbamoyl or a group of formula (Ib):)

wherein:

A is C₁₋₆ alkyl, C₂₋₆ alkenyl, C₂₋₆ alkynyl, C₃₋₈ cycloalkyl, phenyl, heterocycle or heteroaryl, wherein said C₁₋₆ alkyl, C₃₋₆ alkenyl and C₃₋₆ alkynyl are optionally substituted by one or more substituents selected from halo, nitro, cyano, amino, hydroxy, mercapto, carboxy, formamido, ureido, C₁₋₃ alkylamino, di-(C₁₋₃ alkyl)amino, C₁₋₃ alkoxy, trifluoromethyl, C₃₋₈ cycloalkyl, phenyl, heterocycle or heteroaryl; wherein any phenyl, C₃₋₈ cycloalkyl, heterocycle or heteroaryl may be optionally substituted by one or more halo, nitro, cyano, hydroxy, trifluoromethyl, trifluoromethoxy, amino, carboxy, carbamoyl, mercapto, formamido, ureido, sulphamoyl, C₁₋₄ alkyl, C₂₋₄ alkenyl, C₂₋₄ alkynyl, C₁₋₄ alkoxy, C₁₋₄ alkanoyl, C₁₋₄ alkanoyloxy, C₁₋₄ alkylamino, di-(C₁₋₄ alkyl)amino, C₁₋₄ alkanoylamino, N—C₁₋₄ alkylcarbamoyl, N,N-di-(C₁₋₄ alkyl)carbamoyl, C₁₋₄ alkylthio, C₁₋₄ alkylsulphinyl, C₁₋₄ alkylsulphonyl and C₁₋₄ alkoxycarbonyl;

B is —O—, —S—, —C(O)—, —NH—, —N(C₁₋₄ alkyl)-, —C(O)NH—, —C(O)N(C₁₋₄ alkyl)-, —NHC(O)—, —N(C₁₋₄ alkyl)C(O)— or B is a direct bond;

C is C₁₋₄ alkylene or a direct bond;

Q₁ and Q2 are independently selected from aryl, a 5- or 6-membered monocyclic moiety (linked via a ring carbon atom and containing one to three heteroatoms independently selected from nitrogen, oxygen and sulphur); and a 9- or 10-membered bicyclic heterocyclic moiety (linked via a ring carbon atom and containing one or two nitrogen heteroatoms and optionally containing a further one or two heteroatoms selected from nitrogen, oxygen and sulphur);

and one or both of Q₁ and Q₂ bears on any available carbon atom one substituent of the formula (Ia) and Q₂ may optionally bear on any available carbon atom further substituents of the formula (Ia):

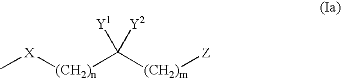

[provided that when present in Q₁ the substituent of formula (Ia) is not adjacent to the —NH— link];

wherein:

X is —CH₂—, —O—, —NH—, —NR^{y}— or —S— [wherein R^{y} is C₁₋₄ alkyl, optionally substituted by one substituent selected from halo, amino, cyano, C₁₋₄ alkoxy or hydroxy];

Y¹ is H, C₁₋₄ alkyl or as defined for Z;

Y² is H or C₁₋₄ alkyl;

Z is R^{a} O—, R^{b} R^{e} N—, R^{d} S—, R^{e} R^{f} NNR^{g}—, a nitrogen linked heteroaryl or a nitrogen linked heterocycle [wherein said heterocycle is optionally substituted on a ring carbon or a ring nitrogen by C₁₋₄ alkyl or C₁₋₄ alkanoyl] wherein R^{a}, R^{b}, R^{c}, R^{d}, R^{e}, R^{f} and R^{g} are independently selected from hydrogen, C₁₋₄ alkyl, C₂₋₄ alkenyl, C₃₋₈ cycloalkyl, and wherein said C₁₋₄ alkyl and C₂₋₄ alkenyl are optionally substituted by one or more phenyl;

n is 1, 2 or 3;

m is 1, 2or 3;

and Q¹ may optionally bear on any available carbon atom up to four substituents independently selected from halo, thio, nitro, carboxy, cyano, C₂₋₄ alkenyl [optionally substituted by up to three halo substituents, or by one trifluoromethyl substituent], C₂₋₄ alkynyl, C₁₋₅ alkanoyl, C₁₋₄ alkoxycarbonyl, C₁₋₃ alkyl, hydroxy-C₁₋₆ alkyl, fluoro-C₁₋₄ alkyl, amino-C₁₋₃ alkyl, C₁₋₄ alkylamino-C₁₋₃ alkyl, di-(C₁₋₄ alkyl)amino-C₁₋₃ alkyl, cyano-C₁₋₄ alkyl, C₂₋₄ alkanoyloxy-C₁₋₄ alkyl, C₁₋₄ alkoxy-C₁₋₃ alkyl, carboxy-C₁₋₄ alkyl, C₁₋₄ alkoxycarbonyl-C₁₋₄ alkyl, carbamoyl-C₁₋₄ alkyl, N—C₁₋₄ alkylcarbamoyl-C₁₋₄ alkyl, N,N-di-(C₁₋₄ alkyl)-carbamoyl-C₁₋₄ alkyl, pyrrolidin-1-yl-C₁₋₃ alkyl, piperidino-C₁₋₃ alkyl, piperazin-1-yl-C₁₋₃ alkyl, morpholino-C₁₋₃ alkyl, thiomorpholino-C₁₋₃ alkyl, imidazo-1-yl-C₁₋₃ alkyl, piperazin-1-yl, morpholino, thiomorpholino, C₁₋₄ alkylthio, C₁₋₄ alkylsulphinyl, C₁₋₄ alkylsulphonyl, hydroxyC₂₋₄ alkylthio, hydroxyC₂₋₄ alkylsulphinyl, hydroxyC₂₋₄ alkylsulphonyl, ureido, N—(C₁₋₄ alkyl)ureido, N′,N′-di-(C₁₋₄ alkyl)ureido, N′—(C₁₋₄ alkyl)-N—(C₁₋₄ alkyl)ureido, N′,N′-di-(C₁₋₄ alkyl)-N—(C₁₋₄ alkyl)ureido, carbamoyl, N—(C₁₋₄ alkyl)carbamoyl, N,N-di-(C₁₋₄ alkyl)carbamoyl, amino, C₁₋₄ alkylamino, di-(C₁₋₄ alkyl)amino, C₂₋₄ alkanoylamino, sulphamoyl, N—(C₁₋₄ alkyl)sulphamoyl, N,N-di-(C₁₋₄ alkyl)sulphamoyl;

and also independently, or where appropriate in addition to, the above substituents, Q₁ may optionally bear on any available carbon atom up to two further substituents independently selected from C₃₋₈ cycloalkyl, phenyl-C₁₋₄ alkyl, phenyl-C₁₋₄ alkoxy, phenylthio, phenyl, naphthyl, benzoyl, benzimidazol-2-yl, phenoxy and a 5- or 6-membered aromatic heterocycle (linked via a ring carbon atom and containing one to three heteroatoms independently selected from oxygen, sulphur and nitrogen); wherein said naphthyl, phenyl, benzoyl, phenoxy, 5- or 6-membered aromatic heterocyclic substituents and the phenyl group in said phenyl-C₁₋₄ alkyl, phenylthio and phenyl-C₁₋₄ alkoxy substituents may optionally bear up to five substituents independently selected from halo, C₁₋₄ alkyl and C₁₋₄ alkoxy;

and Q₂ may optionally bear on any available carbon atom up to four substituents independently selected from halo, hydroxy, thio, nitro, carboxy, cyano, C₂₋₄ alkenyl [optionally substituted by up to three halo substituents, or by one trifluoromethyl substituent], C₂₋₄ alkynyl, C₁₋₅ alkanoyl, C₁₋₄ alkoxycarbonyl, C₁₋₆ alkyl, hydroxy-C₁₋₃ alkyl, fluoro-C₁₋₄ alkyl, amino-C₁₋₃ alkyl, C₁₋₄ alkylamino-C₁₋₃ alkyl, di-(C₁₋₄ alkyl)amino-C₁₋₃ alkyl, cyano-C₁₋₄ alkyl, C₂₋₄ alkanoyloxy-C₁₋₄-alkyl, C₁₋₄ alkoxy-C₁₋₃ alkyl, carboxy-C₁₋₄ alkyl, C₁₋₄ alkoxycarbonyl-C₁₋₄ alkyl, carbamoyl-C₁₋₄ alkyl, N—C₁₋₄ alkylcarbamoyl-C₁₋₄ alkyl, N,N-di-(C₁₋₄ alkyl)-carbamoyl-C₁₋₄ alkyl, pyrrolidin-1-yl-C₁₋₃ alkyl, piperidino-C₁₋₃ alkyl, piperazin-1-yl-C₁₋₃ alkyl, morpholino-C₁₋₃ alkyl, thiomorpholino-C₁₋₃ alkyl, imidazo-1-yl-C₁₋₃ alkyl, piperazin-1-yl, morpholino, thiomorpholino, C₁₋₄ alkoxy, cyano-C₁₋₄ alkoxy, carbamoyl-C₁₋₄ alkoxy, N—C₁₋₄ alkylcarbamoyl-C₁₋₄ alkoxy, N,N-di-(C₁₋₄ alkyl)-carbamoyl-C₁₋₄ alkoxy, 2-aminoethoxy, 2-C₁₋₄ alkylaminoethoxy, 2-di-(C₁₋₄ alkyl)aminoethoxy, C₁₋₄ alkoxycarbonyl-C₁₋₄ alkoxy, halo-C₁₋₄ alkoxy, 2-hydroxyethoxy, C₂₋₄ alkanoyloxy-C₂₋₄ alkoxy, 2-C₁₋₄ alkoxyethoxy carboxy-C₁₋₄ alkoxy, 2-pyrrolidin-1-yl-ethoxy, 2-piperidino-ethoxy, 2-piperazin-1-yl-ethoxy, 2-morpholino-ethoxy, 2-thiomorpholino-ethoxy, 2-imidazo-1-yl-ethoxy, C₃₋₅ alkenyloxy, C₃₋₅ alkynyloxy, C₁₋₄ alkylthio, C₁₋₄ alkylsulphinyl, C₁₋₄ alkylsulphonyl, hydroxyC₂₋₄ alkylthio, hydroxyC₂₋₄ alkylsulphinyl, hydroxyC₂₋₄ alkylsulphonyl, ureido, N′-(C₁₋₄ alkyl)ureido, N′,N′-di-(C₁₋₄ alkyl)ureido, N′—(C₁₋₄ alkyl)-N—(C₁₋₄ aklyl)ureido, N′,N′-di-(C₁₋₄ alkyl)-N—(C₁₋₄ alkyl)ureido, carbamoyl, N—(C₁₋₄ alkyl)carbamoyl, N,N-di-(C₁₋₄ alkyl)carbamoyl, amino, C₁₋₄ alkylamino, di-(C₁₋₄ alkyl)amino, C₂₋₄ alkanoylamino, sulphamoyl, N—(C₁₋₄ alkyl)sulphamoyl, N,N-di-(C₁₋₄ alkyl)sulphamoyl,

and also independently, or where appropriate in addition to, the above optional substituents, Q₂ may optionally bear on any available carbon atom up to two further substituents independently selected from C₃₋₈ cycloalkyl, phenyl-C₁₋₄ alkyl, phenyl-C₁₋₄ alkoxy, phenylthio, phenyl, naphthyl, benzoyl, phenoxy, benzimidazol-2-yl, and a 5- or 6-membered aromatic heterocycle (linked via a ring carbon atom and containing one to three heteroatoms independently selected from oxygen, sulphur and nitrogen); wherein said naphthyl, phenyl, benzoyl, phenoxy, 5- or 6-membered aromatic heterocyclic substituents and the phenyl group in said phenyl-C₁₋₄ alkyl, phenylthio and phenyl-C₁₋₄ alkoxy substituents may optionally bear one or two substituents independently selected from halo, C₁₋₄ alkyl and C₁₋₄ alkoxy; or a pharmaceutically acceptable salt or in vivo hydrolysable ester thereof.

A suitable value for “heterocycle” within the definition of A in group (Ib) is a fully saturated, mono or bicyclic ring that contains 4-12 atoms, at least one of which is selected from nitrogen, sulphur or oxygen, wherein a —CH2- group can optionally be replaced by a —C(O)—, and a ring sulphur atom may be optionally oxidised to form S-oxide(s). Suitably “heterocycle” is a monocyclic ring containing 5 or 6 atoms or a bicyclic ring containing 9 or 10 atoms. “Heterocycle” may be nitrogen or carbon linked. Suitable values for “heterocycle” include morpholino, piperidyl, piperazinyl, pyrrolidinyl, thiomorpholino, homopiperazinyl, imidazolyl, imidazolidinyl, pyrazolidinyl, dioxanyl and dioxolanyl. Preferably “heterocycle” is morpholine, piperidyl, piperazinyl, pyrrolidinyl, thiomorpholine or homopiperazinyl. More preferably “heterocycle” is morpholino.

A suitable value for “heteroaryl” within the definition of A in group (Ib) is a partially unsaturated or fully unsaturated, mono or bicyclic ring that contains 4-12 atoms, at least one of which is selected from nitrogen, sulphur or oxygen, wherein a —CH₂— group can optionally be replaced by a —C(O)—, and a ring sulphur and/or nitrogen atom may be optionally oxidised to form S-oxide(s) and/or an N-oxide. Suitably “heteroaryl” is a monocyclic ring containing 5 or 6 atoms or a bicyclic ring containing 9 or 10 atoms. “Heteroaryl” may be nitrogen or carbon linked (but only nitrogen linked if the nitrogen link results in a neutral compound being formed). Suitable values for “heteroaryl” include thienyl, furyl, imidazolyl, thiazolyl, thiadiazolyl, pyrimidinyl, pyridinyl, pyrazinyl, pyridazinyl, triazinyl, pyrrolyl or pyrazolyl. Preferably “heteroaryl” is furyl, imidazolyl, thiazolyl, isoxazolyl, benzothienyl, quinolinyl, tetrazolyl and pyrazolyl. More preferably “heteroaryl” is imidazol-1-yl, fur-3-yl, isoxazol-3-yl, benzothien-6-yl, quinolin-6-yl, pyrazol-3-yl, thiazol-2-yl or tetrazol-5-yl.

A suitable value for Z in group (Ia) when it is a “nitrogen linked heteroaryl” is a mono or bicyclic ring that has a degree of unsaturation, containing 4-12 atoms, at least one of which is selected from nitrogen, and optionally 1-3 further atoms are selected from nitrogen, sulphur or oxygen, wherein a —CH₂— group can optionally be replaced by a —C(O)—, and a ring sulphur and/or nitrogen atom may be optionally oxidised to form S-oxide(s) and/or an N-oxide. Suitably “nitrogen linked heteroaryl” is a monocyclic ring containing 5 or 6 atoms or a bicyclic ring containing 9 or 10 atoms. The nitrogen link results in a neutral compound being formed. Suitable values for “nitrogen linked heteroaryl” include imidazol-1-yl, pyrrolin-1-yl, imidazolin-1-yl, pyrazolin-1-yl, triazol-1-yl, indol-1-yl, isoindol-2-yl, indolin-1-yl, benzimidazol-1-yl, pyrrol-1-yl or pyrazol-1-yl. Preferably “nitrogen linked heteroaryl” is imidazol-1-yl.

A suitable value for Z in group (Ia) when it is a “nitrogen linked heterocycle” is an unsaturated mono or bicyclic ring that contains 4-12 atoms, at least one of which is selected from nitrogen, and optionally 1-3 further atoms are selected from nitrogen, sulphur or oxygen, wherein a —CH₂— group can optionally be replaced by a —C(O)—, and a ring sulphur may be optionally oxidised to form S-oxide(s). Suitably “nitrogen linked heterocycle” is a monocyclic ring containing 5 or 6 atoms or a bicyclic ring containing 9 or 10 atoms. Suitable values for “nitrogen linked heterocycle” include pyrrolidin-1-yl, piperidino, piperazin-1-yl, morpholino, thiomorpholino, homopiperidin-y-1 or homopiperazin-1-yl. Preferably a “nitrogen linked heterocycle” is pyrrolidin-1-yl, piperazin-1-yl or morpholino.

A suitable value for Q₁ and Q₂ when it is a 5- or 6-membered monocyclic moiety containing one to three heteroatoms independently selected from nitrogen, oxygen and sulphur, or a 9- or 10-membered bicyclic heterocyclic moiety containing one or two nitrogen heteroatoms and optionally containing a further one or two heteroatoms selected from nitrogen, oxygen and sulphur; is an aromatic heterocycle, for example, pyrrole, furan, thiophene, imidazole, oxazole, isoxazole, thiazole, pyridyl, pyridazinyl, pyrimidinyl, pyrazinyl, p-isoxazine, quinolyl, isoquinolyl, cinnolinyl, quinazolinyl, quinoxalinyl, phthalazinyl or naphthyridinyl, indole, isoindazole, benzoxazole, benzimidazole, benzothiazole, imidazo[1,5-a]pyridine, imidazo[1,2-c]pyrimidine, imidazo[1,2-a]pyrimidine, imidazo[1,5-a]pyrimidine; or a partially or fully hydrogenated derivative thereof such as for example, 1,2-dihydropyridyl, 1,2-dihydroquinolyl (all linked by a ring carbon atom), provided that an unstable aminal-type link with the amino link to the pyrimidine ring is not present.

When Q₁ is a 5- or 6-membered monocyclic moiety containing one to three heteroatoms independently selected from nitrogen, oxygen and sulphur, it will be appreciated that Q₁ is linked to the pyrimidine ring in such a way that when Q₁ bears a substituent of the formula (Ia) or (Ia′) the substituent of formula (Ia) or (Ia′) is not adjacent to the —NH— link. Thus, for example, 1,2,3-triazol-4-yl or 1,2,3-triazol-5-yl, are not suitable values for Q₁ when Q₁ bears a substituent of the formula (Ia) or (Ia′). It will be appreciated that there is at least one substituent of the formula (Ia) or (Ia′) in each compound of formula (I), although such a substituent may be borne by Q₂ (in which case, when Q₁ bears no substituent of formula (Ia) or (Ia′), 1,2,3-triazol-4-yl or 1,2,3-triazol-5-yl, for example, are suitable values for Q₁).

When Q₁ or Q₂ is a 9- or 10-membered bicyclic heterocyclic moiety containing one or two nitrogen atoms it will be appreciated that Q₁ or Q₂ may be attached from either of the two rings of the bicyclic heterocyclic moiety.

Conveniently when Q₁ or Q₂ is a 5- or 6-membered monocyclic moiety or a 9- or 10-membered bicyclic heterocyclic moiety it is, for example, 2-pyridyl, 3-pyridyl, 4-pyridyl, 3-pyridazinyl, 4-pyridazinyl, 2-pyrimidinyl, 4-pyrimidinyl, 5-pyrimidinyl, 2-pyrazinyl, 2-quinolyl, 3-quinolyl, 5-quinolyl, 6-quinolyl, 7-quinolyl, 1-isoquinolyl, 3-isoquinolyl, 6-isoquinolyl, 7-isoquinolyl, 3-cinnolyl, 6-cinnolyl, 7-cinnolyl, 2-quinazolinyl, 4-quinazolinyl, 6quinazolinyl, 7-quinazolinyl, 2-quinoxalinyl, 5-quinoxalinyl, 6-quinoxalinyl, 1-phthalazinyl, 6-phthalazinyl, 1,5-naphthyridin-2-yl, 1,5-naphthyridin-3-yl, 1,6-naphthyridin-3-yl, 1,6-naphthyridin-7-yl, 1,7-naphthyridin-3-yl, 1,7-naphthyridin-6-yl, 1,8-naphthyridin-3-yl, 2,6-naphthyridin-6-yl or 2,7-naphthyridin-3-yl.

Particularly when Q₁ or Q₂ is a 5- or 6-membered monocyclic moiety or a 9- or 10-membered bicyclic heterocyclic moiety it is pyridyl, indazolyl, indolyl, quinolyl, pyrazolyl or thiazolyl. More particularly 2-pyridyl, 3-pyridyl, 4-pyridyl, 1H-5-indazolyl, 5-indolyl, 6-quinolyl, 3-pyrazolyl or 2-thiazolyl. [0418] A suitable value for Q₁ and Q₂ when it is “aryl” is a fully or partially unsaturated, mono or bicyclic carbon ring that contains 4-12 atoms. Suitably “aryl” is a monocyclic ring containing 5 or 6 atoms or a bicyclic ring containing 9 or 10 atoms. Suitable values for “aryl” include phenyl, naphthyl, tetralinyl or indanyl. Particularly “aryl” is phenyl, naphthyl or indanyl. More particularly “aryl” is phenyl or indanyl.

A suitable value for a ring substituent when it is a 5- or 6-membered aromatic heterocycle (linked via a ring carbon atom and containing one to three heteroatoms independently selected from oxygen, sulphur and nitrogen) is, for example, pyrrole, furan, thiophene, imidazole, oxazole, isoxazole, thiazole, pyridyl, pyridazinyl, pyrimidinyl, pyrazinyl or p-isoxazine.

In this specification the term “alkyl” includes both straight and branched chain alkyl groups but references to individual alkyl groups such as “propyl” are specific for the straight chain version only. An analogous convention applies to other generic terms.

Suitable values for the generic radicals (such as in R¹ and in substituents on Q₁ and Q₂ and also those in RX) referred to above include those set out below: when it is halo is, for example, fluoro, chloro, bromo and iodo; C₂₋₄ alkenyl is, for example, vinyl and allyl; C₂₋₄ alkenyl is, for example, vinyl and allyl; when it is C₃₋₅ alkenyl is, for example, allyl; when it is C₃₋₅ alkynyl is, for example, propyn-2-yl; when it is C₂₋₄ alkynyl is, for example, ethynyl and propyn-2-yl; C₂₋₆ alkynyl is, for example, ethynyl and propyn-2-yl; when it is C₃₋₆ cycloalkyl-C₁₋₆ alkyl is, for example, cyclopropylmethyl; when it is C₁₋₅ alkanoyl is, for example, formyl and acetyl; when it is C₁₋₄ alkoxycarbonyl is, for example, methoxycarbonyl, ethoxycarbonyl, propoxycarbonyl and tert-butoxycarbonyl; when it is C₁₋₃ alkyl is, for example, methyl, ethyl, propyl, isopropyl; when it is C₁₋₄ alkyl is, for example, methyl, ethyl, propyl, isopropyl, butyl, isobutyl, sec-butyl or tert-butyl; when it is C₁₋₆ alkyl is, for example, methyl, ethyl, propyl, isopropyl, butyl, isobutyl, sec-butyl, tert-butyl or 3-methylbutyl; when it is hydroxy-C₁₋₃ alkyl is, for example, hydroxymethyl, 1-hydroxyethyl, 2-hydroxyethyl and 3-hydroxypropyl; when it is fluoro-C₁₋₄ alkyl is, for example, fluoromethyl, difluoromethyl, trifluoromethyl and 2-fluoroethyl; when it is amino-C₁₋₃ alkyl is, for example, aminomethyl, 1-aminoethyl and 2-aminoethyl; when it is C₁₋₄ alkylamino-C₁₋₃-alkyl is, for example, methylaminomethyl, ethylaminomethyl, 1-methylaminoethyl, 2-methylaminoethyl, 2-ethylamimoethyl and 3-methylaminopropyl; when it is di-(C₁₋₄ alkyl)amino-C₁₋₃ alkyl is, for example, dimethylaminomethyl, diethylaminomethyl, 1-dimethylaminoethyl, 2-dimethylaminoethyl and 3-dimethylaminopropyl; when it is cyano-C₁₋₄ alkyl is, for example cyanomethyl, 2-cyanoethyl and 3-cyanopropyl; when it is C₂₋₄ alkanoyloxy-C₁₋₄-alkyl is, for example, acetoxymethyl, propionyloxymethyl, butyryloxymethyl, 2-acetoxyethyl and 3-acetoxypropyl; when it is C₁₋₄ alkoxy-C₁₋₃ alkyl is, for example, methoxymethyl, ethoxymethyl, 1-methoxyethyl, 2-methoxyethyl, 2-ethoxyethyl and 3-methoxypropyl; when it is carboxy-C₁₋₄ alkyl is, for example carboxymethyl, 1-carboxyethyl, 2-carboxyethyl and 3-carboxypropyl; when it is C₁₋₄ alkoxycarbonyl-C₁₋₄ alkyl is, for example, methoxycarbonylmethyl, ethoxycarbonylmethyl, tert-butoxycarbonylmethyl, 1-methoxycarbonylethyl, 1-ethoxycarbonylethyl, 2-methoxycarbonylethyl, 2-ethoxycarbonylethyl, 3-methoxycarbonylpropyl and 3-ethoxycarbonylpropyl; when it is carbamoyl-C₁₋₄ alkyl is, for example carbamoylmethyl, 1-carbamoylethyl, 2-carbamoylethyl and 3-carbamoylpropyl; when it is N—C₁₋₄ alkylcarbamoyl-C₁₋₄ alkyl is, for example, N-methylcarbamoylmethyl, N-ethylcarbamoylmethyl, N-propylcarbamoylmethyl, 1-(N-methylcarbamoyl)ethyl, 1-(N-ethylcarbamoyl)ethyl, 2-(N-methylcarbamoyl)ethyl, 2-(N-ethylcarbamoyl)ethyl and 3-(N-methylcarbamoyl)propyl; when it is N,N-di-(C₁₋₄ alkyl)-carbamoyl-C₁₋₄ alkyl is, for example, N,N-dimethylcarbamoylmethyl, N-ethyl-N-methylcarbamoylmethyl, N,N-diethylcarbamoylmethyl, 1-(N,N-dimethylcarbamoyl)ethyl, 1-(N,N-diethylcarbamoyl)ethyl, 2-(N,N-dimethylcarbamoyl)ethyl, 2-(N,N-diethylcarbamoyl)ethyl and 3-(N,N-dimethylcarbamoyl)propyl; when it is pyrrolidin-1-yl-C₁₋₃ alkyl is, for example, pyrrolidin-1-ylmethyl and 2-pyrrolidin-1-ylethyl; when it is piperidin-1-yl-C₁₋₃ alkyl is, for example, piperidin-1-ylmethyl and 2-piperidin-1-ylethyl; when it is piperazin-1-yl-C₁₋₃ alkyl is, for example, piperazin-1-ylmethyl and 2-piperazin-1-ylethyl; when it is morpholino-C₁₋₃ alkyl is, for example, morpholinomethyl and 2-morpholinoethyl; when it is thiomorpholino-C₁₋₃ alkyl is, for example, thiomorpholinomethyl and 2-thiomorpholinoethyl; when it is imidazo-1-yl-C₁₋₃ alkyl is, for example, imidazo-1-ylmethyl and 2-imidazo-1-ylethyl; when it is C₁₋₄ alkoxy is, for example, methoxy, ethoxy, propoxy, isopropoxy or butoxy; when it is C₁₋₃ alkoxy is, for example, methoxy, ethoxy, propoxy or isopropoxy; when it is cyano-C₁₋₄ alkoxy is, for example, cyanomethoxy, 1-cyanoethoxy, 2-cyanoethoxy and 3-cyanopropoxy; when it is carbamoyl-C₁₋₄ alkoxy is, for example, carbamoylmethoxy, 1-carbamoylethoxy, 2-carbamoylethoxy and 3-carbamoylpropoxy; when it is N—C₁₋₄ alkylcarbamoyl-C₁₋₄ alkoxy is, for example, N-methylcarbamoylmethoxy, N-ethylcarbamoylmethoxy, 2-(N-methylcarbamoyl)ethoxy, 2-(N-ethylcarbamoyl)ethoxy and 3-(N-methylcarbamoyl)propoxy; when it is N,N-di-(C₁₋₄ alkyl)-carbamoyl-C₁₋₄ alkoxy is, for example, N,N-dimethylcarbamoylmethoxy, N-ethyl-N-methylcarbamoylmethoxy, N,N-diethylcarbamoylmethoxy, 2-(N,N-dimethylcarbamoyl)ethoxy, 2-(N,N-diethylcarbamoyl)ethoxy and 3-(N,N-dimethylcarbamoyl)propoxy; when it is 2-C₁₋₄ alkylaminoethoxy is, for example, 2-(methylamino)ethoxy, 2-(ethylamino)ethoxy and 2-(propylamino)ethoxy; when it is 2-di-(C₁₋₄ alkyl)aminoethoxy is, for example, 2-(dimethylamino)ethoxy, 2-(N-ethyl-N-methylamino)ethoxy, 2-(diethylamino)ethoxy and 2-(dipropylamino)ethoxy; when it is C₁₋₄ alkoxycarbonyl-C₁₋₄ alkoxy is, for example, methoxycarbonylmethoxy, ethoxycarbonylmethoxy, 1-methoxycarbonylethoxy, 2-methoxycarbonylethoxy, 2-ethoxycarbonylethoxy and 3-methoxycarbonylpropoxy; when it is halo-C₁₋₄ alkoxy is, for example, difluoromethoxy, trifluoromethoxy, 2-fluoroethoxy, 2-chloroethoxy, 2-bromoethoxy, 3-fluoropropoxy and 3-chloropropoxy; when it is C₂₋₄ alkanoyloxy-C₂₋₄ alkoxy is, for example, 2-acetoxyethoxy, 2-propionyloxyethoxy, 2-butyryloxyethoxy and 3-acetoxypropoxy; when it is 2-C₁₋₄ alkoxyethoxy is, for example, 2-methoxyethoxy, 2-ethoxyethoxy; when it is carboxy-C₁₋₄ alkoxy is, for example, carboxymethoxy, 1-carboxyethoxy, 2-carboxyethoxy and 3-carboxypropoxy; when it is C₃₋₅ alkenyloxy is, for example, allyloxy; when it is C₃₋₅ alkynyloxy is, for example, propynyloxy; when it is C₁₋₄ alkylthio is, for example, methylthio, ethylthio or propylthio; when it is C₁₋₄ alkylthio is C₁₋₃ alkylthio; when it is C₁₋₄ alkylsulphinyl is, for example, methylsulphinyl, ethylsulphinyl or propylsulphinyl; when it is C₁₋₄ alkylsulphonyl is, for example, methylsulphonyl, ethylsulphonyl or propylsulphonyl; when it is N—C₁₋₄ alkylcarbamoyl is, for example N-methylcarbamoyl, N-ethylcarbamoyl and N-propylcarbamoyl; when it is N,N-di-(C₁₋₄ alkyl)-carbamoyl is, for example N,N-dimethylcarbamoyl, N-ethyl-N-methylcarbamoyl and N,N-diethylcarbamoyl; when it is C₁₋₄ alkylamino or C₁₋₃ alkylamino is, for example, methylamino, ethylamino or propylamino; when it is di-(C₁₋₄ alkyl)amino or di-(C₁₋₃ alkyl)amino is, for example, dimethylamino, N-ethyl-N-methylamino, diethylamino, N-methyl-N-propylamino or dipropylamino; when it is C₂₋₄ alkanoylamino is, for example, acetamido, propionamido or butyramido; when it is phenyl-C₁₋₄ alkyl is, for example benzyl or 2-phenylethyl; when it is phenyl-C₁₋₄ alkoxy is, for example benzyloxy; when it is —NHCOC₁₋₄ alkyl is, for example acetamido; when it is N-phthalimido-C₁₋₄ alkyl is, for example 2-(N-phthalimido)ethyl or 3-(N-phthalimido)propyl; when it is C₃₋₈ cycloalkyl is, for example, cyclopropyl, cyclopentyl or cyclohexyl; when it is C₁₋₄ alkanoyl is, for example, acetyl or propionyl; when it is C₁₋₄ alkanoyloxy is, for example, acetyloxy or propionyloxy; when it is C₁₋₄ alkanoylamino is, for example, acetamido; when it is N′-(C₁₋₄ alkyl)ureido is, for example, N′-methylureido or N′-ethylureido; when it is N′,N′-di-(C₁₋₄ alkyl)ureido is, for example, N′,N′-dimethylureido, N′,N′-diisopropylureido or N′-methyl-N′-propylureido; when it is N′-(C₁₋₄ alkyl)-N—(C₁₋₄ alkyl)ureido is, for example, N′-methyl-N-ethylureido or N′-methyl-N-methylureido; when it is N′,N′-di-(C₁₋₄ alkyl)-N—(C₁₋₄ alkyl)ureido is, for example, N′,N′-dimethyl-N-ethylureido, N′-methyl-N′-propyl-N-butylureido; when it is N—(C₁₋₄ alkyl)sulphamoyl is, for example, N-methylsulphamoyl or N-isopropylsulphamoyl; when it is N,N-di-(C₁₋₄ alkyl)sulphamoyl is, for example, N-methyl-N-ethylsulphamoyl or N,N-dipropylsulphamoyl.

Compounds of Formula I include, for example, 5-bromo-2-{4-[2-hydroxy-3-(N,N-dimethylamino)propoxy]anilino}-4-anilinopyrimidine; 5-bromo-2-{4-[2-hydroxy-3-(N,N-dimethylamino)propoxy]anilino}-4-(pyrid-2-ylamino)pyrimidine; 5-bromo-2-{4-[2-hydroxy-3-(isopropylamino)propoxy]anilino}-4-(6-methylpyrid-2-ylamino)pyrimidine; 5-bromo-2-{4-[3-(isopropylamino)propoxy]anilino}-4-anilinopyrimidine; 5-bromo-2-{4-[3-(imidazol-1-yl)propoxy]anilino}-4-(6-meth2-ylamino) pyrimidine; and 4-anilino-5-bromo-2-{4-[2-hydroxy-2-methyl-3-(isopropylamino)propoxy]anilino}pyrimidine.

It is understood that while a compound of Formula I may exhibit the phenomenon of tautomerism, the formula drawings within this specification expressly depict only one of the possible tautomeric forms. It is therefore to be understood that within the invention the formulae are intended to represent any tautomeric form of the depicted compound and is not to be limited merely to a specific tautomeric form depicted by the formula drawings.

Thus, in one aspect of the present invention, a method of treating Alzheimer's disease is provided comprising identifying a patient in need of such treatment, and administering to the patient a FAK2 inhibiting, Aβ42 lowering, effective amount of a compound selected from those provided above. In preferred embodiments the compound administered selectively inhibits FAK2 tyrosine kinase over other types of kinases. In a more preferred embodiment, the compound administered selectively inhibits FAK2 tyrosine kinase over other types of tyrosine kinases. In an even more preferred embodiment, the compound administered selectively inhibits FAK2 tyrosine kinase over FAK1 and FAK3 tyrosine kinases.

In another aspect of the present invention, a method of treating Alzheimer's disease is provided comprising identifying a patient in need of such treatment, and administering to the patient an amount of a compound (e.g., of those named above or provided according to Formula I, above). Preferably, the compound that is used in the methods of the invention is capable of inhibiting at least 10, 20, 30, 40, or 50 percent or more of the kinase activity of FAK2, at a concentration of 1 μM in an assay of FAK2 activity. In another preferred aspect of this embodiment, preferred compounds for use in the invention are those that have a Kᵢ for FAK2 of 50 μM or less, more preferably 10 μM or less, and even more preferably 1 μM or less. In another preferred aspect of this embodiment, preferred compounds for use in the invention are those that have an IC₅₀ for FAK2 of 50 μM or less, more preferably 10 μM or less, and even more preferably 1 μM or less.

6.4.2. Small Molecule Inhibitors of SCD:

In another embodiment, preferred compounds for use in the invention are those that inhibit the Stearoyl-CoA desaturase (SCD). SCD is a microsomal enzyme that introduces double bonds into fatty acids thus catalyzing the production of monounsaturated fatty acids. As described above, we discovered that a carboxyl-terminal fragment of SCD (comprising amino acid residues 320-359) interacts with the calcium- and integrin-binding protein (CIB), which itself interacts with both presenilins PS1 and PS2 (Stabler et al., J Cell Biol 145:1277-1292, (1999)). Based on its indirect association SCD with presenilins, and knowing that Aβ production can be modulated by local membrane lipid composition (Puglielli et al., Nat Neurosci 6:345-351 (2003)), we hypothesized that SCD activity might affect Aβ production. In agreement with this hypotheis, we have discovered that overexpression of human SCD in neuronal H4 cells expressing APP resulted in increased secretion of Aβ42 (the more pathogenic, but less abundant species) without changing the amount of secreted Aβ40 (the more abundant species). This observation suggested that inhibition of SCD activity might result in reduced levels of Aβ42—a therapeutically desirable outcome.

Recent reports have shown that conjugated linoleic acid (CLA) isomers can inhibit SCD activity (Gomez et al., BBRC 300:316-326 (2003); Park et al., Biochim Biophys Acta 1486:285-292 (2000); Choi et al., J Nutr 130:1920-1924 (2000); Choi et al., BBRC 284:689-693 (2001); Choi et al., BBRC 294:785-790 (2002)). CLA isomers are naturally found in foods such as ruminant meats and dairy products, and levels of CLA in these sources may be modified by dietary changes such as increased sunflower or soybean oils in the feed. Purified CLA isomers are commercially available and mixtures of CLA isomers can be bought as supplements in health food stores. Various studies using animal models have demonstrated the potential of CLA treatment for beneficial health effects such as decreased carcinogenesis (Ip et al., J Nutr 129:2135-2142 (1999); Ip et al., Nutr Cancer 43:52-58 (2002)), decreased atherogenesis (Lee et al., Atherosclerosis 108:19-25 (1994); Nicolosi et al., Artery 22:266-277 (1997)), decreased obesity (Park et al., Lipids 34:235-241 (1999)), and improved glucose intolerance and insulin action in diabetic models (Houseknecht et al., BBRC 244:678-682 (1998); Ryder et al., Diabetes 50:1149-1157 (2001)). Cell-based assay models have shown that CLA generally reduces the levels of cellular monounsaturated fatty acids by reducing SCD activity. The trans-10, cis-12 isomer of CLA appears to specifically inhibit SCD, while other isomers, including the trans-9, trans-11 isomer, do not (Park et al., Biochim Biophys Acta 1486:285-292, (2000)).

We treated 293/APP cells with the CLA isoforms, trans-10, cis-12 (active form) and trans-9, trans-11 (inactive form) and measured Aβ42 levels in the medium to test our hypothesis that SCD activity affects Aβ42 production. Our preliminary results show that treatment of cells with 10, 33, or 100 μM of the trans-10, cis-12 isoform resulted in a decrease of Aβ42 levels by 40-50%, but did not have a significant effect on Aβ40 levels. A non-significant decrease in Aβ42 levels was observed when the cells were treated with higher concentrations of the trans-9, trans-11 isoform (33 and 100 μM). Cells treated by either CLA isoform at 33 and 100 μM appeared normal, but there were some bare patches of tissue culture plastic in these wells, indicating that these concentrations of CLA may be somewhat toxic to the 293 cells, and therefore, the reduction of Aβ42 secretion observed at higher CLA concentrations may not reflect specific inhibition of SCD. Advantageously, our preliminary results support our hypothesis that SCD inhibitors, including (but not limited to) the trans-10, cis-12 isoform of CLA, (and also a thiourea isoxyl anti-tuberculosis drug) may alter APP processing and cause decreased secretion of the toxic Aβ42 peptide. By decreasing Aβ42 levels, the pathology of Alzheimer's disease may be lessened.

To summarize, our earlier studies showed that SCD overexpression increases Aβ42 secretion, and we now report that a known SCD inhibitor, the trans-10 cis-12 isoform of CLA, reduces Aβ42 secretion. A similar compound which differs only in the position and geometry of the double bonds, and which does not inhibit SCD, does not appear to decrease Aβ42 secretion. Although others have suggested the use of CLAs and other SCD inhibitors for the treatment of diseases such as obesity, diabetes, cancer, atherosclerosis, and inflammation, we are suggesting a novel use of CLAs and other SCD inhibitors (including a former anti-tuberculosis drug, isoxyl [Phetsuksiri et al., J Biol Chem 278:53123-53130, (2003)]) for the treatment of Alzheimer's disease via lowering Aβ42 levels.

In another embodiment of the present invention, screening assays designed to detect inhibition of SCD activity are used to identify additional small molecules that can be used to treat AD or its symptoms, or to delay the onset of AD symptoms. Assays for SCD activity are based upon those of Cohen and coworkers (see Cohen et al., Science 297:240-243 (2002), which is incorporated herein by reference in its entirety). Briefly, the SCD activity assays are conducted as follows: These assays measure the conversion of of [1-¹⁴C]stearoyl-CoA to [1-¹⁴C]oleate as in (S3). Tissues are homogenized in 10 volumes of buffer A (0.25 M sucrose, 1 mM EDTA, 10 mM Tris-HCl, 1 mM PMSF, pH 7.4). The microsomal fractions (100,000 g) are isolated by sequential centrifugation. Reactions are performed at 37° C. for 5 min with 100 μg protein homogenate and 6 μM of [1-¹⁴C]stearoyl-CoA (60,000 rpm), 2 mM NADH, 0.1 M Tris-HCl, pH 7.2. After the reaction, fatty acids are extracted and methylated with 10% acetic chloride/methanol. Saturated and monounsaturated fatty acid methyl esters are separated by 10% AgNO₃-impregnated thin-layer chromatography (TLC) using hexane:diethyl ether (9:1) as the developing solution. The plates are sprayed with 0.2% 2′,7′-dichloroflourescein in 95% ethanol, and the lipids are identified under UV light. Fractions are scraped off of the plate, and radioactivity is measured by using a liquid scintillation counter.

Thus, in one another of this embodiment, a method of treating Alzheimer's disease is provided comprising identifying a patient in need of such treatment, and administering to the patient an SCD inhibiting, Aβ42 lowering, effective amount of a compound. In another aspect of this embodiment, a method of treating Alzheimer's disease is provided comprising identifying a patient in need of such treatment, and administering to the patient an amount of a compound necessary to effectively inhibit SCD. Preferably, the compound that is used in the methods of the invention is capable of inhibiting at least 10, 20, 30, 40, or 50 percent or more of the kinase activity of SCD, at a concentration of 1 μM in an assay of SCD activity. In another preferred aspect of this embodiment, preferred compounds for use in the invention are those that have a Ki for SCD of 50 μM or less, more preferably 10 μM or less, and even more preferably 1 μM or less. In another preferred aspect of this embodiment, preferred compounds for use in the invention are those that have an IC50 for SCD of 50 μM or less, more preferably 10 μM or less, and even more preferably 1 μM or less.

One compound that can be used to inhibit the activity of SCD, and can therefore be employed in this embodiment of the instant invention, is 7,8-dihydro-5-methyl-8-(1-phenylethyl)-6H-pyrrolo [3,2-e] [1,2,4] triazolo [1,5-a] pyrimidine (also known as RS-1178). This compound was recently shown to specifically inhibit neuronal cell death mediated by beta-amyloid-induced marcrophage activation in vitro (Uryu et al., Brain Res 946:298-306 (2002)), and even more recently, was found to suppress to basal levels, the upregulation of expression of SCD-1 in macrophages exposed to beta-amyloid (Uryu et al., Biochem Biophys Res Commun 303:302-305 (2003). Another compound that has been long known to inhibit the activity of SCD, and can therefore also be employed in this embodiment of the instant invention to prevent, delay the onset, treat the symptoms, or slow the decline caused by AD, is arachadonic acid (Tebbey & Buttke, Biochem Biophys Acta 1171:27-34 (1992).

6.4.3. Small Molecule Inhibitors of PN7740:

As mentioned above, PN7740 is a novel protein containing a protein phosphatase 2C domain, which likely acts to dephosphorylate specific phospho-serine or phospho-threonine residues on particular protein substrates. Although the precise role played by protein phosphatase 2Cs in AD pathogenesis has yet to be defined, we have discovered that fragments of PN7740 interact with the first PTB domain of Fe65 (also known as APBB1(710) or amyloid beta (A4) precursor protein-binding, family B, member 1, isoform E9 (710)), suggesting that PN7740 may well be involved somehow. Interestingly, a recent study (Ahlemeyer et al., Eur J Pharmacol 430:1-7 (2001)) found that ginkgolic acids, which are known to have concentration-depepndent neurotoxic effects when applied to cultured chick embryonic neurons specifically increased the activity of protein phosphatase type-2C, whereas other protein phosphatases such as protein phosphatase 1A, 2A and 2B, tyrosine phosphatase, and unspecific acid- and alkaline phosphatases were inhibited or remain unchanged. This observation lead the authors to suggest that protein phosphatase 2C plays a role in the neurotoxic effect mediated by ginkgolic acids.

Given the potential involvement of PN7740 in AD pathogenesis, the present invention provides for compounds that specifically inhibit its protein phosphatase 2C activity. Such compounds can have therapeutic benefits for the treatment of AD and its symptoms. The protein phosphatase 2C enzymatic activity of PN7740 has been confirmed, and can be assayed using established techniques (e.g., Ahlemeyer et al., Eur J Pharmacol 430:1-7 (2001); which is incorporated herein in its entirety by reference). Screening for modulators of PN7740 can be accomplished by the methods described in Section 5.1-5.3. Potential compounds that inhibit protein phosphatase 2C, and thus are potential inhibitors of PN7740, and therefore are agents potentially useful for the treatment of AD and its symptoms, include alpha-linolenic acid, which has been shown to be a potent inhibitor of protein phosphatase 2C in alfalfa (Baudouin et al., Plant J 20:343-348 (1999)), and uridine-5′-diphospho-N-acetylglucosamine, which inhibits the protein phosphatase 2C of the aquatic fungus Blastocladiella emersonii (Etchebehere et al., J Bacteriol 175:5022-5027 (1993)).

Thus, in one another of this embodiment, a method of treating Alzheimer's disease is provided comprising identifying a patient in need of such treatment, and administering to the patient a PN7740/phosphatase 2c inhibiting, Aβ₄₂ lowering, effective amount of a compound, such as those described above. In another aspect of this embodiment, a method of treating Alzheimer's disease is provided comprising identifying a patient in need of such treatment, and administering to the patient an amount of a compound necessary to effectively inhibit PN7740/phosphatase 2c. Preferably, the compound that is used in the methods of the invention is capable of inhibiting at least 10, 20, 30, 40, or 50 percent or more of the kinase activity of PN7740/phosphatase 2c, at a concentration of 1 μM in an assay of PN7740/phosphatase 2c activity. In another preferred aspect of this embodiment, preferred compounds for use in the invention are those that have a Ki for PN7740/phosphatase 2c of 50 μM or less, more preferably 10 μM or less, and even more preferably 1 μM or less. In another preferred aspect of this embodiment, preferred compounds for use in the invention are those that have an IC₅₀ for PN7740/phosphatase 2c of 50 μM or less, more preferably 10 μM or less, and even more preferably 1 μM or less.

### 7. Cell and Animal Models

In another aspect of the present invention, cell and animal models are provided in which one or more of the constituent proteins of the interacting pairs of proteins described in the tables, exhibit aberrant function, activity, or concentration when compared with wild type cells and animals (e.g., increased or decreased concentration, altered interactions between protein complex constituents due to mutations in interaction domains, and/or altered distribution or localization of the proteins in organs, tissues, cells, or cellular compartments). Such cell and animal models are useful tools for studying cellular functions and biological processes associated with the proteins identified in the tables. Such cell and animal models are also useful tools for studying disorders and diseases associated with the proteins identified in the tables, and for testing various methods for modulating the cellular functions, and for treating the diseases and disorders, associated with aberrations in these proteins. For example, a cell or animal model may be used to determine if δ-catenin exhibits aberrant function, activity, or concentration when compared with wild type cells or animals. In another example, a cell or animal model may be used to determine if FAK2 exhibits aberrant function, activity, or concentration when compared with wild type cells or animals. A preferred example of a cell-based model for use in the methods of the present invention is provided as Example 8, below. This particular model readily allows for the assessment of whether or not compounds affect the processing of APP, the production of Aβ, and the secretion of the neurotoxic Aβ42 peptide.

Preferred examples of animal models for use in the methods of the present invention are provided as Examples 9, 10 and 11, below. These three examples facilitate the assessment of whether or not an administered compound has a desired effect on levels of Aβ42, neuronal survival and memory, respectively.

### 7.1. Cell Models

Cell models having an aberrant form of one or more of the proteins or protein complexes identified in the tables are provided in accordance with the present invention.

The cell models may be established by isolating, from a patient, cells having an aberrant form of one or more of the protein complexes of the present invention. The isolated cells may be cultured in vitro as a primary cell culture. Alternatively, the cells obtained from the primary cell culture or directly from the patient may be immortalized to establish a human cell line. Any methods for constructing immortalized human cell lines may be used in this respect. See generally Yeager & Reddel, Curr Opin Biotech, 10:465-469 (1999). For example, the human cells may be immortalized by transfection of plasmids expressing the SV40 early region genes (See e.g., Jha et al., Exp Cell Res, 245:1-7 (1998)), introduction of the HPV E6 and E7 oncogenes (See e.g., Reznikoff et al., Genes Dev, 8:2227-2240 (1994)), and infection with Epstein-Barr virus (See e.g., Tahara et al., Oncogene, 15:1911-1920 (1997)). Alternatively, the human cells may be immortalized by recombinantly expressing the gene for the human telomerase catalytic subunit hTERT in the human cells. See Bodnar et al., Science, 279:349-352 (1998).

In alternative embodiments, cell models are provided by recombinantly manipulating appropriate host cells. The host cells may be bacteria cells, yeast cells, insect cells, plant cells, animal cells, and the like. Preferably, the cells are derived from mammals, most preferably humans. The host cells may be obtained directly from an individual, or a primary cell culture, or preferably an immortal stable human cell line. In a preferred embodiment, human embryonic stem cells or pluripotent cell lines derived from human stem cells are used as host cells. Methods for obtaining such cells are disclosed in, e.g., Shamblott, et al., Proc Natl Acad Sci USA, 95:13726-13731 (1998) and Thomson et al., Science, 282:1145-1147 (1998).

In one embodiment, a cell model is provided by recombinantly expressing one or more of the proteins or protein complexes identified in the tables in cells that do not normally express such protein complexes. For example, cells that do not contain a particular protein or protein complex may be engineered to express the protein or protein complex. In a specific embodiment, a particular human protein complex is expressed in non-human cells. The cell model may be prepared by introducing into host cells nucleic acids encoding all interacting protein members required for the formation of a particular protein complex, and expressing the protein members in the host cells. For this purpose, the recombinant expression methods described in Section 2.2 may be used. In addition, the methods for introducing nucleic acids into host cells disclosed in the context of gene therapy in Section 6.3.2 may also be used.

In another embodiment, a cell model over-expressing one or more of the proteins or protein complexes identified in the tables. The cell model may be established by increasing the expression level of one or more of the interacting protein members of the protein complexes. In a specific embodiment, all interacting protein members of a particular protein complex are over-expressed. The over-expression may be achieved by introducing into host cells exogenous nucleic acids encoding the proteins to be over-expressed, and selecting those cells that over-express the proteins. The expression of the exogenous nucleic acids may be transient or, preferably stable. The recombinant expression methods described in Section 2.2, and the methods for introducing nucleic acids into host cells disclosed in the context of gene therapy in Section 6.3.2 may be used. Alternatively, the gene activation method disclosed in U.S. Pat. No. 5,641,670 can be used. Any host cells may be employed for establishing the cell model. Preferably, human cells lacking a protein or protein complex to be over-expressed, or having a normal concentration of the protein or protein complex, are used as host cells. The host cells may be obtained directly from an individual, or a primary cell culture, or preferably a stable immortal human cell line. In a preferred embodiment, human embryonic stem cells or pluripotent cell lines derived from human stem cells are used as host cells. Methods for obtaining such cells are disclosed in, e.g., Shamblott, et al., Proc Natl Acad Sci USA, 95:13726-13731 (1998), and Thomson et al., Science, 282:1145-1147 (1998).

In yet another embodiment, a cell model expressing an abnormally low level of one or more of the proteins or protein complexes identified in the tables is provided. Typically, the cell model is established by genetically manipulating cells that express a normal and detectable level of a protein or protein complex identified in the tables. Generally the expression level of one or more of the interacting protein members of the protein complex is reduced by recombinant methods. In a specific embodiment, the expression of all interacting protein members of a particular protein complex is reduced. The reduced expression may be achieved by “knocking out” the genes encoding one or more interacting protein members. Alternatively, mutations that can cause reduced expression level (e.g., reduced transcription and/or translation efficiency, and decreased mRNA stability) may also be introduced into the gene by homologous recombination. A gene encoding a ribozyme, antisense, or siRNA compound specific to the mRNA encoding an interacting protein member may also be introduced into the host cells, preferably stably integrated into the genome of the host cells. In addition, a gene encoding an antibody or fragment thereof specific to an interacting protein member may also be introduced into the host cells. The recombinant expression methods described in Sections 2.2, 6.1 and 6.2 can all be used for purposes of manipulating the host cells.

In a specific embodiment, an siRNA compound specific to the mRNA encoding δ-catenin is introduced into a host cell in order to decrease the expression level of δ-catenin. In another specific embodiment, an siRNA compound specific to the mRNA encoding FAK2 is introduced into a host cell in order to decrease the expression level of FAK2.

The present invention also contemplates a cell model provided by recombinant DNA techniques that exhibits aberrant interactions between the interacting protein members of a protein complex identified in the present invention. For example, variants of the interacting protein members of a particular protein complex exhibiting altered protein-protein interaction properties and the nucleic acid variants encoding such variant proteins may be obtained by random or site-directed mutagenesis in combination with a protein-protein interaction assay system, particularly the yeast two-hybrid system described in Section 5.3.1. Essentially, the genes encoding one or more interacting protein members of a particular protein complex may be subject to random or site-specific mutagenesis and the mutated gene sequences are used in yeast two-hybrid system to test the protein-protein interaction characteristics of the protein variants encoded by the gene variants. In this manner, variants of the interacting protein members of the protein complex may be identified that exhibit altered protein-protein interaction properties in forming the protein complex, e.g., increased or decreased binding affinity, and the like. The nucleic acid variants encoding such protein variants may be introduced into host cells by the methods described above, preferably into host cells that normally do not express the interacting proteins.

### 7.2. Cell-Based Assays

The cell models of the present invention containing an aberrant form of a protein or protein complex identified in the tables are useful in screening assays for identifying compounds useful in treating neurodegenerative disorders and diseases involving Aβ production or secretion, neuronal apoptosis, neuronal survival or protection, neurotransmission, axonal guidance or nervous system development, signal transduction, calcium homeostasis, mitochondrial function or intermediary metabolism such as such as Alzheimer's disease, Parkinson's disease, Lou Gehrig's disease or amyotrophic lateral sclerosis, and Down syndrome. In addition, they may also be used in in vitro pre-clinical assays for testing compounds, such as those identified in the screening assays of the present invention.

For example, cells may be treated with compounds to be tested and assayed for the compound's activity. A variety of parameters relevant to particularly physiological disorders or diseases may be analyzed.

As mentioned above, the cell-based assay provided as Example 8, below, is particularly well-suited for selecting inhibitors of the interacting proteins of the instant invention that have the highly desirable effect of altering the processing of APP, reducing the production of Aβ in general, and decreasing the secretion of the neurotoxic Aβ42 peptide, in particular. Additionally, as mentioned above, this same cell-based assay can be used to evaluate the descendents of lead compounds subjected to iterative rounds of SAR. Those compounds found to have the most desirable effects in the cell-based assays of the instant invention can then be tested in subsequent pre-clinical trials in animals, before ultimately being tested in clinical trials in human patients in need of such treatment.

### 7.3. Transgenic Animals

In another aspect of the present invention, transgenic non-human animals are created expressing an aberrant form of one or more of the protein complexes of the present invention. Animals of any species may be used to generate the transgenic animal models, including but not limited to, mice, rats, hamsters, sheep, pigs, rabbits, guinea pigs, preferably non-human primates such as monkeys, chimpanzees, baboons, and the like.

In one embodiment, transgenic animals are made to over-express one or more protein complexes formed from a first protein, which is any one of the proteins described in the tables, or a derivative, fragment or homologue thereof (including the animal counterpart of the first protein, i.e., an orthologue) and a second protein, which is any of the proteins described in the tables that interacts with the first protein, or derivatives, fragments or homologues thereof (including orthologues). Over-expression may be directed in a tissue or cell type that normally expresses animal counterparts of such protein complexes. Consequently, the concentration of the protein complex(es) will be elevated to higher levels than normal. Alternatively, the one or more protein complexes are expressed in tissues or cells that do not normally express such proteins and hence do not normally contain the protein complexes of the present invention. In a specific embodiment, a first protein, which is any one of the proteins described in the tables which is a human protein and a second protein, which is any of the proteins described in the tables that interacts with the first protein and is a human protein, are expressed in the transgenic animals.

To achieve over-expression in transgenic animals, the transgenic animals are made such that they contain and express exogenous, orthologous genes encoding a first protein, which is any of the proteins identified in the tables or a homologue, derivative or mutant form thereof, and one or more second proteins, which are any of the proteins described in the tables that interact with the first protein, or homologues, derivatives or mutant forms thereof. Preferably, the exogenous genes are human genes. Such exogenous genes may be operably linked to a native or non-native promoter, preferably a non-native promoter. For example, an exogenous gene encoding one of the proteins described in the tables may be operably linked to a promoter that is not the native promoter of that protein. If the expression of the exogenous gene is desired to be limited to a particular tissue, an appropriate tissue-specific promoter may be used.

Over-expression may also be achieved by manipulating the native promoter to create mutations that lead to gene over-expression, or by a gene activation method such as that disclosed in U.S. Pat. No. 5,641,670 as described above.

In another embodiment, the transgenic animal expresses an abnormally low concentration of the complex comprising at least one of the interacting pairs of proteins described in the tables. In a specific embodiment, the transgenic animal is a “knockout” animal wherein the endogenous gene encoding the animal orthologue of a first protein, which is any of the proteins described in the tables, and/or an endogenous gene encoding an animal orthologue of a second protein, which is any of the proteins identified in the tables that interacts with the first protein, are knocked out. In a specific embodiment, the expression of the animal orthologues of both the first and second proteins are reduced or knocked out. The reduced expression may be achieved by knocking out the genes encoding one or both interacting protein members, typically by homologous recombination. Alternatively, mutations that can cause reduced expression (e.g., reduced transcription and/or translation efficiency, or decreased mRNA stability) may also be introduced into the endogenous genes by homologous recombination. Genes encoding ribozymes or antisense compounds specific to the mRNAs encoding the interacting protein members may also be introduced into the transgenic animal. In addition, genes encoding antibodies or fragments thereof specific to the interacting protein members may also be introduced into the transgenic animal.

In an alternate embodiment, transgenic animals are made in which the endogenous genes encoding the animal orthologues of any of the proteins described in the tables are replaced with orthologous human genes.

In yet another embodiment, the transgenic animal of this invention expresses specific mutant forms of any of the proteins described in the tables that exhibit aberrant interactions. For this purpose, variants of any of the proteins described in the tables exhibiting altered protein-protein interaction properties, and the nucleic acid variants encoding such variant proteins, may be obtained by random or site-specific mutagenesis in combination with a protein-protein interaction assay system, particularly the yeast two-hybrid system described in Section 5.3.1. For example, variants of δ-catenin and FAK2 exhibiting increased, decreased or abolished binding affinity to each other may be identified and isolated. The transgenic animal of the present invention may be made to express such protein variants by modifying the endogenous genes. Alternatively, the nucleic acid variants may be introduced exogenously into the transgenic animal genome to express the protein variants therein. In a specific embodiment, the exogenous nucleic acid variants are derived from orthologous human genes and the corresponding endogenous genes are knocked out.

Any techniques known in the art for making transgenic animals may be used for purposes of the present invention. For example, the transgenic animals of the present invention may be provided by methods described in, e.g., Jaenisch, Science, 240:1468-1474 (1988); Capecchi, et al., Science, 244:1288-1291 (1989); Hasty et al., Nature, 350:243 (1991); Shinkai et al., Cell, 68:855 (1992); Mombaerts et al., Cell, 68:869 (1992); Philpott et al., Science, 256:1448 (1992); Snouwaert et al., Science, 257:1083 (1992); Donehower et al., Nature, 356:215 (1992); Hogan et al., Manipulating the Mouse Embryo; A Laboratory Manual, 2^{nd edition}, Cold Spring Harbor Laboratory Press, 1994; and U.S. Pat. Nos. 4,873,191; 5,800,998; 5,891,628, all of which are incorporated herein by reference.

Generally, the founder lines may be established by introducing appropriate exogenous nucleic acids into, or modifying an endogenous gene in, germ lines, embryonic stem cells, embryos, or sperm which are then used in producing a transgenic animal. The gene introduction may be conducted by various methods including those described in Sections 2.2, 6.1 and 6.2. See also, Van der Putten et al., Proc Natl Acad Sci USA, 82:6148-6152 (1985); Thompson et al., Cell, 56:313-321 (1989); Lo, Mol Cell Biol, 3:1803-1814 (1983); Gordon, Trangenic Animals, Intl. Rev. Cytol. 115:171-229 (1989); and Lavitrano et al., Cell, 57:717-723 (1989). In a specific embodiment, the exogenous gene is incorporated into an appropriate vector, such as those described in Sections 2.2 and 6.2, and is transformed into embryonic stem (ES) cells. The transformed ES cells are then injected into a blastocyst. The blastocyst with the transformed ES cells is then implanted into a surrogate mother animal. In this manner, a chimeric founder line animal containing the exogenous nucleic acid (transgene) may be produced.

Preferably, site-specific recombination is employed to integrate the exogenous gene into a specific predetermined site in the animal genome, or to replace an endogenous gene or a portion thereof with the exogenous sequence. Various site-specific recombination systems may be used including those disclosed in Sauer, Curr Opin Biotechnol, 5:521-527 (1994); Capecchi, et al., Science, 244:1288-1291 (1989); and Gu et al., Science, 265:103-106 (1994). Specifically, the Cre/lox site-specific recombination system known in the art may be conveniently used which employs the bacteriophage P1 protein Cre recombinase and its recognition sequence loxP. See Rajewsky et al., J Clin Invest, 98:600-603 (1996); Sauer, Methods, 14:381-392 (1998); Gu et al., Cell, 73:1155-1164 (1993); Araki et al., Proc Natl Acad Sci USA, 92:160-164 (1995); Lakso et al., Proc Natl Acad Sci USA, 89:6232-6236 (1992); and Orban et al., Proc Natl Acad Sci USA, 89:6861-6865 (1992).

The transgenic animals of the present invention may be transgenic animals that carry a transgene in all cells or mosaic transgenic animals carrying a transgene only in certain cells, e.g., somatic cells. The transgenic animals may have a single copy or multiple copies of a particular transgene.

The founder transgenic animals thus produced may be bred to produce various offsprings. For example, they can be inbred, outbred, and crossbred to establish homozygous lines, heterozygous lines, and compound homozygous or heterozygous lines.

### 8. Pharmaceutical Compositions and Formulations

In another aspect of the present invention, pharmaceutical compositions are also provided containing one or more of the therapeutic agents provided in the present invention as described in Section 6. The compositions are prepared as a pharmaceutical formulation suitable for administration into a patient. Accordingly, the present invention also extends to pharmaceutical compositions, medicaments, drugs or other compositions containing one or more of the therapeutic agent in accordance with the present invention.

For example, such therapeutic agents include, but are not limited to, (1) small organic compounds selected based on the screening methods of the present invention capable of interfering with the interaction between a first protein which is any of the interacting proteins described in the tables and a second protein which is any of the proteins identified in the tables that interacts with the first protein, (2) antisense compounds specifically hybridizable to nucleic acids (gene or mRNA) encoding the first protein (3) antisense compounds specific to the gene or mRNA encoding the second protein, (4) ribozyme compounds specific to nucleic acids (gene or mRNA) encoding the first protein, (5) ribozyme compounds specific to the gene or mRNA encoding the second protein, (6) antibodies immunoreactive with the first protein or the second protein, (7) antibodies selectively immunoreactive with a protein complex of the present invention, (8) small organic compounds capable of binding a protein complex of the present invention, (9) small peptide compounds as described above (optionally linked to a transporter) capable of interacting with the first protein or the second protein, (10) nucleic acids encoding the antibodies or peptides, (11) siRNA compounds specific to the gene or mRNA encoding the first protein, (12) siRNA compounds specific to the gene or mRNA encoding the second protein, etc.

The compositions are prepared as a pharmaceutical formulation suitable for administration into a patient. Accordingly, the present invention also extends to pharmaceutical compositions, medicaments, drugs or other compositions containing one or more of the therapeutic agent in accordance with the present invention.

In the pharmaceutical composition, an active compound identified in accordance

with the present invention can be in any pharmaceutically acceptable salt form. As used herein, the term “pharmaceutically acceptable salts” refers to the relatively non-toxic, organic or inorganic salts of the compounds of the present invention, including inorganic or organic acid addition salts of the compound. Examples of such salts include, but are not limited to, hydrochloride salts, sulfate salts, bisulfate salts, borate salts, nitrate salts, acetate salts, phosphate salts, hydrobromide salts, laurylsulfonate salts, glucoheptonate salts, oxalate salts, oleate salts, laurate salts, stearate salts, palmitate salts, valerate salts, benzoate salts, naththylate salts, mesylate salts, tosylate salts, citrate salts, lactate salts, maleate salts, succinate salts, tartrate salts, fumarate salts, and the like. See, e.g., Berge, et al., J Pharm Sci, 66:1-19 (1977).

For oral delivery, the active compounds can be incorporated into a formulation that includes pharmaceutically acceptable carriers such as binders (e.g., gelatin, cellulose, gum tragacanth), excipients (e.g., starch, lactose), lubricants (e.g., magnesium stearate, silicon dioxide), disintegrating agents (e.g., alginate, Primogel, and corn starch), and sweetening or flavoring agents (e.g., glucose, sucrose, saccharin, methyl salicylate, and peppermint). The formulation can be orally delivered in the form of enclosed gelatin capsules or compressed tablets. Capsules and tablets can be prepared in any conventional techniques. The capsules and tablets can also be coated with various coatings known in the art to modify the flavors, tastes, colors, and shapes of the capsules and tablets. In addition, liquid carriers such as fatty oil can also be included in capsules.

Suitable oral formulations can also be in the form of suspension, syrup, chewing gum, wafer, elixir, and the like. If desired, conventional agents for modifying flavors, tastes, colors, and shapes of the special forms can also be included. In addition, for convenient administration by enteral feeding tube in patients unable to swallow, the active compounds can be dissolved in an acceptable lipophilic vegetable oil vehicle such as olive oil, corn oil and safflower oil.

The active compounds can also be administered parenterally in the form of solution or suspension, or in lyophilized form capable of conversion into a solution or suspension form before use. In such formulations, diluents or pharmaceutically acceptable carriers such as sterile water and physiological saline buffer can be used. Other conventional solvents, pH buffers, stabilizers, anti-bacterial agents, surfactants, and antioxidants can all be included. For example, useful components include sodium chloride, acetate, citrate or phosphate buffers, glycerin, dextrose, fixed oils, methyl parabens, polyethylene glycol, propylene glycol, sodium bisulfate, benzyl alcohol, ascorbic acid, and the like. The parenteral formulations can be stored in any conventional containers such as vials and ampoules.

Routes of topical administration include nasal, bucal, mucosal, rectal, or vaginal applications. For topical administration, the active compounds can be formulated into lotions, creams, ointments, gels, powders, pastes, sprays, suspensions, drops and aerosols. Thus, one or more thickening agents, humectants, and stabilizing agents can be included in the formulations. Examples of such agents include, but are not limited to, polyethylene glycol, sorbitol, xanthan gum, petrolatum, beeswax, or mineral oil, lanolin, squalene, and the like. A special form of topical administration is delivery by a transdermal patch. Methods for preparing transdermal patches are disclosed, e.g., in Brown, et al., Ann Rev Med, 39:221-229 (1988), which is incorporated herein by reference.

Subcutaneous implantation for sustained release of the active compounds may also be a suitable route of administration. This entails surgical procedures for implanting an active compound in any suitable formulation into a subcutaneous space, e.g., beneath the anterior abdominal wall. See, e.g., Wilson et al., J Clin Psych 45:242-247 (1984). Hydrogels can be used as a carrier for the sustained release of the active compounds. Hydrogels are generally known in the art. They are typically made by crosslinking high molecular weight biocompatible polymers into a network that swells in water to form a gel like material. Preferably, hydrogels is biodegradable or biosorbable. For purposes of this invention, hydrogels made of polyethylene glycols, collagen, or poly(glycolic-co-L-lactic acid) may be useful. See, e.g., Phillips et al., J Pharmaceut Sci 73:1718-1720 (1984).

The active compounds can also be conjugated, to a water soluble non-immunogenic non-peptidic high molecular weight polymer to form a polymer conjugate. For example, an active compound is covalently linked to polyethylene glycol to form a conjugate. Typically, such a conjugate exhibits improved solubility, stability, and reduced toxicity and immunogenicity. Thus, when administered to a patient, the active compound in the conjugate can have a longer half-life in the body, and exhibit better efficacy. See generally, Burnham, Am J Hosp Pharm, 15:210-218 (1994). PEGylated proteins are currently being used in protein replacement therapies and for other therapeutic uses. For example, PEGylated interferon (PEG-INTRON A®) is clinically used for treating Hepatitis B. PEGylated adenosine deaminase (ADAGEN®) is being used to treat severe combined immunodeficiency disease (SCIDS). PEGylated L-asparaginase (ONCAPSPAR®) is being used to treat acute lymphoblastic leukemia (ALL). It is preferred that the covalent linkage between the polymer and the active compound and/or the polymer itself is hydrolytically degradable under physiological conditions. Such conjugates known as “prodrugs” can readily release the active compound inside the body. Controlled release of an active compound can also be achieved by incorporating the active ingredient into microcapsules, nanocapsules, or hydrogels generally known in the art.

Liposomes can also be used as carriers for the active compounds of the present invention. Liposomes are micelles made of various lipids such as cholesterol, phospholipids, fatty acids, and derivatives thereof. Various modified lipids can also be used. Liposomes can reduce the toxicity of the active compounds, and increase their stability. Methods for preparing liposomal suspensions containing active ingredients therein are generally known in the art. See, e.g., U.S. Pat. No. 4,522,811; Prescott, Ed., Methods in Cell Biology, Volume XIV, Academic Press, New York, N.Y. (1976).

The active compounds can also be administered in combination with another active agent that synergistically treats or prevents the same symptoms or is effective for another disease or symptom in the patient treated so long as the other active agent does not interfere with or adversely affect the effects of the active compounds of this invention. Such other active agents include but are not limited to anti-inflammation agents, antiviral agents, antibiotics, antifungal agents, antithrombotic agents, cardiovascular drugs, cholesterol lowering agents, anti-cancer drugs, hypertension drugs, and the like.

Generally, the toxicity profile and therapeutic efficacy of the therapeutic agents can be determined by standard pharmaceutical procedures in cell models or animal models, e.g., those provided in Section 7. As is known in the art, the LD₅₀ represents the dose lethal to about 50% of a tested population. The ED₅₀ is a parameter indicating the dose therapeutically effective in about 50% of a tested population. Both LD₅₀ and ED₅₀ can be determined in cell models and animal models. In addition, the IC₅₀ may also be obtained in cell models and animal models, which stands for the circulating plasma concentration that is effective in achieving about 50% of the maximal inhibition of the symptoms of a disease or disorder. Such data may be used in designing a dosage range for clinical trials in humans. Typically, as will be apparent to skilled artisans, the dosage range for human use should be designed such that the range centers around the ED₅₀ and/or IC₅₀, but significantly below the LD₅₀ obtained from cell or animal models.

It will be apparent to skilled artisans that therapeutically effective amount for each active compound to be included in a pharmaceutical composition of the present invention can vary with factors including but not limited to the activity of the compound used, stability of the active compound in the patient's body, the severity of the conditions to be alleviated, the total weight of the patient treated, the route of administration, the ease of absorption, distribution, and excretion of the active compound by the body, the age and sensitivity of the patient to be treated, and the like. The amount of administration can also be adjusted as the various factors change over time.

### EXAMPLES

1. Yeast Two-Hybrid System

The principles and methods of the yeast two-hybrid system have been described in detail in The Yeast Two-Hybrid System, Bartel and Fields, eds., pages 183-196, Oxford University Press, New York, N.Y., 1997. The following is thus a description of the particular procedure that we used to identify the protein-protein interactions of the present invention.

The cDNA encoding the bait protein was generated by PCR from cDNA prepared from a desired tissue. The cDNA product was then introduced by recombination into the yeast expression vector pGBT.Q, which is a close derivative of pGBT.C (See Bartel et al., Nat Genet, 12:72-77 (1996)) in which the polylinker site has been modified to include M13 sequencing sites. The new construct was selected directly in the yeast strain PNY200 for its ability to drive tryptophane synthesis (genotype of this strain: MATa trp1-901 leu2-3,112 ura3-52 his3-200 ade2 gal4 Δ gal80). In these yeast cells, the bait was produced as a C-terminal fusion protein with the DNA binding domain of the transcription factor Gal4 (amino acids 1 to 147). Prey libraries were transformed into the yeast strain BK100 (genotype of this strain: MATa trp1-901 leu2-3,112 ura3-52 his3-200 gal4 Δ gal80 LYS2::GAL-HIS3 GAL2-ADE2 met2::GAL7-lacZ), and selected for the ability to drive leucine synthesis. In these yeast cells, each cDNA was expressed as a fusion protein with the transcription activation domain of the transcription factor Gal4 (amino acids 768 to 881) and a 9 amino acid hemagglutinin epitope tag. PNY200 cells (MATα mating type), expressing the bait, were then mated with BK100 cells (MATa mating type), expressing prey proteins from a prey library. The resulting diploid yeast cells expressing proteins interacting with the bait protein were selected for the ability to synthesize tryptophan, leucine, histidine, and adenine. DNA was prepared from each clone, transformed by electroporation into E. coli strain KC8 (Clontech KC8 electrocompetent cells, Catalog No. C2023-1), and the cells were selected on ampicillin-containing plates in the absence of either tryptophane (selection for the bait plasmid) or leucine (selection for the library plasmid). DNA for both plasmids was prepared and sequenced by the dideoxynucleotide chain termination method. The identity of the bait cDNA insert was confirmed and the cDNA insert from the prey library plasmid was identified using the BLAST program to search against public nucleotide and protein databases. Plasmids from the prey library were then individually transformed into yeast cells together with a plasmid driving the synthesis of lamin and 5 other test proteins, respectively, fused to the Gal4 DNA binding domain. Clones that gave a positive signal in the [symbol] -galactosidase assay were considered false-positives and discarded. Plasmids for the remaining clones were transformed into yeast cells together with the original bait plasmid. Clones that gave a positive signal in the β-galactosidase assay were considered true positives.

Bait sequences indicated in the tables were used in the yeast two-hybrid system described above. The isolated prey sequences are summarized in the tables. The GenBank Accession Nos. for the bait and prey proteins are also provided in the tables, upon which the bait and prey sequences are aligned.

2. Production of Antibodies Selectively Immunoreactive with Protein Complex

The δ-catenin interacting region of FAK2 and the FAK2-interacting region of δ-catenin are indicated in Tables 53 and 54. Both regions, or fragments thereof, are recombinantly-expressed in E. coli. and isolated and purified. Mixing the two purified interacting regions forms a protein complex. A protein complex is also formed by mixing recombinantly-expressed, intact, full-length δ-catenin and FAK2. The two protein complexes are used as antigens in immunizing a mouse. mRNA is isolated from the immunized mouse spleen cells, and first-strand cDNA is synthesized using the mRNA as a template. The V_{H} and V_{K} genes are amplified from the thus synthesized cDNAs by PCR using appropriate primers.

The amplified V_{H} and V_{K} genes are ligated together and subcloned into a phagemid vector for the construction of a phage display library. E. coli. cells are transformed with the ligation mixtures, and thus a phage display library is established. Alternatively, the ligated V_{H} and V_{K} genes are subcloned into a vector suitable for ribosome display in which the V_{H}-V_{K} sequence is under the control of a T7 promoter. See Schaffitzel et al., J Immun Meth, 231:119-135 (1999).

The libraries are screened for their ability to bind δ-catenin:FAK2 complex and δ-catenin or FAK2, alone. Several rounds of screening are generally performed. Clones corresponding to scFv fragments that bind the δ-catenin:FAK2 complex, but not isolated δ-catenin or FAK2 are selected and purified. A single purified clone is used to prepare an antibody selectively immunoreactive with the complex comprising δ-catenin and FAK2. The antibody is then verified by an immunochemistry method such as RIA and ELISA.

In addition, the clones corresponding to scFv fragments that bind the complex comprising δ-catenin and FAK2, and also bind isolated δ-catenin and/or FAK2 may be selected. The scFv genes in the clones are diversified by mutagenesis methods such as oligonucleotide-directed mutagenesis, error-prone PCR (See Lin-Goerke et al., Biotechniques, 23:409 (1997)), dNTP analogues (See Zaccolo et al., J Mol Biol, 255:589 (1996)), and other methods. The diversified clones are further screened in phage display or ribosome display libraries. In this manner, scFv fragments selectively immunoreactive with the complex comprising δ-catenin and FAK2 may be obtained.

3. Yeast Screen to Identify Small Molecule Inhibitors of the Interaction Between δ-Catenin and FAK2

Beta-galactosidase is used as a reporter enzyme to signal the interaction between yeast two-hybrid protein pairs expressed from plasmids in Saccharomyces cerevisiae. Yeast strain MY209 (ade2 his3 leu2 trp1 cyh2 ura3::GAL1p-lacZ gal4 gal80 lys2::GAL1p-HIS3) bearing one plasmid with the genotype of LEU2 CEN4 ARS1 ADH1p-SV40NLS-GAL4 (768-881)-FAK2-PGK1t AmpR Co1E1_ori, and another plasmid having a genotype of TRP1 CEN4 ARS ADH1p-GAL4(1-147CTNND2-ADH1t AmpR Co1E1_ori is cultured in synthetic complete media lacking leucine and tryptophan (SC-Leu-Trp) overnight at 30° C. The δ-catenin (CTNND2) and FAK2 nucleic acids in the plasmids can code for the full-length δ-catenin and FAK2 proteins, respectively, or fragments thereof. This culture is diluted to 0.01 OD₆₃₀ units/ml using SC-Leu-Trp media. The diluted MY209 culture is dispensed into 96-well microplates. Compounds from a library of small molecules are added to the microplates; the final concentration of test compounds is approximately 60 μM. The assay plates are incubated at 30° C. overnight.

The following day an aliquot of concentrated substrate/lysis buffer is added to each well and the plates incubated at 37° C. for 1-2 hours. At an appropriate time an aliquot of stop solution is added to each well to halt the beta-galactosidase reaction. For all microplates an absorbance reading is obtained to assay the generation of product from the enzyme substrate. The presence of putative inhibitors of the interaction between δ-catenin and FAK2 results in inhibition of the beta-galactosidase signal generated by MY209. Additional testing eliminates compounds that decreased expression of beta-galactosidase by affecting yeast cell growth and non-specific inhibitors that affected the beta-galactosidase signal generated by the interaction of an unrelated protein pair.

Once a hit, i.e., a compound which inhibits the interaction between the interacting proteins, is obtained, the compound is identified and subjected to further testing wherein the compounds are assayed at several concentrations to determine an IC₅₀ value, this being the concentration of the compound at which the signal seen in the two-hybrid assay described in this Example is 50% of the signal seen in the absence of the inhibitor.

4. Enzyme-Linked Immunosorbent Assay (ELISA)

pGEX5X-2 (Amersham Biosciences; Uppsala, Sweden) is used for the expression of a GST-FAK2 fusion protein. The pGEX5X-2-FAK2 construct is transfected into Escherichia coli strain DH5α (Invitrogen; Carlsbad, Calif.) and fusion protein is prepared by inducing log phase cells (O.D. 595=0.4) with 0.2 mM isopropyl-β-D-thiogalactopyranoside (IPTG). Cultures are harvested after approximately 4 hours of induction, and cells pelleted by centrifugation. Cell pellets are resuspended in lysis buffer (1% nonidet P-40 [NP-40], 150 mM NaCl, 10 mM Tris pH 7.4, 1 mM ABESF [4-(2-aminoethyl) benzenesulfonyl fluoride]), lysed by sonication and the lysate cleared of insoluble materials by centrifugation. Cleared lysate is incubated with Glutathione Sepharose beads (Amersham Biosciences; Uppsala, Sweden) followed by thorough washing with lysis buffer. The GST-FAK2 fusion protein is then eluted from the beads with 5 mM reduced glutathione. Eluted protein is dialyzed against phosphate buffer saline (PBS) to remove the reduced glutathione.

A stable Drosophila Schneider 2 (S2) myc-δ-catenin expression cell line is generated by transfecting S2 cells with pCoHygro (Invitrogen; Carlsbad, Calif.) and an expression vector that directs the expression of the myc-δ-catenin fusion protein. Briefly, S2 cells are washed and re-suspended in serum free Express Five media (Invitrogen; Carlsbad, Calif.). Plasmid/liposome complexes are then added (NovaFECTOR, Venn Nova; Pompano Beach, Fla.) and allowed to incubate with cells for 12 hours under standard growth conditions (room temperature, no CO₂ buffering). Following this incubation period fetal bovine serum is added to a final concentration of 20% and cells are allowed to recover for 24 hours. The media is replaced and cells are grown for an additional 24 hours. Transfected cells are then selected in 350 μg/ml hygromycin for three weeks. Expression of myc-δ-catenin is confirmed by Western blotting. This cell line is referred to as S2-myc-δ-catenin.

GST-FAK2 fusion protein is immobilized to wells of an ELISA plate as follows: Nunc Maxisorb 96 well ELISA plates (Nalge Nunc International; Rochester, N.Y.) are incubated with 100 μl of 10 μg/ml of GST-FAK2 in 50 mM carbonate buffer (pH 9.6) and stored overnight at 4° C. This plate is referred to as the ELISA plate.

A compound dilution plate is generated in the following manner. In a 96 well polypropylene plate (Greiner, Germany) 50 μl of DMSO is pipetted into columns 2-12. In the same polypropylene plate pipette, 10 μl of each compound being tested for its ability to modulate protein-protein interactions is plated in the wells of column 1 followed by 90 μl of DMSO (final volume of 100 μl). Compounds selected from primary screens or from virtual screening, or designed based on the primary screen hits are then serially diluted by removing 50 μl from column 1 and transferring it to column 2 (50:50 dilution). Serial dilutions are continued until column 10. This plate is termed the compound dilution plate.

Next, 12 μl from each well of the compound dilution plate is transferred into its corresponding well in a new polypropylene plate. 108 μl of S2-myc-δ-catenin-containing lysate (1×10⁶ cell equivalents/ml) in phosphate buffered saline is added to all wells of columns 1-11. 108 μl of phosphate buffered saline without lysate is added into all wells of column 12. The plate is then mixed on a shaker for 15 minutes. This plate is referred to as the compound preincubation plate.

The ELISA plate is emptied of its contents and 400 μl of Superblock (Pierce Endogen; Rockford, Ill.) is added to all the wells and allowed to sit for 1 hour at room temperature. 100 μl from all columns of the compound preincubation plate are transferred into the corresponding wells of the ELISA binding plate. The plate is then covered and allowed to incubate for 1.5 hours room temperature.

The interaction of the myc-tagged δ-catenin with the immobilized GST-FAK2 is detected by washing the ELISA plate followed by an incubation with 100 μl/well of 1 μg/ml of mouse anti-myc IgG (clone 9E10; Roche Applied Science; Indianapolis, Ind.) in phosphate buffered saline. After 1 hour at room temperature, the plates are washed with phosphate buffered saline and incubated with 100 μl/well of 250 ng/ml of goat anti-mouse IgG conjugated to horseradish peroxidase in phosphate buffer saline. Plates are then washed again with phosphate buffered saline and incubated with the fluorescent substrate solution Quantiblu (Pierce Endogen; Rockford, Ill.). Horseradish peroxidase activity is then measured by reading the plates in a fluorescent plate reader (325 nm excitation, 420 nm emission).

5. Effects of Antisense Inhibitors Ribozymes or siRNAs on Protein Expression

The effects of antisense inhibitors, ribozymes or siRNAs on protein expression can be measured by a variety of methods known in the art. A preferred method is to measure mRNA levels using real-time quantitative polymerase chain reaction (PCR) methods. Real-time PCR can be performed using the ABI PRISM™ 7700 Sequence Detection System according to the manufacturer's instructions. The ABI PRISM™ 7700 Sequence Detection System is available from PE-APPLIED Biosystems, Foster City, Calif.

Other methods of measuring mRNA levels may also be used to determine the effects of anitisense inhibitors, ribozymes or siRNAs on protein expression. For example competitive PCR and Northern blot analysis are well known in the art and may be performed to determine mRNA levels. Specifically, methods of RNA isolation and Northern blot analysis may be performed according to Ausubel, F. M. et al., Current Protocols in Molecular Biology, Volume 1, pp. 4.1.1-4.2.9 and 4.5.1-4.5.3, John Wiley & Sons, Inc., 1993.

The effects of antisense inhibitors, ribozymes or siRNAs on protein expression may also be determined by measuring protein levels of the proteins of interest. Various methods known in the art may be used, such as immunoprecipitation, Western blot analysis, ELISA, or fluorescence-activated cell sorting (FACS). Antibodies to the proteins of interest are often commercially available, and may be found by such sources as the MSRS catalogue of antibodies (Aerie Corporation, Birmingham, Mich.). Antibodies can also be prepared through conventional antibody generation methods, such as found in Ausubel, F. M. et al., Current Protocols in Molecular Biology, Volume 2, pp. 11.12.1-11.12.9 and 11.4.1-11.11.5 John Wiley & Sons, Inc., 1997. Furthermore, immunoprecipitation analysis can be performed according to Ausubel, F. M. et al., Current Protocols in Molecular Biology, Volume 2, pp. 10.16.1-10.16.11, John Wiley & Sons, Inc., 1997, and ELISA can be performed according to Ausubel, F. M. et al., Current Protocols in Molecular Biology, Volume 2, pp. 11.1.1-11.2.22, John Wiley & Sons, Inc., 1997 or as described in Example 4, above.

6. Detection of Amyloid Beta with Biosource Elisa Kit (Camarillo, Calif.)

The present invention provides compositions and methods for lowering Aβ42 levels. To test whether compounds and compositions are capable of lowering Aβ levels, a sandwich enzyme-linked immunosorbent assay (ELISA) is employed to measure secreted Aβ (Aβ42 and/or Aβ40) levels. In this example, H4 cells expressing wild type APP(695) are seeded at 200,000 cells/per well in 6 well plates, and incubated at 37° C. with 5% CO2 overnight. Cells are treated with 1.5 ml medium containing vehicle (DMSO) or a test compound at 1.25 μM, 2.5 μM, 5.0 μM and 10.0 μM (as well as other concentration if desirable) for 24 hours or 48 hours. The supernatant from treated cells is collected into eppendorf tubes and frozen at −80° C. for future analysis.

The amyloid peptide standard is reconstituted and frozen samples are thawed. The samples and standards are diluted with appropriate diluents and the plate is washed 4 times with Working Wash Buffer and patted dry on a paper towel. 100 μL per well of peptide standards, controls, and diluted samples to be analyzed are added. The plate is incubated for 2 hours with shaking on an orbital plate shaker at RT. The plate is then washed 4 times with Working Wash Buffer and patted dry on a paper towel. Detection Antibody Solution is poured into a reservoir and 100 μL per well of Detection Antibody Solution is immediately added to the plate. The plate is incubated at RT for 2 hours with shaking and then washed four times with Working Wash Buffer and patted dry on a paper towel. Secondary Antibody Solution is then poured into a reservoir and 100 μL per well of Secondary Antibody Solution is immediately added to the plate. The plate is incubated at RT for 2 hours with shaking, washed 5 times with Working Wash Buffer, and patted dry on a paper towel.

100 μL of stabilized chromogen is added to each well and the liquid in the wells begins to turn blue. The plate is incubated for 30 minutes at room temperature and in the dark. 100 μL of stop solution is added to each well and the plate is tapped gently to mix the contents, resulting in a change of solution color from blue to yellow. The absorbance of each well is read at 450 nm after having blanked the plate reader against a chromogen blank composed of 100 μL each of stabilized chromogen and stop solution. The plate is read within 2 hours of adding the stop solution. The absorbance of the standards is plotted against the standard concentration and the concentrations of unknown samples and controls are calculated.

7. Detection of Amyloid Beta with Innogenetic Elisa Kit (Gent, Belgium)

The present invention provides compositions and methods for lowering Aβ42. levels. To test whether compounds and compositions are capable of lowering Aβ levels, sandwich enzyme-linked immunosorbent assay (ELISA) are employed to measure secreted Aβ (Aβ42 and/or Aβ40) levels. In this Example, H4 cells expressing wild type APP(695) are seeded at 200,000 cells/per well in 6 well plates, and incubated at 37° C. with 5% CO2 overnight. Cells are treated with 1.5 ml medium containing vehicle (DMSO) or a test compound at 1.25 μm, 2.5 μm, 5.0 μm and 10.0 μm (or other concentrations, if desirable) for 24 hours or 48 hours. The supernatant from treated cells is collected into eppendorf tubes and frozen at −80° C. for future analysis.

130 μl per well of samples, standards, and blanks is added to a 96-well polypropylene plate. 200 μl of samples, standards, and blanks from the polypropylene plate is added to the antibody-coated plates. The strip-holder with an appropriate number of strips is applied to the antibody-coated plates and the strips are covered with an adhesive sealer. The plate is then incubated for 3 hours at room temperature with shaking on an orbital plate shaker.

The primary antibody solution is prepared with Conjugate Diluent 1 at 1:100 ratio. Each well of the antibody-coated plates is washed 5 times with 400 μl washing solution and 100 μl of the prepared first antibody solution is added to each well. The strips are applied to the plate, covered with an adhesive sealer, and the plate is incubated for 1 hour at room temperature with shaking on an orbital plate shaker.

The secondary antibody (conjugate 2) solution is prepared with Conjugate Diluent 2 at 1:100 ratio. Each well of the antibody-coated plates is washed for 5 times with 400 μl washing solution, and 100 μl of the prepared second antibody solution is added to each well. The strips are applied, covered with an adhesive sealer, and the plate is incubated 30 min at room temperature with shaking on an orbital plate shaker. Each well of the antibody-coated plates is then washed 5 times with 400 μl washing solution.

A substrate solution is prepared by diluting Substrate 100× with HRP Substrate Buffer. 100 μl of the prepared substrate solution is added to each well of the antibody-coated plate. The strips are applied, covered with an adhesive sealer, and the plate is incubated for 30 min at room temperature. 100 μl Stop Solution is then added to each well to stop the reaction. The strip-holder is carefully taped to ensure through mixing. The reader is blanked and the absorbance of the solution in the wells is read at 450 nm. The absorbance of the standards is plotted against the standard concentration and the concentration of samples is calculated using the standard curve.

8. Aβ Secretion Assay

The present invention provides compositions and methods for lowering Aβ42 levels. To test whether compounds and compositions are capable of lowering Aβ levels, H4 neuroglioma cells expressing APP695NL and CHO cells stably expressing wild-type human APP(751) and human mutant presenilin 1 (PS1) M146L are used. The generation and culture of these cells has been described. See Murphy et al., J Biol Chem, 274:11914-11923 (1999); Murphy et al., J Biol Chem, 275:26277-26284 (2000). To minimize toxic effects of the compositions and compounds, the H4 cells are incubated for 6 hours in the presence of the various compositions and compounds. To evaluate the potential for toxic effects of the compositions and compounds, additional aliquots of cells are incubated in parallel with each composition or compound. The supernatants are analyzed for the presence of lactate dehydrogenase (LDH) as a measure of cellular toxicity.

After incubating the cells with the compositions and compounds for a pre-determined time period, a sandwich enzyme-linked immunosorbent assay (ELISA) is employed to measure secreted Aβ (Aβ42 and/or Aβ40) levels as described previously. Murphy et al., J Biol Chem, 275:26277-26284 (2000). For cell culture studies, serum free media samples are collected following 6-12 hours of conditioning, Complete Protease Inhibitor Cocktail is added (PIC; Roche), and total Aβ concentration measured by 3160/BA27 sandwich ELISA for Aβ40 and 3160/BC05 sandwich ELISA for Aβ42. All measurements are performed in triplicate. Antibody 3160 is an affinity purified polyclonal antibody raised against Aβ 1-40. HRP conjugated monoclonal antibodies BA27 for detection of Aβ40 and BC05 for detection of Aβ42 have been previously described. Suzuki et al., Science, 264:1336-1340 (1994).

9. Treatment of Animals with a Compound to Determine the Compound's Effect on Levels of Aβ42 and Alzheimer's Disease

To determine the effect of a composition of the present invention on levels of Aβ42 and AD, an animal is treated with the compound and the levels of Aβ42 in the brain are measured. Three month-old TG2576 mice that overexpress APP(695) with the “Swedish” mutation (APP695NL) are used. Mice overexpressing APP(695) with the “Swedish” mutation have high levels of soluble Aβ in the their brains and develop memory deficits and plaques with age, making them suitable for examining the effect of compounds on levels of Aβ42 and Alzheimer's Disease. “Test” TG25276 mice are treated with the compound and “control” TG25276 mice are subjected to a “sham” treatment lacking the test compound. The brain levels of SDS-soluble Aβ40 and Aβ42 for “test” mice are compared to those of “control” mice using ELISA. Test mice that show a reduction in Aβ42 levels suggest that treatment with the compound prevents amyloid pathology by decreasing the ratio of Aβ42 to Aβ40 in the brain. Histopathological analysis can be conducted to examine the amount of amyloid plaques formed in the test animals versus the controls.

10. Neuroprotection Assay

The present invention provides compositions and methods for slowing the death or decline of neurons. To test the ability of compositions of the present invention to protect against neurotoxicity, adult female Sprague Dawley rats are obtained and injected intraperitoneally with various doses of a composition of the present invention. At the same time, the test animals also receive a subcutaneous injection of MK-801 (0.5 mg/kg), which has been shown to consistently induce, in all treated rats, a fully developed neurotoxic reaction consisting of acute vacuole formation in the majority of pyramidal neurons in layers III and IV of the posterior cingulate and retrosplenial (PC/RS) cortices.

Control animals are administered the liquid which was used to dissolve the test agent and the same dosage of MK-801 (0.5 mg/kg sc). The animals are sacrificed four hours after treatment and the numbers of vacuolated PC/RS neurons are counted on each side of the brain, at a rostrocaudal level immediately posterior to where the corpus callosum ceases decussating across the midline (approximately 5.6 mm caudal to bregma). The toxic reaction approaches maximal severity at this level and shows very little variability between different animals.

Percentage reduction in neurotoxicity is calculated by dividing the mean number of vacuolated neurons in a given treatment group, by the mean number of vacuolated neurons in control animals that were treated with MK-801 but not the protective agent. The result is subtracted from one and multiplied by 100, to calculate a percentage. Linear regression analysis can be used to determine an ED₅₀ (i.e., the dosage of a given compound that reduces the mean number of vacuolated neurons to 50% of the value in control animals), with the 25th and 75th percentiles defining the confidence limits.

11. Treatment of Animals with a Compound to Determine the Compound's Effect on Memory and Alzheimer's Disease

The present invention provides compositions and methods for treating or preventing AD. To test the effect of compositions of the present invention on memory and AD, TG2576 mice that overexpress APP(695) with the “Swedish” mutation (APP695NL) are used as an animal model for the disease. Mice overexpressing APP(695) with the “Swedish” mutation develop memory deficits and plaques with age, making them suitable for examining the effect of compounds on memory and AD. The test compound is administered daily for two weeks to test groups of the TG2576 micein age groups of: 1) 4-5 months, 2) 6-11 months, 3) 12-18 months, and 4) 20-25 months. Groups of control TG2576 mice of corresponding ages receive a “sham” treatment that is identical to the experimental treatment except that it excludes the compound being evaluated. Both control and test groups then have their memory tested in a version of the Morris water maze (Morris, J Neurosci Meth, 11:47-60 (1984)), which is specifically modified for mice. The water maze contains a metal circular pool of about 40 cm in height and 75 cm in diameter. The walls of the pool have fixed spatial orientation clues of distinct patterns or shelves containing objects. The pool is filled with room temperature water to a depth of 25cm and an escape platform is hidden 0.5 cm below the surface of the 25-cm-deep water at a fixed position in the center of one of the southwest quadrants of pool. The test and control mice are trained for 10 days in daily sessions consisting of four trials in which the mouse starts in a different quadrant of the pool for each trial. The mice are timed and given 60 seconds to find the escape platform in the pool. If the mice have not found the escape platform after 60 seconds, they are guided to it. The mice are then allowed to rest on the platform for 30 seconds and the amount of time it takes the mice to find the platform is recorded. Probe trials are run at the end of the trials on the 4th, 7th, and 10th days of training, in which the platform is removed and the mice are allowed to search for the platform for 60 sec. The percentage of time spent in the quadrant where the platform was located in previous trials is calculated.

In training trials, the time it takes test group mice to reach the escape platform is compared to the time taken by control group mice of corresponding ages. In probe trials, the percentage of time spent by test group mice in the quadrant where the platform was located in previous trials is compared to the percentage time spent in that quadrant by control mice. Quicker location of the escape platform in training trials, and/or an increased percentage time spent in the quadrant of the maze previously containing the escape platform during probe trials are indicative of spatial learning and memory. Because memory loss is a hallmark of AD, test mice that exhibiting better learning and memory, when compared to control mice, support the hypothesis that the test compound may be effective in treating or slowing the onset of AD and/or its symptoms.

12. Clinical Investigation of Compounds of Formula I for Alzheimer's Disease

According to this example, a compound of Formula I is examined for its actions in healthy subjects as well as subjects with mild to moderate Alzheimer's disease (AD). Evaluation of the compound for treating Alzheimer's is accomplished in a three-group parallel design; each group having 53 subjects for a total of 159 subjects. Subjects are treated with a compound of Formula I or a matching placebo twice a day for forty-eight weeks.

Test AD subjects are selected based on the following criteria: Subjects (1) have a diagnosis of dementia according to the DSM IV (TR) and meets the NINCDS-ADRDA (McKhann et al., Neurology 34:939-944 (1984)) criteria for probable Alzheimer's disease, (2) have CT or MRI since onset of memory impairment demonstrating absence of clinically significant focal adhesion, (3) have MMSE (Mohs et al. Int Psychogeriatr 8:195-203 (1996)) score ≧15 and ≦26, (4) have a modified Hachinski Ischaemic score <4, (5) age ≧45 years and living in the community at the time of enrollment, (6) signed patient informed consent form and willing/able to attend for duration of study, (7) read and understand English, six years of education or work history sufficient to exclude mental retardation. Subjects can have no unforeseen aspirin use other than for cardioprotective therapy (e.g., <325 mg aspirin/day). Subjects taking acetylcholinesterase inhibitors may be enrolled as long as they have been on a stable treatment dose for at least three months. Subjects must have a reliable English speaking caregiver or informant to accompany the subject for clinic visits and be prepared to supervise medication.

Subjects are excluded according to the following criteria: treatment with memantine in past 4 weeks, current evidence or history in the last 2 years of epilepsy, focal brain lesion, head injury with loss of consciousness and or immediate confusion after the injury, or DSM-IV criteria for major psychiatric disorder including psychosis, major depression, bipolar disorder, alcohol or substance abuse, chronic use of NSAIDs at any dose more than 7 days per month for the two months prior to Study day 1, history of, or evidence of active malignancy, except basal cell carcinoma and squamous cell carcinoma of the skin within the 24 months prior to entry, chronic or acute renal, hepatic, or metabolic disorder or any other condition, which in the Investigator's opinion, might preclude study participation, use of any investigational therapy within 30 days, or 5 half-lives whichever is longer, prior to screening, major surgery within 12 weeks prior to Study Day 1, patients with uncontrolled cardiac conditions (New York Heart Association Class III or IV), and anticoagulant therapy such as warfarin with 12 weeks prior to randomization.

Therapeutic Endpoints: The primary efficacy endpoint is the rate of decline in the ADAS-cog score based on either a slope calculated for each patient or on a Generalized Estimating Equations (GEE) model. Secondary efficacy endpoints can include scores on the CIBIC+, NPI, ADCS-ADL, and CDR sum of boxes. Efficacy analyses for primary and secondary endpoints can include the baseline score as a covariate, and will also include a term for the stratification variable:use or nonuse of acetylcholinesterase inhibitor baseline. A modified intent to treat approach can be used in which all randomized subjects who receive any study treatment and have post-baseline efficacy assessment can be included in the intent to treat population using a last value carried forward approach. A per protocol analysis population can include all subjects in the intent to treat population who did not have any major protocol violations.

Subjects consist of men and women, ages 60-85, who are diagnosed with probable AD using the National Institute of Neurologic Communicative Disorders and Stroke-Alzheimer's Disease and Related Disorders Association (NINCDS-ADRDA) test (McKhann et al., Neurology 34:939-944 (1984)) or have mild to moderate dementia as determined by the Mini-Mental State Examination (MMSE, Mohs et al. Int Psychogeriatr 8:195-203 (1996)). MMSE scores in the range of 15-25 indicate mild to moderate dementia. AD subjects have caregivers that can ensure compliance with medication regimens and with study visits and procedures.

Control subjects consist of men and women ages 60-80 that lack significant cognitive or functional complaints, or depression as determined by the Geriatric Depression Scale (GDS), and have MMSE scores in the range of 27-30. Control subjects have the same general requirements as AD subjects with the exception that caregivers are not required. Both AD subjects and control subjects have good general health, i.e., subjects do not have serious or life-threatening comorbid conditions.

Subjects who have medically active major inflammatory comorbid condition(s) such as rheumatoid arthritis are excluded from the study. Those who have contra-indications to lumbar puncture, such as severe lumbar spine degeneration, sepsis in the region of the lumbar spine, or a bleeding disorder are excluded from participation in the study. In addition, subjects who currently or recently use medications such as NSAIDs, prednisone, or immunosuppressive medications such as cyclophosphamide that could interfere with the study are excluded. Recently is defined as within one month before undergoing the baseline visit (see next paragraph). Subjects undergoing acetylcholinesterase inhibitor (AChE-I) treatments for AD are not excluded if these subjects have been on stable doses for at least four weeks. Similarly, AD subjects taking antioxidants such as vitamin E, vitamin C, or Gingko biloba are not excluded if they have been on stable doses for at least four weeks. Subjects who use NSAIDs or aspirin on a regular basis are excluded. If needed, analgesics such as paracetamol (Tylenol) are provided during the study.

The study procedure consists of three in-clinic visits: an initial screening visit, a baseline visit, and a follow-up visit at fourteen days. During the screening visit, information needed to assess eligibility is obtained and MMSE is administered.

During the baseline visit, which takes place within two weeks of the screening visit, physical examinations and lumbar punctures are performed. Blood samples are drawn for laboratory tests such as APO-E genotyping and for plasma preparation. At this time, subjects or caregivers, in the case of AD subjects, are given a fourteen-day supply of study a compound of Formula I along with instructions about timing of doses and potential adverse effects. (For AD subjects, caregivers are required to accompany subjects to each visit, and are responsible for monitoring and supervising administration of study compound.) A calendar is provided on which times of medications and potential adverse symptoms are recorded.

The treatment regimen consists of a 48 week treatment with a compound of Formula I in the form of a capsule or tablet taken once a day with meals. High and low study doses of the compound of Formula I are used (i.e., 200 mg and 20 mg.) The compound of Formula I is pre-packed into a day-by-day plastic medication dispenser.

During the follow-up visit, twelve or fourteen days after beginning treatment, vital signs and adverse side effects of study compound are assessed. Surplus compound is returned and counted. In addition, lumbar punctures are performed and blood samples are drawn for laboratory tests and for plasma preparations.

Visits during which lumbar punctures are performed and blood samples are drawn are scheduled for mornings with overnight fasting to avoid obtaining post-prandial or hyperlipemic plasma samples, which can influence levels of Aβ40 and Aβ42. The following paragraph summarizes the biological markers that are analyzed from plasma and CSF samples.

Plasma and CSF biological markers Volume Assay Method Volume of CSF of Plasma Protein, glucose, 1 mL cells Aβ40 ELISA 100 μL×2 100 μL×2 (in duplicate) Aβ42 ELISA 100 μL×2 100 μL×2 (in duplicate) Aβ38. Mass Spectrometry 1 mL Isoprostanes Gas Chromatography/2 mL Mass Spectrometry M-CSF ELISA 50 μL×2 (in duplicate) MCP-1 ELISA 50 μL×2 (in duplicate) Tau, ELISA 50 μL×2 P-tau181 (in duplicate) 50 μL×2 (in duplicate) Plasma drug levels by HPLC 1 mL. The assessment of these markers are within the skill of an ordinary artisan.

Patients having mild-to-moderate Alzheimer's disease undergoing the treatment regimen of this example with a compound of Formula I doses of about 0.1 mg to 800 mg can experience a lessening in decline of cognitive function (as measured by ADAS-cog or CDR sum of boxes), plaque pathology, and/or biochemical disease marker progression.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference. The mere mentioning of the publications and patent applications does not necessarily constitute an admission that they are prior art to the instant application.

Although the foregoing invention has been described in some detail by way of illustration and example for purposes of clarity of understanding, it will be obvious that certain changes and modifications may be practiced within the scope of the appended claims.

What is claimed is:

1. An isolated protein complex comprising a first protein interacting with a second protein, wherein said first protein is selected from any one of the bait proteins identified in tables 1-71, and wherein said second protein is selected from any one of the corresponding prey proteins identified as interacting with said first protein in tables 1-71.

2. A method of identifying a modulator of the isolated protein complex of claim 1, comprising contacting said isolated protein complex of claim 1 with a test compound and determining if the stability of the interaction between said first protein and said second protein is altered in the presence of said test compound relative to the stability of the interaction between said first protein and said second protein in the absence of said test compound, wherein a test compound that alters the stability of the interaction between said first protein and said second protein is identified as a modulator of the isolated protein complex.

3. The method of claim 2 further comprising testing the identied modulator of the isolated protein complex for its ability to reduce the secretion of Aβ42 by human cells when contacted with said human cells, wherein a modulator that reduces the secretion of Aβ42 by human cells, is identified as an Aβ42 lowering compound.

4. A method of treating a neurological disorder or disease in a human patient in need of such treatment, comprising administering to said patient a therapeutically effective amount of an Aβ42 lowering compound identified according to the method of claim 3.


SEQUENCE LISTING (695 sequences)

SEQ ID NO: 1 - DNA, 2740 bp, Homo sapiens
cgagaatttccagcaggcaaggcagtggccgctttgactgcttgcttcggagatccgagacgacggagaaggcactcttatttaccgaccaagaaagctcctcccccgtcctccgttagctaattaaaacatttttcagggacgtagccatccagagacattccattattgttccattgacctttccctcatcactgagtcctttggagctgagttatgtcaacagctgccttaattactttggtcagaagtggtgggaaccaggtgagaaggagagtgctgctaagctcccgcctgctgcaggacgacaggcgggtgacacccacgtgccacagctccacttcagagcctaggtgttctcggtttgacccagatggtagtgggagtccagctacctgggacaattttgggatctgggataaccgcattgatgagccaattctgctgccacccagcattaagtatggcaagccaattcccaaaatcagcttggaaaatgtggggtgcgcctcacagattggcaaacggaaagagaatgaagatcggtttgacttcgctcagctgacagatgaggtcctgtactttgcagtgtatgatggacacggtggacctgcagcagctgatttctgtcatacccacatggagaaatgtattatggatttgcttcctaaggagaagaacttggaaactctgttgaccttggcttttctagaaatagataaagccttttcgagtcatgcccgcctgtctgctgatgcaactcttctgacctctgggactactgcaacagtagccctattgcgagatggtattgaactggttgtagccagtgttggggacagccgggctattttgtgtagaaaaggaaaacccatgaagctgaccattgaccatactccagaaagaaaagatgaaaaagaaaggatcaagaaatgtggtggttttgtagcttggaatagtttggggcagcctcacgtaaatggcaggcttgcaatgacaagaagtattggagatttggaccttaagaccagtggtgtcatagcagaacctgaaactaagaggattaagttacatcatgctgatgacagcttcctggtcctcaccacagatggaattaacttcatggtgaatagtcaagagatttgtgactttgtcaatcagtgccatgatcccaacgaagcagcccatgcggtgactgaacaggcaatacagtacggtactgaggataacagtactgcagtagtagtgccttttggtgcctggggaaaatataagaactctgaaatcaacttctcattcagcagaagctttgcctccagtggacgatgggcctgattaccagctgggacttagagtttctgtgcaacagtttttcactgagcatgtcaagaaactgataagatcaaaaaggtctcctaactcactagatcagcgcacaagtcagtgtaaaccacttagatagtagttttttcataaatgctcatcatatttatgttccgctgtacatgttcagtataaatatatgtgtagtgaagctactgtgagtctttaaatggaaagagcaaatgagaagtggtttggatacacttgatgagagatgagagtgtcacattaataatttttaagactcttaggcagctatgggtttcttttgatcatttttgttctttattcatttgaacacgtttttgaagttcttcaaaactagtcagtttgaattttgacagctattcaatatgtgatctccaagtttaaaaaaatttttttccagacttccctaatcctaaaatgcgagtttttatttttaataactgtaccaaggaataagtatgaaaacagttctctgttaccatattttgtattctggaccacttactggtgaaagcaaccatgcaaaagaaattaatttggccaggcacagtggctcatgcctgtaatcccaaattgctgggattacagcactgtgccctcctaggaaattattttttaagtgaaattttatttttattttttttaggattttggtagagaatgagtaggcctactcatcaatatcaaacaggacatttagtttctttccttagaacagacataaatttaatttcatggtaatatgataataagaaaatgcttctatttttctttagcacctccatggttctcatatacccatgtctgtaaaaagtgacatgagaattttgttgggttacattttattgtatttattagattcgcttatatagatgacttaggcagaaataaagtcatgtctttagaaggtgaacaagccaacttgtgatggcctgccttttgcttttggcagttgggatgagaacaattgactctcccattggttgttagatagttgaaatggtgcgttggtggtcatacttagtgttctaggctgtgaaatcatggagttcttccacttccaagaatgactcatttgctgttggattctagtacagaatttagcagcctgatgtgtccccaaactgatttaatttctactgaagtgcccttgtgtacatttgttttgtaatttaccaaagtactacctgagtgtataatgactcctgcagtgagttaatgtaattgctgctttgaccattgttttaaatctgtgtactagagtaactgtgagcagaatgaaatcacattatctcagtgttcaaaatatcattctaataaagtacatgcattaaacaattttaaaaaaaacaaaaaaaaaaaaaaaaaa

SEQ ID NO: 2 - protein, 372 aa, Homo sapiens
MSTAALITLVRSGGNQVRRRVLLSSRLLQDDRRVTPTCHSSTSEPRCSRFDPDGSGSPATWDNFGIWDNRIDEPILLPPSIKYGKPIPKISLENVGCASQIGKRKENEDRFDFAQLTDEVLYFAVYDGHGGPAAADFCHTHMEKCIMDLLPKEKNLETLLTLAFLEIDKAFSSHARLSADATLLTSGTTATVALLRDGIELVVASVGDSRAILCRKGKPMKLTIDHTPERKDEKERIKKCGGFVAWNSLGQPHVNGRLAMTRSIGDLDLKTSGVIAEPETKRIKLHHADDSFLVLTTDGINFMVNSQEICDFVNQCHDPNEAAHAVTEQAIQYGTEDNSTAVVVPFGAWGKYKNSEINFSFSRSFASSGRWA

SEQ ID NO: 3 - DNA, 20 bp, Homo sapiens
ttaggctccatggccgacag

SEQ ID NO: 4 - DNA, 20 bp, Homo sapiens
aggctccatggccgacaggt

SEQ ID NO: 5 - DNA, 20 bp, Homo sapiens
gctccatggccgacaggtct

SEQ ID NO: 6 - DNA, 20 bp, Homo sapiens
cggagctccatggcggggcc

SEQ ID NO: 7 - DNA, 20 bp, Homo sapiens
gagctccatggcggggccgg

SEQ ID NO: 8 - DNA, 20 bp, Homo sapiens
gctccatggcggggccggcg

SEQ ID NO: 9 - DNA, 20 bp, Homo sapiens
ccgcgatccatctccgcgac

SEQ ID NO: 10 - DNA, 20 bp, Homo sapiens
gcgatccatctccgcgacgg

SEQ ID NO: 11 - DNA, 20 bp, Homo sapiens
gatccatctccgcgacggtc

SEQ ID NO: 12 - DNA, 20 bp, Homo sapiens
cgcggcgccatcatcccccg

SEQ ID NO: 13 - DNA, 20 bp, Homo sapiens
cggcgccatcatcccccgct

SEQ ID NO: 14 - DNA, 20 bp, Homo sapiens
gcgccatcatcccccgctca

SEQ ID NO: 15 - DNA, 20 bp, Homo sapiens
tgagaagccatcctgccaag

SEQ ID NO: 16 - DNA, 20 bp, Homo sapiens
agaagccatcctgccaagaa

SEQ ID NO: 17 - DNA, 20 bp, Homo sapiens
aagccatcctgccaagaatg

SEQ ID NO: 18 - DNA, 20 bp, Homo sapiens
ggaacagacatggccttggc

SEQ ID NO: 19 - DNA, 20 bp, Homo sapiens
aacagacatggccttggcag

SEQ ID NO: 20 - DNA, 20 bp, Homo sapiens
cagacatggccttggcagct

SEQ ID NO: 21 - DNA, 20 bp, Homo sapiens
gctgttgacataactcagct

SEQ ID NO: 22 - DNA, 20 bp, Homo sapiens
tgttgacataactcagctcc

SEQ ID NO: 23 - DNA, 20 bp, Homo sapiens
ttgacataactcagctccaa

SEQ ID NO: 24 - DNA, 20 bp, Homo sapiens
aagtggttcatggtgggagt

SEQ ID NO: 25 - DNA, 20 bp, Homo sapiens
gtggttcatggtgggagtcg

SEQ ID NO: 26 - DNA, 20 bp, Homo sapiens
ggttcatggtgggagtcgga

SEQ ID NO: 27 - DNA, 20 bp, Homo sapiens
gaggtggtcatggtggaagg

SEQ ID NO: 28 - DNA, 20 bp, Homo sapiens
ggtggtcatggtggaaggtg

SEQ ID NO: 29 - DNA, 20 bp, Homo sapiens
tggtcatggtggaaggtgtt

SEQ ID NO: 30 - DNA, 20 bp, Homo sapiens
gagttttccatccctccagc

SEQ ID NO: 31 - DNA, 20 bp, Homo sapiens
gttttccatccctccagctc

SEQ ID NO: 32 - DNA, 20 bp, Homo sapiens
tttccatccctccagctcag

SEQ ID NO: 33 - DNA, 20 bp, Homo sapiens
aactctgtcattggagcaac

SEQ ID NO: 34 - DNA, 20 bp, Homo sapiens
ctctgtcattggagcaactg

SEQ ID NO: 35 - DNA, 20 bp, Homo sapiens
ctgtcattggagcaactgta

SEQ ID NO: 36 - DNA, 20 bp, Homo sapiens
tcgtcggccatggtccggag

SEQ ID NO: 37 - DNA, 20 bp, Homo sapiens
gtcggccatggtccggaggg

SEQ ID NO: 38 - DNA, 20 bp, Homo sapiens
cggccatggtccggagggga

SEQ ID NO: 39 - DNA, 20 bp, Homo sapiens
caccaaagcattttgaaaag

SEQ ID NO: 40 - DNA, 20 bp, Homo sapiens
ccaaagcattttgaaaagtg

SEQ ID NO: 41 - DNA, 20 bp, Homo sapiens
aaagcattttgaaaagtgta

SEQ ID NO: 42 - DNA, 20 bp, Homo sapiens
gagccccccatcgccccgcc

SEQ ID NO: 43 - DNA, 20 bp, Homo sapiens
gccccccatcgccccgccgc

SEQ ID NO: 44 - DNA, 20 bp, Homo sapiens
cccccatcgccccgccgcgc

SEQ ID NO: 45 - DNA, 20 bp, Homo sapiens
acaagcttcatggtcacaag

SEQ ID NO: 46 - DNA, 20 bp, Homo sapiens
aagcttcatggtcacaagcg

SEQ ID NO: 47 - DNA, 20 bp, Homo sapiens
gcttcatggtcacaagcgaa

SEQ ID NO: 48 - DNA, 20 bp, Homo sapiens
cggtgggccatggcagtcgt

SEQ ID NO: 49 - DNA, 20 bp, Homo sapiens
gtgggccatggcagtcgttc

SEQ ID NO: 50 - DNA, 20 bp, Homo sapiens
gggccatggcagtcgttcac

SEQ ID NO: 51 - DNA, 20 bp, Homo sapiens
gcaaaagccattgctggagt

SEQ ID NO: 52 - DNA, 20 bp, Homo sapiens
aaaagccattgctggagttg

SEQ ID NO: 53 - DNA, 20 bp, Homo sapiens
aagccattgctggagttggc

SEQ ID NO: 54 - DNA, 20 bp, Homo sapiens
agctccgccatcttcccgcc

SEQ ID NO: 55 - DNA, 20 bp, Homo sapiens
ctccgccatcttcccgccgc

SEQ ID NO: 56 - DNA, 20 bp, Homo sapiens
ccgccatcttcccgccgcag

SEQ ID NO: 57 - DNA, 20 bp, Homo sapiens
accttccccatggtgtctga

SEQ ID NO: 58 - DNA, 20 bp, Homo sapiens
cttccccatggtgtctgagc

SEQ ID NO: 59 - DNA, 20 bp, Homo sapiens
tccccatggtgtctgagcga

SEQ ID NO: 60 - DNA, 20 bp, Homo sapiens
aatgtgagcatagccctgcc

SEQ ID NO: 61 - DNA, 20 bp, Homo sapiens
tgtgagcatagccctgcctc

SEQ ID NO: 62 - DNA, 20 bp, Homo sapiens
tgagcatagccctgcctctg

SEQ ID NO: 63 - DNA, 20 bp, Homo sapiens
aatgaagtcatttttaaccc

SEQ ID NO: 64 - DNA, 20 bp, Homo sapiens
tgaagtcatttttaaccctt

SEQ ID NO: 65 - DNA, 20 bp, Homo sapiens
aagtcatttttaacccttat

SEQ ID NO: 66 - DNA, 20 bp, Homo sapiens
gccccagtcatggtacaagt

SEQ ID NO: 67 - DNA, 20 bp, Homo sapiens
cccagtcatggtacaagtta

SEQ ID NO: 68 - DNA, 20 bp, Homo sapiens
cagtcatggtacaagttaca

SEQ ID NO: 69 - DNA, 20 bp, Homo sapiens
atctcccgcatgaactacag

SEQ ID NO: 70 - DNA, 20 bp, Homo sapiens
ctcccgcatgaactacaggt

SEQ ID NO: 71 - DNA, 20 bp, Homo sapiens
cccgcatgaactacaggtac

SEQ ID NO: 72 - DNA, 20 bp, Homo sapiens
agaatatccatgtctaagcc

SEQ ID NO: 73 - DNA, 20 bp, Homo sapiens
aatatccatgtctaagccag

SEQ ID NO: 74 - DNA, 20 bp, Homo sapiens
tatccatgtctaagccagaa

SEQ ID NO: 75 - DNA, 20 bp, Homo sapiens
ctcttgtacatagtctccag

SEQ ID NO: 76 - DNA, 20 bp, Homo sapiens
cttgtacatagtctccaggc

SEQ ID NO: 77 - DNA, 20 bp, Homo sapiens
tgtacatagtctccaggcca

SEQ ID NO: 78 - DNA, 20 bp, Homo sapiens
atagctttcatttcatcgtt

SEQ ID NO: 79 - DNA, 20 bp, Homo sapiens
agctttcatttcatcgtttt

SEQ ID NO: 80 - DNA, 20 bp, Homo sapiens
ctttcatttcatcgtttttt

SEQ ID NO: 81 - DNA, 20 bp, Homo sapiens
tcctcctccatgggggaggg

SEQ ID NO: 82 - DNA, 20 bp, Homo sapiens
ctcctccatgggggaggggc

SEQ ID NO: 83 - DNA, 20 bp, Homo sapiens
cctccatgggggaggggccg

SEQ ID NO: 84 - DNA, 20 bp, Homo sapiens
gacccgtccatcaccgccgc

SEQ ID NO: 85 - DNA, 20 bp, Homo sapiens
cccgtccatcaccgccgctc

SEQ ID NO: 86 - DNA, 20 bp, Homo sapiens
cgtccatcaccgccgctccc

SEQ ID NO: 87 - DNA, 20 bp, Homo sapiens
ccatttgccatagctagcaa

SEQ ID NO: 88 - DNA, 20 bp, Homo sapiens
atttgccatagctagcaacg

SEQ ID NO: 89 - DNA, 20 bp, Homo sapiens
ttgccatagctagcaacggg

SEQ ID NO: 90 - DNA, 20 bp, Homo sapiens
accccagacatcctctcagg

SEQ ID NO: 91 - DNA, 20 bp, Homo sapiens
cccagacatcctctcaggca

SEQ ID NO: 92 - DNA, 20 bp, Homo sapiens
cagacatcctctcaggcagc

SEQ ID NO: 93 - DNA, 20 bp, Homo sapiens
gccaggggcatggcggcgcg

SEQ ID NO: 94 - DNA, 20 bp, Homo sapiens
caggggcatggcggcgcggg

SEQ ID NO: 95 - DNA, 20 bp, Homo sapiens
ggggcatggcggcgcgggcg

SEQ ID NO: 96 - DNA, 20 bp, Homo sapiens
ccggatcccatcctgcgccc

SEQ ID NO: 97 - DNA, 20 bp, Homo sapiens
ggatcccatcctgcgcccgg

SEQ ID NO: 98 - DNA, 20 bp, Homo sapiens
atcccatcctgcgcccggcg

SEQ ID NO: 99 - DNA, 20 bp, Homo sapiens
tgggccggcatcttggctct

SEQ ID NO: 100 - DNA, 20 bp, Homo sapiens
ggccggcatcttggctctcc

SEQ ID NO: 101 - DNA, 20 bp, Homo sapiens
ccggcatcttggctctccga

SEQ ID NO: 102 - DNA, 20 bp, Homo sapiens
cgggtgcccattgcgcggcc

SEQ ID NO: 103 - DNA, 20 bp, Homo sapiens
ggtgcccattgcgcggccga

SEQ ID NO: 104 - DNA, 20 bp, Homo sapiens
tgcccattgcgcggccgagg

SEQ ID NO: 105 - DNA, 20 bp, Homo sapiens
gcagctgccattattttgct

SEQ ID NO: 106 - DNA, 20 bp, Homo sapiens
agctgccattattttgctag

SEQ ID NO: 107 - DNA, 20 bp, Homo sapiens
ctgccattattttgctagat

SEQ ID NO: 108 - DNA, 20 bp, Homo sapiens
gggccgggcatggcgggcgg

SEQ ID NO: 109 - DNA, 20 bp, Homo sapiens
gccgggcatggcgggcggga

SEQ ID NO: 110 - DNA, 20 bp, Homo sapiens
cgggcatggcgggcgggacc

SEQ ID NO: 111 - DNA, 20 bp, Homo sapiens
atagacgactcgcacgacat

SEQ ID NO: 112 - DNA, 20 bp, Homo sapiens
tgtgcggaaatccattactt

SEQ ID NO: 113 - DNA, 20 bp, Homo sapiens
actgggccatcttgttgttg

SEQ ID NO: 114 - DNA, 20 bp, Homo sapiens
gggtccaccatggcgcggcc

SEQ ID NO: 115 - DNA, 20 bp, Homo sapiens
gtccaccatggcgcggccgg

SEQ ID NO: 116 - DNA, 20 bp, Homo sapiens
ccaccatggcgcggccggcc

SEQ ID NO: 117 - DNA, 20 bp, Homo sapiens
agacagtccatgttgggggg

SEQ ID NO: 118 - DNA, 20 bp, Homo sapiens
acagtccatgttggggggcc

SEQ ID NO: 119 - DNA, 20 bp, Homo sapiens
agtccatgttggggggcctg

SEQ ID NO: 120 - DNA, 20 bp, Homo sapiens
tgcttgtgcatggcactgcc

SEQ ID NO: 121 - DNA, 20 bp, Homo sapiens
cttgtgcatggcactgcccc

SEQ ID NO: 122 - DNA, 20 bp, Homo sapiens
tgtgcatggcactgcccccc

SEQ ID NO: 123 - DNA, 20 bp, Homo sapiens
tgtggggccatggcgcacgc

SEQ ID NO: 124 - DNA, 20 bp, Homo sapiens
tggggccatggcgcacgccc

SEQ ID NO: 125 - DNA, 20 bp, Homo sapiens
gggccatggcgcacgccccg

SEQ ID NO: 126 - DNA, 20 bp, Homo sapiens
tggggattcatcttcggcca

SEQ ID NO: 127 - DNA, 20 bp, Homo sapiens
gggattcatcttcggccact

SEQ ID NO: 128 - DNA, 20 bp, Homo sapiens
gattcatcttcggccacttc

SEQ ID NO: 129 - DNA, 20 bp, Homo sapiens
tccttggccatggtggaagc

SEQ ID NO: 130 - DNA, 20 bp, Homo sapiens
cttggccatggtggaagcaa

SEQ ID NO: 131 - DNA, 20 bp, Homo sapiens
tggccatggtggaagcaatt

SEQ ID NO: 132 - DNA, 20 bp, Homo sapiens
gccgccgccatcttccggat

SEQ ID NO: 133 - DNA, 20 bp, Homo sapiens
cgccgccatcttccggattt

SEQ ID NO: 134 - DNA, 20 bp, Homo sapiens
ccgccatcttccggatttta

SEQ ID NO: 135 - DNA, 20 bp, Homo sapiens
ccgggcagcatcgcgaccct

SEQ ID NO: 136 - DNA, 20 bp, Homo sapiens
gggcagcatcgcgaccctgc

SEQ ID NO: 137 - DNA, 20 bp, Homo sapiens
gcagcatcgcgaccctgcgc

SEQ ID NO: 138 - DNA, 20 bp, Homo sapiens
ttgagggccatcgcggcggc

SEQ ID NO: 139 - DNA, 20 bp, Homo sapiens
gagggccatcgcggcggccc

SEQ ID NO: 140 - DNA, 20 bp, Homo sapiens
gggccatcgcggcggcccac

SEQ ID NO: 141 - DNA, 20 bp, Homo sapiens
aggattcgcatttccagacg

SEQ ID NO: 142 - DNA, 20 bp, Homo sapiens
gattcgcatttccagacggt

SEQ ID NO: 143 - DNA, 20 bp, Homo sapiens
ttcgcatttccagacggtct

SEQ ID NO: 144 - DNA, 20 bp, Homo sapiens
tgagtagccattctcctgca

SEQ ID NO: 145 - DNA, 20 bp, Homo sapiens
agtagccattctcctgcagc

SEQ ID NO: 146 - DNA, 20 bp, Homo sapiens
tagccattctcctgcagcag

SEQ ID NO: 147 - DNA, 20 bp, Homo sapiens
gggggcctcatggctgcagg

SEQ ID NO: 148 - DNA, 20 bp, Homo sapiens
gggcctcatggctgcagggc

SEQ ID NO: 149 - DNA, 20 bp, Homo sapiens
gcctcatggctgcagggcag

SEQ ID NO: 150 - DNA, 20 bp, Homo sapiens
accaggaacatggctgcgac

SEQ ID NO: 151 - DNA, 20 bp, Homo sapiens
caggaacatggctgcgactc

SEQ ID NO: 152 - DNA, 20 bp, Homo sapiens
ggaacatggctgcgactcac

SEQ ID NO: 153 - DNA, 20 bp, Homo sapiens
aatgcatccatgttttctgg

SEQ ID NO: 154 - DNA, 20 bp, Homo sapiens
tgcatccatgttttctggaa

SEQ ID NO: 155 - DNA, 20 bp, Homo sapiens
catccatgttttctggaagg

SEQ ID NO: 156 - DNA, 20 bp, Homo sapiens
ttctgggacatggcgggacc

SEQ ID NO: 157 - DNA, 20 bp, Homo sapiens
ctgggacatggcgggaccgg

SEQ ID NO: 158 - DNA, 20 bp, Homo sapiens
gggacatggcgggaccggga

SEQ ID NO: 159 - DNA, 20 bp, Homo sapiens
cccagcggcatgagcagcga

SEQ ID NO: 160 - DNA, 20 bp, Homo sapiens
cagcggcatgagcagcgaga

SEQ ID NO: 161 - DNA, 20 bp, Homo sapiens
gcggcatgagcagcgagaag

SEQ ID NO: 162 - DNA, 20 bp, Homo sapiens
ggcggcttcctcgcaaacat

SEQ ID NO: 163 - DNA, 20 bp, Homo sapiens
attggcattttgagtgcatc

SEQ ID NO: 164 - DNA, 20 bp, Homo sapiens
gccgggtagtggctcgtttc

SEQ ID NO: 165 - DNA, 20 bp, Homo sapiens
atctgggccatggcgctgtc

SEQ ID NO: 166 - DNA, 20 bp, Homo sapiens
ctgggccatggcgctgtcgg

SEQ ID NO: 167 - DNA, 20 bp, Homo sapiens
gggccatggcgctgtcgggg

SEQ ID NO: 168 - DNA, 20 bp, Homo sapiens
ggggctgccatggggccggt

SEQ ID NO: 169 - DNA, 20 bp, Homo sapiens
ggctgccatggggccggtgg

SEQ ID NO: 170 - DNA, 20 bp, Homo sapiens
ctgccatggggccggtgggg

SEQ ID NO: 171 - DNA, 20 bp, Homo sapiens
tccattgtcattccctgact

SEQ ID NO: 172 - DNA, 20 bp, Homo sapiens
cattgtcattccctgactcc

SEQ ID NO: 173 - DNA, 20 bp, Homo sapiens
ttgtcattccctgactccaa

SEQ ID NO: 174 - DNA, 20 bp, Homo sapiens
tttttatccatgactggatg

SEQ ID NO: 175 - DNA, 20 bp, Homo sapiens
tttatccatgactggatgtt

SEQ ID NO: 176 - DNA, 20 bp, Homo sapiens
tatccatgactggatgttct

SEQ ID NO: 177 - DNA, 20 bp, Homo sapiens
tgaccattcattgtgtcttt

SEQ ID NO: 178 - DNA, 20 bp, Homo sapiens
accattcattgtgtctttca

SEQ ID NO: 179 - DNA, 20 bp, Homo sapiens
cattcattgtgtctttcact

SEQ ID NO: 180 - DNA, 20 bp, Homo sapiens
aatacatccatggctaatga

SEQ ID NO: 181 - DNA, 20 bp, Homo sapiens
tacatccatggctaatgaat

SEQ ID NO: 182 - DNA, 20 bp, Homo sapiens
catccatggctaatgaattc

SEQ ID NO: 183 - DNA, 20 bp, Homo sapiens
ctccgacacgctgctgccat

SEQ ID NO: 184 - DNA, 20 bp, Homo sapiens
tgaccccacgttggcctccc

SEQ ID NO: 185 - DNA, 20 bp, Homo sapiens
ctgcatacgaatggccccat

SEQ ID NO: 186 - DNA, 20 bp, Homo sapiens
gcgtgcgccatccttcccag

SEQ ID NO: 187 - DNA, 20 bp, Homo sapiens
gtgcgccatccttcccagag

SEQ ID NO: 188 - DNA, 20 bp, Homo sapiens
gcgccatccttcccagagga

SEQ ID NO: 189 - DNA, 20 bp, Homo sapiens
agaatagacatggtgaactt

SEQ ID NO: 190 - DNA, 20 bp, Homo sapiens
aatagacatggtgaacttct

SEQ ID NO: 191 - DNA, 20 bp, Homo sapiens
tagacatggtgaacttctag

SEQ ID NO: 192 - DNA, 20 bp, Homo sapiens
tggtggcgggaccccgggcc

SEQ ID NO: 193 - DNA, 20 bp, Homo sapiens
tgacacggccctgggggccc

SEQ ID NO: 194 - DNA, 20 bp, Homo sapiens
ctcgaacctcctcaaacacc

SEQ ID NO: 195 - DNA, 20 bp, Homo sapiens
gccgcagtaagtaaagccat

SEQ ID NO: 196 - DNA, 20 bp, Homo sapiens
gttagagtcaatggccccaa

SEQ ID NO: 197 - DNA, 20 bp, Homo sapiens
gctcatggacttgggccttt

SEQ ID NO: 198 - DNA, 20 bp, Homo sapiens
gagtgaggcatggtgacagc

SEQ ID NO: 199 - DNA, 20 bp, Homo sapiens
gtgaggcatggtgacagctc

SEQ ID NO: 200 - DNA, 20 bp, Homo sapiens
gaggcatggtgacagctccc

SEQ ID NO: 201 - DNA, 20 bp, Homo sapiens
gagaagggcatggcggcggc

SEQ ID NO: 202 - DNA, 20 bp, Homo sapiens
gaagggcatggcggcggcgg

SEQ ID NO: 203 - DNA, 20 bp, Homo sapiens
agggcatggcggcggcgggc

SEQ ID NO: 204 - DNA, 20 bp, Homo sapiens
cagcagtccatgctggtgtc

SEQ ID NO: 205 - DNA, 20 bp, Homo sapiens
gcagtccatgctggtgtcgg

SEQ ID NO: 206 - DNA, 20 bp, Homo sapiens
agtccatgctggtgtcgggg

SEQ ID NO: 207 - DNA, 20 bp, Homo sapiens
gcggatgccatcttgccctg

SEQ ID NO: 208 - DNA, 20 bp, Homo sapiens
ggatgccatcttgccctgca

SEQ ID NO: 209 - DNA, 20 bp, Homo sapiens
atgccatcttgccctgcacc

SEQ ID NO: 210 - DNA, 20 bp, Homo sapiens
cgtcctacagcagccgccat

SEQ ID NO: 211 - DNA, 20 bp, Homo sapiens
accaaatatttccccttaaa

SEQ ID NO: 212 - DNA, 20 bp, Homo sapiens
cttctgaaagtactctttgg

SEQ ID NO: 213 - DNA, 20 bp, Homo sapiens
ctcatcgccatagcaaaccc

SEQ ID NO: 214 - DNA, 20 bp, Homo sapiens
catcgccatagcaaacccgc

SEQ ID NO: 215 - DNA, 20 bp, Homo sapiens
tcgccatagcaaacccgcgg

SEQ ID NO: 216 - DNA, 20 bp, Homo sapiens
cagcccgacatccgcaccgc

SEQ ID NO: 217 - DNA, 20 bp, Homo sapiens
gcccgacatccgcaccgccc

SEQ ID NO: 218 - DNA, 20 bp, Homo sapiens
ccgacatccgcaccgcccga

SEQ ID NO: 219 - DNA, 20 bp, Homo sapiens
gcccaggccatcctggctcc

SEQ ID NO: 220 - DNA, 20 bp, Homo sapiens
ccaggccatcctggctcctt

SEQ ID NO: 221 - DNA, 20 bp, Homo sapiens
aggccatcctggctccttcc

SEQ ID NO: 222 - DNA, 20 bp, Homo sapiens
ccacgggccatgccgaccac

SEQ ID NO: 223 - DNA, 20 bp, Homo sapiens
acgggccatgccgaccacca

SEQ ID NO: 224 - DNA, 20 bp, Homo sapiens
gggccatgccgaccaccaac

SEQ ID NO: 225 - protein, 20 aa, Homo sapiens
CSEKMALSTASDDRCWNGMA

SEQ ID NO: 226 - protein, 20 aa, Homo sapiens
RGRYLPEVMGDGLANQINNP

SEQ ID NO: 227 - protein, 20 aa, Homo sapiens
EVEVDITKPDMTIRQQIMQL

SEQ ID NO: 228 - protein, 20 aa, Homo sapiens
KIMTNRLRSAYNGNDVDFQD

SEQ ID NO: 229 - protein, 20 aa, Homo sapiens
TNRLRSAYNGNDVDFQDASD

SEQ ID NO: 230 - protein, 50 aa, Homo sapiens
CSEKMALSTASDDRCWNGMARGRYLPEVMGDGLANQINNPEVEVDITKPD

SEQ ID NO: 231 - protein, 50 aa, Homo sapiens
DGLANQINNPEVEVDITKPDMTIRQQIMQLKIMTNRLRSAYNGNDVDFQD

SEQ ID NO: 232 - protein, 20 aa, Homo sapiens
MDRGPAAVACTLLLALVACL

SEQ ID NO: 233 - protein, 20 aa, Homo sapiens
GCAVVTCQQGYFKCQSEGQC

SEQ ID NO: 234 - protein, 20 aa, Homo sapiens
IPSEYRCDHVRDCPDGADEN

SEQ ID NO: 235 - protein, 20 aa, Homo sapiens
CNYPTCGGYQFTCPSGRCIY

SEQ ID NO: 236 - protein, 20 aa, Homo sapiens
SDERQDCSQSTCSSHQITCS

SEQ ID NO: 237 - protein, 50 aa, Homo sapiens
TLLLALVACLAPASGQECDSAHFRCGSGHCIPADWRCDGTKDCSDDADEI

SEQ ID NO: 238 - protein, 50 aa, Homo sapiens
RDCPDGADENDCQYPTCEQLTCDNGACYNTSQKCDWKVDCRDSSDEINCT

SEQ ID NO: 239 - protein, 20 aa, Homo sapiens
RGLPALLLLLLFLGPWPAAS

SEQ ID NO: 240 - protein, 20 aa, Homo sapiens
QLWEKAQRLHLPPVRLAELH

SEQ ID NO: 241 - protein, 20 aa, Homo sapiens
KYGLDGKKDARQVTSNSLSG

SEQ ID NO: 242 - protein, 20 aa, Homo sapiens
EKVHEYNVLLETLSRTEEIH

SEQ ID NO: 243 - protein, 20 aa, Homo sapiens
VSHQGYSTEAEFEEPRVIDL

SEQ ID NO: 244 - protein, 50 aa, Homo sapiens
AHEKLRHAESVGDGERVSRSREKHALLEGRTKELGYTVKKHLQDLSGRIS

SEQ ID NO: 245 - protein, 50 aa, Homo sapiens
ARQVTSNSLSGTQEDGLDDPRLEKLWHKAKTSGKFSGEELDKLWREFLHH

SEQ ID NO: 246 - protein, 20 aa, Homo sapiens
LLLLCLAGLVFVSEAGPTGT

SEQ ID NO: 247 - protein, 20 aa, Homo sapiens
WEPFASGKTSESGELHGLTT

SEQ ID NO: 248 - protein, 20 aa, Homo sapiens
EEEFVEGIYKVEIDTKSYWK

SEQ ID NO: 249 - protein, 20 aa, Homo sapiens
ALGISPFHEHAEVVFTANDS

SEQ ID NO: 250 - protein, 20 aa, Homo sapiens
GPRRYTIAALLSPYSYSTTA

SEQ ID NO: 251 - protein, 50 aa, Homo sapiens
LLCLAGLVFVSEAGPTGTGESKCPLMVKVLDAVRGSPAINVAVHVFRKAA

SEQ ID NO: 252 - protein, 50 aa, Homo sapiens
ESGELHGLTTEEEFVEGIYKVEIDTKSYWKALGISPFHEHAEVVFTANDS

SEQ ID NO: 253 - protein, 20 aa, Homo sapiens
LESGDLSMSSIKVDGIRMSF

SEQ ID NO: 254 - protein, 20 aa, Homo sapiens
QNLLAKICFHHHFSVVERVD

SEQ ID NO: 255 - protein, 20 aa, Homo sapiens
SCASMYSRSIQGHHVCLLVK

SEQ ID NO: 256 - protein, 20 aa, Homo sapiens
KGENSVSVDGKCSDSTLLSN

SEQ ID NO: 257 - protein, 20 aa, Homo sapiens
SDSTLLSNLLEEMKATLAKC

SEQ ID NO: 258 - protein, 50 aa, Homo sapiens
NLLAKICFHHHFSVVERVDSCASMYSRSIQGHHVCLLVKKGENSVSVDGK

SEQ ID NO: 259 - protein, 20 aa, Homo sapiens
WDNFGIWDNRIDEPILLPPS

SEQ ID NO: 260 - protein, 20 aa, Homo sapiens
LYFAVYDGHGGPAAADFCHT

SEQ ID NO: 261 - protein, 20 aa, Homo sapiens
ATLLTSGTTATVALLRDGIE

SEQ ID NO: 262 - protein, 20 aa, Homo sapiens
GGFVAWNSLGQPHVNGRLAM

SEQ ID NO: 263 - protein, 20 aa, Homo sapiens
NFMVNSQEICDFVNQCHDPN

SEQ ID NO: 264 - protein, 50 aa, Homo sapiens
LLQDDRRVTPTCHSSTSEPRCSRFDPDGSGSPATWDNFGIWDNRIDEPIL

SEQ ID NO: 265 - protein, 50 aa, Homo sapiens
GQPHVNGRLAMTRSIGDLDLKTSGVIAEPETKRIKLHHADDSFLVLTTDG

SEQ ID NO: 266 - protein, 20 aa, Homo sapiens
RTLDSEPKCVEELPEWNFDG

SEQ ID NO: 267 - protein, 20 aa, Homo sapiens
SSTLQSEGSNSDMYLVPAAM

SEQ ID NO: 268 - protein, 20 aa, Homo sapiens
MDMVSNQHPWFGMEQEYTLM

SEQ ID NO: 269 - protein, 20 aa, Homo sapiens
RACLYAGVKIAGTNAEVMPA

SEQ ID NO: 270 - protein, 20 aa, Homo sapiens
FPGPQGPYYCGVGADRAYGR

SEQ ID NO: 271 - protein, 50 aa, Homo sapiens
LQSEGSNSDMYLVPAAMFRDPFRKDPNKLVLCEVFKYNRRPAETNLRHTC

SEQ ID NO: 272 - protein, 50 aa, Homo sapiens
HPWFGMEQEYTLMGTDGHPFGWPSNGFPGPQGPYYCGVGADRAYGRDIVE

SEQ ID NO: 273 - protein, 20 aa, Homo sapiens
TTTPQQNKMDKLIEILNSMR

SEQ ID NO: 274 - protein, 20 aa, Homo sapiens
AVSDRSFAFTAAKLCDKMAL

SEQ ID NO: 275 - protein, 20 aa, Homo sapiens
LCEVFGTMRSSTGEPFRVLV

SEQ ID NO: 276 - protein, 20 aa, Homo sapiens
EMMTELLASARDKMLCPSES

SEQ ID NO: 277 - protein, 20 aa, Homo sapiens
EVIELHANSWNPLTPPITQY

SEQ ID NO: 278 - protein, 50 aa, Homo sapiens
FTAAKLCDKMALFMVEGTKFRSLLLNMLQKDFTVREELQQQDVERWLGFI

SEQ ID NO: 279 - protein, 50 aa, Homo sapiens
RSSTGEPFRVLVCPIYTCLRELLQSQDVKEDAVLCCSMELQSTGRLLEEQ

SEQ ID NO: 280 - protein, 20 aa, Homo sapiens
VRMMQAQEAVSRIKMAQKLA

SEQ ID NO: 281 - protein, 20 aa, Homo sapiens
PLRTISYIADIGNIVVLMAR

SEQ ID NO: 282 - protein, 20 aa, Homo sapiens
AQSIGQAFSVAYQEFLRANG

SEQ ID NO: 283 - protein, 20 aa, Homo sapiens
KQKGEILGVVIVESGWGSIL

SEQ ID NO: 284 - protein, 20 aa, Homo sapiens
TCQSIIKGLKNQSRVKLNIV

SEQ ID NO: 285 - protein, 50 aa, Homo sapiens
ADIGNIVVLMARRRMPRSNSQENVEASHPSQDGKRQYKMICHVFESEDAQ

SEQ ID NO: 286 - protein, 50 aa, Homo sapiens
GVVIVESGWGSILPTVIIANMMHGGPAEKSGKLNIGDQIMSINGTSLVGL

SEQ ID NO: 287 - protein, 20 aa, Homo sapiens
MAFANLRKVLISDSLDPCCR

SEQ ID NO: 288 - protein, 20 aa, Homo sapiens
QNLSKEELIAELQDCEGLIV

SEQ ID NO: 289 - protein, 20 aa, Homo sapiens
ADVINAAEKLQVVGRAGTGV

SEQ ID NO: 290 - protein, 20 aa, Homo sapiens
LVMNTPNGNSLSAAELTCGM

SEQ ID NO: 291 - protein, 20 aa, Homo sapiens
DEGALLRALQSGQCAGAALD

SEQ ID NO: 292 - protein, 50 aa, Homo sapiens
QATASMKDGKWERKKFMGTELNGKTLGILGLGRIGREVATRMQSFGMKTI

SEQ ID NO: 293 - protein, 50 aa, Homo sapiens
SASFGVQQLPLEEIWPLCDFITVHTPLLPSTTGLLNDNTFAQCKKGVRVV

SEQ ID NO: 294 - protein, 20 aa, Homo sapiens
TDGVKHSMNPFCEIAVEEAV

SEQ ID NO: 295 - protein, 20 aa, Homo sapiens
VIAVSCGPAQCQETIRTALA

SEQ ID NO: 296 - protein, 20 aa, Homo sapiens
LLGKQAIDDDCNQTGQMTAG

SEQ ID NO: 297 - protein, 20 aa, Homo sapiens
VTADLRLNEPRYATLPNIMK

SEQ ID NO: 298 - protein, 20 aa, Homo sapiens
AERLGPLQVARVLAKLAEKE

SEQ ID NO: 299 - protein, 50 aa, Homo sapiens
DDCNQTGQMTAGFLDWPQGTFASQVTLEGDKLKVEREIDGGLETLRLKLP

SEQ ID NO: 300 - protein, 50 aa, Homo sapiens
PRYATLPNIMKAKKKKIEVIKPGDLGVDLTSKLSVISVEDPPQRTAGVKV

SEQ ID NO: 301 - protein, 24 aa, Homo sapiens
GKVKVGVNGFGRIGRLVTRAAFNS

SEQ ID NO: 302 - protein, 21 aa, Homo sapiens
KAENGKLVINGNPITIFQERD

SEQ ID NO: 303 - protein, 20 aa, Homo sapiens
ISAPSADAPMFVMGVNHEKY

SEQ ID NO: 304 - protein, 20 aa, Homo sapiens
DNSLKIISNASCTTNCLAPL

SEQ ID NO: 305 - protein, 20 aa, Homo sapiens
AKVIHDNFGIVEGLMTTVHA

SEQ ID NO: 306 - protein, 50 aa, Homo sapiens
FGRIGRLVTRAAFNSGKVDIVAINDPFIDLNYMVYMFQYDSTHGKFHGTV

SEQ ID NO: 307 - protein, 50 aa, Homo sapiens
NGNPITIFQERDPSKIKWGDAGAEYVVESTGVFTTMEKAGAHLQGGAKRV

SEQ ID NO: 308 - protein, 20 aa, Homo sapiens
VGVGKVIRGWDEALLTMSKG

SEQ ID NO: 309 - protein, 20 aa, Homo sapiens
EKARLEIEPEWAYGKKGQPD

SEQ ID NO: 310 - protein, 20 aa, Homo sapiens
DAKIPPNAKLTFEVELVDID

SEQ ID NO: 311 - protein, 50 aa, Homo sapiens
VIRGWDEALLTMSKGEKARLEIEPEWAYGKKGQPDAKIPPNAKLTFEVEL

SEQ ID NO: 312 - protein, 20 aa, Homo sapiens
LKELRDHADSNIVIMLVGNK

SEQ ID NO: 313 - protein, 20 aa, Homo sapiens
SDLRHLRAVPTDEARAFAEK

SEQ ID NO: 314 - protein, 20 aa, Homo sapiens
NGLSFIETSALDSTNVEAAF

SEQ ID NO: 315 - protein, 20 aa, Homo sapiens
QTILTEIYRIVSQKQMSDRR

SEQ ID NO: 316 - protein, 20 aa, Homo sapiens
ENDMSPSNNVVPIHVPPTTE

SEQ ID NO: 317 - protein, 50 aa, Homo sapiens
TILTEIYRIVSQKQMSDRRENDMSPSNNVVPIHVPPTTENKPKVQCCQNI

SEQ ID NO: 318 - protein, 50 aa, Homo sapiens
LKELRDHADSNIVIMLVGNKSDLRHLRAVPTDEARAFAEKNGLSFIETSA

SEQ ID NO: 319 - protein, 20 aa, Homo sapiens
ADLAGNSEVILPVPAFNVIN

SEQ ID NO: 320 - protein, 20 aa, Homo sapiens
AMRIGAEVYHNLKNVIKEKY

SEQ ID NO: 321 - protein, 20 aa, Homo sapiens
KEGLELLKTAIGKAGYTDKV

SEQ ID NO: 322 - protein, 20 aa, Homo sapiens
VIGMDVAASEFFRSGKYDLD

SEQ ID NO: 323 - protein, 20 aa, Homo sapiens
KSFIKDYPVVSIEDPFDQDD

SEQ ID NO: 324 - protein, 50 aa, Homo sapiens
WQKFTASAGIQVVGDDLTVTNPKRIAKAVNEKSCNCLLLKVNQIGSVTES

SEQ ID NO: 325 - protein, 50 aa, Homo sapiens
QANGWGVMVSHRSGETEDTFIADLVVGLCTGQIKTGAPCRSERLAKYNQL

SEQ ID NO: 326 - protein, 20 aa, Homo sapiens
QQQQQQQQQKHEDGDSNGTF

SEQ ID NO: 327 - protein, 20 aa, Homo sapiens
NGTFFVYHAIYLEELTAVEL

SEQ ID NO: 328 - protein, 20 aa, Homo sapiens
TEKIAQLFSISPCQISQIYK

SEQ ID NO: 329 - protein, 20 aa, Homo sapiens
QGPTGIHVLISDEMIQNFQE

SEQ ID NO: 330 - protein, 20 aa, Homo sapiens
EACFILDTMKQETNDSYHII

SEQ ID NO: 331 - protein, 50 aa, Homo sapiens
QQQQQQQQQKHEDGDSNGTFFVYHAIYLEELTAVELTEKIAQLFSISPCQ

SEQ ID NO: 332 - protein, 50 aa, Homo sapiens
CQISQIYKQGPTGIHVLISDEMIQNFQEEACFILDTMKQETNDSYHIILK

SEQ ID NO: 333 - protein, 20 aa, Homo sapiens
GKVKVGVNGFGRIGRLVTRA

SEQ ID NO: 334 - protein, 20 aa, Homo sapiens
AINDPFIDLNYMVYMFQYDS

SEQ ID NO: 335 - protein, 20 aa, Homo sapiens
KAENGKLVINGNPITIFQER

SEQ ID NO: 336 - protein, 20 aa, Homo sapiens
ISAPSADAPMFVMGVNHEKY

SEQ ID NO: 337 - protein, 20 aa, Homo sapiens
ITATQKTVDGPSGKLWRDGR

SEQ ID NO: 338 - protein, 50 aa, Homo sapiens
GNPITIFQERDPSKIKWGDAGAEYVVESTGVFTTMEKAGAHLQGGAKRVI

SEQ ID NO: 339 - protein, 50 aa, Homo sapiens
MFVMGVNHEKYDNSLKIISNASCTTNCLAPLAKVIHDNFGIVEGLMTTVH

SEQ ID NO: 340 - protein, 20 aa, Homo sapiens
KLSIPQLSVTDYEIMEQRLG

SEQ ID NO: 341 - protein, 20 aa, Homo sapiens
EFEEIEGRWKKLSSQLVEHC

SEQ ID NO: 342 - protein, 20 aa, Homo sapiens
SEILKKQLKQCRLLVSDIQT

SEQ ID NO: 343 - protein, 20 aa, Homo sapiens
MCQQVYARKEALKGGLEKTV

SEQ ID NO: 344 - protein, 20 aa, Homo sapiens
KRAKEEAQQKEAKVKLLTES

SEQ ID NO: 345 - protein, 50 aa, Homo sapiens
VAQEALKKELETLTTNYQWLCTRLNGKCKTLEEVWACWHELLSYLEKANK

SEQ ID NO: 346 - protein, 50 aa, Homo sapiens
ADFVAFTNHFKQVFSDVQAREKELQTIFDTLPPMRYQETMSAIRTWVQQS

SEQ ID NO: 347 - protein, 20 aa, Homo sapiens
MKQALIASARRLPGVNMFEQ

SEQ ID NO: 348 - protein, 20 aa, Homo sapiens
QASLSPSYIDLTECPYMWPY

SEQ ID NO: 349 - protein, 20 aa, Homo sapiens
NGDNIEVAFSYSSVLWPWSG

SEQ ID NO: 350 - protein, 20 aa, Homo sapiens
EQTSTVKLPIKVKIIPTPPR

SEQ ID NO: 351 - protein, 20 aa, Homo sapiens
QGHGKLDLLRAYQILNSYKP

SEQ ID NO: 352 - protein, 50 aa, Homo sapiens
IDLTECPYMWPYCSQPIYYGGMPTVVNVTILNGMGVTGRIVDKPDWQPYL

SEQ ID NO: 353 - protein, 50 aa, Homo sapiens
NGDNIEVAFSYSSVLWPWSGYLAISISVTKKAASWEGIAQGHVMITVASP

SEQ ID NO: 354 - protein, 20 aa, Homo sapiens
ADDLTDPAPATTFAHLDATT

SEQ ID NO: 355 - protein, 20 aa, Homo sapiens
NIVGSEHYDVARGVQKILQD

SEQ ID NO: 356 - protein, 20 aa, Homo sapiens
YKSLQDIIAILGMDELSEED

SEQ ID NO: 357 - protein, 20 aa, Homo sapiens
KLTVSRARKIQRFLSQPFQV

SEQ ID NO: 358 - protein, 20 aa, Homo sapiens
VAEVFTGHMGKLVPLKETIK

SEQ ID NO: 359 - protein, 50 aa, Homo sapiens
ADDLTDPAPATTFAHLDATTVLSRAIAELGIYPAVDPLDSTSRIMDPNIV

SEQ ID NO: 360 - protein, 50 aa, Homo sapiens
ILGMDELSEEDKLTVSRARKIQRFLSQPFQVAEVFTGHMGKLVPLKETIK

SEQ ID NO: 361 - protein, 20 aa, Homo sapiens
EIDALAVAYGVAMNKLTLPI

SEQ ID NO: 362 - protein, 20 aa, Homo sapiens
EQSALFQMPLESFHSLQEDW

SEQ ID NO: 363 - protein, 20 aa, Homo sapiens
RREQELAEREMDIVERELHL

SEQ ID NO: 364 - protein, 20 aa, Homo sapiens
EHKITVQASPTLDKRKGSDG

SEQ ID NO: 365 - protein, 20 aa, Homo sapiens
SSSGGSGTWSRGGPPKKEEL

SEQ ID NO: 366 - protein, 50 aa, Homo sapiens
LESFHSLQEDWKLEIQHMFDDLRTKEKELRSREEELLRAAQEQRFQEEQL

SEQ ID NO: 367 - protein, 50 aa, Homo sapiens
PTLDKRKGSDGASPPASPSIIPRLRAIRLTPVDCGGSSSGSSSGGSGTWS

SEQ ID NO: 368 - protein, 20 aa, Homo sapiens
HHSFPYDYSASEYRWHINFN

SEQ ID NO: 369 - protein, 20 aa, Homo sapiens
TFFIDWMAALGLTYDRKKVS

SEQ ID NO: 370 - protein, 20 aa, Homo sapiens
SKAAILARIKRTGDGNYKSG

SEQ ID NO: 371 - protein, 20 aa, Homo sapiens
ASEYRWHINFNTFFIDWMAA

SEQ ID NO: 372 - protein, 20 aa, Homo sapiens
TYDRKKVSKAAILARIKRTG

SEQ ID NO: 373 - protein, 50 aa, Homo sapiens
HHSFPYDYSASEYRWHINFNTFFIDWMAALGLTYDRKKVSKAAILARIKR

SEQ ID NO: 374 - protein, 50 aa, Homo sapiens
ASEYRWHINFNTFFIDWMAALGLTYDRKKVSKAAILARIKRTGDGNYKSG

SEQ ID NO: 375 - protein, 20 aa, Homo sapiens
DTGGANIPALNELLSVWNMG

SEQ ID NO: 376 - protein, 20 aa, Homo sapiens
ANHDMYYASGCSIAKFPEDG

SEQ ID NO: 377 - protein, 20 aa, Homo sapiens
IVLYGDSNCLDDSHRQKDCF

SEQ ID NO: 378 - protein, 20 aa, Homo sapiens
EGNHLHRYSKVLEAHLGDPK

SEQ ID NO: 379 - protein, 20 aa, Homo sapiens
PLNETAPSNLWKHQKLLSID

SEQ ID NO: 380 - protein, 50 aa, Homo sapiens
GCSIAKFPEDGVVITQTFKDQGLEVLKQETAVVENVPILGLYQIPAEGGG

SEQ ID NO: 381 - protein, 50 aa, Homo sapiens
DDSHRQKDCFWLLDALLQYTSYGVTPPSLSHSGNRQRPPSGAGSVTPERM

SEQ ID NO: 382 - protein, 20 aa, Homo sapiens
CVHDNYRNNPFHNFRHCFCV

SEQ ID NO: 383 - protein, 20 aa, Homo sapiens
NTYQINARTELAVRYNDISP

SEQ ID NO: 384 - protein, 20 aa, Homo sapiens
LATDMARHAEIMDSFKEKME

SEQ ID NO: 385 - protein, 20 aa, Homo sapiens
LEEYFMQSDREKSEGLPVAP

SEQ ID NO: 386 - protein, 20 aa, Homo sapiens
QPLWESRDRYEELKRIDDAM

SEQ ID NO: 387 - protein, 50 aa, Homo sapiens
NEMLSCLEHMYHDLGLVRDFSINPVTLRRWLFCVHDNYRNNPFHNFRHCF

SEQ ID NO: 388 - protein, 50 aa, Homo sapiens
DREKSEGLPVAPFMDRDKVTKATAQIGFIKFVLIPMFETVTKLFPMVEEI

SEQ ID NO: 389 - protein, 20 aa, Homo sapiens
SELEKEGLRCRSDSEDECST

SEQ ID NO: 390 - protein, 20 aa, Homo sapiens
FKVGDDCRQDMLALQIIDLF

SEQ ID NO: 391 - protein, 20 aa, Homo sapiens
GRQTDFGMYDYFTRQYGDES

SEQ ID NO: 392 - protein, 20 aa, Homo sapiens
GHIIHIDFGFMFESSPGGNL

SEQ ID NO: 393 - protein, 20 aa, Homo sapiens
PYMDAVVSLVTLMLDTGLPC

SEQ ID NO: 394 - protein, 50 aa, Homo sapiens
YDYFTRQYGDESTLAFQQARYNFIRSMAAYSLLLFLLQIKDRHNGNIMLD

SEQ ID NO: 395 - protein, 50 aa, Homo sapiens
FMFESSPGGNLGWEPDIKLTDEMVMIMGGKMEATPFKWFMEMCVRGYLAV

SEQ ID NO: 396 - protein, 20 aa, Homo sapiens
YSNDFNSGEANTSDAFNWTV

SEQ ID NO: 397 - protein, 20 aa, Homo sapiens
LSPSCLSLLHLQEKNWSALL

SEQ ID NO: 398 - protein, 20 aa, Homo sapiens
TAVVIILTIAGNILVIMAVS

SEQ ID NO: 399 - protein, 20 aa, Homo sapiens
LEKKLQNATNYFLMSLAIAD

SEQ ID NO: 400 - protein, 20 aa, Homo sapiens
LMSLAIADMLLGFLVMPVSM

SEQ ID NO: 401 - protein, 50 aa, Homo sapiens
LYSNDFNSGEANTSDAFNWTVDSENRTNLSCEGCLSPSCLSLLHLQEKNW

SEQ ID NO: 402 - protein, 50 aa, Homo sapiens
ALLTAVVIILTIAGNILVIMAVSLEKKLQNATNYFLMSLAIADMLLGFLV

SEQ ID NO: 403 - protein, 20 aa, Homo sapiens
VIHEVLHHQRHVRTIWQHRK

SEQ ID NO: 404 - protein, 20 aa, Homo sapiens
LHRARALQKRHEDFEEVAQN

SEQ ID NO: 405 - protein, 20 aa, Homo sapiens
VRRVEQRKILLDMSVSFHTH

SEQ ID NO: 406 - protein, 20 aa, Homo sapiens
TLQVTVNVIKEGEDLIQQLR

SEQ ID NO: 407 - protein, 20 aa, Homo sapiens
ELFQERKIKLELFLHVRIFE

SEQ ID NO: 408 - protein, 50 aa, Homo sapiens
QKRHEDFEEVAQNTYTNADKLLEAAEQLAQTGECDPEEIYQAAHQLEDRI

SEQ ID NO: 409 - protein, 50 aa, Homo sapiens
KEGEDLIQQLRDSAISSNKTPHNSSINHIETVLQQLDEAQSQMEELFQER

SEQ ID NO: 410 - protein, 20 aa, Homo sapiens
RILINPGNHLKIQEGTLGFF

SEQ ID NO: 411 - protein, 20 aa, Homo sapiens
IASDAKEVKRAFFYCKACHD

SEQ ID NO: 412 - protein, 20 aa, Homo sapiens
DITDPKRIKKCGCKRLEDEQ

SEQ ID NO: 413 - protein, 20 aa, Homo sapiens
PSTLSPKKKQRNGGMRNSPN

SEQ ID NO: 414 - protein, 20 aa, Homo sapiens
TSPKLMRHDPLLIPGNDQID

SEQ ID NO: 415 - protein, 50 aa, Homo sapiens
IITELVNDTNVQFLDQDDDDDPDTELYLTQPFACGTAFAVSVLDSLMSAT

SEQ ID NO: 416 - protein, 50 aa, Homo sapiens
VSILPGTPLSRADLRAVNINLCDMCVILSANQNNIDDTSLQDKECILASL

SEQ ID NO: 417 - protein, 20 aa, Homo sapiens
VVYYHNIETSNFPKLLIALL

SEQ ID NO: 418 - protein, 20 aa, Homo sapiens
MLLLVEVNVIRVRRYIFFKT

SEQ ID NO: 419 - protein, 20 aa, Homo sapiens
PREVKPPEDLQDLGVRFLQP

SEQ ID NO: 420 - protein, 20 aa, Homo sapiens
FVNLLSKGTYWWMNAFIKTA

SEQ ID NO: 421 - protein, 20 aa, Homo sapiens
HKKPIDLRAIGKLPIAMRAL

SEQ ID NO: 422 - protein, 50 aa, Homo sapiens
VVYYHNIETSNFPKLLIALLVYWTLAFITKTIKFVKFLDHAIGFSQLRFC

SEQ ID NO: 423 - protein, 50 aa, Homo sapiens
RFLQPFVNLLSKGTYWWMNAFIKTAHKKPIDLRAIGKLPIAMRALTNYQR

SEQ ID NO: 424 - protein, 21 aa, Homo sapiens
EMEEDQRWLEKEERFLKPDVR

SEQ ID NO: 425 - protein, 26 aa, Homo sapiens
RGSIDREDGSLQGPIGNQHIYQPVGV

SEQ ID NO: 426 - protein, 25 aa, Homo sapiens
GVKLQPQEISPPPTANLDRSNDKVY

SEQ ID NO: 427 - protein, 22 aa, Homo sapiens
LRTLLATVDETIPLLPASTHRE

SEQ ID NO: 428 - protein, 24 aa, Homo sapiens
LNSDLGELINKMKLAQQYVMTSLQ

SEQ ID NO: 429 - protein, 50 aa, Homo sapiens
GSLQGPIGNQHIYQPVGKPDPAAPPKKPPRPGAPGHLGSLASLSSPADSY

SEQ ID NO: 430 - protein, 50 aa, Homo sapiens
PPPTANLDRSNDKVYENVTGLVKAVIEMSSKIQPAPPEEYVPMVKEVGLA

SEQ ID NO: 431 - protein, 20 aa, Homo sapiens
IDLDPHFNDILGQHSRKRWE

SEQ ID NO: 432 - protein, 20 aa, Homo sapiens
NFIHSENRHLVSPEALDLLD

SEQ ID NO: 433 - protein, 20 aa, Homo sapiens
KLLRYDHQQRLTAKEAMEHP

SEQ ID NO: 434 - protein, 20 aa, Homo sapiens
YFYPVVKEQSQPCADNAVLS

SEQ ID NO: 435 - protein, 20 aa, Homo sapiens
EQSQPCADNAVLSSGLTAAR

SEQ ID NO: 436 - protein, 50 aa, Homo sapiens
IDLDPHFNDILGQHSRKRWENFIHSENRHLVSPEALDLLDKLLRYDHQQR

SEQ ID NO: 437 - protein, 50 aa, Homo sapiens
LLDKLLRYDHQQRLTAKEAMEHPYFYPVVKEQSQPCADNAVLSSGLTAAR

SEQ ID NO: 438 - protein, 20 aa, Homo sapiens
AHAIRLLLEFTDTSYEEKRY

SEQ ID NO: 439 - protein, 20 aa, Homo sapiens
YLLDGKNKITQSNAILRYIA

SEQ ID NO: 440 - protein, 20 aa, Homo sapiens
EKLKPQYLEELPGQLKQFSM

SEQ ID NO: 441 - protein, 20 aa, Homo sapiens
EKLKPQYLEELPGQLKQFSM

SEQ ID NO: 442 - protein, 20 aa, Homo sapiens
SMCKMPINNKMAQWGNKPVC

SEQ ID NO: 443 - protein, 50 aa, Homo sapiens
GKNKITQSNAILRYIARKHNMCGETEEEKIRVDIIENQVMDFRTQLIRLC

SEQ ID NO: 444 - protein, 50 aa, Homo sapiens
QYLEELPGQLKQFSMFLWKFSWFAGEKLTFVDFLTYDILDQNRIFDPKCL

SEQ ID NO: 445 - protein, 20 aa, Homo sapiens
LRVNDSILFVNEVDVREVTH

SEQ ID NO: 446 - protein, 20 aa, Homo sapiens
NEVDVREVTHSAAVEALKEA

SEQ ID NO: 447 - protein, 20 aa, Homo sapiens
QHIPGDNSIYVTKIIEGGAA

SEQ ID NO: 448 - protein, 20 aa, Homo sapiens
LKVAKPSNAYLSDSYAPPDI

SEQ ID NO: 449 - protein, 20 aa, Homo sapiens
PAEKVMEIKLIKGPKGLGFS

SEQ ID NO: 450 - protein, 50 aa, Homo sapiens
REVTHSAAVEALKEAGSIVRLYVMRRKPPAEKVMEIKLIKGPKGLGFSIA

SEQ ID NO: 451 - protein, 50 aa, Homo sapiens
IYVTKIIEGGAAHKDGRLQIGDKILAVNSVGLEDVMHEDAVAALKNTYDV

SEQ ID NO: 452 - protein, 20 aa, Homo sapiens
STPKLNGSGPSWWPECTCTN

SEQ ID NO: 453 - protein, 20 aa, Homo sapiens
NGSDGMFKYEEIVLERGNSG

SEQ ID NO: 454 - protein, 20 aa, Homo sapiens
DCVLRVNEVEVSEVVHSRAV

SEQ ID NO: 455 - protein, 20 aa, Homo sapiens
FITKIIPGGAAAMDGRLGVN

SEQ ID NO: 456 - protein, 20 aa, Homo sapiens
TSDMVYLKVAKPGSLHLNDM

SEQ ID NO: 457 - protein, 50 aa, Homo sapiens
PPTRYSPIPRHMLAEEDFTREPRKIILHKGSTGLGFNIVGGEDGEGIFVS

SEQ ID NO: 458 - protein, 50 aa, Homo sapiens
DLSGELRRGDRILSVNGVNLRNATHEQAAAALKRAGQSVTIVAQYRPEEY

SEQ ID NO: 459 - protein, 20 aa, Homo sapiens
LVLSLRRRLREELEKGAKAT

SEQ ID NO: 460 - protein, 20 aa, Homo sapiens
ATERTLVYPLELPKEPTSPP

SEQ ID NO: 461 - protein, 20 aa, Homo sapiens
FRPCPEPDEKLWDPVGYYYS

SEQ ID NO: 462 - protein, 20 aa, Homo sapiens
DGSLKIVPGHARCQPGGGPP

SEQ ID NO: 463 - protein, 20 aa, Homo sapiens
SPPPGIPGQPLPSPTRLHLG

SEQ ID NO: 464 - protein, 50 aa, Homo sapiens
SNANGYVRLQLGGEDRGGLGHPLPELADELRRKLQQRQPLPDSNPEESSV

SEQ ID NO: 465 - protein, 50 aa, Homo sapiens
YYYSDGSLKIVPGHARCQPGGGPPSPPPGIPGQPLPSPTRLHLGGGRNSN

SEQ ID NO: 466 - protein, 20 aa, Homo sapiens
SFHRGSTSSSPYVAASHTPP

SEQ ID NO: 467 - protein, 20 aa, Homo sapiens
INGSDSILGTRGNAAGSSQT

SEQ ID NO: 468 - protein, 20 aa, Homo sapiens
GDALGKALASIYSPDHTSSS

SEQ ID NO: 469 - protein, 20 aa, Homo sapiens
FPSNPSTPVGSPSPLTGTSQ

SEQ ID NO: 470 - protein, 20 aa, Homo sapiens
WPRPGGQAPSSPSYENSLHS

SEQ ID NO: 471 - protein, 50 aa, Homo sapiens
LQSRMEDRLDRLDDAIHVLRNHAVGPSTSLPAGHSDIHSLLGPSHNAPIG

SEQ ID NO: 472 - protein, 50 aa, Homo sapiens; identical to SEQ ID NO: 471

SEQ ID NO: 473 - protein, 20 aa, Homo sapiens
NILKKANQKRMLGDMAIEGG

SEQ ID NO: 474 - protein, 20 aa, Homo sapiens
NFTTAYFKQQTIRELFDMPL

SEQ ID NO: 475 - protein, 20 aa, Homo sapiens
EEPSSSSVPSAPEEEEETVA

SEQ ID NO: 476 - protein, 20 aa, Homo sapiens
SKQTHILEQALCRAEDEEDI

SEQ ID NO: 477 - protein, 20 aa, Homo sapiens
RAATQAKAEQVAELAEFNEN

SEQ ID NO: 478 - protein, 50 aa, Homo sapiens
DGFPAGEGEEAGRPGAEDEEMSRAEQEIAALVEQLTPIERYAMKFLEASL

SEQ ID NO: 479 - protein, 50 aa, Homo sapiens
AEQEIAALVEQLTPIERYAMKFLEASLEEVSREELKQAEEQVEAARKDLD

SEQ ID NO: 480 - protein, 20 aa, Homo sapiens
MGGSGSRLSKELLAEYQDLT

SEQ ID NO: 481 - protein, 20 aa, Homo sapiens
FLTKQEILLAHRRFCELLPQ

SEQ ID NO: 482 - protein, 20 aa, Homo sapiens
EQRSVESSLRAQVPFEQILS

SEQ ID NO: 483 - protein, 20 aa, Homo sapiens
LPELKANPFKERICRVFSTS

SEQ ID NO: 484 - protein, 20 aa, Homo sapiens
PAKDSLSFEDFLDLLSVFSD

SEQ ID NO: 485 - protein, 50 aa, Homo sapiens
TATPDIKSHYAFRIFDFDDDGTLNREDLSRLVNCLTGEGEDTRLSASEMK

SEQ ID NO: 486 - protein, 50 aa, Homo sapiens
TRLSASEMKQLIDNILEESDIDRDGTINLSEFQHVISRSPDFASSFKIVL

SEQ ID NO: 487 - protein, 20 aa, Homo sapiens
ADLQFSQLLGNLLGPAGPGA

SEQ ID NO: 488 - protein, 20 aa, Homo sapiens
PPPPPPPPAPEQQTMPPPGS

SEQ ID NO: 489 - protein, 20 aa, Homo sapiens
GSSESIAAFIQRLSGSSNIF

SEQ ID NO: 490 - protein, 20 aa, Homo sapiens
LQPQLRSFFHQHYLGGQEPT

SEQ ID NO: 491 - protein, 20 aa, Homo sapiens
FLQEQFNSIAAHVLHCTDSG

SEQ ID NO: 492 - protein, 50 aa, Homo sapiens
QDIQSQRKVKPQPPLSDAYLSGMPAKRRKTMQGEGPQLLLSEAVSRAAKA

SEQ ID NO: 493 - protein, 50 aa, Homo sapiens
VSWLTTMMGLRLQVVLEHMPVGPDAILRYVRRVGDPPQPLPEEPMEVQGA

SEQ ID NO: 494 - protein, 20 aa, Homo sapiens
PNMYDNVNKLNASLQETSVS

SEQ ID NO: 495 - protein, 20 aa, Homo sapiens
ISSTKGMFPGSLAHTTTKVF

SEQ ID NO: 496 - protein, 20 aa, Homo sapiens
KVFDHEISQVPENNFSVQPT

SEQ ID NO: 497 - protein, 20 aa, Homo sapiens
IDSAYPASGSIMENEQRSNQ

SEQ ID NO: 498 - protein, 20 aa, Homo sapiens
MNNILRIIADLQVSCSYDHL

SEQ ID NO: 499 - protein, 20 aa, Homo sapiens
DLAGPSAGSGSARFSRRPTC

SEQ ID NO: 500 - protein, 20 aa, Homo sapiens
FPSEKARHLLDDSVLESRSP

SEQ ID NO: 501 - protein, 20 aa, Homo sapiens
LGSSESSEFSEEMSSGLESP

SEQ ID NO: 502 - protein, 50 aa, Homo sapiens
CSLGNSAAVPTMEGPLRRKTLLKEGRKPALSSWTRYWVILSGSTLLYYGA

SEQ ID NO: 503 - protein, 50 aa, Homo sapiens
HYKSTPGKKVSIVGWMVQLPDDPEHPDIFQLNNPDKGNVYKFQTGSRFHA

SEQ ID NO: 504 - protein, 20 aa, Homo sapiens
ALSMCKSVVAPATDGGLNLT

SEQ ID NO: 505 - protein, 20 aa, Homo sapiens
STFLRKNQCETRTMLLQPAG

SEQ ID NO: 506 - protein, 20 aa, Homo sapiens
SLGSYSYRSPHWGSTYSVSV

SEQ ID NO: 507 - protein, 20 aa, Homo sapiens
GRWFSAGLASNSSWLREKKA

SEQ ID NO: 508 - protein, 20 aa, Homo sapiens
VETDYDQYALLYSQGSKGPG

SEQ ID NO: 509 - protein, 50 aa, Homo sapiens
MATHHTLWMGLALLGVLGDLQAAPEAQVSVQPNFQQDKFLGRWFSAGLAS

SEQ ID NO: 510 - protein, 50 aa, Homo sapiens
EDFRMATLYSRTQTPRAELKEKFTAFCKAQGFTEDTIVFLPQTDKCMTEQ

SEQ ID NO: 511 - protein, 20 aa, Homo sapiens
MFLVNSFLKGGGGGGGGGGG

SEQ ID NO: 512 - protein, 20 aa, Homo sapiens
GNVLGGLISGAGGGGGGGGG

SEQ ID NO: 513 - protein, 20 aa, Homo sapiens
ILGGVISAISEAAAQYNPEP

SEQ ID NO: 514 - protein, 20 aa, Homo sapiens
ESEEVRQFRRLFAQLAGDDM

SEQ ID NO: 515 - protein, 20 aa, Homo sapiens
MNILNKVVTRHPDLKTDGFG

SEQ ID NO: 516 - protein, 50 aa, Homo sapiens
IDTCRSMVAVMDSDTTGKLGFEEFKYLWNNIKRWQAIYKQFDTDRSGTIC

SEQ ID NO: 517 - protein, 50 aa, Homo sapiens
GFHLNEHLYNMIIRRYSDESGNMDFDNFISCLVRLDAMFRAFKSLDKDGT

SEQ ID NO: 518 - protein, 20 aa, Homo sapiens
HVIEDSHKKITAQIKFLYEE

SEQ ID NO: 519 - protein, 20 aa, Homo sapiens
KPQHYPSIHITAYENDERVP

SEQ ID NO: 520 - protein, 20 aa, Homo sapiens
NSNPELVRAVTLEAPFLDVL

SEQ ID NO: 521 - protein, 20 aa, Homo sapiens
RGGGELGLQWHADGRLTKKL

SEQ ID NO: 522 - protein, 20 aa, Homo sapiens
SKDGKLVPMTVFHKTDSEDL

SEQ ID NO: 523 - protein, 50 aa, Homo sapiens
ACGFIMDTNSDPKNCPFQLCSPIRPPKYYTYKFAEGKLFEETGHEDPITK

SEQ ID NO: 524 - protein, 50 aa, Homo sapiens
MNWDLFFTMKRNTKVIDLDMFKDHCVLFLKHSNLLYVNVIGLADDSVRSL

SEQ ID NO: 525 - protein, 20 aa, Homo sapiens
KEGILPERAEEAKLKAKYPS

SEQ ID NO: 526 - protein, 20 aa, Homo sapiens
LGQKPGGSDFLMKRLQKGQK

SEQ ID NO: 527 - protein, 20 aa, Homo sapiens
YFDSGDYNMAKAKMKNKQLP

SEQ ID NO: 528 - protein, 20 aa, Homo sapiens
SAGPDKNLVTGDHIPTPQDL

SEQ ID NO: 529 - protein, 20 aa, Homo sapiens
DLPQRKSSLVTSKLAGGQVE

SEQ ID NO: 530 - protein, 50 aa, Homo sapiens
KEGILPERAEEAKLKAKYPSLGQKPGGSDFLMKRLQKGQKYFDSGDYNMA

SEQ ID NO: 531 - protein, 20 aa, Homo sapiens
GSPTGRIEGFTNVKELYGKI

SEQ ID NO: 532 - protein, 20 aa, Homo sapiens
AEAFRLPTAEVMFCTLNTHK

SEQ ID NO: 533 - protein, 20 aa, Homo sapiens
VDMDKLLGGQIGLEDFIFAH

SEQ ID NO: 534 - protein, 20 aa, Homo sapiens
KEVEVFKSEDALGLTITDNG

SEQ ID NO: 535 - protein, 20 aa, Homo sapiens
AFIKRIKEGSVIDHIHLISV

SEQ ID NO: 536 - protein, 50 aa, Homo sapiens
QSLLGCRHYEVARLLKELPRGRTFTLKLTEPRKAFDMISQRSAGGRPGSG

SEQ ID NO: 537 - protein, 50 aa, Homo sapiens
ATVEDLPSAFEEKAIEKVDDLLESYMGIRDTELAATMVELGKDKRNPDEL

SEQ ID NO: 538 - protein, 20 aa, Homo sapiens
LAAPQGGSPTKLQRGGSAPE

SEQ ID NO: 539 - protein, 20 aa, Homo sapiens
ATATLQRPGSLAAGSRASYS

SEQ ID NO: 540 - protein, 20 aa, Homo sapiens
RSLSQSQGVPLPPAHTGTYR

SEQ ID NO: 541 - protein, 20 aa, Homo sapiens
ATFQRASYAAGPASNYADPY

SEQ ID NO: 542 - protein, 20 aa, Homo sapiens
DNKIKAEIRRQGGIQLLVDL

SEQ ID NO: 543 - protein, 50 aa, Homo sapiens
LWNLSSCDALKMPIIQDALAVLTNAVIIPHSGWENSPLQDDRKIQLHSSQ

SEQ ID NO: 544 - protein, 50 aa, Homo sapiens
SPGEEARRRMRECDGLTDALLYVIQSALGSSEIDSKTVENCVCILRNLSY

SEQ ID NO: 545 - protein, 20 aa, Homo sapiens
KKAPPLVENEEAEPGRGGLG

SEQ ID NO: 546 - protein, 20 aa, Homo sapiens
GGGGSGGPQMGLPPPPPALR

SEQ ID NO: 547 - protein, 20 aa, Homo sapiens
HGSPTGRIEGFTNVKELYGK

SEQ ID NO: 548 - protein, 20 aa, Homo sapiens
TLNTHKVDMDKLLGGQIGLE

SEQ ID NO: 549 - protein, 20 aa, Homo sapiens
SEDALGLTITDNGAGYAFIK

SEQ ID NO: 550 - protein, 50 aa, Homo sapiens
NGQSLLGCRHYEVARLLKELPRGRTFTLKLTEPRKAFDMISQRSAGGRPG

SEQ ID NO: 551 - protein, 50 aa, Homo sapiens
RLRSRGPATVEDLPSAFEEKAIEKVDDLLESYMGIRDTELAATMVELGKD

SEQ ID NO: 552 - protein, 20 aa, Homo sapiens
AILYSKFKPQKMREHLELFW

SEQ ID NO: 553 - protein, 20 aa, Homo sapiens
AAEQAHLWAELVFLYDKYEE

SEQ ID NO: 554 - protein, 20 aa, Homo sapiens
HPTDAWKEGQFKDIITKVAN

SEQ ID NO: 555 - protein, 20 aa, Homo sapiens
FKPLLLNDLLMVLSPRLDHT

SEQ ID NO: 556 - protein, 20 aa, Homo sapiens
PYLRSVQNHNNKSVNESLNN

SEQ ID NO: 557 - protein, 50 aa, Homo sapiens
EKRECFGACLFTCYDLLRPDVVLETAWRHNIMDFAMPYFIQVMKEYLTKV

SEQ ID NO: 558 - protein, 50 aa, Homo sapiens
KEEEQATETQPIVYGQPQLMLTAGPSVAVPPQAPFGYGYTAPPYGQPQPG

SEQ ID NO: 559 - protein, 20 aa, Homo sapiens
GSRSRKVKEQYQDVPMPEEK

SEQ ID NO: 560 - protein, 20 aa, Homo sapiens
SNPKGVEWLWHSIVIRMYLS

SEQ ID NO: 561 - protein, 20 aa, Homo sapiens
LIAKSVRNYTQEASLGALQN

SEQ ID NO: 562 - protein, 20 aa, Homo sapiens
LTAGSGPMPTSVAQTVVQKE

SEQ ID NO: 563 - protein, 20 aa, Homo sapiens
SGLQHTRKMLHVGDPSVKKT

SEQ ID NO: 564 - protein, 50 aa, Homo sapiens
AISLLRNLSRNLSLQNEIAKETLPDLVSIIPDTVPSTDLLIETTASACYT

SEQ ID NO: 565 - protein, 50 aa, Homo sapiens
MPEEKSNPKGVEWLWHSIVIRMYLSLIAKSVRNYTQEASLGALQNLTAGS

SEQ ID NO: 566 - protein, 20 aa, Homo sapiens
ALKAAFDVNNKDVSVMMSEM

SEQ ID NO: 567 - protein, 20 aa, Homo sapiens
DVNAIAGTLKLYFRELPEPL

SEQ ID NO: 568 - protein, 20 aa, Homo sapiens
FTDEFYPNFAEGIALSDPVA

SEQ ID NO: 569 - protein, 20 aa, Homo sapiens
KESCMLNLLLSLPEANLLTF

SEQ ID NO: 570 - protein, 20 aa, Homo sapiens
LFLLDHLKRVAEKEAVNKMS

SEQ ID NO: 571 - protein, 50 aa, Homo sapiens
TFLFLLDHLKRVAEKEAVNKMSLHNLATVFGPTLLRPSEKESKLPANPSQ

SEQ ID NO: 572 - protein, 50 aa, Homo sapiens
ALKAAFDVNNKDVSVMMSEMDVNAIAGTLKLYFRELPEPLFTDEFYPNFA

SEQ ID NO: 573 - protein, 20 aa, Homo sapiens
SELVQKAKLAEQAERYDDMA

SEQ ID NO: 574 - protein, 20 aa, Homo sapiens
HELSNEERNLLSVAYKNVVG

SEQ ID NO: 575 - protein, 20 aa, Homo sapiens
WRVISSIEQKTERNEKKQQM

SEQ ID NO: 576 - protein, 20 aa, Homo sapiens
IEAELQDICNDVLELLDKYL

SEQ ID NO: 577 - protein, 20 aa, Homo sapiens
GLALNFSVFYYEILNSPEKA

SEQ ID NO: 578 - protein, 50 aa, Homo sapiens
LKMKGDYFRYLSEVASGDNKQTTVSNSQQAYQEAFEISKKEMQPTHPIRL

SEQ ID NO: 579 - protein, 50 aa, Homo sapiens
CSLAKTAFDEAIAELDTLNEESYKDSTLIMQLLRDNLTLWTSENQGDEGD

SEQ ID NO: 580 - protein, 20 aa, Homo sapiens
MDKNELVQKAKLAEQAERYD

SEQ ID NO: 581 - protein, 20 aa, Homo sapiens
DMAACMKSVTEQGAELSNEE

SEQ ID NO: 582 - protein, 20 aa, Homo sapiens
RNLLSVAYKNVVGARRSSWR

SEQ ID NO: 583 - protein, 20 aa, Homo sapiens
IEQKTEGAEKKQQMAREYRE

SEQ ID NO: 584 - protein, 20 aa, Homo sapiens
CNDVLSLLEKFLIPNASQAE

SEQ ID NO: 585 - protein, 50 aa, Homo sapiens
MKGDYYRYLAEVAAGDDKKGIVDQSQQAYQEAFEISKKEMQPTHPIRLGL

SEQ ID NO: 586 - protein, 50 aa, Homo sapiens
SPEKACSLAKTAFDEAIAELDTLSEESYKDSTLIMQLLRDNLTLWTSDTQ

SEQ ID NO: 587 - protein, 20 aa, Homo sapiens
PFFWLENKDVIGVLEKGDRL

SEQ ID NO: 588 - protein, 20 aa, Homo sapiens
PKPDLCPPVLYTLMTRCWDY

SEQ ID NO: 589 - protein, 20 aa, Homo sapiens
CSLSDVYQMEKDIAMEQERN

SEQ ID NO: 590 - protein, 20 aa, Homo sapiens
RPPPQTNLLAPKLQFQVPEG

SEQ ID NO: 591 - protein, 20 aa, Homo sapiens
QMVEDYQWLRQEEKSLDPMV

SEQ ID NO: 592 - protein, 50 aa, Homo sapiens
PEKEVGYLEFTGPPQKPPRLGAQSIQPTANLDRTDDLVYLNVMELVRAVL

SEQ ID NO: 593 - protein, 50 aa, Homo sapiens
PEGYVVVVKNVGLTLRKLIGSVDDLLPSLPSSSRTEIEGTQKLLNKDLAE

SEQ ID NO: 594 - protein, 20 aa, Homo sapiens
LFTPLNMVVQATGGPELASS

SEQ ID NO: 595 - protein, 20 aa, Homo sapiens
MGATMEQDLYQLAESVANVA

SEQ ID NO: 596 - protein, 20 aa, Homo sapiens
NIYTRGSHLDQGEAAVAFKP

SEQ ID NO: 597 - protein, 20 aa, Homo sapiens
EILDDRKQWWKVRNASGDSG

SEQ ID NO: 598 - protein, 20 aa, Homo sapiens
PYTHTIQKQRMEYGPRPADT

SEQ ID NO: 599 - protein, 50 aa, Homo sapiens
SSDSGGSIVRDSQRHKQLPVDRRKSQMEEVQDELIHRLTIGRSAAQKKFH

SEQ ID NO: 600 - protein, 50 aa, Homo sapiens
ITYDSTPEDVKTWLQSKGFNPVTVNSLGVLNGAQLFSLNKDELRTVCPEG

SEQ ID NO: 601 - protein, 20 aa, Homo sapiens
LLMEKIRDPFIHEISKIAMG

SEQ ID NO: 602 - protein, 20 aa, Homo sapiens
MRSASQFTRDFIRDSGVVSL

SEQ ID NO: 603 - protein, 20 aa, Homo sapiens
IETLLNYPSSRVRTSFLENM

SEQ ID NO: 604 - protein, 20 aa, Homo sapiens
IRMAPPYPNLNIIQTYICKV

SEQ ID NO: 605 - protein, 20 aa, Homo sapiens
CEETLAYSVDSPEQLSGIRM

SEQ ID NO: 606 - protein, 50 aa, Homo sapiens
LTTTTDYHTLVANYMSGFLSLLATGNAKTRFHVLKMLLNLSENLFMTKEL

SEQ ID NO: 607 - protein, 50 aa, Homo sapiens
FIGLFNREETNDNIQIVLAIFENIGNNIKKETVFSDDDFNIEPLISAFHK

SEQ ID NO: 608 - protein, 20 aa, Homo sapiens
SRLAKSYSTSSPINIVVSSA

SEQ ID NO: 609 - protein, 20 aa, Homo sapiens
YSKHSQELYATATLQRPGSL

SEQ ID NO: 610 - protein, 20 aa, Homo sapiens
RSLSQSQGVPLPPAHTGTYR

SEQ ID NO: 611 - protein, 20 aa, Homo sapiens
SPYSKSGPALPPEGTLARSP

SEQ ID NO: 612 - protein, 20 aa, Homo sapiens
LRNLVYGKANDDNKIALKNC

SEQ ID NO: 613 - protein, 50 aa, Homo sapiens
NLSSCDALKMPIIQDALAVLTNAVIIPHSGWENSPLQDDRKIQLHSSQVL

SEQ ID NO: 614 - protein, 50 aa, Homo sapiens
VSSPGEEARRRMRECDGLTDALLYVIQSALGSSEIDSKTVENCVCILRNL

SEQ ID NO: 615 - protein, 20 aa, Homo sapiens
PINIVVSSAGLSPIRVTSPP

SEQ ID NO: 616 - protein, 20 aa, Homo sapiens
VHASEQYSKHSQELYATATL

SEQ ID NO: 617 - protein, 20 aa, Homo sapiens
SPGVDSVPLQRTGSQHGPQN

SEQ ID NO: 618 - protein, 20 aa, Homo sapiens
AAGPASNYADPYRQLQYCPS

SEQ ID NO: 619 - protein, 20 aa, Homo sapiens
IQMLQHQFPSVQSNAAAYLQ

SEQ ID NO: 620 - protein, 50 aa, Homo sapiens
SSCDALKMPIIQDALAVLTNAVIIPHSGWENSPLQDDRKIQLHSSQVLRN

SEQ ID NO: 621 - protein, 50 aa, Homo sapiens
NVSSPGEEARRRMRECDGLTDALLYVIQSALGSSEIDSKTVENCVCILRN

SEQ ID NO: 622 - protein, 20 aa, Homo sapiens
NVSSPGEEARRRMRECDGLT

SEQ ID NO: 623 - protein, 20 aa, Homo sapiens
QGQHMGTDELDGLLCGEANG

SEQ ID NO: 624 - protein, 20 aa, Homo sapiens
LWHPSIVKPYLTLLSECSNP

SEQ ID NO: 625 - protein, 20 aa, Homo sapiens
LRIDNDRVACAVATALRNMA

SEQ ID NO: 626 - protein, 20 aa, Homo sapiens
TAVCCTLHEVITKNMENAKA

SEQ ID NO: 627 - protein, 50 aa, Homo sapiens
ISLKERKTDYECTGSNATYHGAKGEHTSRKDAMTAQNTGISTLYRNSYGA

SEQ ID NO: 628 - protein, 50 aa, Homo sapiens
SAQPVPQEPSRKDYETYQPFQNSTRNYDESFFEDQVHHRPPASEYTMHLG

SEQ ID NO: 629 - protein, 20 aa, Homo sapiens
MALLTAAARLLGTKNASCLV

SEQ ID NO: 630 - protein, 20 aa, Homo sapiens
LAARHASASSTNLKDILADL

SEQ ID NO: 631 - protein, 20 aa, Homo sapiens
IPKEQARIKTFRQQHGKTVV

SEQ ID NO: 632 - protein, 20 aa, Homo sapiens
GQITVDMMYGGMRGMKGLVY

SEQ ID NO: 633 - protein, 20 aa, Homo sapiens
ETSVLDPDEGIRFRGFSIPE

SEQ ID NO: 634 - protein, 50 aa, Homo sapiens
GMKGLVYETSVLDPDEGIRFRGFSIPECQKLLPKAKGGEEPLPEGLFWLL

SEQ ID NO: 635 - protein, 50 aa, Homo sapiens
MALLTAAARLLGTKNASCLVLAARHASASSTNLKDILADLIPKEQARIKT

SEQ ID NO: 636 - protein, 20 aa, Homo sapiens
LAAVYKALSDHHVYLEGTLL

SEQ ID NO: 637 - protein, 20 aa, Homo sapiens
KPNMVTPGHACPIKYTPEEI

SEQ ID NO: 638 - protein, 20 aa, Homo sapiens
AMATVTALRRTVPPAVPGVT

SEQ ID NO: 639 - protein, 20 aa, Homo sapiens
FLSGGQSEEEASFNLNAINR

SEQ ID NO: 640 - protein, 20 aa, Homo sapiens
CPLPRPWALTFSYGRALQAS

SEQ ID NO: 641 - protein, 50 aa, Homo sapiens
ALNAWRGQRDNAGAATEEFIKRAEVNGLAAQGKYEGSGEDGGAAAQSLYI

SEQ ID NO: 642 - protein, 50 aa, Homo sapiens
IKYTPEEIAMATVTALRRTVPPAVPGVTFLSGGQSEEEASFNLNAINRCP

SEQ ID NO: 643 - protein, 20 aa, Homo sapiens
VDNPGHPYIMTVGCVAGDEE

SEQ ID NO: 644 - protein, 20 aa, Homo sapiens
LDPNYVLSSRVRTGRSIRGF

SEQ ID NO: 645 - protein, 20 aa, Homo sapiens
EAEQQQLIDDHFLFDKPVSP

SEQ ID NO: 646 - protein, 20 aa, Homo sapiens
EDRHGGYKPSDEHKTDLNPD

SEQ ID NO: 647 - protein, 20 aa, Homo sapiens
ARGIWHNDNKTFLVWVNEED

SEQ ID NO: 648 - protein, 50 aa, Homo sapiens
TRFCTGLTQIETLFKSKDYEFMWNPHLGYILTCPSNLGTGLRAGVHIKLP

SEQ ID NO: 649 - protein, 50 aa, Homo sapiens
KHEKFSEVLKRLRLQKRGTGGVDTAAVGGVFDVSNADRLGFSEVELVQMV

SEQ ID NO: 650 - protein, 20 aa, Homo sapiens
MDCCTENACSKPDDDILDIP

SEQ ID NO: 651 - protein, 20 aa, Homo sapiens
LDDPGANAAAAKIQASFRGH

SEQ ID NO: 652 - protein, 20 aa, Homo sapiens
MARKKIKSGERGRKGPGPGG

SEQ ID NO: 653 - protein, 50 aa, Homo sapiens
AKIQASFRGHMARKKIKSGERGRKGPGPGGMARKKIKSGERGRKGPGPGG

SEQ ID NO: 654 - protein, 20 aa, Homo sapiens
MASATDSRYGQKESSDQNFD

SEQ ID NO: 655 - protein, 20 aa, Homo sapiens
YMFKILIIGNSSVGKTSFLF

SEQ ID NO: 656 - protein, 20 aa, Homo sapiens
RYADDSFTPAFVSTVGIDFK

SEQ ID NO: 657 - protein, 20 aa, Homo sapiens
VKTIYRNDKRIKLQIWDTAG

SEQ ID NO: 658 - protein, 20 aa, Homo sapiens
QERYRTITTAYYRGAMGFIL

SEQ ID NO: 659 - protein, 50 aa, Homo sapiens
VKTIYRNDKRIKLQIWDTAGQERYRTITTAYYRGAMGFILMYDITNEESF

SEQ ID NO: 660 - protein, 50 aa, Homo sapiens
TDSRYGQKESSDQNFDYMFKILIIGNSSVGKTSFLFRYADDSFTPAFVST

SEQ ID NO: 661 - protein, 20 aa, Homo sapiens
HAPAVTQHAPYFKGTAVVNG

SEQ ID NO: 662 - protein, 20 aa, Homo sapiens
EFHDVNCEVVAVSVDSHFSH

SEQ ID NO: 663 - protein, 20 aa, Homo sapiens
LALRGLFIIDPNGVIKHLSV

SEQ ID NO: 664 - protein, 20 aa, Homo sapiens
SPTIKPSPAASKEYFQKVNQ

SEQ ID NO: 665 - protein, 50 aa, Homo sapiens
RASVARHVSAIPWGISATAALRPAACGRTSLTNLLCSGSSQAKLFSTSSS

SEQ ID NO: 666 - protein, 50 aa, Homo sapiens
EVVAVSVDSHFSHLAWINTPRKNGGLGHMNIALLSDLTKQISRDYGVLLE

SEQ ID NO: 667 - protein, 20 aa, Homo sapiens
MAMSSGGSGGGVPEQEDSVL

SEQ ID NO: 668 - protein, 20 aa, Homo sapiens
KPKTTPKRKPAKKNKSQKKN

SEQ ID NO: 669 - protein, 20 aa, Homo sapiens
ETCVVVYTGYGNREEQNLSD

SEQ ID NO: 670 - protein, 20 aa, Homo sapiens
DNIKPKSAPWNSFLPPPPPM

SEQ ID NO: 671 - protein, 20 aa, Homo sapiens
PIIPPPPPICPDSLDDADAL

SEQ ID NO: 672 - protein, 50 aa, Homo sapiens
YGNREEQNLSDLLSPICEVANNIEQNAQENENESQVSTDESENSRSPGNK

SEQ ID NO: 673 - protein, 50 aa, Homo sapiens
KPKTTPKRKPAKKNKSQKKNTAASLQQWKVGDKCSAIWSEDGCIYPATIA

SEQ ID NO: 674 - protein, 20 aa, Homo sapiens
GETSYIRVYPERSTSYGYSR

SEQ ID NO: 675 - protein, 20 aa, Homo sapiens
SRSGSRGRDSPYQSRGSPHY

SEQ ID NO: 676 - protein, 20 aa, Homo sapiens
RDSPYQSRGSPHYFSPFRPY

SEQ ID NO: 677 - protein, 20 aa, Homo sapiens
MLPGLALLLLAAWTARALEV

SEQ ID NO: 678 - protein, 20 aa, Homo sapiens
TCIDTKEGILQYCQEVYPEL

SEQ ID NO: 679 - protein, 20 aa, Homo sapiens
EFVSDALLVPDKCKFLHQER

SEQ ID NO: 680 - protein, 20 aa, Homo sapiens
GVEFVCCPLAEESDNVDSAD

SEQ ID NO: 681 - protein, 20 aa, Homo sapiens
NVDSADAEEDDSDVWWGGAD

SEQ ID NO: 682 - protein, 50 aa, Homo sapiens
QYCQEVYPELQITNVVEANQPVTIQNWCKRGRKQCKTHPHFVIPYRCLVG

SEQ ID NO: 683 - protein, 50 aa, Homo sapiens
PDKCKFLHQERMDVCETHLHWHTVAKETCSEKSTNLHDYGMLLPCGIDKF

SEQ ID NO: 684 - protein, 20 aa, Homo sapiens
MARGNQRELARQKNMKKTQE

SEQ ID NO: 685 - protein, 20 aa, Homo sapiens
ISKGKRKEDSLTASQRKQRD

SEQ ID NO: 686 - protein, 20 aa, Homo sapiens
SEIMQEKQKAANEKKSMQTR

SEQ ID NO: 687 - protein, 50 aa, Homo sapiens
MARGNQRELARQKNMKKTQEISKGKRKEDSLTASQRKQRDSEIMQEKQKA

SEQ ID NO: 688 - protein, 50 aa, Homo sapiens
KNMKKTQEISKGKRKEDSLTASQRKQRDSEIMQEKQKAANEKKSMQTREK

SEQ ID NO: 689 - protein, 50 aa, Homo sapiens
PQKNVVTHRLHLNRFSVSGTATTYSQSSASTYVPTVCNGREVLDSTTSSL

SEQ ID NO: 690 - protein, 40 aa, Homo sapiens
QKNVVTHRLHLNRFSVSGTATTYSQSSASTYVPTVCNGRE

SEQ ID NO: 691 - protein, 35 aa, Homo sapiens
KNVVTHRLHLNRFSVSGTATTYSQSSASTYVPTVC

SEQ ID NO: 692 - protein, 30 aa, Homo sapiens
NVVTHRLHLNRFSVSGTATTYSQSSASTYV

SEQ ID NO: 693 - protein, 25 aa, Homo sapiens
NVVTHRLHLNRFSVSGTATTYSQSS

SEQ ID NO: 694 - protein, 20 aa, Homo sapiens
THRLHLNRFSVSGTATTYSQ

SEQ ID NO: 695 - protein, 18 aa, Homo sapiens
HRLHLNRFSVSGTATTYS